SEQUENCE LISTING (596 sequences)

SEQ ID NO: 1 - DNA, 135313 bp, Homo sapiens
agtccccagtctttgcttaggcccctacgtacacaaactgaacctagtgacccagcatggcctctaatttctcaacacttctgtacttctgtaatgattaacccatgcttctcacagatccatgccccaaatttctgtgaataggccctgactggcccagctaagatcatgtgactgcacatgaccagtccactttggcattaacaagcctactgcagactcttcccttggtgttggagtcactcctagaaaagagcaaatctttgtgagccaggcagtcaacctgctggcagcttccactcagccttggagttttttctatgtgtaactttcataaactgagccttatttatttattttttgcactatcatctcatgaaatattattgcgtaagctgaggaaacatgttattcatgatgactggagtttcaagttttaattgtacaatgatttagttttgagtttggtagaaataaaatcaaatttaaaaatcagatatttttcatcttacattatgatgtcccaaaactgcctttatgcttgtgacatagattcataatgtcttctcattccacctgtaatcactgtttgaaataaacatatgtctaatgatatatttggggacattctattttcttcagcttgttgcaagtgaattgatggtgatcttttggtattggtttcattatcaaatttatctccactccaaaattacagtaatttcaaagtaatttagtctatatatttttccatagcttttcttccaaatagaaactgtaaaaagttataaattacttctctccactactgaatttttgtttgcagaataactgatgtaagtagcagaatgcctcttcctagttcaaccctcaggaatagaagtgagaagatctttaaaacttcaccattttccttgacttgtttataattctgaatgtaaatgtgaattgatatggtctatcgcttaacaccacaactcttaatctatgtgcagggtcgtagctcaaaactactgccaggaccacatcaatttcatattcaccctgatcaatgtgtattaatggtgataactatgagaatgaaatgtacagttatcagtatcatttttgactcactaggtatatcctcagaaatatatgaaaaaactaaacacagcttttagtttgacataatttttaaacaactggagttaccttgggagaaaaatcctaccaaatatctataatattgaaagagtaaaaaagagttaaatgtccttaacatcattaatcattagggacatgcaaatcaaaaccacagtgaaataccatctcacaccctttaggatggtggtgataagaagaaaaacagagcataacaagtgttggccaggatgtggaaaagctggaaccattgtgcactgctgattggaaagtacaatggtgcagctgctaagggaaatagtatggtagttcttcaaaaaataaacagttataccatttgattcagcagtttcactcctaggtatataccccaaagaattgaaagcagaatctcaaatatttgtacacctatgttcatagcagcattactcacaatagccaaaaggtggaaacaacccgaatgaccctggatggacgaatggataaacaaaatgaggtctatactgacaataggatattaattgaccttaaaaaggaaagaaattctggccgggcacggtggctcacacctgtaataccagcactttgggaggccaaggcaggcagatcacctgaggttgggagtttgagaccagcctgaccaacatggagaaacctcatatctacttaaaatacaaaaaaaaaaattagccaagcatggtggcgcctgcctgtaatcccaggtactcagtaggctgaggcaggagaatcgcttgaacaggaagcagaggttgcaatgagctgagattgcaccatcacactccagcctgggcaacaagagtgaaactgcatctcaaaaaaaaaaaaaacaaaaaaaacaaaaaaagaaattctgacacatctgctatggtctaaattatgtgttcctctaaaattcataaattgaaatcctaacccccaaggtgatggtattaggaggtgaggctttgtggaggtgattaggtcatgagggtacaaccctcgtgaatgggactagtgccctcataaaaagaagcccaagagagaccccttttcccttccactggatgaggtcacaccaagaagttaccatctacatgtcaggaaataggccctcaccagacaccaaatctattggcaccttgatcttggacttcccagcctgcaaagctgtgagaaataagttcctgttgtttataaaccacccagtttatggtattttgttatagcagctcaaacagattaagatggcttgctacaacatagatgaactttaaagattatgcttttctatgattccacttagatggggtaccaagagtagtcaagttcattgagacagaaaatagagtggttggcaggggctagggggagggaactctggggagttagtgtttaatggatacagagtttcagttttgcaaaatgaaaaagttctgaagatggatggtgctgatggctgcacaatatgaatgtatcaacactactcaactgtacacttaaaataaggttcaaatgatacattttatttcatgtgtgtgtcaatctcaacaaacagatttgttcaggcaaggaaactggttagatgcgaataatactattagagcatcatcaattgaatattaacaaagtgctcatagtttaactttctagctcaaggaagaatggaccattttgaaactatgacagaacattacttatatagctgatgtctttgggaattggaaggaggcatattccttcaccagctgtggctccccttcagcaacctcatatactctccaagcttctctttcctgggtcacctgtttaatcactcccgggacttaatcttccacctatatgttgaccactcacaaatctatgtctccatctcacaagcttattcttgactccagacccaagtattcaactgcctgctgaatacgtgtggtcagatgtcatagaacttcagcttcagtatatcaaatgcaaacccctgttccccccaactgcctcctactccccactggccttcctctggcattccctcctcagttatgagcaccaccgtctcactagccagccagtcaagccccaaactccatctagctgacttctgcctcttcctcaccaccctcttccagtaactcatcaggcactgctgtgtctcattccttcctatccctccagtccctccccttctctccatcatggctgtcactgcatggttcaggctctctggctccccccaaaccacccccacattgctgccgaggtgaactgactactcttggcagccactggattaaaatctttcatcatcttcagcatgataaaacccatatcctttagcatgtaacaaggtcttaatgattctgccagagcttgcttgggggtagcctgcacttgtgggccactccagtcacttcacaggtgctcagtaaatctcagttgaatcagtcatcatcatcatcatcatcatcatcatcatcatcatcatcaatttttcagtctggttcctgtctccttttccagcatcctccattcatagcctcatagccttcactccagccatgtttcacttgtggttttcctgggcaagataagctattcctccctgtctttgcagagtttaaatgactcacttgttcaagtacccaccgttgccatgtgggaccgtgagcaaagtacttaatctcactaagcttcacgttcctcatctgtaaaacagcaaatatggacctcacaaaattgtagtgaggctaaaatgaaataacatatgcaaaagcagtttataaataataaacttactataaaatattattttgtaattctgcaagcttgtcttaaatgccatcacctccaaggagcctttttgccatcataagcagaaactatctctctcttcttggaagctccaccatgcacagcctatgggccctcatcacactccttgagttattcgagttcaagtcccgtgtttacaaccagaccgcaaactctatgaagtcagcatccattcctctctgtggttctccctccgccccatccaggtctcaagggtctagagtctttcaaagagaacacattctgagatttgaggaggcagagacaaaaagttccactgcgaagtgccagggaggcttctgtttggggtgtcccttgggatcacagatcccccacctggtgatgagtcaacccagcaccaccccattgcagggctggaatgacagtaatgggcccacctgctgcctctcctcatacccgcaccccagtcagacattgcaagtcagtcacggctctgtcctgctgggcctggagtgttccagtgccttttccatcacagcaccaagcagccactactagtcgatcaatttcagcacaagagataaacatcattaccctctgctaagctcagagataacccaactagctgaccataatgacttcagtcattacggagcaagataaaagactaaaagagggagggatcacttcagatctgccgagtgagtcgattggacttaaagggccagtcaaaccctgactgccggctcatggcaggctcttgccgaggacaaatgcccagcctatatttatgcaaagagattttgttccaaacttaaggtcaaagatacctaaagacatccccctcaggaacccctctcatggaggagagtgcctgagggtcttggtttcccattgcatcccccacctcaatttccctggtgcccagccacttgtgtctttagggttctctttctctccataaaagggagccaacacagtgtcggcctcctctccccaactaagggcttatgtgtaattaaaagggattatgctttgaaggggaaaagtagcctttaatcaccaggagaaggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaaggtaacagttactgtctgtggtttaaaaatgaggtgtggagcaaataaacaggttggaagtgtggggtggggtggtggggtagggtggtggggcagggtggggggttgtgagcagtcagtgggcttgtcgccgattagcactgaagcagtgtttagctggacggcctttctgtgggcccctctgacagtgcccttcccaggaagatgtgtttctctgtcctcagccacatgaaaatcttttgcctaccgtgcctgtcaatccattgcctgcccgcccctcccccaccccccgttttacacctgcctgtccagtctaccgctctctagggcatccacgctgagcagtgggaagaactttaagccctgaagagcaggccaaaggcaagcaagaaccccctcgaacagcttcccagcttagtgaggccttatttcattgattctctgaggcacattgttttttcacatgttagcatttctgaaattgggatgcagctcacgatcaagtcacagtttaactggacacattatttttctttcttagtggtgcagaaaagtaacagtgtgtcttacaattgactgcgtcctagattctgtgagatgcaatacgttattaaccatcacgcacatttcctgaactctttcaatgagcagacaccagcctgggttagactggagccctaaaagcacgacacagattccaccctggactggcttctgttctgcctgggaaaacccaaagtacgtttggagaccaagagcaacataaagtagcataggtggaatagtccatgagaagtgcgagcaaaaggtgccggagatcagagaacaccaagactgtacttgtaaatgacaactggctttgtgcaattttttctgggaaaggataaggagtgactatagaactgtaaagaaagaatgcactttgctacagccttgcagagttgtgcaaatgccgatgactaaaggagctgaaagaggaaggaggggataagggatgggggctgggtaggggtgagattaggaccctgggagctgcaagccactggagagatcaggaggaaagggagggagacctgctttaggcgagaagagaacagtatttgttccaaatctcggttcagaataagttcatgtaggtgatggggccaactggaacaggtgaaggcctatgaatgagtgtctcagttagggtctccttagagtttaatatgaaaaggtgttagctaagtacagagctggtacctgagagagtaaaaggaaactctaaggtatcatggaggtagcaattgcaggacacagctcccacccctagggctgagagaaccaagggaagagacaggaattattaagacttggagcatagatgagaggtctgtggagctgacattaggacttgggaggaaggcgtgcatggaggctgctgctggatctctgaacctgacctcgggtctggacccctgaggagaaagccctggcaggttggtgcatgtggggccgagggacaatagcttaacaaccagcataaaagagagcagcatgggacacgcttcaaccatgcgcatggatggctccaaaacctgtgtgtggctggcccaggacgcagggaggctgcagggggaagagacaagttaaacctgacttgtctgggaagcaccattgtcctcaggtcactttcctctgtcaagcctggtgctgaagttatctgttgtctccaggggccaagtattaagagtaatcagaaactcagtcctttcttctaggagcttcccttcttgcatgaaaatcctgataaaactggaaaaaaaaacctcatgattaaattttttcatgtattcattctttccttctatcaaaaaataatctccaggcaccgtgctaggttcattggtatacaatggcaacaagacctcccagcccctgcctatgtgaggcatctgtggactgcggaggaaaatccaatatgccattgttctctctttcccataagaaattacaattctcagttcattttattctcactgtgctctttgtgaccctcaaagggggtcacatgataacaggactgtagctgctggcctaaaatgagcccattcctgtggcgctcatgtcgctgtgacagagaataaccctgttttcagaatgctctggtgccctccctctcaatctggcctttcgctggcatgggtgggcgactcctgctcagggactctgccttctccacagtgtgctcccagggagatggagccactcgggctgagggccttggccagggcacctcccagggctgggcctggtctgggctggcgttcactggatgccatcctgatggcctggaaattgagatttctgtctggcacgcctcccgatggctccccacctgctaccacattccaggagcttccaggatgtctgggtaagacagaggcacccccaacagattcagtagctctgagagggatctgtggctccttcctaagcttgcggttcttctggaaacttctgcctctagaagatggtccctctaagaaaagtacaaccacccagcccataattcagctcccaggttttccctcaaacctccatgtctcctgtaagcagagcaagagtaaaatcagataccaaatttcctcattcctcagctcccaatccctaagggcataagatgaaaatcttcagatctctgctttcctccctctttttttcttcctctgttaacatttgtcaagtgttactaagtgtctggcactgtactaagtgcatcacctccctgaactctccgaacagttccacgagagaggcctctctgtgatccccccggtactgatgaggtcactgaggctccagagaaggattagtaactggtggggttggacctgggattcacacccatgctgcgtgacccaggacaggcaggcatggccgttacaccacactgacccccgtggatcgagatctatccaatagtctggtcactgatatcactaagatagagtggccatataatttatcatccaatcagggcagttttgcaagtgaaagggagcactattaataattgcactgggacaataaatgtaaaccaacactggacctggaaaactgggacgtgtgtttgccctataccaaggtaagctagacacagccactgccttcatggagttcagaaccaggcaggggcggctcccacgtataattactgtgcagcacaacgtggagaccgtggagtagaaggaaacacggatgggaggtgaggaggaggtctgtgagctcagaggaggcaccggggctggagagggtgagagaagacttcccaaggagttcatcctgataacgtgcattcccaatgacgagcgctctctccactgcacaagacaagtatacatctgcccgtgttggctgtggacctggcgctgtgtcagggagggtttatgaagatcactaggtgggtctcttggtgtcatcccttcatcccagcttctgggttaggatggatatctgtgggggggcctgaggactcatgaaagtggggcgctaatcatgttttggacaccacaccctggagcacctgggacagctgtggcctttgtcctgggttcagcatcaagccgaggatgtggcaagtaaagagaggctgggcaccaactccagtgtacccaggctccgggtcatgtttgtccaggctaagaattctgtcctggttctcagtgcagaaggaagaatcatggggctcattttaggccttggctgccttctgttaaattgaaaacagagcaggaaggaagaaaatttaacaggctcagttctaaaacaacaagcacaactgtgcccttgccagaaacccctcctccccatgttgattgaatggtaaagagaggaggggaggtgagagggagagagagagagaggaagagagagagaaaggaaagaaaggaaagaagaagaaagaaagaaaaggaaagaaagaaagaaagaaagaaagaaagaaagaaagaaagaaagaaagaaagaaagagaaagaaagaaaggagggagggagggaaggggaaaagaaaagaaaagaaaaagaaaaaaagaaggaaataccagtttgggaaaaaagaattttccaccagcccttctgagccttggctgggcttaattaaagttacagacatgtgtaaagggcagggtagggggagtctgagctgctgagaaaacatgtttttaattatactgtggaatttctccctggggtatgcctgtacgcagttaagcgtcaaggacagggatgccgctctggggaggggaagctgagcatgattttggaagccggcagaagaggctattgtgaaaaccagacctgtcaggctaggaaaagaatggctggtggtctttgaccagggagtgacgcgtgaaatgcagcaaccgcccccgccccccgccaaaaacaaacacactctcacagagttagaacaacagtgacctctcaacaaatatttttcaaagattaccaaccaaccattacctagagcagcggttctcaaccttggctgcacggtggaactacctgagacgtgttaaaaagaagaaccctgatgtcccatgccccaagattctgatgtagttgatctggggtatgatctgagaccccggcatgttttcagcctgcagccacatgagaagtgctgacctaatcaacaggggtgatgatttgaggggcggggactataggcaaaaaaaaacagcctaattcaaggatgagaagagggcacaggtgaggtgggaacagtcctagggccagacaaagaaggaagggagaaaggaggtgctgatccctcccctactcctgagaggaggcctttaagtcaccgtgccttgtggagaccagattcttcaaaaatacaagaatgagtgagtgagggagtgggtggatgccaggagagtgcgtgacaagccttgcaagggaggatgacaatgcactagcttggtttggaaattttacccctggaacaggcaggccaagctggctggtcccctccctgatacacagccctccctctttatatatggagcaggggacggtgtgtggctggtttcttagcaagcaccatggttccaagttggcaactggggagttctgaatccaaaaaggagggagatgaacgtaagtggagggcaggcctacaaggttgcagataagcttaattctgtctccttactcttctgcctttgcaacaaccctgtgatcttgcgacaaccctgtaaggcaataacaaatggctcatgtttattgagtgttacctcatgccatattgtgctttcgtgtttaacacaattgtctcatttcaccctcacgactgctctgggaggtaggtcctggtatcacatccatttcacagatgagaccatttggcacggaagagttgagtgggctgcccaaggtcacatagctaagatggaacaggctggataggaaccccagtaacttgacctcagagtaaccttctcttaaccctgagtgtacactgtaggaaaaatgagcagtcccatttcagagaggacaaaactgagactcagaggttaagcaagccccaaagtggttgttaacccagatcttcccactaactcccaaatcagcatcagtgtttaacgtaccagacctctcccagatagatgttgccgcatggaagacagccgatctacgtgatagaaagccaatattgcaagcagtcgtctaaaggagtcaaatgtgttggatttgaactggatgtctcatttctttggtgaagacactggaaacaacttccaggtttcatcaattgctcctatcactcaacgttgctatcttactgaacttgttccccagccttacccactgatggaatgatccagaatggaagacaagacaccaatgtacatgaccctgggggaggctgtttcttaaatctacagactgttggtgacctgagccccatgtcaccaaaggctttcctggagaagcctcctagaccagtcttgacaaaggctcactcattccgtggatatttattgggcacctattatgagttctgccccatgtggggtgctggaatcacagtagtgacaacgacagatgaggttcctgtcctcaggaagcttactgcccttgagggcttcacttacttggaggagtgatgaacctgaagtgcggtgtgtgttaagaagcggaagtccagggccaggcgcggtggctcacgcctgtaatcccagcactttgggaggctgaggcaggcggatcaccaggtcaggagatcgagaccatcctggctaacatggtgaaaccccgtctctactaaaaatacaaaaaaattagccgggcatggtggtgggcacctgcagtcccagctactcaggaggctgaggcaggagagtggcgtgaacctgggaggcagagcttgcagtgagccaagatcgtgccactgcactccagcctgggcaacagagtgagactccgtctcaaaaagaaaaaaaaaagtgcctcacggagagtctattcttttcttcccatattgtgtgtgtgtgtgcgcgcttcctccaacacatcctccctatatatattttgagtaaaacatcttgtaaaaagttacagctacataatcaccacctgtccctaaatagtttttgctttttctttcttcaatgcacgatcattttcccccatcaatttattttttagtttcttataatcttgttgccagtaggctgttttttaaaaagcagaacatggtttgttcttactagcaggaaaggagcatttattgagcctctgctatggtgtcttttattttgctgagagcctatttacatttctttgagaggaaaacaacaaagggttacatgaaagaccatgtgaatagcccctagctgatctattaaacttgctattccccggccagctgcttcagatctccttcagatcttatgtgtttccttcctaaggtccctggagtaagggttgcatagacctattctactctccaactcacatgtccctctccctcttcctctccataattccacatctccaacccccacccctatgtgcaatgccacagggtgtggactgccacagccactggatctgcttttggaatcaagagtccttaagctccaaatggaaccgaaatttaaataccaactttcaaccatatgttaacatcagcagcctcttccaatgtaaaaacccatggcagtgtgccctgctttgtttctttaagcaatagaaacttgaaggaagcatgttggtaggccagatttttgttggctttgcaatggatcacagtcatttattcactcattcattcactgattcattaaatgaccacatttgcaagggcaaggtaatggggagggccagaaaggacactggccccagaaacaggaggctggattttggttctgatgctgccactgctgatgtgacactgcacaggtcacctgcctcctctgagcctctttccttaactgcagagtgagtggctacagagaaatctttactacctgttagatcagcattacctgggagcttgttagaaatgcaagctctggtggggccatactgaacccaaatctgcattcatgtgcatagtgacagctaaaatgcactgaagcagatgatcttgatgatcctttatgaaagtctcatgctaatgcagttttctaaaatagaggcagagtggaacccagatggacacaaaatctggttgatataataaaacaaggtagagggtgtatggtggggagggggtaaaggaaggaaactgtttaggtaaagataccacaaccaaagtcctactgcacacatgggatctgaggagggctgtgtctgctctggttacgttttctataatctcttagcaccactgaactttctctctttttgttttgtttttccagattcgctttgtggtggaactcgtgtggcctttatctttatttctggtcttgatctggttaaggaatgccaacccgctctacagccatcatgaatgtaagcatagcagggtagcttgggcaagccctgaagagactttggtctgggccttttgtctagaaagatcttggggtgggagtgtggggatcagatctgcttatcatcatttcatgtctatgatgcatgtaacagatttatcaatgttacacaaattataatttttaaaaagtctttagagacagggtctcactctgttgccgaggctggagtacagtgttaggaccatggcacactgcagcttctatctcttgggctcaagtgatcctcctgcctgggcttccaaagtgctggaattataggcatgagccactgctcccagctaatttttttgttttttgtggagacagagtcactacattgcccgggctggtcttgaactcctggcctcaagtgatcctcccacctcagcgttctaaagcactgggattacaagcatgagccaccttgtccagcccaaattttcatgttttaatcctacacattctaagcaaatacttgtgtgtagttactaagggactgtgcacttatttttgtttgctttgttgttgctagtttttatttttttatacctaaactctctcgttttaaagagaacagatttgtagatgagttctcgaaaatatttcaggaatcaatatagagaatatgttatacatggtgccagagaaaaatgaggacaagagatgctatacaatcgtactgaagaaaaattttatttcttggacccctgaggtgtctgcagacctgaaaggaacctagtgagagcctcttttacactctgcccctgtgggaaagccttcacctggtttccggccctctatgtggtgaatgtggaagcctcaagcgttatgcaaatctgcccagtcctctattcttgatcttcaccttctcgttcatgagtttcaggccccagttctgaatcagcctcctgtccatcagactcttctttacctctccccgaggagcccataacctgcagccctactgcatgcttggggtaggtgctcagttcaccgtggttgaaggaatagacgagcgtctgctcaagcagcagcagcaactgcgtggagtcttcttgaactaacactcctatgcccctctcggcacaaaatgacgtgtccccccttgcttccccttcacatttccacccatgcctattacaacatccgtctgtctccccactacaccgggagcttgagagaagaggccatgtctctagcacccagcacagggactggcacacatgagatgctcctgcttcttaaatgctgagaatgaaggaggacatcagaggggcccgggccccttcccaaaaaggccaactcctaggtctgcatcctgcttggtctccatgactaatcccgtcttgtcctcattttctgttttaaaggccatttccccaacaaggcgatgccctcagcaggaatgctgccgtggctccaggggatcttctgcaatgtgaacaatccctgttttcaaagccccaccccaggagaatctcctggaattgtgtcaaactataacaactccatgtaagtgttgagatccctaccatgcaggggaggaagttgcacaccccttcacgtgctgaaatgcacacgtgcgtgcacggagcatggagcactgagtgttcttgtggctttgctgagcccctaacctcttaggagcagagcaggtttcctctctggaacattctgttaactgtcagggcacttggggagaaatctccaagctaaggccacgtgcacaaaatttcttggtccttatatccccagaatgtgacctggagtctgatggcagcccgctgcagagatgtgtccactgccttctggtcattgacctgcttgggtggagtgaatcattgtaggagaaaaactcagttccctcaccctgatcaacctggacagatctctcttcctttaaaagctttcttggacatctaagggctaggaaaaatgtcagggagcattgggaaggtaaatgaagtcaggtttacaaagtcaagtttacttcttgggagaaaaatacaatttccaaatcctctgttataattgccatcggccccctggagtggtgagatctcggaatatggctcgggtgcagtggctcttcactgtgggcctgcaggctattctgaaaagctgatgaaaaccaatgacccctcttccaagaaaaatggccacataccaaacattacactgtacatctgatttcagggaattgtagatgccaggttagtagcctcaggtctagggtcaaaattcaagtcgaatcccacaggaagagggtctgccttcggaattccctttcagagcattgggagaacatcatgggagcatattctagagacagaggcttagggtgtggacagggccatccctcacccactgtgctgaccttaagcagcaccttgtgcagcccatacctgaaggccaccagcaaaggcctgttggggagcaggctttacccgacctgtataaacaccaggctaggtgaaaactgagatacctggttactttagttttttccttgggggagctcagtatgattcttccaggagaagcctgcttttagactaaaaagaaaaaaagtttgataggtcaacctaatgattggaggtggccttccccactgtgaacaaactatggctgcatgtgccctacaatggcagagttgagtagttgtgatagagactgtatgatctgtaagcctgtaatttttatgtttgctgacccctggattaccagatgatagaagaggaaacatctgtcttcctagcaaagtcaaggaagtggcatttagcaggactcatattgctgcaagcactgccttgcagttttagtttacaactgcactttcagcttaagaaacacctgcccatccagagagatcgtgtggggtcacatggtgggatcagggaggcctgaagacagctcagtggaggctgcatggagctttggtgggaacggccctggcagtgtctatagatgttattgcggaaaactgaggggtgggagttggagaagggggctccagactctagctgtacttggcatttgaacccggaaagttgggtttcatgttttgcactcacattatgagtgaaatattggcttattcaaggttcttttgcttgcaaggcacggaaacccattcaagcaatcttaaaccccagaaggaaatctatgatttggatactagacattctcacagagccaagggcagcaaggcggggctcaggagaggcaggccaagacctggagagctgtcaggagctgcttcctcaactctcttccatctgggcctgccagccctggcctctgtatctactccattcacctctctccatggaccagtctcccctgctcctcaatgcctgggctgccattgttcatgcaattcacaatacctcggcctgggcaatcagaagctcatctctgaacaccatccaaattcctgggaacaaatcgggttgacccagctttattctccctgtcccatcagccttggcagaggcgtgcatgtgcatgcgtgccaatgtgtgtgtgcagggaggtccttgtggatgaagcatggctgtcagagcctacctgcgtgaatgggtggaagggcaggtctcagagaattgggtaaaaactggataaaccctccagtgatatccaccaatgtcaccctgtttaaggcttctctgggcaagagacacacagagcatgggaccgagaggcgagcagaccctgccaaaactgggagactgaatagatcgctcaccatccttgtcagttagcctatatgtacaaggaagtaaaattatctctttctcctgccttggcagtattgtaaggatactcaatgtagtagctaggccagacacatagtatctttaaatatagcatgagatggccaagcacggtggctcatgcctgtaatcccagcactttgggaggctgaggcgggtggatcacgaggtcaggagatcgagaccatcctggctaacacgatgaagccccgtctctactaaaaatataaaaaattagctgggtgtggtggcgggcgcctgtagtcccagctactcgggaggctgaggcaggagaatagcgtgaacccggggggcagagcttgcagtgagccgagatcacgccactgcactccagcctgggtgacagagcgagataaaaaaaaaaaatagcatgagatattattactgttataaaaataacagctatttccttattaatgaggctttgtccttacagcttggcaagggtatatcgagattttcaagaactcctcatgaatgcaccagagagccagcaccttggccgtatttggacagagctacacatcttgtcccaattcatggacaccctccggactcacccggagagaattgcaggtaagcatgactgcagtgctctcaagcatcatttccctcacctatggagagactgaagatataggaaagaacagggagagttggtgaaaaatatactagcggaggcaggaagggatggggtctggaggcggcttgaacatcaccttggtgaagatgcctcttcctccacagaagcctggaaggtaggaagttgggaaggaaggcaggaaaggtctcatccacgttaagtctagagacagaaagaatgctaagagagatggcactatgggaagtatgaggctaggtcaagggctagaagcaggggagacgagtttacagagtttcgtaaagatatagagcaactctcacagagttctagagcgagagctaaccaggaacatgaagcagcaaggccaactatcattaaggagccagggaggtcagagatcatgtattatcatgacataaatatgcataattgtactatttctcccagtaatatttagcacccaggccccgaggcagagcaagtggagagtgggtgatgcagggctgggggtgtgtatggaggcaccacagaaggtcaacaggcagcgggctgaaggcagggactggactacatgcatcaagtccaggctgcacgaggaaggatgagaaggcagatgagcacggaaatggactgggggaaatgaagaggcaagggaatagaagtctcagtgggtgccatgaccctgtttaagtgattgagaaaatgaacaagatgaaaaggttaatggctgtggtcagaaagtgaaatatgtgaattcaggatttcgaaggtagggtgggtgatgactggcccccagatgcggccatggtgaagtggggcaaaggtgcaggtgcatggtgaggggaaggaggaaatgggaggtgatgatgttggccccacacggacaccacggttgtgcaggaagatggcaggagctgggcaccagggtgggagccacctggagtcaggaagagtgaagagaaaggatgaagaggctccctctcctgtgtctctcctccccaggagaagaacaagaaacaatccgaaagtaataacaccaatgtgcctttacaaagtgtgagtgggtgttgtgtgctgtcacgtgtgtagtaggctcctctgtggatggctagagggactggacatggccactggatcccacttgcaagagcagaggaaaagagtggtcgtgaggaagtaaagccccccaaaatccaggggttgctgcagctttgggtgtggagcgtgccctctgaggaaaggctgctctgggggagattgcccaggaaacggggctcagaggccacgaaagcagctgttaggggcttctgggagatgtgtgctcctaggattagggagttgactctaaggatgaccttagaggttaacagggatgagaaaggggtcaccaaggggtctaccaggggaatgggagaggctgtattgatagaacagcttctgctgcaggttccaaacaagaaatgtgggagaatggttgaaatcagccccgggggcaccttcccgtgcatgcgtgcagctccttcaacattcagtcgaccttcagtgcctcctgtgagccaggcactgggctagtctctgggggtggagagatgagtcaggcaaatgccagccctcagagggctcacagggcagaaggtgagagatgagtgagcagaaaatgaccacagcgcgtgtggggcccagtggagggaaggaggggattcaggagcacaggagagtcaacaggggaaacttctccgaggagaatctgatcctcctcccatctggccaccttctgaagccctctctccccatccaagtgagaaaggacaggcgtatgaccagattggtgtatgaagatgctgaattacgttctcattgtttcaaactagtaaaccatagattttatgtagtaacttctacaaactgcattacaaacactccattctttgttgccctgggtagaagtttattttagtgagcccaagtttgaggaaccttatatggtatgagtacaattaccattttaatagtaagaaatcccccttcccctgtgtaccaaccagaaggtgtttttttcctaatttaaacaaacagatgcagacgtgggctgtccagctcctggcgggatgacatacctcatgcatccagtgggtttgatgatgaggcagacatttcacttaagtgcctgatcatcagattgagtcctgctgggaggaagtgtgaaggaagtaatttcaaaccacagtttctctgtggcttttacaatgtggatatgagaaccaaaatcactacttcttaaccccagagcaggactgattttgaattggtatgcaggcggttccttctgcaggcttcgggctgtgagaagtccctaacagagcaaatctggggacaagggctcaggaaaggttggccacggccccctaggaatgggggctctgcaagatccctggccttagaggctgtgagagggaacaggggtccatccccaagtaagggacacggtctttgaggaaatcccaggccagggcctgaagggcactgtcaggaacacaggctgtttcagtctgttgagattcaccggggcgctgctcactgtgagcacggactcctcaggccaatgtggcagaagagcccacctttgaaagcgagcgggtgggggtggcggggctggtgctggtgcgtgcttctgcacagccacctgggaaggtatgccgctggttgacccaggcagaggttttctttcatggcaaacctgcagtactgcattctcagcagggaggattaatggtaaaagaccaggcatggagcccccttccctctccctcgaagcaagctctgtggtctctcaatcatctttaaaacaccttcttcccgggagcctcctacattctcctggcttccctcccacccccaccctcagctcctggggcctcagcagccccacccccaagcctctaatcttcccagggaagggaacaagaagaaccacattttaaacgaaatttatttttctttcctcaggctcccagttcacatttctccctcaggagtctagggaagcttctgtctggtatcggcctcctcttcacctgggcccccgccctcctcaggtgtaccagaagccagcacactcccccttcccccccagagccacagcagccctgtctcctgggtggtcttgtgtgccaagcctgggcaacatcactcccagcttttcttgttttgccccttctccccagcaagatatttgtatgtaaggtcaggtgagtgagttaaagaataacgaagagataaacagtcaaatggagtcctgactgtcaggtcaagacaacagttatttactgaatgcctcatgtcattcaacagacatttattgagactctgattggatgtcagtctttaatgctgggtgtcagagagaggtgacttcaagggcttgcatctgtgcacccagcattgctaggtacaatgaggagtataataaaagcaggagccatagcccccaactctcaagagatctcccatgtgtgtatgtctgcatatgcgtgcgtgtgcatgtgtgcgcatgtgtgcatgtgtgtgtgcatgtgtgtgcatgcgtgtgtgtgtgcgtgtgttggggatggtgttggtggagtgagagtgtacaaggctgtgtatgaaggggtaattgggaaaagaacaatggagctggcacccagggacaggaggaaaagcaggagggctgggtttggaagacagccggatttatgtttttgaagagggaagactagaatataagggagcagcccttctcagagccctcctcctcccttcgggccctgtgtccagctttccccaaagtccttggatctttcctatgcaaaggggagtgacagtgggcaccactctcagggaacccattactgtgagagaagccactgtgccactgtgtggtcgaacttcaagaccggcttcccctgccccagctgcatggacaggcctgtggggttggcgcaagacccttccagaggaaactagctgcaacataaatccggatatggtgctgttcagggaaaggcacaacctggggatgagaagggtggctgtccagcacacaggggcaggcctcttggccactgggggaggggagaatttggagaggaagaggatgggatgccgtggaattgggaccaggaaagaatggggacatgtgatggttaaagctagttagagaagaactgggagataaacagtcacccatgcccctgaagcactcggggtgaagagattggcattttcacgcaccccagtgctttccctttgtgttgaagtcccttcgtagacatccaggcccataaggctcttctctggccagagcctcatgaactatagcactagcagggttgaggccaagcattggccctggaagccagccgaggaggagggtgcttgtgtgaatctcccaggaggggtaagaattatattaattcgatcataataagcatttattgagtgctgttttgaggcctgggagctaagcacttcacattccttaccccgcatcaacaatcctatgaggtagatgtggaaaatgcagacacggggacaggctcaatcacttgccccaaggtcaccttaactgttaggtgttctttatgcctccttataaagaaaccctgcttcccacaggtgttgagaggagctggagggagcttgactagggctcatcaggcaagccccggcatgtgcctggctctcctctttctacctggagcttttcctgcccttaatggccccaactcatttctcttagtccatgtcagtgccctgagcatctcagcccaagctgagatgatagaaacacccagaggggtcctctaccctgtgacagctgcggtgtgggaagagcacgtgtctcctccaatcctagaccagagtttctcagcctcagcatcactgacacttggggctagataatcctttgtgtgggggagggaggagtgtcttgggccttgcaggatgtttagcagcatctctggcctctacccaccagcacctccccagttgtgacacccagaaatgtctttagatcttgccaaatatttccaggaggatgaaattcccctgtttcagttccccagccccacctcaatgagaagcactgtcctagaccaaccccacaaagcatctgacacccccatccagccctggctaactttttccaccttcttactaaattgggcccagctgcttcagcagtcaatgtgttgggggcagcccactggcaagagcctcacctctaggggctcccagagaccccaagaacagaaccttcctctgagagttgagttacaagtgtttccaatcgactctggctgttttcctttttttgacccatttccccttcaacaccctgttctttctcttattcatatgtaggaagaggaatacgaataagggatatcttgaaagatgaagaaacactgacactatttctcattaaaaacatcggcctgtctgactcagtggtctaccttctgatcaactctcaagtccgtccagagcaggtagggggatgtcactggccagtggtccctggaggggagggaagcacccagcctgagaaaggcaagaaatatattggcttttttcttctttcttccttgtgttcacattcagaatccatcacttaatgccttgtatttagaaaaaaaccgggggatcacttgagatcgtgatcattttcaacataggattcgaagctgtacacatcctggtgaccttaaaacatctcaggtttttataactggaaggaaccttagagatcatggggcacaaccttctctttatagatgaggaaacagaaatctattcatttattactcaaatatttagggacagttgtaggtactagaacacagtgtgaaccagacaggcaaaaccccaggccagggagcttccattccagtggggccacaggcgatgctcaggtaagcagagactccgctgtgtgacttctggctgtgatgggtgctgcaaggaaaatccggtagagtcgagggttagagagggacggaggggcaggtttaagggggatgctcaggaaggccttcctgaggaggtggtatttgagcagagttgtctgtcagccacacagtaagtgagaggggagttccgggcttggaagctgccagcacagtgctggcaagtgctggggtggcgtcccgaggctacagaacctgagatgctgcagaagagcccacttctgctttcctggaccacttccttctcagcaccaggcaaactccttcttctatcccctggcacatttctgacctgtgtatacgcccccaatttatctaacccctttaaataatctcctctatttatgcagagcattcttaccactaactcacgacttgcacatcccttagctcccttactcctcacaacaatcctgagatgggtcagagaaggaggcttgcgcgtctggtgatggggtgatttgtgcacagttacagggctagaaattgtcagagccagatggaatccaggtcctctcaatcctaatccagtgtttcttacttcagtcctgtggctctcaaagcccagagaccagcagcatcagcgatgcctgggagcttgttaggaatgcaaattatcagggcccactccaggtgaactgggtccaaagccctgggataaggcctagcaatctgtgcttcacaagccctccaggtgattccgcaggctcaggtgtgagagctgcagctgtcctctgggccttctgggctccccgcccagcttcttcagtgtgatgaacacagcgagaatgctagatctgcagcagctgatatcccagacaccctcccgactccctcctggctgggtctgatcctcctccagactccaggagagaacgagacataaacagaacttcagagcctgtgttaaccctgagatcaaggtctgcacagggtgctgtctgagtccagaggagtgagggaccccaccccacctggtcagcaccagctcctggaagcaggttctcacactggttccctgcacaatgaaggagctcatacctgcttttctggcttctcagaccctgaggttttcaccgaaactagacaaggggaacctagggtcagcctggaggcagggtgagcttggcgcctgcagtgcccaggccctgggtggtgcggctccggccaggccctgtttagcttcctctcccacccccacagagggggtgctgtcggcaccgattgctcattttcccctttgctttctcttcagctcgtaaaactcaagtcctgacaatgccttgatgacttccagttggtaataaaagggagatgaagataaggacaggaatttcggggaaatttctttccagttccttactaatgtgacatttagatctctagtactgtgcttctggcatcagtgccaaggcctttcatgttggagaatggaggccggggtcaccaggttgtgcctttatttcatgttgctggctctgatgagctgatgctctgctgattagcaaacgctgagccatctgcgcttcgcagaggcacgttccagccaacccggccctccctgcccacttcccaggatgctttgccttgtgggctcacctgtcttctagctcctgatctgtatctccacctccatccagttccggggctccttatcagcactgttcccagaactgtccatcacgatggcaacgttctctctgggcgctgtccaacatgggagctcgcctctgtgttgtcactcatgctcattgaacatggatttgtgtcctttaccatcaggactggatacccctcctggtcctttctgcctggggtcttagcacagctcagaaggaacctcaccattccctctctccatctagggaattagaagatgacaggggcacagttctctggctcacccccagcccagtaaactcctggacatgcttcaaggcccagctcagatgttgcctcctcagtgaaataatttataaacccacccttctttgtcctgccttctccctcttccctactcactggagagttaacaggtgatggttaagctctgggttcaaatctcacaaggccacacacttagctatgtgacttcaggcaagttaattaaccactctgtgcctctcgtttcctcatttgtaaaatggaaatagtaaaagtgcctaccagcatggcagttgaagttaaaagaaataatatatgtgaacacttggaagggcgcctgacacatagtaaactctcagtaaatactagctgcttttagtggctattcttaacacaccctcttcagtgctctggtttcactatgttttatgggtccctgagatcgaaagtgtccacaccgactcatggtcagctgtaacctgtgcctcgtgtggggaccaggctgccatgtgtagtctggacagtgtaggaggtggcagagctcaggcctgttctgccctccagcccagagagccacgtcgttagatgtcatgggagactgtggtgccccgggaatctcacgaatttgcccacggtactcagtgtctgtccaatgctatgggagtccaggactctaggagccagttaaggtgctgggtggccacaggtccctggccaaggtccaggcctctcccctgccacctgatcctcgagaggccatcacgagggttgtacttcaagaaccactatccttgagctacctaggagctgcagaatgtgcactctgcagggcttagggcctgcagacaagatagatgcagggtgtctagttaaattcgaacttcagataaacaacaaataattttttcaaataattgtgttctattcggtccctatttgggacatatttgtactaaaaagtattcatttatctgaaattcagattcgactgggcatctggtgcttttgtttgctaaatccaagagcaaatttgttctagctacttctcaaccccaccttcagagaggaagccttgatggtactgtaacatcatgctgtaagaaggggatcccttgaattgtaaatggcactctgataagatgaggtatggggattgtattggtttcctgttgctgctgtcataaattaccacaaacttagtggcttcaaacaacacagatgcattatcttacagttctggaggtcacaagtctgaaagttagggcatcagcaggactgcattccttactgcggagttctagagaaaaatccattttcctgcctccttcagcctccagagacacgccacattctttggctagtggtctgcttccatctccaaggccagtgggggcttatcaagtctttctcacatcacatgactctgtttcttctgcctccctcttctacatttaagggacccttgtgattacacaggggcccacctagaaaagccaaaataatctccttattttaaaatcagctaatcagtggctttaatcccatctgcgatcttaattcctgtcgccatgtaacacaaggtattcccaggttctgtgggttaggacgtgggtgtctttcctaccacagggcagtttctagtgttgcctcttctccctgcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtggtaagggaggggtttggctgctcgccaattgcaaggtgattcctggggtagcagagcctcacgaattgaccttggggagggcgtgagcctggtgttctggacaatccttgcaaaagctccaggctcccagggctcaaaaaatcacaactgatagtatttctagaacagtggcccagggacccagaagtcactatgaggttcaccattaggtatgtggctgtggcatgtttgtgtccactctaaatgtggggataatcccctttacctcctctaacagagtggtaaaggaaggaggaggcctggtttgactccctgacctgctatttcctagccaggtgatcatggtaagatattgaaccttttctggtcccagtactcatctataaaacaaatataatactttacagagtggtaggaattatacaagaaaagtatacgcaaaacatttcataaattttaataaatgatggccccatgcttcttcctctggaaatggtctcaacctcaatggttggtgtttctagagagaaaaaacgacagagaaagtttcatagtctcaaaaatttggaaagccctgatctagctcaaccctttgttctagaactgcatcccagacagactgcttgggacctgaaaatatctcctcctttgctagaaggataagatgagaaggaattagataaaggaggtgtagagcagaggttttcacactgcaaagtgcataaaaaccatcagagggccgggcgcagtggctcacgcctgtaatcccagcactttgggaggccgaggcgggcggatcatgaggtcaggagatagagaccatcctggctaacacggtgaaaccccgtctctactaaaaaacacacacacacaaaaattagccaggtgtggtggcgggcgcctgtaatcccagctactgaggaggctgaggccggagaatggcgtgaacccgggaggcggagcttgcagtgagccgagattgcgccactgcactccagcctgggtgacagagcaagactccgtctcaataaaaacaaacaaacaaacaaaccaaaaaaacccatcagagaagttggtaaaagatgcaagtgctaaatccccacccccaatcactgtgattcagaagaaccaggccaggcccagaatctatcctgttaccttaggcgattctgatgaagaccattgtaggccacactttcagaaacactcaaaattagaatccttcagagaaggtggcatatataatatttctagcatggaattatgttttttttcttttgcctacattttaatttctagaactgtgttgtagggaatgtcagtcactaagaacttgattgaggaactgtgttttgtctgtttcatgactgctctctcaagtcccaggaaactcactttcagcttgtcttaaaaagcaagctgaaggcttttaaaaatgaagcaacatgaaataagacaccgcagtttctggcacggtccacgcttaatccccttcaatgtgtgactttccgtggaaagttactctacgattttcccagctcgtcagggtggggccccagagtgagtatgaagggtcagagcctagggatgccaccatcagtgagagcccaggaccccagaaaaggtctcttggctcaccacactgtaggaaaaataaaaagcaatgtagtccaaatgtctctatccaaagtttcaaaaagaacttgattttagacacgctccttgacttgttttcagaatcagacagaagagtgaggcaacaaaggtcccttattccaggcagctgaataccagcacagccaggagtccagtgctggtgtttgcagagccaccagaggctccctctcaggtgtccagggcccgcatgctttgtagaatgggcagaatgagcaatgtctgtgcacctgggctttgcaggcagggcctgggtacccaggttcgtgcaatcctctcgtcaccatgaagggagcagcatcattcttcccttcttgaagcaccttggccaccagtataggtaaatttacctcccaggacatgaccattgattctgggatgtcaatgccagagatagtagggtaaatcggcacctgggtaaaactttccattggagactagaaccaaaactcaggacactggcttccaaatgtttctttatcagacaagaaagaccaagtctttccttacgtcttcacatgctgccttggcaaatgctagcattcacaaaccctgggctaccttgacctgtcacccttgcagacctcagacgggtcctgggggcttgctttctcggtttctgtatgcaggcactcaaacctgcatcaggcacctgtgaagggccgggcactgtgctgaggccaaggctccaaatgtgaaccttccaccctcactgaactcacagccagaccagagacaagcaaacaggacatttcacagcagtgcagcctagaaagggccaacaccagcagcatttgtccccccgagcggtagcttttagaagcttccccagtgattcaatgtgtcctacaaatgcctggcccccactcccagagattctgagtcagctggcctagggtgcagccttgacttcactgtgttaaaaagcttcccagataagtccaatgtccggccaagattgagaatcactgacctagagtttaatttaccacctcagtctctatagaccacgcataataatagtaccccacacacctctgagggtccaaagaactttcatttgatcacccatgagaccaccgtggtgtggagatgctttctctctcctgttctcttaacaaagctggtgagcgacagagcctgcagtggaccgggagatggcccagaggagaaagctctgccgtagtcggcctcagttaaccacggagcaccacccctacctgctctcctctcactcctgcttccgtctcggtggagaaagatccaaccgaagcaggacacatctagtcttctggtgcctttaaaatgtacttttccatttgacaaatggattacactaaaaacaaaaatttacaaaaaaaaaaaaaaaacctgaaagaaattgcaggcattaaaatgggactttgcctttattgctcctgggcccatcctatttgggtttttagaaaaacaagcctgaggcaggcccagaaaggctcagggcagaccctccgatcctctgaaaggagcatcaggcaggcaggggttgctccggggccagggaaggggccccgctgggacgcggctgttattgcagctggttggcgcgcagccatgcttagctgcagtgcgggaatgctgggccttctgttctgggctgtttctcatacgcacgtaggccagtgtataaataaggttttattaaatgccaaatgagttctcattaacaaagaaagagggaaaatctcagtaaaccaccgtgacggcatctacccactttgagtcaggagctgggggtgtgagtgcaacctccgagacaagggaacctgtggagcccagagaatcggaggggggcgctggggttagcaccgactgagaccagctgtgttttctctcggttccttggagatcagaagtgagtgttgtcatcttcaaacaatccaaaggcagtacccatggccttactacatccctcccacaccatcccacccatccccgcgcgtacactcacacgctcatttgcacactatcgcacacgctcacttgcgtgcgcacacacagattggtgacctaggtggactgggagagaaataagagccaaatgactggattttctccaaggaaatttattaatagcccctcttggtttcacctgaaggagcttgtcttcacctgcggcctttgcaggcttaacgcccccagcttgaaacccagaagctcagacttgggcccaaggtattattagtgccaacactacctgaaatgtttcgcacctcataaaaatggtgtgtcagtttcgggtgagaggttgggacgcttcccatctgatttggcccaaggcatgcatgcccctccttctccttcccctcctcctccccctcttccccctaccatccttcctgttttctctccaactctggtgcacagctttgaaatcttgctgagaagcaaatctgtcccttctgctttgaatgtttatttgtggaagttcggcaggggaaccgaggcgggtgccaagacctgccatgctgctgggaagtctgagtctccctcccttccccctcctaaatgcttgttgatagagaaaagtcagcctcctcggcatttgggctcacggttttcctttgaaaatgcttccagtgtggcatgattcagctttcttttctgtcccccaaccactgctctgttgtcatttttacttttctgattgcattttatccgtgtctctttgactacggggtggctggacgttgagttccaggaagaaaagggcccaatcttggggttctgactacatgcgcccatcaatgtcctgtttcattcttggctctggctccctgaattcctgagtcactggggagaagcgtgggtggaccgccccctacccagtgagagttgccacagttgctgctctcctgggtcattggttgcagattgttaaacttcacctatgcatttcaactttcgggtggatattgctacgtcaagtgtctgggaaagcccccacagctacaggattttacagtgaggtcccactaatgacttgatgtcatgacttcctcattctttccaatttctcccacttctccataagggttttgggaaggggagaagagaaaggagtgattcctgagtgccagtaccagggaacagcagggctgttgggaggaaacaaaactaaatcaggaaggtttttgttgttgtttttggggggttttatgaaaatattcaagccacagcaaatatatttgatttatagcattagtattttttctgcctgcatctacaaaaatctttacctattaccatcaaaatatcctctgggtgaatggatttcaacaaagaagaaataaaaatgaaatagaagagaggccccttcgtgcacattgagcctactggctggattgtcacttgcctgccttgatgtcttttcagctccaggcaggcagtaggccagggcttattttcatgacagatcagatgttcttttatggatttacaaagaaagaaatactgagaagtcaaaactgaagtcacttaagacaagagcaggcccctgggaaggctgccattgaggataatgagtcctggggtcctggcctttgttcagtaaatacgcactaggcgcctacaatgtgtgcaccaatgtgtgaggcgtcaggttctctccagggtcagttggttttaagaaaggttttggcttctgatatgttttatctctacagaacagtagctcttaacctttcttatgggttaggattaccttcgagaatctgactacagctctagacctgttccctaaagaaaactaagttcacagggacacacaggatggggctcatggagcagctgaagccagaccccaggttaatagcctttacattaaaatgtttttctacctaccactaatatgcattctttagtaagcggtctcaatatacaccgattcttccttaactctgtttatgaagtattcagcatcctccctgcccccttcagcatcctccctgcccctgagcacaggatccaatggcgtgaggaccacaggcctgggcagctgctggggcatacaggcatctcttagtggctgagagactgggccctggctctatgttggctcctaacttgctgccatttaaaggaaatcttagcctcccatccgtaaaatcgagaaaataagacttgtcctacacagctcatgaaatagtaatgaaattcacattagagaagagatggaaaaacactttgaacaaaaagcattttgctcttataaaagcacagcctcttttgagaggccctttgctccccatttctccttcttcagacccccccagactaggagaaggtctgtctcatggagtgaccttttggctgcctctagattccaagctcagttttgctttcattaaccacagatactgggacggacagaaaaagacctagtttctgttgagccaaagagtctcataacttgtctgttcacatacccaagagcccaccctctagttgagacactcagttccctctcattctgggagactgcatgtctctgtgacctcctggtagagaccgtttgacatgtcccccaaccccccagtgattgagtctgaattctccactgatgacgcatttcctagcactcagggtgtcccctcctggttgccccctcaccactgaagcccgcttcctcccttttcatttgatgcttaacaactgtcagtttgcaagaaacatgcttcaaatccacattctcccagttgcctagcaacaacttccctcccggataaatgtgggtttcctgtagctcagcccaggactgaacacagcagcacacacttctgtccactgcttcaactgcttttcacctctggtctgcatgccttcaagactgcagctcatccctcccttcagaaccttccatagcctgcagaggccatgtctgccccaaaaagacacattgaacctgaggctacttatttacccttgtgttaggtatatcctcaacttagaaattaatactgtttccagattgtcttctttgaatcacagaaagtaaaacaacaaaacattcaatgcttaagacatttcatgtgcggttgggtgacatctgtttgatgaacacatttgatccaaagcatcagaaatactatgccaacaagactttttaggaggtgataaacatgtctgttctaccttaagaaaaaaatattacacagtcccaagggagagacatggttttgatcccagacaacccaagcagagacctcttagggccggaatcatcttggctgctgcctaggaccttatatcaatttcttaagcacaggatcaaggcctaaaggccccttagactgacctcagttagtagaggcagatcccttcacagccttatcttccttagaggtctagtctgaccttgaacttcggctggcagtgctgtcagttgtgatgtgtgacatggaagagttatttgttacttggaaaattaagagaacttatttggcataggaaattgtgtgtgtgtgtgtgtgtgtgtgtgtgtgtgtgtgtgtgtgtgtgtgagatgatgtttgccattttgatctgtgacttttttttccagaaatagtttctcagttccattccaactaaacttacagtctcttccggttctttgacagaaacaattcatgtgaatttgaacagataatagggaagggggaaccaaaagaagaggagagccctgggaaagttattttataatttatggcaacctcagtcaggcaactgtgaacaggtacatatggagggctccctcgggactaggcagtattcagagatgtaaggtgtgaggaccggaccctcatcatttaccattcccactaaaaagagctgggaaggaaattgtagctgtagcaccaggcacgtaactggagcttagtaactatttggtgaaggaatattattaaattattaacaagatggaaaaaagggtattaaccacacaaaaatacatctcaagctattgtttctctgttccctttcccccaaattcctagtcttgctcttatctggctgtctctctagtcactctttcttgctgactctcttcacgttcctttctccacctggaattcctgggccctccccttttactgacagacactgtcctcactctcacagtcatcagtttgtctctttacaaacctcagctcaagtgtcacttccccgtccccaggtgaaactgactgctccctccctgtaagtcaccatgatgactgctatatatagccctcatggaacctaaaacctcaacagacacagtctctttcctactctgttatagtttatttactcattaattaccacaacacgtattattgagcacctactgtgtaccatgcccagaagataaaagacaaacaaaataaaacctattcctatgcttaatgagtttacagtctagtggagagatagatacattaaaaaataacagcaaaccaaaataaaagtggtaaataaatgcactgagaaagacaggaatagctaggaggggcacctaatccctagggaaggaaagctggaagagcatggtgatgggggaagaaggctttctggagaaggtgaggtagtttgaaatgagttgactctggccagtaggggtagagtgagaatggggtgagacagggtgggttggtcattttgatccattagtcctcaaagtgataggactagtggctaaggactgcaggctttacagaagcctacaaaactatttgagatttgaagtttttttttttttttaattggctccaaaagaaaatgaaaaaactttagaattataatgaatgaatattaaatgaatatttaaggaaggtaattttattcaacttcattgttaaatttagttaaaacaagcccttgagtttcattcaacactgttttatcataccgttgatgagagaaaacaaaactgattcctggccagggccactgtcagcgtggggtttgcacatctttcccatgtctgcttgggttagctccaggtactcctgtttcccccacatccccaagatgtgcccattagtggaaacggtgtgtctgcatgattccaacgtgagtgagtgtgggtgtgggagtgagtgcccctgccatgggagggcatcctgtccaggttagattcctaccttgtgccctgagctgctgggatggaatccagccacccatgactctgaactgaaataattgggtgaataattatcttactttttaattaatctttgaaaatgtatgtatagttcacatgtatttcaatatttaatattagaagtattttagtctttattttgaagtttggtgatttattgtaaccagaaacaagctatagaaacttaattttgggccaagtgcagtggctcacacctataatcccagcattttgggaggccgaggcagacgcatcacttgaggtccggagttcaagatcagcctggccaacatggtaaaaccctgtctctactaaaaaatacaaaaattagccagatgtggtgggcacctgtagtcccagctacttgggtggctgaagcaggagaatcacttgaacccgggaggcggagcagtgagcagagatcgtgccactgcactcccacctaggcgacagtgtgacactccatctcaaaaaaaaaaaaaaatagaaaagaaagaaacttaattctggtttatatcaattagcctgtggtaaaattggtttcattatagccatttcacttagttgaagtttccaataacctgtggatgaattaagtgaggatttactatattcataaaatcttaaattccaaagcctgtttgcagttcaggtttttccactttacaaacacttctaagtattcacaatgattgcttaaaattcataccagataaatcattaaataagttgttcaaagtcaaataatttcataagtaaaaattaggagcttttagaaaactatacctacatagacctagacctatagatagacagagatctgaatagatatggacacagatgctttccaaagtgttcatgtgatgtgtggtggagtttcaagaccagagtgtgcctggggcctgcagaagtaaaggagaggggatggagagaagattgtccacatggccatgggcaatctcccacccacactcaagtgaggaagacaggaaacaaattcagaaagaagagaaaataatcaaaactgatgggagcttgtgactgatttacttatgcgcagcctccctggagacatgagtgtggctgttccttaggttgtgcctctgggctcctaccccctcttagatgccttcctattatctaggacctggttgctttttgtctgcatagcttctttggattccagtctttgatgccagcttcctcctaaagtagcctttcagatgtcccttggttaccctctgctatctaagggctcatcctaccccacactcattcccagcaccaatttctggatctccaggctggagatttagacaatgggatgggaagaacccatgatgggtcccagacagaaagtggtgccagccacagaaagggcacacaggcacagaagttggtttggggtaagacgatgtggtcagttcagaacacgctggatctaggcagatgcccagcagacagttggatatgtaagtctgaagctctggggagaggtctaggttggaggtacagatttagaagtcatcaacaaaaaggtagcagattaaatgataaaggaaatgagactatccggggagtgtgcagagtgagaggagcaagggaggcccttgggaacctcagcacttcaggggaaggtagaggtacagttgctggtgggaaaggcagagaagtagcaaagcaaaccaggcaaaagcagtgtcacagacgaccagggaggaaaaggacatgatcaaaatgttgagaaaagcagagaggtttgaaaatacaagaagcaaaaatgtccactagacttaaaaaccaggagaaaactggggggttcttgataaagcatcttagtaggatggtgagggtagaagccagggaagtgttggtgaggaagtgaagtcactgattacggactatgcttaaaagaatgtgggaatgaagggtggaagagagaaattagactgtagctagggagacataagcgatcagaggtagattctttctctcctgtgggagaatcttgcacgtatacacagcatgacgacagtgatggaagggctggcgaagcctcagggagactcttggaggtaaaccccatgaagggaggactttgtttcattcactgccgtgtccccagcacctggcacaatagcagacactcaatacatatttgtcaaatgtgggattttatcatttagaaactgcacctggctgtgagtaacaaaagtcagagaaaccgtgggtttcatttttctccccaggcagagtctggagctgggtcctccaagaggggtttggagcaccacaggtttcctcaagacccccaggctgccctgtgtttccctccttcatccccagcatatgcctgtcatctggtgacctccaaacacctgtgctgcctcctccagcacatccatgttgcaggcagggaccaggcaaagggcagaggggcctacttcaaaagaccatttccagaaaccccatcctatgacttctcctggtgtcttggttaccattgtgccataggctcaccctgtatgcatgggaggctgggccaggcattatgacttttagcaatattgcatagataagcatcaatctttgtcactgtgacgaagcctagtcactcagtgctaggcaaggttaatggaatgggttggtgtgtgcattattcttgaggtctttcttatgcttcatgttatacatttattaggacgtttaggcaacagggggataaaaatgaagaggagatgcatgctatgatctgaatgtttgcatcctccccaaaattcatatgttgaaatcttcatccccaagatgatggcattaggaggtggggcctttcggaggcaattaggtcatgactgggattagtgcccttgtaaaaccccagaaagccagcttgccgcttccaccatatgaagacacagagagaagatgccatctacgaatcaggaaatgagcccgcaccatgcaataaacctgctggagccttgatcttagacttcccagctgccagatctgtgggaaatagatttctgttgtttacccagcttatggtattttgttgtagcagccagagtgaactaagacagtgctgatctcgtattcttggagggaacccttagtctttagggaaagcaaagccaccatttggggcagggtgttctccaagtgctgccacatatgctgatgtggttaaactgcaaactatggtaaaaatgtggaggtctgtggaattgtcaatcaggaaaaagatataaaaagaagttaaagtcttcgtgcttctggaaggatatgtgccaaattgttaacattgattatccttgggtagagatgtggggaagtttgcagagacagttttgccttgtactttatataagtaaacagctactacttcgttgtcttaaaaaaaaaaaacagcctatgtgctcttcatgtgactcagaactacctaggcaatacgattaattgaattagtaaaattgagtgattatgaattttcaggaagtcattaatttaccacttctttattacatccacttctaacaggacttcaatataggggaatttgacttcaagataaaaagaccaaatttatttacccttttaaaaaaagacaacttaaaagcagacttgtcttacagaaccttccttagttggacatcgatgagtgtacagaaaatgcaatggataaaaagcttggtgatacaaagataaaaagtggggtcctgtccttaatgaacataccatttcatggagtatcaggtgtataaacaattataatcaatctgcttgttattctgataagatcatttactcacacatcaaatactgagtgcccaccacatgcccagcatacctagaagtcatccagtatgatttctgtctacatggagcatagagtcttacaggggagatagatgacaagtaaacaccagaataattaccaatggtgaagagcacaaggaaggaaacagaactcctaaagagagcgtggctgggcaggggtgagcaagaggcatagaaaaaggggcatctaaatctacttgggaggaagctgtttctcacataggtcatcatgttaggaatgagacttgagggatgagtagaagtttgccaggcaaagaaggaatgggggggaatagagagcagagctaggggcaggagacagctgacgtgtgagcagacataaaaagaagtccactgtggcagcagagaagcaggagagaaggcaagtgagggagccaggcaccagctcacagaggtcatgtgtgtcaaaacgtagtaatggccttctcttctggagacagtagggagccatggaagatgtttgagcagggaaagcgacatgactggattggcctgttgggtaactcagaccacaatgcattggaagggagggggctagaggcaaggggactggcaagaaggccagtcctttttctatgcctattttgatgaaatattctagaagggaagtgaacaaaggtagtcctagagaggaagaacaaaacagataggatacttccttagtatttgctcattcgacaatttatttttgcatatacactaaaaccttttttattattaaaacgttttattgtaggaaaaaagtatgaaagtagagtgaataataaaatgagctcccatggatctatcacccagcttcaactattatcaatatttggctgttcttgttttaactgttctccacctttttttcctgaagtttttttgaagcaaatcacagacaacatatcatttcaccatatgtacttccctctgtatctctaacatgtaagaacttgttttaacaaaatcaccatgctatgatcatacccaacaaaatttatcataatgtcttaataatacctaatacccatttcatgtccactttcccccaattgctacagctggtttgttcagatcagaatcaaaatccacctgtggccattttactgctatgtctctcaggtctcttttcatctctaataatctcaggggagacaggagggaggacgggcaggacttggggctaacttgcttatcgacacacagttttgcctacttgcttcctcccttcacacccactcttcttctcagccccacccttgtatggaaaaaacagaaattaaagtgctttgcccagcacccactgaagctatttcgaaggagtttgaagagtactcccggcaagacaaatgcctcggtccagtgctcaggtcaaagaggggagacgcttctcagtgatgtggtgtcaatagcagcttagttgttctttcctctggaaaattctacccatctgctttgtaactcccatacctaacaaggccttttatttcacaattagaaaataagcctgaaatatgaatgctgcctgagtgtacctacatttattctagagtttcagggtcaaaaagaatacaaggacctctgcatctacagccaagaggagaggggcaaagacacacagctacaaatgagaacctggctggtcaaagcctaactccacctgtttgtcagcactgatgcaagttaggtcagcccaatgatcatttaggagaactgtgctggcaaataaaaagcagaggcttttggtccccagatacttggatgagaattacaagtccagctggttaaaaggcacatgcccagtgctcacttcacacctactcaggaagcacacttgagttggaaaaccactgtctttacacttagaactcagtcctacatgactcctctaggatcagtgattccatcagttttgaaacatgaagcatgaagtcaaacaggacatgaccttggtttccagaaaaccagatgttcacatcagtctctggagcttggaggcagcacacctggggacttccacatcccctgccgaggtggcaaaagcaggagcagtggtgagttcacatgggctggggtttcctgaacactgctggcaattggagaatctgcaagggaacttctccgactcctaccagcagctgctttaaaataaaggtgatgtagctggtcaaatcctccatgagagagcagtgttgaatggaggaagagacacaacctgtctgaaaatggcacaaaggaagaaagatgtaaacaatgacgagaagactgcagtgtctacaaagctccgaggtgaacagatgggcaccccaggcccgcagcacttccttcagtctctgccagctgcactctgttttccttcctccaggaatcttgtttggtgtcactaaaacagcaattagaatcactttgaaatagtgatagtatttaatataactatgaaactatctgtgattgacaagtgcagcaaggagtcttggaatgagagcctttattttttcaattaaataaaagagttttttgtttctaaaagtaatcttgcagaaaagatcctgcgatcagaaagaaggagggggggagttttcaaacatataggagatcagactgtgcctatgtgtgtatatacctacaaacatatatatatttaaaaaattgttttactgtcaattacagcttcccacactcctagacagccgttctcaaggtatcaatctgagatcttggggaggaatattatctgatatgtcaccaagaattcaagaggtgagtagcctgatggtagtaattataatttcattatgtctttccaccatttaccccacttatgtcaaataatttaattgtatttcaaacctgttcaaggaaaagtacatttgatctttccatctagcaatttcaaagcacctgttcacatcccaaattatctgtgctcttaagtaagaggcagaaagaaaggaaccacccttctgatttcacatcaaaaaagaaatgccactggcaataagcaacttgcctggtgtggcataaatcatcagaagacttacagttgaatctaagtcttttcagtactgaggtggttcattattctgttacagtcttaaaattcacataaatatatactgccaataataatagcatacacctttatagcttacaggcactcttcttctaagtgttttacctatgttggcttatttcatcataaagaaaacaatggacttttgtgttgttttgtaaaaagatgcgcacattttaattaacatctgattgcacaagtctcctcccatatagaaatggattcttccacgcaatagataagaggtgctggggatatgatgatgaacacacagatttggtcatgaccctgtgggaaagagagatgggaaaaaaacaattctcttcaagtgtgatgagtgttacgaaagggagggaaaagttgaaacaggtttttttccaaacttttctccctccattattcgcagctgacttgggctccaccaacctggaaaactgcatggttggaatctgtctttataaaacgcatctcaacctgggccgagtatgcacactgatgtgggaaagttagagaagagcccattgtactaatgctcacctgctacagtgggagtctctgttaaacagtcttttcttcatagcattaaaaaaatttatatcactacaataaggttgaaattgatagagaatgtacaaacaatccccaaagtatatcaacactcttagttctgagtagaagttccagaaggcttcttgactgtctagatagcaagtctaatcatttgtgaactaagttaaagcagaaggcccagtttatatgaattggtattacaccatttgacctgagaacagccccttcatctctgagtgctttgactaaatgagcaacataataatagtaataaccccttacaagatgtcataagactcactgttgttgaagcaatttgagattttgactttattgaagcatagatggtgattataggcatgactcactgtgtggattctccctgggctcatcagtttcagagggcaagtgttggcatgtggacaaagagagggatgacacgtaaacatggcttattgcaatggggaaatattttcagtctcactgattgaatcctaatggttttataaattccccagtaccactgaaagcaaagcaagtaatcaggtgtgttttaggaataaaagcagcattattttaatttcgtattttcccctaaagcaaagccaaatggcattatgggagccaagctactggcagctccaccagccttctcctgagttctcggcattacagatctaccctcaaaggatgaggccagcaagcaccacagggtgcccacatggagaagagaaggccaccaacctcctcttagctggcacagaattgaaaaagtgtttttccaggaatggatacttcatctgttctgtatttgctagaattttaaaacgcacacacagacacacacaggcgtgcacacacacacgcacacacacacgagaaaaccacaaaccacacatttcaaggaaatggaagaattcattggtaaaattaagctaataagattattttccaaatataagaaactaaattttagactatttagccaaagaaatttgctctgatcttgcttttctacaacagaatcattccccaatcattttatttccctctttttctccccagtatccccatcttggtgggacaacagaacccaagaactggcttaacagtaaaatattttctgcatttgcccaaggacacattcccaacgaattcaaataaaggagactagaagaagagaggctatactacagtgctctaggggtcactctgtgatttgttgttgttgttgttgttgttttgagacggagtattgctcagtcgcccaggctggagtgcagtggcacgatgtctactcactgtaagctctgccccccaggttcacgccattctcctgcctcagcctcccgaatagctgggagtacaggggcccgccaccatgtccggctaatttttttgtatttttaatagagacggggtttcaccatgttcgccaggatggtctcgatctcctgacctcgtgatccgcccgcctcggcctcccaaagtgctaggattacaggcatgagccactgcgcccggccactctgtgattttctttaaggctcatcctagtattctcctagtccctaagtagatggcagtaggttttgttttttgtttttcgcagctggattaaggattgctgagaatatatggatgttttcttttaaatgtggaagtcaaaccaaacgttggagcattggcctcacagcagattatgactctagctgccttaaaataacctgaagactttgccttgccctagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaagtaagttttctgagtcctgcttataaattggcctctcatgttggttaagttgatggtttaacacttctaggtgaaaccaaacctggggttgcatctgtcttgtcttgctgagtggccttaggtaaagagacttctcccagaaagtccacttccccttgcagaaagggggcattgcttataagcaattctggacatgaaccacagaaagaactgaggcccacttggaaagggaacagaggggccatttcccactgatgtaattgaactagggctaagttcaagaggaagagaatgatccgcaaggaagcaacccagagttccaggtgaagctcaggtcagaagggccctggcaagtaaacacggctgtgggatgcttttacaaacacaatatcgtgaaaatctatgtgtgtagtactgaattacattccaaatggcaaattcctggcaaatcatcttccccacctttcactatttttttttttttggtcttctatggggtaaaggaggatggggtggggaagaaatgtaactggctgcccctctagttaaaaactgaaaagaggcagcaagggacatgccaaaagtagttggactctaagatagctacacacaacaaagcagctaagcagctaattgaagggaaattactgaggctcaagctgagattccaagcgggggccttgtttggcctctcagtccctttcatctgagaaaggcctcagttcctagcagtaatcagaggcaggcttctcagcctccttctcctaaagcagaataaaccacagggcaagtcgcatcctttgtttctctgatgaggccattactgagagtcactgtggcattttgctactaatgatgagcttgttattggtggggtacagcctattaatttaggttattcatcaaatcctccagcatggagttgaatgagacatgtgatgtggatacactaatgactatattgagttacaagcaatggggagtttctgtaaaatctgtcccttgtctcctggcagcatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgtaagatcccagctgggcttgccttgtgtaccctggacctcccagaagtgtgtgtgtgtgtgtgtgtgtgtgagagagatgtgccttcctggtagcacatctcatgtttgttttttgctaagtggactcttgcgtttcctcccccatccacagtcatcactggaatgctttgcttcagtgcccctgcctgggccctcccctctctactgcagcctacaatgaggttttctttcccattgcttgaattatatccctaatggaagggttcacaattctctgaatcctggctactcagataaagacagggaggaagggaggaagggtattttctcccagggggtccaaatctagctttaacgagggaggttctgagaaaataatatcatcaatattacatggacttctgagatactaagaaattagattctgtcagcccaggaagttgggagatggtgaattgttctgggaaatagcaatagactgagaaaataaaaacacttccttgaaaagcctttccctaacactaagtgataggggcagaaaagacacaaccaaaagttctctctcacttttctctctgttcgtgtctctgtcttgatctctgtctggttttaggccaactcaacttttgaagaactggaacacgttaggaagttggtcaaagcctgggaagaagtagggccccagatctggtacttctttgacaacagcacacagatgaacatgatcagagtaaggggggttggaggatggggaggggaggggaggaggaagcggtgggggcaagaaagttccacttgtttccttttcccaggaaagagttaatcgctattggagttagatcaaaatacaacaagcaggccccaaaggccttcattccaagcagtcaccaagtggggtcactgactttggatgagaaatatgtttcttgaattctgggagaagtctaaaagctgccacaagaccagtggcttcctggagtttcctacttttatgaattcactcaagggcctcaaattcaaagaggcatctccccaaggggccagctctgtaactccaaagatggtggaatgtgtttgtctggtctcattttcagctttgcaaaatgaagacaagagttctatatatcagggacactcaaaagaaaacaaaaatatccataagcaaaagaaagctttttatacaccatattcaatgacccccatctggcccctcctttgcccctacacatcttccctctattctagagacccatggacttggggaaatgggatatagataggtatgtttcatagtggaacaagctcaccagctcttcagggagccttagcatctctatcctcaatcactaaaaattagaaatggctgaagaacaagaccaaagatcctatggaatttctaagcagagcagtgactgtatttcttcttcccaaggataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggaggtaaggggctgcaagccccacagtgggccccttgaagatagccccatgagtggggccagagctcccttagcaagtcaagtggtcttgaatttaagctttcattttccccactgaagaaacaagaatccctacatcccctgtacagttctcattctctaacagcttatccatacttaaaacttatctatgctgaaaacggtttcctcttcacatctcctacttctcatgctgggcacctcctcctgtagccccctttaagcatctgtgtctgtcctcaaccctcttctgtctgacattgcttgagtggccatctatggccagtgtcccctcaaccccacagtccattgcttgctggacactcctgccctcaagttctacaagcacatcagcctcaacatgtcccctccaaaaactgtatgttctccttgcccatagaacatatccttctcctatatttcctatcctaattaacgtcctcagcatttgcccgaattctcaagtgagggatttcagggtcatccctaattttccttcttcaccctccacacagtagctgtcacttactgagtgttactttatgccaagtactgtgccaactgcttttacacacatatgcttcatttaattctcacagctccatgaggcttgcaccattatcattgccaatttgcagatgagaagccagggcttaaagaggttaaataagatcccacgcatgaccattaagaggagcgaacaggatccagctctgggggtgcctgagttcagagcctgcctttctgatttctcttaccaagctttgtctcctctccctcctaaatatctctcaactctgcctcttgcattccaggctctctgaggactagaggccttgtcatctctgcgccagcccattccaagggcttccttcctggaatccagggtccagcctctgttggcccaggcatttctctacactggcaccagagttacattccgcacacctgcttacgttgctctctcacttaaaatcttaatgactcgacccccaaataacacaggtcccttccaaatctgtcctaccccaccttcccagcccttgctcaactctgctacctggccctttcacgcctacaggcattcccattccatgacctcttgggattctaccctttgcaaatgctgttttcattgcccatttattagagcgcttttggtcacaagctttttgcttaaccaaaaagaaagcatttattggtggacataaataatgaagttcaggaggatccaagagttggaagccaccatgagacccctgtgtccttccacctcacttttctactcgcctctgctcagcttcatctctggccaggccctctcctctgctgatgccctagctgcttacagcccttagcagtcatctatacaccaaaaatccctttcccatagcagaagcaatgctcctagagagtttccctgttggtctggctcctgtacccacccctgtgtactctgattgggaggcctgggtcagctgcccaccatggggccatttctatgagcaggattactgtgaagtggaggaagatgtttccccaaaagaagaaacacaaggtagaaaagtgtatgtccaccaatgcctgaaatgactgtccctttcctcatctgctgagcttctactcattcattctttgagactcagcactcagctcttaaatgtcacttctgctttgatggaggtttagtcattcactcctctgtgcttcctggccctctcttcacacctctctcagacccctctcccagatagattagagttggctgttgacatgtccatctctggctgggcagctaaactggagttatttagaatcagggagcacatgtcagtcattttcaaattctcaacctcatactcccagtaaatgactccatctaagggtggaccactcttgcccatgggccaggtctgggtctgtgtcatctagaactgttggaaggtaggggcttctgtgagcagtaggagagggaataaactcgagggccctcgggagcatgccctcttgtctcagacttgtgagtcctgaggataacaaactagtgaagaaaagcctcgttctatctgtcacctggtgctcttgaggactttctgttgccctggtgccaccacaattttccagagtgtgtgaccctcgctctccaaactctggaagtggcagccgaggctccccagtggcctttcagaaggtgccagtcatgacagcagcaccaaactgcaggcaactactaagcgatcaccaacttgtctgaagataagaatgaccttgaatgcattttataaaacaggattttttttttaatttttagattttctttctttattttaccttaagttctgggatacaagtgcagaatgtgtaggtttgttacataggtatatgtgtgccatggtggtttgctgcacttgtcaacccatcatctaggttttaagccccacatgcattagctatttgtcctaatgctctccctcgcctcgcccctaccccaccccaacaggctccggtgtgtgatgttcccctccctgtgtccatgtgttctcattgttcagcttccacttacaagtgagaacatgtggtgtttagttttctgttcctgtgttagtttgctgaggatgatggcttccagcttcttccatgtccctgcaaaggacatgatctcattcctttttatggctgcatagtattctatggtgtatatgtaccatattttccttatccagcctatcactgatgggcatttggattggttccatgtctttgcaattgtaaacatacatgtgcatgtatttttatagtagaatgatttatattcctttggttatatacccagtaatgggattgcctggtcaaattgtatttctggttctagatccttgaggaatcacactatcttccacaatggttgaactaatttacattcccaccaacagtgtaaaagccttcctatttctcaacagcctcaccagcatctattgtttcttgacattttaataatcaccattctgactggcatgagatgatagatacccatttgtcagatgggtagattacaaaaattttctctcattctgcaggttgcctgttcacgctaatgatagtttcttttgctgtgcagaagctctttagcctaattagatccatttttcaattttggcttttgttgcaattgcttttggtgttttagtcatgaagtctttgcccatgcgtatgtcctgagtggtattgcctaggctttcttctagttttcatgattttagattttacatttaagtctttaatccagcttgagttaatttttgtataaggtgtaaggaagggatccagtttaagttttctacatatggctagccagttttcccaacaccatttattaaatagggaatcctttccccattgcttgtgtgtgtcaggtttggcaaagatcaggtggttgtagatgtgtggtgctatttctgaagcctctgttctgttccattggtctatgtgtctgtttacaaaacagattcttaagcatcaacccagatcgactggctcagaatttccagggaagaggcctggttatctgcatgtttacagacctattagatttgtgggacctgcagttcccttgtacagttagttactcaattaacatctccctcctctcatggtgcctctacctgctaagcccttattcccagccaggcccaccaccatccacccactgctgttataacataagcaggacctgtgcgagggggtgtggacggaggagagaggctctgttgcttcatttgtgcagcatggagttcagtggttctcacaatgtttttgcaaagtatataaagaatactccttgtctacttgacattcgtatcgtgacataaatgtcttgttttccagaaggattattttttccaagcagcttgttcctaatgcagccccaggcaccaaacagatacttaaaatatattaattgcttaaatggttaagaattcagtctctggacccacactgcctgggttcaaattcctattatctgtgcccagtttccaagtctataaaatagggatattaatagcacttacctaataggctcgttatgagaattaaatgagctaattcatgcaaagcactgacatatagtaagcacttaataaatattagctttttaacaaaatacaagccaaaaaacactgcttaggagaggaaatgatgttagtgcctcctgtaaataggcccagcctccaagctggtgctcctctaggaatcacaacgctgcaaatcacatcctccggggccgccaggacttcacgagggcctctgagcagaggggtatgatgggagcagaagcccagcagctgtgatgatgtggtttctgatcttcctgcccttggggtgggggaggaggaaagcaaggggcaatgaacagaaaggagaagatagcggggaggaaatgtgtgaggaagaaacacatcactgtggcttgtcctggatttttctgcttctgttctcgtgttttgggaagtctggaggagacttgaaaatcattcatgtccccaccctgaggatggcttagtagcagagaggccatgaaaactctttgctgatggctctgaaagcaaggatgttgcttcactgggctgctgaaggcctgcctgggggttctgagcagagagtacaggcccctcccaggagggcggcctaaccaccatgctggcatttctgtggaccatggtctgctgtctcagaccccctccacaatagggtctgcaatctcattcaccccataaatacattctgtctttcctctgatcccctcccattagcagggggaaataaatggaagtcagacggcccagttagaaggcaggcagtggagtaggaaaatagatgatggtggtttggggagcctcacatcactcatggggagacattcattcccatgggccttccaatcacccttttctccaaatctaaggacacaggacaaatgggtcctcatacaggcaaatatcttaaactggtatgtgtattcatttatagttctaatttatatgtgtctttattcacatatattttgcttctggagaaaagctcaattagaaaaattaatacattattcttcttattgcccttcagctaaaacaagcatacacacccctcccctttggattttttgtttagcaaaaggttaggcctggcacagatgaaatactattcagagttcacagtgtattttcatttcataatatatttgattttcaggtcttgaatttcacatcaggaagctgatataggaagctgaattcagccagattttaatacgaaaatacctctgatcaaggcataaaattgtactttaaccagtaaccactgtatttctctaagctgtgaaaaaacatgcattcattaactgctttttcctctgctgtcaacacagtcaatacatgtgcataactccttattgtctacatggtgattatcttgctgatgaattctcaaaggccagagatttggactattttttctctgtaaccttgcatgttcctggccacatgccaccaccacccaaacagaatgtacgcagggaatgtatttttcaggataacctaagaaaaaataggattaagaagataaagctgctgatcatgtaatgtactttagactcagatatataaatatttgtgaattatctgtcctatttctttcttctattaattcattgactctagatgtgcattggaaggctagggagaaatcaggggatcgtgagaaagagcacagaagtctgcatcacacaaacaatattatttcaagagccatgaactagatcctaagcaactcataggcaatgacctcatttcatacctctagtctctaagaaacatataactggcctgaggaaggaaaatgtgggcaaggggtagaccggggtcatgggtggaggtccaaatagtaatcaatggagctcatagggtggactgatattgaagctgctatgagccagccacatgctgggcactgttacatgtcatctcatgcaatactcccaattacctgcctagtaagcataattgtcattttatagaattaaaaacagactcaaagaggttgacagtctaatgtaacacaacagctaaatgggggatctggaattataatccagagctgcctggctctgatgagaaagctctttctgctgtcatatgcagcccacattaatagggggctcagaaagtattctctggataaattatataatgaatccaatgaaggaagacattattttataatatgcagcataataggcactattatgattggattttcctgcttgaaagtagctagattagagtaggaaaccaaaaagatgtgaattcattcagtcattcatgcatttgcatggattgagctacctacatttgaataaatgctgttaatccctgattccttggaagctcacattggagagataagcatgtcattaaataatgccataatagtggtatctcagaggactagcagaacataattcaatctgacagagtagaaacagattgtacaaatccaattcaaaacatcataaatcctctaagcactgtcaattcttcctccaaattatctctgaaattcctccttctttcccatttatggcctccatttacagaagcgtgtactgtctctcttagctgtttgccaggccgccagtctcttgctgttcagctctcaactgcttccagcaagatctttctaaaatcccaggcttgccaagacttagcgcccacagctccacagtgactcctcattgctgttaaggtaaaggccttcccagtctagcccttcatgcttcttccatgttctatgggactgccccaggcttcccacctggtaccactgagcctttccatccttcccccactcgactgccaggtcaacacccacacccacgcttcaggactcaggtcctatgtttcgggccttcttctgtgcaccattcccttccctgtagcccttgatcatgatttgtttatacgcctccgcaccttcatggccctgaacccctcaagggccgaaactgccttacttttctttttgacttcccaacttaccttagtggagctgtagtcacatagaatagacgctcataaatgcttctctgggctgtaaaggttgaattttccagctaagcaaggaagaaagacaatttcaggcaggaggaagggcataagcaaagtgcagagatgtgaagctcaagagaaatggatgggctgggcagaggtgtggctgcagcatcaggggagaagaagtagtgcctggagtcagcaggcacggcttgcaaaagcttcacctataggtgaaaggacaccatctcttgcaccaataggctctgtgattggaggcaactttgctgttttactgccagaaaactgaggatgataacccaaactgcagttcaagtggcattcactggtgtggctgaaatgggtgtttgtggccagaatgtggtctgattggtcagtgcccagctctgttgattagcagatgttttgaatatagtagcatccatgtgcccaagttgttgggatgattcaacaagaaactttaagagctcaagtgccctgcagttgtcagccaggtgattctcttcctttggacccagttagacgcaggcattacctcgtggctttgccccagtgtgaatctttgtcctccaacttgatctttttatttgtttcattattgtatttaagttgtttattttagagacagacattttttaacagctgtgcatttcctgtccctttgttttccagtcgtcatgtgtttccttactctctgtgggtgaacgtttcagatgtctgtttgcggtgcccagcgtgcaagataaaatttattgcagtgccttcggcctctaactcaccattccaaccaattcagatagcccaaggctgttttatccagtggatttttccatgtagtgggaaataaatcttgaatgttactgtttagattagccaggaaactcattctgggatgtttgcccacatccattggcatttctcaaaaggaaccccaggtgtctaccttgacaccagcagggccacttgagccctccgctggcattcatcgcccgctttgttctcagcctgagtttaggagttacagatgtgagaggcgggattatacagccaacatctctaagcgggcagtggctcccttaccctcgaagacctcactcctagcacgtcctggatgtattcgtcaaaatatgtcctcttatgccacgtcagcacagggttgctccccactttgatcatcaagtttaaacaaaaggaaagattttctttctttctctgcctctactggacatcatttcccacctaacagataatttaatgtatctgttactgaatgtgtttgaattacagacagagaggtcacagttaaagaaggaagcctgctgctactgcagcttgtcctcccaaggaggtgtttgatttagctgtgtaaacaaatgactgcattctccagaggtcctgaacacagctgcctgcgctggagagggctcaaacctcttccgccagggtgaactctgcttcctggtgagtgccagcaaaacaaccaacaaagagctgtaggacttgtgtggacttcaaatggtggtggtcctgccacttgggctcagccacagcagttaggaaactaaaggggaggaggaaagccctttccttgctttattgtcattggctgtcatagggcattacaatggttctctttgagattctgagctccggctataacatttgcccagaatctgcctctgaggccttaagacactgtgtttttattcagcaaagatgccctttgactccttttcccactagtggtgctaggtttgagcaccttacactggccccttacaatagccagttcttgtctacctacattcttccctaacattcatgattgcatagttactcttagtgtagaagcagacagcttttacacatagactccatggccgtagcctcatagaacctactatattctaacttgcaagctaatcagaccaaatatatcaaaatcaaaaacctctgctgagagtttattcattcatctctgtctcccaaacgtacttatgtacatacgtgcactaatatacatgtccattagccaagattttgatttcagggatcaaagcaagtaccaatagggaatgaggtcacttgctgcatggcaggtggcttccccatgagaatgcaaggccacctcatgactcatacttcagagggtgacccaggaacttctgattcatgtccaaagcagcttctacaattgctctaccttgatctagggaagatgtggggaggatgacattcgggattagctttataaggccttcctgtgggcagagttgtctgactttcacctagtgatcaacaagcagctagcaagcatcagtgtgtgaggccccacgccctctcagctcccctactgcccacctgggacatgggctttggcatctgtccatagcattgttctaaccaaatgaggtgttatggatcagctcaggatgggatatgttcccagacatattatttaaagaaaatagctcccttcctcccctgataaacagctgccatggctaaaaggtaacctggctggggcttaaaagtctgttgactttcaagatattttgcaaaaacagtcataaaaatggtatttatcagatcctaactatttgtgagacggtttggtataccatagtggttaaaaacacaggctctttccagaggaggtttactttgcttagtcgtgtctcctaagtgaacttggacctcataaggttgttgtgagaatgaaatgggtgaatatgagtaaagtccttggaccagttttggccgtatagtaagccttcagcaagcatctgcttttattcctacagggaggcaattgtaagcccttcacaaacagcgtctaatgtgatccttagaacaaacctatgagatagggcatatctcaattttgtaggtagggaaacagaagccacacaattaggaaatggcaacagatctgttagactcttaaacactatgctacaccaatttgcaaggcaaggaagacaaagcacctttgaaaatgggtcagatgttttagggtaaatgaacgtttgagaatcttttaagttttttttcccccagagattatcaaggtatcattgtagggggatgcatcaggaaacatgactatgaatcagctgcctgataaaccagccaggatggagcccacgtcatcacagcagtcagcaatgccactgaaaaacatcagctgcttattcccgtatagatttccccttaagacatgaaaagggagttcaaagagaatgggccagatatctctgagagtcatattactaaaatatatttatttttactagcttttttgttttaagaggtatactgtcattagcactgtagcaaaaattcacgttttattaatttctcctagtttatcatgtgattctagggtaggatgcagagttatattcaaaatacacaaatcaactcaactcagtaaacatatatcgaggccctatcatgacaaaatgctattctagagaccacggcgaacaagccacggccccagcctcaaagaatgtactatctttggaactgtgctggccaatacagtaaccagcagccacgcagggctatttaaatttaaattaattaaaagtaaaaacacaatgcctcagatgcattagccacattttaagtgttcaatagatatttgtggctcctgcctgccatattggacagggcagatatagaacaattccatcactgcagaaagttctactgaacaatgctgctctggagcagaagatcttcttgttcagggatgttacacccccgcttgtggctagagtgtggcttatcctcagagcaaggataggggaaccatggcactctgcaggctcagcactgaagacacggatgcaggctctgcttctgacctagattgaccttgggcaaggccctttgctcctctgatcccaatttcttcaccagccaagtaagaacatcagaccacaagccctctagggctctgtccaaatgccccatgactgagtgaactggtagaacattctatgtgtgtgtcacaacatgaagagcaaagactttcatctccccaaataattttgtttttcgttttaggaattaaatttcagattcactctaattgccaatactaaaattctctatatgcagttctaaacttgacaaaccaataaaaaaagattatttgactacttatctttgtacaacattgaggtctccctaaagcaaatttaaatgcatattttaaaaatgtattctagcagttcagttcagaagccccctggcccaagcatcacactgtcaatcctttgtcctcaagcagcatggttgggtgggttaagtactgacaaacactgggtgtcaggcccatggtcagggactgtgctaacagtctacatattagatgccacctacccccaccctcaacagacccaaactatttatccaatagcaaaccttgcattatttctgtccagaagaaacaaacatttattgacaacttttggtgtgtgacctgtttaagtcctacatctcatttaaggactggtcaatgttaggctaggcaatgcctgtttgtgagagaatcactgcctaaagaaaattctccatttcccttagctctatggtgggtgactacacatactggtatttcttaaagaaataccaattccatttccttttaacataattattaatatctcattagcatggtgtcactgaagcctgggcccaaagaaataccaattccatatcattttaagatcattattaatatctcatcagcgtggtgtcacttaagcctgggccctttagaatttttcatgtacctgtgttcctctgcccatatcagctggaacactaatagttttcttcctttttatctagaagactgagaacattacatgggacctgcccccagggcatggaggctgaggtgggacagtttagttcaggaggcccaagaagtgttgggtgtgcagccccttgttcaaacacagcctctgaatcgccagaggcttccggtgcatactctgaggcgcaggtgggactcgggagtgagaggtttcggcgaatgaattgggattgcctacttcttcccagtgcagtggagcttggttctgtggtcaggtccttacgccctgtctgcctttctcgtttctttatttctcgggtagtagttgtggaatcaaatgacctggggtttgatacctactctaccacgcctctgggggagtcactcagactcgttgaacctaagttccggggctgccaagtgaggataagtagtaattgctgatccacctacttgacaagatagtagtgagggccctgagcgccaggctgtggatccagcctttcccacggttcctggtgtggcaggaagaactctaggcctgaaggtgaaattggggagggagtcccagctctgccactgtctctctgggtgacctcaggcaggtctcctcaaaaaaataagatactttataaagctcagtttcctcttcagtaaaatgaggattccaggtaactcacagatagtttgtggggatgaatctgttccttaaagcctgcagtacatcaataacccagtcttcctgcttgctttcccccctctccactaccagtgatcatagtctgatcccataggtgatatcccagctcaaaaccctacattagcttctgtggctgtttaaggcctgcccagaactcccctggtcttagcactgaaagcacgtgtccggggaagccctgcattggtcgttcatactactgagtcccgcagggcaaaccgtccggtcccaccctcctttctagtgctgctgtcacactcacctcccttcaccctacactcccttctgtgccttgcaattacctagggagttttttacaagatatggatgccctggccctgccactagagattctgatttaattgcttggggtagggcctggcataggtatcttttaaagctccgcagtggttctaaagcacagccacagatgggaaccactgatctattcttgtaggtccccagatacctcatgtgctgttccctgtgcctgagctgacctttcccccactttcctctcctcggctaattcctgcttatcctcctactcaggaggctcttcctccaggcagccttccctgatccctccaggaagacttagctgcgtccctccgctgggcttccccaatacactgggcttgctttcattagaacctgatccttccacattatggttgttggtttgctccaatcctctccctcattagctctcaactttctttcaggaagagatgtttatctttccttcttgtattcctagagtcgaccaggctctggcacattgcagattctcagtatgcattcagggaacaacttaatcaagacaagaccatctgacttcttgtgagttacatgctaagaaagaaatgtcgacaccaatagccctcacaatgataggaacaggaggttaaagaaaaggaaatagatgcaaatagcaatataagtgctttaacaaatctatacaggaggacaaccatcatattcaaattttcaaacattcttagttctgctcttttgtgggtaatggtttttttttttcctcttccaggagaagaaaagaggcatattatagaaattcctcctcccccagcattacttgtcacagaattgtaattggaagtgatttccctgactaagttattttggctgtctgttattttctctcttcctccttgctcttccctcagctggccatcctgtgtgtttggagagagccagaaaggttcaaggctaggaatgtttctctctctctttaaagctctttaatcgtcaggctttctgatcttcaaagcaggctgtagccagtgtgaccccactccctcgcctccccatgctggagagtaaaagcctggagtatttttgtcattttgaagacttgcatatttggacagccttggacatctggaaagtgtggtcctcactagctctgcagggataagagcacgtcagcacttccaagctctctggcgcccctacatctggacacgttgaaaaattaacaccagactctggagttaagcaaacattaagtttataggcctccttgcatttgaccatttcctgggacagcagcccttatcctgtgactttctgtgtgtagagttgagtctttgcagttggtcctcctcacactctctcaactttgtgactctctgcagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtgatgtttcaggaagggctcgctgcatttctccaaagtcagtgggaaattacatttggtagagagaaagggattgagactggactcataaatcaataaaattaagttaaataagaaaaaataagatattttataaagctcaacaaagagtccttgaatgaaagcaattacagagtcacattgtggctaatattcaaaactgagatttaaactgaggactaggaaatagaattggatccttttgaagcgtttaggagaaagattttaagagaatgagttccgagtcaccctgtggtcgggaggtgtgagtgagctatccaagcccgttcccatcctttgtccctctgtgtcttctcaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattcgtgagtctgaagttcgcgatcctcctccatgacacgctaatgggggtgctggagtgggctggggtgggctgggggtgccctcaaggcttccatgtctttagagagagccccagggaccagagccaaattggagagcatggagctctgactgaggaacctgcttctcccaagctccaggcaggcacagatgagtcagtgcagtggtgggaaagggaaaagagttgatgttgtagctggaaaagggaaggggaaaattaaagcaaggaaagtgaggctgggggaggggacaaattccccactatgtagtatgtttggtatgtggaagggttctggtcagaatgtttgcccaatgattgccacatcagcattcattttggactctgtatggccagtaggtctggttcctgggagccctggaataatgcagccccttccctaactaacatttccatgatgtatgctcaatgacaaggcagaggaatgtgttggatgagctcaggacctgcctccctggacactcccatcccaggcctgtatatctgttgaccaggaataagccaagcaagcagcctactgtttgactgaatatggatttggggggtggtagagaaagggccggggtggagggttgggaggctcatttgtcattatagatggggtcagacacactaccaaaacagcagcagagatctacaattgagttcacctaaaactcagtgtggacacaggaaaccctcttttaataactgtccaatgggttttccagcctcagctctacagaaaacttgagataacagtggccagtctgcagttagtttgggttcggacaataggcagagctgggaaatggagccaggggcgaaagcccaggtccactttaggatcaggacgggagtggctggtggggaagtgaggtgggtgtggggaggcaatagggagctgggtcatttggtatgggagagtcctctggtggctagtcccagaagtgcatgctttacgaacatatgcttctctccctagggccaccttgagtgaaaccctcccatgctggaattgggccctttcagtgacaacacacaacagttttcaatagataataatcccaagggctttactagcacatgaaacacagggaaaacgtgtaaagttcacaagaaagtcgttccagtgtatcaaatctatcctgtttgccaggtggatataccagggtctcctccacctgtgcatggctggtggtgggtccagtggctgttggataactgatgtattgatggatcattcgccttctgaaagtgccaaactgattagttattttgtgtgtctttttgtgtaactagggtttgaccttccagggcagactgtgctggggcggctgaccccttggggagccaagttattgctcttaccaccaccacttgcccttgtcagtcctccaccctcttgggtttcagtgtcagcatgtagctgtctactcagatcccatccacatcatcaagtctgcagttttttccttgcaaggccttacagggaagatctttgacatagaggatataattttattgacacattttacttgcagagcattcacccgggctaaccagaaagccagcactctgctataaacaaaaaataatgcttcagggctaacatggaatgtgttaaaagattccagcccattaaatgtccaggggaggttttcctgttttcctttccctccatctgggctttgttctcaacacattcattcaacaaacatttattctgcctctaccaggtacagagcactctactattctgcttctctccttttgctttagtttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatggtaagccaaatggagaaggcccagaaaatcttgaatactttggttcctttcccctttcctcctgttcatgtgcctggattagtcatgtggccaccaaggagagcgtgacatctagcttcccagcccttccttttagccaacgtgggagacactcaaagagacgaaatctcctgaaggagccactgtatcacagcatcctcccatctcccacttcctgcccaggggtccatggtccacacagacttcccagtcccattccgtgaccatctggagaagctgctattagcagagccctgcacagggtgatagtgtaattaaagtggtcttctctttccaaacacagaaaaaatcagttcagggagtgttttcctgggcttacaattttaactactggctagagttgaaatggggaaagccttttgccttttcagtagcagtaggggaggagatctggattatttacttatcatcatcatggtcacctcctacatggcttcaccaaaaaacattctgctgcctgaaaaagctccaacacctctctctcttttaaaggatggaatttggagtccatccttcctcagtgataaggagtttttatagccacaggcagcatctattggtctgtcctctgcaaacttgcaactcctctgagagctagacttggaaatgaaacattattttgcaatgcgctgctatccttcatttttagctcctccaccgtagatgatagtttgtacttgttaaatgataaggatataaatttaggtcattttttatattttattgggtggaatttggtataatttttagacttcaggctttacaggctcctgagatggactgattgagcttgttctacttcttccccatcatgataggaagtgctgtaccacactaggcagtgtgtgtagtgaccacagactggctgagtgtctcccatcccatgctggcccatatctggtacccacctgatccacaaatgttccatcagatcctgttcaaacaacacatctccagttaagccaaatcttgccctttctccttacggtaaaatgtactaaatctgaaggttttgtctttttaatgttgctccatgatccagtgatctgtggccttggttatgctctgtgctagagtcctaacaagacaaatgctaaggtagaggtcattctgctcaaacaacctgaccccacctggatgtgggcttacatttgcaaagggcaccaaagttctaagagatgaggggaggagctgagccccttgtccttatctaggtttcccttgttctttcccatccctcagtctgcttcttttcccagtaccaacatgtttgtgtcctcagaattaaaggagtaaaaatgtgtaaacatctgactagcaacagccatgagattttgcctggcttgttgataagcagcattgagatctgccctcctaagaatgggccattaggtcttcaaagcttttacgatgtgaggtaaagaatgttcaccaggagtttcatgcacaaaagggtttctctttgtgggaactagaacattgttccagtgatgacggaaacagggctttccataccaaaacagggttttcctttgaatgactctcccacctttcccttgtctcttcctccccacctcaacaacacaggaaagaagctggaagcagggacaatgggaaggtccctttgttactcgagctattagaaacaaaaagaaaagtggccatctgaggaagccacagctggtgaaactgtagggtcacagagtgaattacacctctggcttaagtcagtgaaaagtcctagaagtttgtggtcctagaagtcctaaaagtttatgggactttgttttgagcaaggataagaaattgatttcaggctgggcgtggtggctcacgcctgtaaccctaatactttgggagacagaggcaggtggatcacttcaggtcaggagttccagagcagtctggccaacatggcgaaaccctgcctctcctaaaaatacaaaaattagccaggtgcggtggcacatgcctgtagtcccggctactcaggagactgagcaaggagaatcccttgaacccaggaggtggaggtctcagtgagctgatatcatatcactgcactctagcctgggcaacagagcaagactctgtctaaaaaaataaataaataaaaaagaaattgatttcattcttctgagaactgcaacaactaccttaaagtgattccatccaaaacccacatgttcagccatggacttgcttttatggagctgcgtgtgggtgacacacaaaatcaggagctctgagtcctaatttagacttttatttagatttcctcaaatttgggttccagttaagcgtgggtctcttctgtgccccgctcccctttgccatttgttttatctgttcttcagtctgttctgtcagtacccacaggcaggagagcagaaaggagaaatggcagccacagcagacaaatggcacattcgttccactcagctctcgcatgcccatcacagatacagctcattggtctcttttctatgagaggaagccagagctccagggaactactgccaactgatcagaactcatttaggacatggacctatttgttcctttatgttcctgggaagagcacaggatgaattctatgtactcatttacgtgttcagagagtaaagtgcctcataggatgcctccagcaaaagataaccaagaaggtctaatacctttgacaatctcagtttatcctatagtgtaattggatagcagttcccctagcaaaagttgctagtttggtcctattttctacatagccaaagtgattgattcattggttaatgtgaaagttactgagtactgccagcaggttctaggaaatatatttgtgtgatattcatggatggggaggatcaatccacttccaagtgatttggattaattactggtattttcacctgtgtgggtagcaaacctcagaaaatcaagtatagatgacggcataggacaggccaggccccaggcaaaatgttgaagctcctctggagttccctcccatctccctcttttgttttccatatacctggtttatccagggccctggagatgctccaagaccccctacccaggtcttcctcccttgtcccagctatatttctccatattaccactcttctcaccgaggatttgcttacttaacacataataaatactattaaaagagaaacttaggcacattaaaatgttagagttgattccagcaaacagtgattcacaggaggctccagatcacaagtggttcagggccccactgaggggtagggaagcaagacaaagaaaaacaaagcaaatatttgattggttcaagtggaaagtccctgattacaggttagtgggcagtttgtgattagttaagtttctctaagttgggttttggtttgctgatgtaggaacacagaatgctggggccgtttcaacctaatggtctcccaattaatttttttaacattactgatgactgttaggagtctaatgtgctactcctcccagggaaaatggcattcctaggattaaaggaactcagcacatggagtgtgcgtagaaatttagacactaactgcaggctggtgggagagagccctttagggcagaatgagaaggcgtccggccaagggcaggagttactgacgcatggcctcttggtttcagcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtggtgaggcccttgggctggcccctgtcctacaacacgtttccttggaagggtccgtagcagtcctggaggcccagcctgccctctgagggggtccactttgcctttgacctaaggttaaaaagttcacgtgaggctaaaatgtacaggggcaaaagtgggagcagtcctcaccccgagcgatgcaacagtgactcctcaccacgcctgcttgattcatctgccctggaaagtcattaaaaaaccagttcaactcatgggtccctttatttactcacaagagagagccagcagcccatttcactagttttcctttcctactctttgagaagaatcagaagggagggagcttgccactttactatctgtctaaagagatgtttccattaattaaaggtttttgttttgcttcaaaaaaacttgaattggagtatttccacaagtatctttaacatgctctaccaatgtttgcagaaagaagtgcagaaatgagactgtccacagagtcaggctcgctggccaggagaggactcccgaagctgacttctgatggcctgagaaacttcctagttcacaattcccagacccagacaaagagcactgtcttttctctaattgttttcaaatgggccatttccaccctctaatcagcctctggccctggagggtgcagttccccttgtcctccggagtctccctgtctctgtgctgtagagtcaagaagggacaaccacctgccctcactgggaaaagacagaaagtctgacttgttctcacgactcacacttattaggctccagaggtgtcagggcatctgcctttcatttcttaggttaaataagaaatcaattgctgccatttgtagtacccaattttctaaaatgatcacaatggataagtggcaagaaatccttatgactcatctgtgggcagagttgggctattttggtaatccttgagtaggcagatggaatttgaggccatcttcttgggtacatagatcactaggaagctataggtctagcaactgtggattagggctgggctgagaattgtttcatgttttttgtgactgtatagctagagactctcttgtttgcagagagacactctgaactccccctggccgtcaagggaaagactgccttcaccctcctgagctgaccttacactgagagacaatggggaccctcttttggccctcccctctacctcgagggcatctgggtgctgttgcattggataaaaggcactgctctttttctgtgccctctccgcctcactgcagagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggtaagcatcctcctctatagggtaaaggtaattgagttcttcagatccccagccctctccattcatctagtttaaatttcatttcttccaagctctttgtcagaaccagcatttgaagtttaaatctagaagttaaaaatccaccagcaaatcctactggctctacttgagaaacaaatccagaatctgatctcttgtcaccacctccaccacaaccttcccaatgccagtctcttccttccactaccacctcccatcagtccattctgcacactgtattcagggagatcctttcagaatcaaggtcatgtggtgtcagccctctctgtcaaatgcttgcactggcttttcctctctttcagagtaaaacccagtgtctcaaccctggcctccaagctgcttcattatccggcctccaactctcttcttcatcttacgattttccctactcctccatgttcctctgctccagccacgtcggcctccttactgactgtttaatacaccgagcgcatttcctcttcagggcctttccacctgctgttctcatgccagaagcacatttctctccccacaacctgcaacccgcccctcatatctgcaggcttgcttccttactttgttaaggtctctgttcaaatgtcccattatcacagggatctttccagactgaagagatctacataactatggctctgtaaacaacattcctccagggttcctgtccccttaccctactttattttggggaacattcttcaccatctgatacaatgatgtatcttatgcatgtatttactgactctctgcccttagtagaatatgagcccagagagcatgcatgtggtctattttgttaactgtgacagtcccagtgcccagaatagtgcctgacctttggtgggcactgaataaatatctaagtaatctgtagcatggaaaatcagcttctgaaaattggctgtttgcacggtcgtgtatttgcttggtagaaaatcaaattttccttcaaattagcattttctggtaactagagctgccccatcttcctctgagtggtctccaagtcagccaatagccttgtgctgtggcagccatgcctggctcttgatgctgtagccaaaagcaggcaggggatggtgaggctggtccagtccatggggagggacaaactcacagctctcagatcatctcagggcagcctttgttggcagaaataggtaggcagccaccctgaataggaggaaggcttctagactgggtcaggaggcctgggtttgcatcctagtggcaagcgtgcattcatttactagggctgccataacaaaataccactaactgggcagcttagacaacagccatttatatctcacagctctgaaggctggaagtccaaaatcaaggtgttggcagggccatgctccctctgaaacctgtaggtgcttgggcactccttgacttgtagatgcttcctgctgatccttcgtctgcacatggcattctgcctgtcttacatggccatcttataaggataccaactggattggattaggtgcctaccttgctcccatgtgacctcatctcaactaatcacatctgcaatgaccctgttcctaaacaaggccacattatgaggtacctggggttagcactctggtatcttttttcttgacagcacttctgacaccaaatgtgtgttttggttttttgttgttgttgttttggcaccaaccaattctcctatattaatgggttgtccaagaattcaattgaattctgacactatccagaattcacacagactccacgggttcagtcccacaaggcttccccgtcttcagatgccagctggaaatgtggtgcccaggctacccacacttttgccaaaatcctgtacttacaatcacagctttaaaatgaaggatgcagctcaggaactgccacatggaagagaagcacagtatggggtcgggggaagagtttctatgctctctctagacgcaccactctcccagcacctcaaagtgttcagcaacccaaaagctctccaaatcttgttgttcgagagtttttataaccctatctccagctccatactcccccattggaggttgagggttgggactgaaagttccattcttcacatgtgtggtgtttctggtgaccagtccccagaaactgcagctatcttggggctctaccctgagtcacatcattagcataaactcagatgtggtagaggaaggggcttattatgaataaaaaaagacactcctttctgccaggaaattccaagggttttaggagatctgtgccctgcacaggagctggggacaaagaccaagtatattttgtgttatgccacagaccccaacatgtctttttggagggagaccaaattcaacccatgacagtgactttgaacaagacatttgaacttagtctgttttttctatcctactagattgttggaaacagatataatagatgaaaattagttgattaaaattgaaatttgtgcataattcaaaagttttattttagccaagctaaagctttcatttattcaacagctatttactgagcagcacctgtgcatgaggctcagcagggccaggttctgggaacagagcggtggagataaagatccagacctgccccgaggaatagacagtccagtggcagcaaaggccatgaaacatacggcaactcttaaaaaaagccgagaccatgattttacaaaatcaacattttgtagggagcagaactttcaaagagaactggactagaaatttgggagtctttttcttggaaccctggtagatccagtagaatgagggatgggggtgtagggttaaaaacactgacattagaactggattacctgtgttggaattcctacatttctgtttcactatctgtgacggggggcagatggctgaatctcagtgtgcctctgtttcctttctcacaagaataatattactacctatctcctggggttgttttgaggtttagattatttaacacatggaaagcactcacagcaatgcctgccacagaaagaatatccagtacatcttagtgatgatcaccattattattatctgactcctggaaaaggacttgatttaattctctcatgaaacgttttcttggaaaactgatgtcaaccaagattattggtcttgctgttgcttataacaccccaaaaacatgactgtgtggataaaaatatgttggaaggggtagtctttctgggagcctgagaatagccatgtaataataactgcaaatatctatagttacaatttgaggttcaggtaaataaactctagatcttatagaactgcggtaaggtaggatagggagactccttcgactttctctgtttatttgtctctatttttaggagactatggaaccccacttccttggtactttcttctacaagagtcgtattggcttggcggtgaaggtgagtcctttaaaacacaaatcttaatgtttgaaatcaactccttgggctctgtgcaagatgtatatggatcacagaggtggccctctatgtaaacggtgtgattcctgatgagtcagctgcctcctggggctctgccccttgatgggcattgcagcgtctgggggaccacctttcacaagttgctgggccctgtgtgatcatgaatggctgatcatggatgaagccctgggtcctgtacaccttgtccagtagactaaattgccctatttaaaaaaggccaagccacttcagggttcaaagaacttttgcagcttttcagtataaagcagaaatccagggaatcatgaaggaacctttgcattcatctcccattgccttccttgtgcctttttattcttctctgccttttcaaaatataaattagtttattctcccaagatgaagactcctcctggggctgaggcagagctgttatcttcagggcaatacctcagattctcctggtgttgatctttcttaggggtggggaaaaaggctgaaagggcatttgcccacaacacatcttaggtaaaaggcacctttactactgaaccaaacaggaggcctagctagagaaagttctagaagcagggaaaagcacagactcttttgtgaggtctgagaaagcaaagaaattccagggtgaaagcgggggactcccctagagctgaagtactctcccatctgtttgttgctcacctacctattctttactttgtattattgggcctgggccaggacttatcctgcaagcactgagatggatgtttgttttctctgggggattagtctttttttttctttttttcttttgttttttgcttttgttttcactgggtcaaacaaacaacactttaacagctcaggattttttcattgtattgacttgtctacctgtaaacttgttaattttttactataataaaattatcatataataaatgaaaaatttcaacacagggcttgtgggcattttatttttctctacaatcccaacagatactctgcctcttaagaaaaaaagaaatcataaggaaaatatgctccttcaaaagtgaatcacaaatatgtttgccaacggaaggcaaatatttttcacctgtctcataggctggactgaaatggatttctaaaactctctaaaaccagaaaagagctgagtgtctccacccaacctccctcctttcacagattaaaaaataaaaaatggagcccaggagacatccagtatcttcccctattggtcacctgggacaaaatctggaacatgcacatgcattgcctggcaggaactcattccagtgattaaactcttcaggaggatgtttcctcttgctatttcattacctatttgtgcagtttgatagctagtaaagtgatcaaaggaactgtggggcatagattcaaaagtccttcaggaagcagaaatagaagaacagtactagaggcagcaggtccctgaccagcaggcccactacctgctgctccagcacacatcctgcacattttcagagggtgggggacagaggggccctgggtggctgttgcattgagaaatctcgccctgctcctgtatgtgcacttgaggccgagagcccttggatgcctggtgacagtggtttcctcctgcccctgccttcctctctggcagactgactggcccttctgctcctcttccccttccaggatgtcctgatatctttttaaaccaaatgccaagtttgccaaaaagtgtctgtttgtgtgtgtgtgtgtgtgtgtgttcaatgcgtgtgtttataccacacttcacaatttgtccaggcttgtattaataccatcaccaggctcaaccctggtgttaattccaagatacttaaatgcccatctaggtgaatttctcaggtaaaccatatattcaagctgtagtttaagctggctgcccgtcatagcactttgaatagactttgtttttgtttttgttttttgagacagagtctcactctgtcggccaggctggagtgcagtggcactatctcggctcactgcaacctccgcctcccgggttcaagcgattctcctgcctcagcctcctaagtagctgggattacaggtgagcgccaccccacccggctaatttttgtatttttagtagatacggggtttcaccatgttggtcagactggtctcgaactcctgacctcatgatacgcctacattggcctcccaaagtgctgggattacaggcgtgagccaccacatccggcccctgaatagacttttactcaaggttcaccatgactttcacatgttttgtattggagtaaaatgtgccagtggtgggctaaagaaaattaactcatttcaaattcaaacctggttttcttaatttttttaaaatcacagtttctgaaactgtgggctcctcatggcacattgagaggaggaggtgaaactctccaagtctgaagctcctgttataaatcttcctctggcaaagattgtgtgatcaggcttgagtacctcacagtcctagagcaggtcaaaggctggctaggaaactcatttgctccctgtacctctcccctcctttcctgcctttgctcgttctcagctcccggtggtagagtaacactggcttctgattggtgcagggtgttcaaccagagaagaaagagccctggaaaagaccgagcccctaacagaggaaacggaggatccagagcacccagaaggaatacacggtaaaaccccgataaagaatacacagcagaggcgaggaaaaggctctaagcactgcagagggccagagcaaaacatctcatggcaagggtggaaagaagcctaggaaactgactctctctgtggacaagtgttaaaccagatcccttctcagaggtccatctgcatgtgtgtggaatgaatggttcagcccagacattagcgcatatttcctggagaaagcaaataccaactatgtagtgtgcctgtgcccttgttaggcaaatcccaagtgagttgcacaaatgtgctgacttccgaggatttagcaagaacaataactttggtcactgggacttaaagcggatatgagctataaggaaagacaaaaataaatgcttctgtgtccagggggaaagagactccaggggagctgactacacttcacttacggcttacaaatctagaaggccattcattgaaaccatcagaagcctttcctgacagtggaagttacctaataatccctaaactgacgacccagatttacaagttttgttttcctggcttttgctgccctcatcttctctcttaaactagttctgtatttctcccaaggcttttcattccctaagcatacgcatttctctgtggccaaaatgctctgggtttagacaggcagcacagcccctgggctctgcctgacagggcaggagagggtctggcctttatccctccagcccaccccaggggccatttcataaaactaaagccagagacctgcagcccctcccagagttagactgcagtacaccatgcctctggcaagatcctcctcccacagtggaaagtctaagccaaatcaggaggctggggactggttccacctcagttgcaggcaaggccaggaggcacggatagaagaaacagtggactttttccccctagggaaagaaatgcttagagctacagtattaagatgacaaattaagctgtgccatatagggtgaaatgaagcagggatagatgggaggtcagggagaagtgagagcactcggtgagggtctgcactggagggggcatgggaggaagaaggaggggagtggggtttgagggatggtgatgaggaagcgtggactgccctacccacctattggaaaacctgggagttctgaggagcaagaagccttagtcaaagtcaactcaaagattcaagccaaggtgactaagagaatggcggtccagaaaaggtcatgggagaatctgaaggcagatgttgttttgggaagatgaagaacctaagccgcttccagaaattcatgaggaaatgccccgtggactgttggcaatgagggcctaggaccaaggttgagcttggggccaactctccctatagacagtgagtgcattctgacaagcatgggctctgggttcaaatcccaactctgccactcatgcctatgtgtccttaataggacgcttgatgtctctgtgtctaaggtttcctggactatggaaatgagcctaataaatgtctaccccttaggaccattgtaagagtacattgaggtaatttgtgtaaagcagtcgaagcagtgcctggcatataggaggtgctgtataaacgtttgatgctagtattactattattattctggagtcttccttgcaacggtgatagccgaagccacaggggcaggtgacgttataggcagaatacaagggcctggagacagagccctggggccatgtaattaggcattatgtttacatcatgttcattttttttcctccaagactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtgagtcttccagcagagaagctggctgccatgctagcctgtcatttcctggcttagtctttccctatcagcggctgtctactctttcccacaaattttagtgacaaatatttgcggccccaaaaatgtgtaaaagctttctgcagtattcaaagatcactaatatgtattctcttgatggggaggtagaatacgtttattgccccttttgtgtgccggggaagtggacattcattcagagagttgaagtgactttcctgaagccaccaagttgtcatggctcagcgggggcaaaagccaggcaccacagttgcctcttgtttctcacaccttgagtctttccccccatctcaacagtccatggtggtgatcaagtcatggccactgtcatcatgtgcatggaagctatagagtcctcctatttcctttctcttttcttttctttttttttttttttttttttgagatagtaaccattacccatgctggagggcagtggtgcgatcttggctcactgcaacctccgcctcccaggatcaagcgattctcccacctcagcctcccaagtaggtgggactacaggtgcataccaccatgcccagctaatttttgtattttttttttttttttttttttttagtacagacagggtttcaccatgttggccaggctggtctcgaactcctgacctcaggtgatctgcccgcctcagcttcccaaagtgctgggattacaggcgtgagcgaccgcaccaggccgagtcctgctattttcaaggaacattccttttcctaccaatcattaggcaggcttcaacatcagctgatgagggttagtggtcgttctggagaaagtgaaaaaagaatcagtctctagaggggcttgtggagtaaccgcctggtaacagaaggtcagggcagggaaggcaaaggggctctgcgcggatctctcagctccgcaggcgccccactctcctccaagggacccgagcgccatctgctgagaggagaacacggcccgccatggtttcccaaggagcagcagacacggacctcgcagggggcagcgaacccacgtgacacagtcttcaagtcctttggagagccccaggaaggaacaacagcgtgtacaccctgtgatggaatgttctctagggcggttcagtgtgaatggaatgtggggccggtgccattctaattggttctgtttccctctagtggttgatcgcggagatttcggcttctccatcaggacaagttcagatagcctgagatggtatcagaactcagggacagagctgggtgtggcggccctgcatccatctgctttctctccatgctaactgatatggtcagagagctggaagcaaattccaggaccccagggctccgcaaaggcaaacacattacttcatcggctgctgacatgcaacttcccccaggggttaaaacaatgtttaatactaacagtaataatatttttgagttttactttatgctggcgctgttctaatgttgtaagtgtattaactcatttaagccttacaacaacctaaggacatgggagtcatagttcccatttaaaaaaaaaaaaaaaaaaagcccaccattgctctgaggctttttatgttttggatccaaagctaatattggtggtggtaattcccatgcctggcttcgatcaattaatcagcaaatgcctaggactgcttagggttctggccttcatcaagaccttacccgggctttatgatgatgacacctggcttttcaatagccatgactgctcacccaggaggcaacgcctcgagtcatgcaccgaacaccttttattgatcctctccaacaccaggctccgtgatggctgagctggggacacctgtgactgcacgtgaacattttgaggctgggaatcccaaaggccctcggcgttggcctgggagcaccatgaaacaagtagaagcagagaaggatggcagaggtggccctctgcattagggcctggatgtatacactggtgctaagggggccccacagctaataggggtttgagtttgactgacagccccaggcaggaatctgtgagagttctcactgaacctggtgtgggggtggccctcctaaggcatgttgctaaaggccatctcttctgccactgacgcctgtgttctgcaggtccatcctgacgggtctgttgccaccaacctctgggactgtgctcgttgggggaagggacattgaaaccagcctggatgcagtccggcagagccttggcatgtgtccacagcacaacatcctgttccaccagtaagcgacacaggaactgagaccgccccatcccctctcctcacctctgcccccagcacacttctctagagcccagctcaggggtgccaggcctgggcacaggcagagatacagactcttatttggtttcccctatgtttaaagtcctttgtcctacttgcagtgagaattgtccctgagaatatgggactctgcctctgctgctcagagctgagggctcctccctcagaagggtgaggctgccttcgctctgacagagcagctgatcgatccccgagccccttgtgcagccctgaagtacttcctctctgggaccaaagacaggagaaccattgttcctttttcctgttgaagccacggcctgaaaggcaaacttttcagggggcttttcagttactttttttccccaataagatatcttttatttcttatctaagaagctacgcatagtcattgtgaaagaaaaaaaaggaagggaggaaggaagggaggaaggaaggaaggaaggaaggaaggaaagaagggagggagggaggggagaaggaagcgagggagggagggaggggagaaggaagggaacaggagggaggaaaagggaaggggaaggaggaaggaaagggaaggagggaggaagtaaatataggtaaacaaaaaattgaaaataaaagtcacctgtaatttcactactcagagataaccgctgagttataacattggtatataattttttagaactttctcctatacatgtatagatagataaacacatatacttcaaaatgataaagaatagtaaaactatgcatacaattttataacctgacttttttttcaaaaaaaaggattgcttttttaaacataagatatcaggaacatctttcatgtcattacatattcttctataaaataatatttaatgtttacagattattccattgtatgcatgaactatgtaagccatcctcttattagatatttaagcagggtctgctatttttgtattgtatcataaacaccaccacagtgagcatcttgattgccaaatcaagaatacttgtcctcaattatttctgtaagatcagctgctggaagtggaagtgctaagccactgcttttctcgttgtcccatcctcctagcctcacggtggctgagcacatgctgttctatgcccagctgaaaggaaagtcccaggaggaggcccagctggagatggaagccatgttggaggacacaggcctccaccacaagcggaatgaagaggctcaggacctatcaggtgctcagagctggatggagacagggccacagatggcaaatccatggctccccagtgcacccaggaggcaggggaggcttggagcaggagagcttctaagggtgggaacacctctgtgaagttacaccaaaaatctaagagcagcccccagatcattttccctgcagagcactgtctcacagcagcctgggttttatttgtcctgagattgatgtgcttgaacagtcttcaaagggtctgatccgaggaggtgagggttgccctttctgcatttacaaagcctgaacagtattagggctttgaacgctataaacatctaagaggcagcaccaaaccactgctgggttaaggtacccccacaatgccacttgccctgggcctttctcttcctcaccctccacagccccttaactctcccgtccttcttgtgcctccaggtggcatgcagagaaagctgtcggttgccattgcctttgtgggagatgccaaggtggtgattctggacgaacccacctctggggtggacccttactcgagacgctcaatctgggatctgctcctgaagtatcgctcaggtaacagctgctgctcagtctcctgggctgggctctcactgcagccctagctgtggtccccactctctcacctgccattttgtagctgagtacaggaaccacaatgactacactcagaagggggtttatcagtgacttggtgaatctaagttccagctaaagcctcctgaggtttttacaaatataaacagagaatcactgatgatgcaacctacttcccaaaatattttagaaaattctcttgacctgcagcccttctgtctggaataatggatgctactctaggtgaatgtcttctctgaccatggggacccaggtcacctgcaaacatacctagaagctccatagctgtcagatgaccactcaggaccagtgtgagggtgacctgctgggcattcagtgctccagagggtggccacagatggaagtggctcctctgtcatggcacctctcagacaaggggctcagatcagaagagacagcaagcagagctgagtgcccatagaggtaacagcacggttcaaccccgtggtcaagccagagctttcccccttgctctactcacacagcgttgccccgtgcctttctctgagggtttgtcatcctgaaatcctcattgctattttctttctttcttttcttttttttttttttttttttttgagacagaatctcgctctgtcgcgcaggctggagtgcagtggcgcaatctccactcactgcaagctccgcctcctgggttcgagccattctcctgcctcagcctcctgagtagctgggactacaggtgcccgccaccacgcctagctaattgtttttgtatttttagtagagacggggtttcaccgtgttagccaggatggtctcgatctcccgacctcaggtgatcctcccgccttgtcctcccaaagtgctgggattacaggcatgagccaccgtgcccggcctgctgttttctgttaatgacatctccagttagtgagagtatgcacgtgtgtgttctttatgaagagtataaatccagagcttaatgatccagaaaatgtacatatgaaactccctagatgctgaccataatacatgagcccctaatatagagatttatttgaatcagatcctatgctggatacagagacactgtgtgtggcaatgctttacagtatgtaggaagctatgaaatgttagttattattgtcctaatatgctggaatttgctgctgaattagttcccttgggtttttttttttagttaactcctgatttttgcaactatatagccaggaaattgctgtacaccctttaccaacaatgcccaacccagggcaggcctggtgattgccctggcccctaccttgcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggtaggtgtctgcccagggaaggaccctggcctgggtgagaaggagcacacagcacggggctgccactccagacatggctactcacacaggctctcgccaccagaatcagtgtctttgttctgggaccatttgcagaagatttcgatgaacacattctgaagcctcctcctacagagatgctttagccaaaatgaaacaactagctttaaatggtctgcaagtattacatgccagattacacaccagtttggtgcggtttggtgcaacatagaagtgagtgtcttattctgtaaggttaggctgttttaagagcaattggttgagcttcatttcaacattaatattccctaattaaacctgaatttcagtggtaagtgaaaactaagaagaggcctccttgggtgctataacataaaaatgatgaaggcaaaaagtaccaaccagcagagaccacttcagcacatcaggagacccagttttatgtctgtgctgcgaagtgaacaaactgtgtcatcctaggcaaattatttaattcctccttttttttagtatttttttcttcttcacatggaacatgaagctaatgacctctgcttctatttcttagggatgtgaagataagtgagataaagtattataaatgtgctctgggcttcttaagaacaggcattgctcacattcaaatggtcatgattatgatatggcagcattatttatgcctctggtttaagtgtctggctgccgctggggtttcctatgtccatccacggggagggaggcacagaatgtctcccacaggcagaacctacagctgccacataattgatgacaagccaaagggacccttggaggttctgctcctctctgtgtgtgactcacacactctctaggataaaatcaagcgactacaccctcaaaatgctcagatgaattaacagattaaacagtgaagaaaaaaatgtgttgactacacttggcagtgagaaataaataaagcgggcggtgacagcagctggcatcagggagaggctgtcatggaagggatgtgcatcttgtcagtcatcccatccatctgttgcaggggacctgcagctgctcgtctaagggtttctccaccacgtgtccagcccacgtcgatgacctaactccagaacaagtcctggatggtaaggactggacgggccatacttgggttccgtctggcagccatctcccagtattgctgggtgtgtcctgttgtgatgcattttaatgggagcaaagagaacactgggtacttctgcaggtcacacagttgttcttttgctttgagcttctttctcctcttccttcttccttcattcccaaagggattttaaaagtcatgcacctaaaggccctctccctttaatgaggaatacactctgtgctcttacccttagtaagccatcattcctggggtccccctgccctggctccaggccacattccttagtgtctggggagagcttcttctacatgtgtgccgtggcgccctctagtggaagcatggtgatgcacggctcttccagtgaattcgtggagtcagagattgcacatgtggatggcaagtctggaaatagcatacacccctgttatactcctgattctcccctcagcttcccaatttcccagtgattctccctttaattaggatgcactgaagctctcaggggtgcccccatctccaaggagctgcagtggagaggctatcccctctctatgtgagagaatgtgtgagaagcgtattcccacacaggagcaaaactaaacttacgtactgatgcaggttaatgaatggggaaagtatctgcttatcaaagaaaaggcatatttttctatttagcacaaactttttcaaatgttaagaatttactaactgaaatctggtgaagcaagagaaccgggcaatatttgcgttgtctgatcattacaactggagggaacatgctcagagaggcatcatcactgttcatgcacctgccctctctttacactgagagaccctgtgatgaacagaaaacatctttttaggatgacatctctgggtctttctcctagcctgccttgctgtgggtacctatctccctgctctctgaaccttggtcaagaagtttatatttgttttaaattgatactaatatgttaagttactgtgatttgccaaaatcagattggaaacagggcctgcatggctgaatgattcttttttttaaattactttatttctaaataaaggttttctttgtatagaatcgggatgctgtgaatggtgggaaatgcactaaatagttatgccccaaataagaaagggaaaatcatttgaatccccagttagctccttgaaagtcttttcacttaaacacacccacataccacacacacactcacagacctccctcccagatgcccaaagccctgctgacctacagagctacttctggaaaggctgacacatgcctaagacacaattcctgggaatccagcagctttgggttcaatttccttcctaaaagaacaatgaatatgacccctggagagctattagggcagagctgcttccttaacgtaaaggactctccagcctccgtatgaagtcatctcagagctaaagacaatcaagtccaacttgcagatttgacataaagcaagacttccaatccggctaggcagaaggattttggttgaaaaccatgaaatcccttcatatggatcattttttaaacaacaaaaaaagaaaagaacctactgggtgtccacaactctgagagctgctttctgaagagtcatgttttgagtcctggaatccctctccctttgacctgcctctcaagacaatgtgcgagagaactctctcttcaagtgcatgcaagtgaggttttcacagttagatttttaattttaaagtaatacacatttgtacataaaattcaattctgactgtatacatgtgtcagataaacagttgatacctgacacttgttcacagtctatgatacgcaccgcatatcctaccctctcccccagcctctctccatggcttctcaaccccccctctgcatttcctgtgacctgaggattcagttttgtttgtggaggcaggtgcaatcccaagagaaactgtgcaatcttctgagaagttagagtaggcatgtgtgtgtgatttagggaaggtacttctcactcagcttggtcaccggttccaggtttgtgtcttgggcaagtcccccatagctggtgacagaccagaaaaatgaaaacaactttgacttagccctcaagttttcagtgaatgagaatgaaaaacaaccatgagtaagagatttcttaccgagatgatgtaaaggataataatagcagccagcactcacctatgtgccaggtatttctctaactgctttgtgtagtttgactcatccagtcctcaaaaacaacaatgaagtggataccagtattttccccttttcacagatgaggaaagtctaatgtgacccacccaacataacatagtttgaggggacagagcatttcgttgaacagaggaggaactggcacaggaaagttgcatgacccccccaccaacctccgcccccaggttgcacagctagctagtcgggaggactttgcttccgtttccctctgcctctcaatgatgatctcagggccaactaagctaaaagcagacttgatggagcatcagtcctctgaaagagtcactgccgagatacaaaatacctcttcttcaaaggggaagtggagagaagtaggaaatctgggtaacctcacagtcttccagtttctggaaaacagagctggcatcagtcttttttcttgtcctaggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagaggtaaagtagagattccagctggtttctgtcaagtgccagaagtggcggttctttgaaaaagtctaacattagagcaaagttttgtaaaagcaaaaagccatcgttccccacccaagcatagcaactatctttatttttggcatagttcccccatctctgcatgcatacaaattttatgtacttgtggttactgtgtgcttacgtttttgtatttatagaagatgatgttctcagatagagtcgtaatggattttcttcccattatgaagcaatacccaacaaaacagagcttgggttagatttttctgagaataagaatgactaaacaaaattctctctttttttcttcttgacagatttttctgaaggtcacggaggattctgattcaggacctctgtttgcgggtatggtgctggagccagtggcttgttcccttccttgcctccctcccaagttccatctcgaaagtctaaggggctgggcacagtggctcatgcctgtaatcccagcaatttgggaggccaaggcagatggaccacctgagttcgagaccagcctggccaacatggtgaaaccccatctgtactaaaaatacaaaaattagctaggtgtggtggcgcgcacctgtaattccagctactcgggaggctgaggcaggagaatcacttgaacctgggaggcagaggttgcagtgagcagagattgtgccactgcactgcagcctgagcgacaagagcaaaatccatctcaaaaaaaaaaaaaagtctaaggaaaaagtcatgaaacaacaaagcaggcaaatactcctccatagtatctgactccccagtagtaggcattttgcatcctagatggctttgagtgacaaaggaataacagactgagttaggtctagatggggacactttggatgaatgaggattcttacggaggtcaggttggtagcttcatccctcagctcctcatgctgtatccccagtctctcggcctgccatgtcatcatcctcatctcctcctgtcatctccaccaggcctctgatccatctctgtctgcatgagtgacagctggcagagtccttaatgtttatcaaatacaactcagacgtcagtctcctggcccctttgagatcaacataaaatcattttgaacccttatttagtggtctatgggctttgaaaacatggggaccaaaattcctgtggattctagaagtctctcttctacatgtgtcagcctgggcaccaactagctccttccatgaacttttatcaaacccacagccacacaaagcatgtgtgagtgtagcagagtttacagcagagggtggagggtggggagatagatgtgtggaagggttacctgccacacaaacagaaaccacttctgatagaacacgaggtgtccacccacactgtaaaatcctctcctggtacaggcaaagctttgcagcgattctcctttgctgcccctgggctcctaacacctcctaaaccaccagttacctccttctttccagtgtggcatatttcagtgttttcctgttggagtgtttcctttctatgtggattctggaatcagctcttaagataacttggttttcatctttcttcataatgatcccaaacatctatctactatgcctagaactaccaatggacacatataccagcccagatatgcttcagcccatcccagtacatcgcatggtgaccaaaagatgtagtcgtcctggcacagtgggtgtggggcaggaagcagtcctctccaggggacagcagcaattcaccacagaacccaagtttctttcaagctctgctgacacagaaattgaataatctcagctcacccaatgtcaaagactcatattaaccaagaccagaatgaaaatatgctaatttatatcagaagctttgctggattcaagagttagggccttttacctgtgcagaatattccttcttgataaataggccctctcaggagaataaattacacatcagaggactgtttagtcagcataggcatagaacaggatgttccaaagatacagtcaaggggagtgggtaagagtgtagcctctggagtgaggccgaccaaatatcaaacctgagcttcataatttgcaaactaactggctttgggtaagtacatagcctctttgtacctgtttccccatctgcaaaatggagataataatagcatctacctgtagcattgttgagagaattaagtgagttaatgcttgccgacttataacacagtatacgatcactgattaagacttagcaactctaaactaaatgtttacaaaccatctcttacctcaaagcacttaacatccattgtcttatttgattatcactgtaatcttatgaagcaggcagggcaggggtctgccccatctggggggaactgagctcacagaggttggagggtttgcctaaagtcacccaggccactgggtctcactctctggtcttagctctgtaatctaggatgctcaatgccacactctcagccacttttcagatggctaagtacatttgttttgagttagctcagtctcagaggatgacattttctgatcttgtctccagtgtttaaatgaacctgtagctgtgcattggggtcacacaatgcgtggcatggagagggtctgtggctgactgccacggttactacgtgaaaccatcattacagcagttactactgttactgcctgagaacatcattacaagactgaacgaagggatcaacatggaaatgataacaaaaaaaccaaagtaactgttttaaggaaaggctagcatcgggaagaagaagagagaagaagagaagaagaaaagggctccctgcttctaatgagtaaaggcagctccctaagcttctgcagcccttcattatttattgggtaacaggaggaaggagcaggaggtaatgattgggtcagctgcttaaatgatcacgggttcatgttgttactgacagatttcaattatgcctaatcataagaaacatttgtgcagcctccaacaagggtcaatgccacttctgaaggggtgactcatagtcagtaactagaaagcagcagatagctagggacaaactggcgattctgaataggcctggaacccttagctctggccaggtcagtgggctccagtcaggatggagccttcagggagagatcaaagctcagaggtttgagatgatatcagccagcaaagaggaggggcagtagggatcctcccagagggagggccagccatagaagacatcaaatctgagcccggatcaggagaaggagcctgcagaactggggctctggcaccgagaacctgcagaacttcgcccctctgagtgcaggtgccagggctggggctgccacccagccttcgcatcccaggcctggcacgtcataggtaaatgtagttgaaaggatgactgagctgatccaattccctttacaactgtccttgtcctgggggacttgaggagggttaagaaagcagctggggaccaaccaacagtcctctaggctctccatgtccagcaatagttgttcagcaaatgagcattaatcagtgactataaactgtagcttcaacataaccgacaacttgcaatggtttctagagcatgctcccatgtgttatctcatttaaatttccaaaccaatcctgtgaaatgttctttttttttttcttttttttttttttgagatagagttttgctctgtcacccaggctggaatacagcggctcgatcatagctcactgcagccttgacctcctgggcccaaggggtcctcccacctcagcctcccaagtagctgggactacaggcacacgccaccgtgcctggctaatttcttttctagttgtttgtagagacagggtctccctatgttgtacaggctgatctgaaactcctggggtcaatcaatcctcctggcttggcctcccaaagtgctgggattacaggcatgagccaccatgccttcattttacagataagaagtctgagaaaactcagatttaggcagattgagtcacttccccaaatttatgtatcttgtaagaatccatattcaaacctcagtcccctaactcttagttcattactttttctaccacttctcagtatcctctaagaattcagaaagaaccacatcgactctgatttttcatttgtttaagtacacaggtaataggtgaatgtattttgttgtttaaaaattcatataatacacaaaaggctaaagtctcgcttcccacttcctctcccctttctacccaactctgcctccccagggagagcttctgctgacagtcggtggacattctttcagagttttacaattatgtgtgtgtgtgtacataagatgtcagtttttctttgtgtaggatacatgaacatgaattttaaacataaatgtgagtgtattacacatattgaccagcaccttagtttttttgtttgtttgtttggttttctttgtgctgtttgagaaggagtcttgctctgtcacccaggctggagtgcagtcttgcaatctcggcttacgcaacctccacctcctgggttcaagtgattctcctgcctcagcctcccgagtagttgggattacaggtgcctgccaccatgcctggctaatttttgtatttttgtagagagggggtttcactatgtaggtcaagctggtctcaaactgctgacctcaaatgatccatccacctcagcctcccaaagtgctgagatgacaggcgtgagcctccgtgcccagccagttttgtttttttattaaccaagttacgtattttaaacttctccatgtcaatgcttttagagctattttgttctctttaatgttaatagagaattttaaggcaatttcaggtgaatctatacaatttctctgtataagtaatttacactagaaatagatttttataaagatgattaagctaccagcctggtatttcattgctgacttaaatgaagaggaaaatcaatgctgtaagggaaaaaaaaaatggcattagagatccagaccttataggcattttccaaattattaattcaatctctcaaaacaggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcaggtactattgtcggtcggtgtttagctgagctcagtggctcctctcccagccttcccctcctctcctgagtgttccttcaggcatgggttataactcagcaaggagcaccctctttagattctgctggttttgtttcctgctttccaaacccttatcttgattcttggtaacatgaatcttctttgtaagttggacctcccctagcaaagaaaatagaataatagtgaaaatgttaatattgtttttatttttacagtgagggataaagtcatgttttcattcatttttgcagtgaccctacatatcaaaatcattgccctcttttttcttttaatgttgtttaatttagaaaaagaagctctggtttaaagaacagtgagtcacgtgacttgctctttgaaatgccctttgaagtctggctgaacactgggctgcattcagattcttcagtggccaccagaacattctgttttcttctgcacatcttacctttgcacaccctgcttattatgttcccccagaagcccaaccctctccaccaggggctgattaggaggctgcaggataaatgtttaaaagaatgaagatgtgtgtgcacgcgcacgtgtgacatctccatgccacagtcatgtttattccacgtctattctcccacagatcgtgctcccggctacctttgtgtttttggctctgatgctttctattgttatccctccttttggcgaataccccgctttgacccttcacccctggatatatgggcagcagtacaccttcttcaggtgcgcggactcggggtcaccattctcctctgtgggtttggggcacctgggtcacatgctgcttagaagggccctgaccttcccacttcactgggaccttcaccaatgagagaggggaggggtctttgggctgcctgcagaaaggaacttaatgtatctgccactgcttggaaaggcgatcctagtggacaggcaggactgcttgggaaggccgaatggggaaaggaatgcaaagcttaggtgaatgggttgaagcgccatctttttgaggcataggtgacatgccatcagaccactgcgagtgttcaggcagcctaccgcactcccaggagagctagcgccatcccaaggcagcattcggtgcctccaatacatacctggcacacagcagctatccagtaaaggctctgagttgcatgatgttggcacgcgcctgctctgtcccagtcacatgtctcactctgtctagcatggatgaaccaggcagtgagcagttcacggtacttgcagacgtcctcctgaataagccaggctttggcaaccgctgcctgaaggaagggtggcttccgtaagtgcctacgcgcccctgtcctaagaagactagctcccctgggaggacccaacggtgggttcaagatggcaggcgttggggaggccccactcaatcctgctctgctggtcacttccatgtctctgaccagcactcccccaacctctccttccacacttgtgtgcagggacattcactacctcctaggaagcccccacaccactggacagctctatatttctcagcatagaagttctatgttgagttgacagatgattccccataacttatttgaaaggcctctgagcagggagggagggaaatagggttatgctattgtgtgattgggccttgaatggcgtgagtgacacagtggccagtactttgtgatagttgtgagtctggagaagggagttagcgaaggccattgacatccaccaggaatcctaaaagttcaatataattttaacttttctccctcagtctttttcaaagctgtcaataaggaccaaaacagactaatttcaaattcctcttctggttgctgtgtctctcaacagctagagctgctaggaataaaaagggagacaaaacgatccacaagctagagatggttattccccagccccacacctagtcagtcacaaaaccctagttttgatattgcttgagcagaaaccagcctccaagagaataagaagaaagggcctgggtctaaagaggaggaggaaagggttgggcacaatttcttatgcctagggatttgtcagcaactttgaggctgattatggaatattttcttgtcttccatgagggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccaggtacctgacctccaaacaacggggccccaggtctgcctgccacagagggactaggggagtccctggtatctcctgagtctctcacaaactaacatttcaaactggcagttgagtaggggactaaaccaaactccctgcaccctctgggaggggctccccacagggcgctgtggctgccaactggaggaagccactcaccaaaagcttcattttccaccagatacttcctatttgatctagtagaaaaaatgtgtttaagcactaaaaaaaattaagtcatatgtgctcattatagaaaaattagaaaacacaggtaagtcagaaggaaaaaaaatcatcgcttggatataaacacagataatgtttggtttgcagccacccaaacagattatattccaaatattgtcttaaaatctgatttactgcataatttactaggaacatgcatccatgtcaataaatagacatctgcatcacttttaatatctgtatattatcccattgtttgaatttcttttttttttttttttttttttttgagacagagtctctctctgtcacccaggttggagtgcagcggtgtgatctcggctcactgcaacctctgcctcccaggttcaattcttgtgcctcagcccccccgagtagtggggattacaggcatgcaccatcatgcccgcctaatttttttggtagttttagtacagatggggttttaccatgttggccaggctggtgttgaactcctggcctcaagtgatctacccacttctgcctaccagagtgctaggattacaagcgtcagccactgctcctggcctaaagttactttaaattaactgatctcccattattcgccacttaggttttttagttttcaccattataagcaatgctatgatgtacattcaaatggaaatgtgtttacacacttattaacagtcttaattaagaagctctccatgtgctgtgtctctaacatctgcaggtatgtacacaaatacatgcacagccagcatccatcttttgcagggacattaatgatcttggctctgagcagcaccctgtcctgggagttctaaagtccagaacagattacagtgagcatctcctgggggatttagagacatcaaagaaggctgtgtccgtggttgataatgggcctcccagctgacttgccagggctgggccttagacagccctgtccaatgatttgtcaatgaataaactgttcccaaacaggctatgcagttcagtgggaaagcacaggtatgggacacggagagccccaggtggactacttgacctctctgagccttaattttatcacctgtgaattgggaataactgcttatttcataatattattatgaggatttaatgaaatcatgtgggcaaggaattatttagaattagattcaactcaagtgatgacaaccccaaactaacagcagataaaacaagacacaacttgtttctcactcatctaaaagtctacgtgggtggtgcacgatgttctattctctttctcctccacactaaacaggcctcagcctcatcagccaataaggcaggagctgccttccaggcagcggaatggaagaaggatgaagcaaaacagagggcagagtgtgcacatgtgctatgtttagggaaggttttctgaagttcccacatagtacttccacttacaaacccaacaaaaaaggctatggctaaggcagcagggaggagcaaataatgggagcaactagattttgccacagcacctatcacagtctggtttataaatggttctaggccaagaacacccgatccctgctcttttttatattctaaagcatgtatctttatatttctcaagcaatattttctctctttgaatcacagctcatctgctgcatcatagggatcccaaaagaaggacccaaggaacttgtctcagtcctctgtgccccaagaggaagctttgcttgtttgctttgctgtcaatgctgagggctcctgtggctgcctccactcaaaaccctccagcatcaggacgtcaaggctgtgatactgtaccctgagctcttggccagggcgagggaggggaggccaagcctacctacatggtgtttcatttcctaaacgaacccttacttccacgcggtctgtccagcttagaaacttattttcagtagtgttggtccttggtccctggacaaaatgtaacagccaaagtcctagaaaaaggcaagccagttcctgccattttctttcacttctgcatttcctcactattatacgtgccttccattggagcaaaactgaatgccacgcatatgcacaggagctgtgcgcgctctgtctctctcactcactctttttctctctctctctttctctctcaatctctctgtctctatctatctcttactctttatctctcactctctcactctttctcactctttctctcaatctctttctcattctctctctatctttctctctctctctctttctcacacacacacactcacaaacccacactcttattcacatctgctcaccctagccactcaaacacaatccctcattcagcctggaataagtccagagggcgtgggcctgattcagagacaatcagttgttctcatctgggaaatggggcaatgtggtcatctctagggaccctccctgctctaacattctttgaatgtggtgggtcctgaggtggaagcactctgtccctgacttctagtatatgtggagatagggttacacaaatattttattgggcagaacttttataaaacaatttatcataagctatcgcagccagcagcaatttttccaacctggattccaccaggggagcttggccggtgtctgagtgccactttcagcttgagaagcaggtgactcagtgaaaagagcaaggaggagacagaggcagattcagttcctaggccctgggccacccacctgcaagtttgcagcccagtcagtgcaagtcagctaactgttctgaacctcagtttctctgtctgtaaattaagctaaaaattcttctttcaaagagtgtcaggatgaagtgagatcgtgtatgtagggcatttaacatagtgcccgacacacagggagcattcggtaggtgccagctctcctcctggcaggagagagagaaacaaggtgaaaagagtgaattaaagaagaggaaagtcaaatgggaaaacagggggaggagatagaaagtgtatgaaaaggaaagaatggtgcgcaataacggcggtgtaatgccaccaaaatcccctcaactacttctgggcagcacccttgacagagtgaatgcttttatgagaatgtaagcggaatgtgttcccagatttgcagtaatattgccacctggtggacaaacccatgcacctttgaattttccaaaatatttcgatgaactagcttccagtcctagatgtattttgaaagtgatttgtaaattgtaaggaactattcaaattctttcattaatgtcacaaatcaactgtgtcatctgtatgccacccactattctgggtgctggggacacaacagctcacaaatcaggcaaagtccctgctctcaccaaaatgatatcctacgggggattacagatacaaatacgtaaacagatccatcgggaggaaactctcagatggaaatgagagctatgaagataacacaacagtacatgacaatacagagtgactggaaccaggaacatttctccgaggaataaaatttgaagcgagccatgagagggtctacaggtagagttcccaggcagagtgaacagccaagcacaaagctgcaccaggagagagaggtgctcgccgagagacagggaggggagtgtggcaggtgagctcagagaggggcagggccacacacatcggccacatgggccttggtagtgagtcgagatttgatcccagggtttattggagtggataagtaagcaaggtgactgaggtgctcgggtttacatttttatagttcaagctggctgctgggtggaaaacggaagttggcagaccaaggacagaatcaggcagacccatgtggaagtttctctagtggtctaggtggtggcttgggtagcgtggcagtattggagctggagaaacgcagatggattggagatttgttttggagtgacgccattctgtcttgtcaatggattggcgaaaaaagaggcatcaaagatgagttacacatcattgaagtgagaactagggagatgccagtactttatttagtattttctcagcagctcaatccataaataatttttggaagacaacaagcagtttcacaaactacttataagtcctcaagttccaaggtaattaacgtgggtgtctcattgcctcagagaacacagcgcagcacggaaattctacaagacctgacggacaggaacatctccgacttcttggtaaaaacgtatcctgctcttataagaagcaggtaagaagaaatccttttatgctttttatcctggctccctgtagaagatattaactagggacagaagataattttctctctcaatttatgtatgatcagggcagtagatttttttcttttttatctgatttgagggccccattcaacataaaaagcaattgaggcacatacaagtaaaatgtaacttaagattaattctttttttgttgtttgtttgtttgtttttacatttagggcaagcagtcttaaattttaacccacgtattattaaaagttatatcagaagaccatagaagttattcaaaaatgcagccacatattttaactagttaaaagagagagtaaaaatttggagggaggtggaggagtataggggaaaaggtagaagaaaaagagaaaataagtaagtggcaaaaaagagaaaggaaaaagatagggtgggaaagaggcagcgggacagtgtctgagtccagcacacgccagggcgagccaggtcaactgcagctgtcatattctaactgtgaattatcatctttgatcactgccctttgagatgccaatgaacttttcaagaaatatctagttctcttggctctccagctgttcttatcagccccatccaggatggaacagctttggcagcccgtatcagaacaagcagcttgacaggggcatgccatgccaggagagaggatcctaaggaagcgtggtccagtccgcacaggctctggggctttaagataaaacctcctgtctaactttagtaggactttctgttgcttcacctgccagagccctgaacgagggataaattgacttaattaactagaacacactgcaaatggtgaaagcatttagcaaaacaaagaatgccatccaagccccaaaataaaagcagaataaatagaatgcaataaacagcaaccatcccaaactgagttctcagcagcaaatctccagtatgaaattttggattttgtgcgtgtgtgcttaaaggtggatgacaatgacagttcatgggattgagctctggggtccagagttggcatctgttcatttcccattttgtcattttacccttgattgactgaatgtcagtgccttaactttgggctgtggagtgagtcggaactcccccgaggtgtgcaggtggttgttagagtctcatttttgcagggtggaagacaggagggctgcagccttcattccacactgacatggtcattgccgtgtgttctgggtccagatcaggcatattgacctgacatatgacctgacaacaggaccactcagaaagtccagcatgcgggatatgatttggagagccagtgggggaaatcataggtcctttctctgcatgtgtattcaggcaatgtcccagggctgggcggcttccgcattgcttggatatcggaaaatgcaaaaatgcccctgaagactgagacttcagtcttcaaaatgaatgtttgggaaagaaagttaacggcactgctgtacttgtggtattcattgcattattttattttggctttcagcttaaagagcaaattctgggtcaatgaacagaggtaagaaactatttttatcagaattaaaatctcagattgattcattgttgaaataattgcacacttttaaaaggcacacctcacagccatgaggaggggctgttctgtaggtgctcaggaagtcacaagacacgtcctgaagaatatgtggctagggacatcccagactcagaagacactcagtggtgcctcttcttggaggacataagtgggggtggcattccctgatgtggcgtttcagagcattctcacccaaaaaaagcttctaaaacctccaagtatataacagtttataatactccaacaagagggccttgtagcctaaacccgggacactccttggcccattccttttaagcttcagggagtgtgggccagccccagactcaccccattcctgaggcatcctggaggttgaaatatttccagaggtttagaacctcaccaagtgggactctaggagcctgctgcctcccagcctccctcaggaactgcacctccagaacaggtgcggggctgacatgtatgtgctttcctgggcagattctagaccgtacacatgaaatctggctttcaggattgctctccagagggacctgtggggcctcggctgagacagagagtaggagtgaggcagtgattcaaggccctgagaaagagctcctcctctgcttggtataaccagctaattcattctgttctgttgactttggcttctgccctgcctttgaagggtttgaggccagggagtgatgcactcagactggtgtttccacacagtcacttcagacttccagggcagtacaggagatagatcccagggccagtgaagaagcagagcacaagtccaggcaggagaggctaagggcctccctgaacaggtgtgaggcacagaagccccgagaggtagggatgacaggatgaagatgggtcctgtgctgctagaagtacctgcaaagcacagaggtggcacagaaaaggagtccttggctgggatgggaggagatgacatgtgacatgtgaaagaggacctggagttggctcgatgctcccaaaagggaaaggtgccgaggggagctagcagccatgcaaaggcagagacatgcaggcagtctgggccatgaggagctctggaagtgactcgatatgtccagaataggccactccagggaagggctgaggaaggatgaagttggagaggggcacagaccagatgcagaagggcctcagaggccaggatgagggtttggactccttcctggaggcagcagcagtgggaaaagagttaaaagctggtttgtaaagtggagccatgttgctcgctggtccaggcaattcccccgaaagttcatgtttccctacaaaacccgagagagctactagtaggcgtgaagttcgtggccctggtctgaggatttcctgtttccttgtcaggtatggaggaatttccattggaggaaagctcccagtcgtccccatcacgggggaagcacttgttgggtttttaagcgaccttggccggatcatgaatgtgagcggggtatgtaaacagactggagatttgagtaggatttttgacttgcttaactaccatgaatgagaaactctcatgagtgataacaggaaaaaaaaattaaaaccgtcttgtttgtttgtttacatggtttttagggccctatcactagagaggcctctaaagaaatacctgatttccttaaacatctagaaactgaagacaacattaaggtacttgacctatgtataatctgctctggagctaaaaatttacctgagctggttattttatttttactttcctaccttcattaaattccatccctcctcctgctgaaatctagcaaggaatgtcttccagctaccaaacccttcctgcttctcaaatttcctttccttcactgatttctgctttaactagctgttagtgcagcgtctcagatgtcctctccaccctctaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgtaagccaccacagccccagcctcaccactttcttgtcaccttctccactctttgaacatcctgagaggattctcaccaccgcgaagtgctgatttggatggtaatgctgtttagtcaggcacatatgaacatccgactttcaaataagtgcctcacacttcacataccagacctcttggtcattctttctccccaacatttatgtggcaagtaagtttacatttggttccattcccttttggcttttgatagcaagttgctcctggagcttatacaattattatctttgctatgtgcaaagcagctgccaggaactggcaaagttcagtaaacctttcagctccctcggagtaattatcttagattccaggaatttcctcagaagagcatactttggagatgtcgacagagctttgctaccctcaagctgaggctcttcttgcacagtttcagccagtggagacagtggccttgtgcgttttgtagtatgttcactctatttgaggcctacatggaggaggggttggtaggagcacctttgttagtgcaaacttcagcaacgttgtggggtcctgattttactatcctagcacacgctgagtgccagtgaacatgcccagggtcatccactaaaacctgggccttggctccttggtgtcttcctctggacaccctagggccctagactgtcctctgttaattctcactcagccacactttcgtgtgtctccttccagtcatttgttctaagcttactacgtgtatggatgatatgatctgtagttttatcaaggtagtgactaccacataggatacctttgtggaaattagtaaaaatgctcttttctgcaggtggacactgtcccatgccaggggttatggcttgtacataaagttcaggctggctttagccccaacttacccctcagccagatgccttctatttgtccgaggaaagaataaatagagccaagtccctgtacaacttgcctgccctcttttcacttaaatttacatcatgaacatttccttgtgttacgatgtacttcttgaaaatgtgatttaacaagatgattattaacaaaagataaatctcacagaccgtatgtctgtcaacatagaaaattcaagagactctatagacagattattagagctaatgagagcattgcagtacataagattaatataaacatctatttctatacaccataaaaataattagagaatataataaaaagaaaggttgtctagaaatattcacatgaaatagaaaggcaacccgcaaatacccatttaaccttggtccatatggattaagacagtttagtggagtgacagcttcaaggtagagaagaggaacctggaggccacacctgggcgggtgtaaggccttcccaaagcctgactttgtatcttctcctccttctgctcttccctcttcatcgccctctccctgtgtctctggccctgctgcaggctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcgtaagtgtcagtttacagcgcctccctcccctccgtgggcccaaggtggagcttgtgtgtgctctgaaggaccagaccaagaggggaggggttctcacggtgccagggctgctgaaaggcactgggccaagggccttgtgtatctgctgtcccttgacatcttctcagaaaggcacagaactaggagcccgaagctaggaaaggctgtggggtgcagcttaacaactggtgaacgggggctctctatgtcctgcactgaggggtcttctgacccatcaaataatcactgcaccgcaggcatgagtctggccttcctggcatcagtctggcgctgagaaggtaatatgaaggggtctttcaccccaagtccccttctcaaatcctgccccaccttcaaaagggtaaaggtaaaactttccctgtggtagggtcaccagataaatacaggacacccagttaaatttaatttcagatgatgaataatttttagtataagcatatgctacttcaaatattgcacaggacatatctacactaaaaaaaaaaaaaaaaaaaaaaaaaacctggttgtttatctgaaactcaaatttcactaggcatcctagatttttatttgccaaatctggcaaccccagccagtggccaaaataataagaccttcacttattagattaaccaccgctacagggaaaaatgaagaaaaaatatttattaaatcaatagcacactaccaccttcctgacaaccaaggttggtgggggtagggaggggtcaggatagcgtaccctattacaggctgcagggtcaaaggaattggtagtaaaggcctagttataatgtaacagggatcattatgacatcaaccccaatttattctaggtgtcttgagtagtaaaatctcaacattttaagaccaacatgagcctccatttcatgtgatgataagatataccaactgatggagaccaacacaaatgaccttctcatccatggttttttaaaatgatggtgaatattggaattcctgaagatatgatttctatcttactcagcttagtaagcagctatcacttaacaatacaaaaccagagattatcagtagcaactaaattatttcctctctcttctgtctacacgaggaaacactcataaatgcacggggaggaggtcagaacctgaaagcctttctttggataagagcatcaactgcaggtaccacattggccctgtgatgctaatataaaaggagctaggcccaccggtaccgaaaagttacttagaaaagtgcggaggcttttaattttactttttttaaaagataagaaatagaatttacacacttggggctggcccacgtgtttctgtgtgtgtgtatgtgtgcacgcacgcgcgtgtgcgcttacagggatctctgagcctatggagagagatgtagctaggatagagtggacatctgaggtgggaggtgatactagctggcagtccaatgaaggggtagaagatggtaggcatcatgttagcaggctttctgatgctccagaattttaaagctggcctggaatctcacctccgcgatccatcattttggaacttaggaccaccattagccagtggcaaaaaaaaagttgaatgaaggaacaaacaattattgcttatgtaattcacttagcacatatatgatgttttaaattcttatatgtgtcatctatttttctttactttaaaattttgcaacagttacagacttatggaaaagtcacaagtacagttgaaacctttttttcttagtcatttgaaagtaacttctcagcaagatgccccttctcatttatttctctcttcctgtctctctctctcacacccctcagcacgtccgatgtatacttcctacaaacgaggatacaccccatacaaccacaacacaaactgtcaacatgaggaaaccagcactgatgtgtcatcaccacctaatcctcacaccccactcctctttcgcccattgccccagtgatgtctttcagaaaaaaggatctagctcagaatcatgcatgacatttgattgtgctgtttctttagtctcgttcagcctggaagagttccacagtcttttgttaacactcatggtcttgacactttgaggactgcaggctggttattttgcagaatgtcccttggtctgagcttgtctgaggtttcctcttgcccaggttgagggtgtgcatcttggcagcagtatcagcaaacagatgctgtgttctcactgcatcctatcaggtggcttctgatttcaatttgctctgttactgatgatgttcaattcggtcacttaagaaggtgtctgctgagcttcttcactgtaaaattactcttttcccctttataataaatacaaatttcaggtagaggcacttcaaagatatataaatatcctattcattatacaattttccatttattcatccatttatttatctctgtatgcagtcatggttcatgtgttaatcaatggactatgatccaagactatcattatttattttgatattcacattatccccactgtggtcagtggggggccgttgaagctggcttctgtatcgtcttgacttgggtcctcatgcccctggacctcctccatgctcaatggcacagcaagatattccaggctcatccttccattatccccattcctaccctctccccaagaagccctggttcctgccagtgggaagtggccctcagaagccaaggtctgagtgctagatatgttcattgcctctggagcaccattggtcccaggccttctcagtgatagaactagggaagatatggatgtacacacacaggtatgcacacacctctatctatagttctctatctacctatacagtgaacactatgagctctccaaaaccaactccacagggctcattctagttttttttctttccacatctgtaactcccttctccaacagtgagacgctggcttctctcactcccaactcatttatctaccggacctatacacctgaacagtgcccaactctgccaccatcccctccccatgtggatgccgtcctctccctgctccagctgcctctgctgcatgcaggtcctcctcgttctgctctggctctgataccctgcaccagatcagcctcctgtaaggatatctttctcatcccgttgaggcctccacaccccacggcaggttgccccctgaggaagcccgtctctggttcttgccctgctcctgatcaccatggctcctcccctaaccccactgttgccgtcccctttctgtgcccagtatagtggctgtaggactaaattgtttaaaaagggtatcattatttatttgagctttgtgaagccaagaactaggctttaagtttttctgaattctgaagacatgcttagaaagaagaatcaacaaaactttatgaccaaatagaaagagtgagagaccaggcagaattttgtaattgatcctttcaaaagatacaaactaaaggttcccttggcagggaggtagggcatggggtggggtaggaggactagtgacagcttaacatatgtttgccaaccaagaactgtttaaaaagcaagtcgaatcagaatcccagaccctacgagctggaggagcctggccccacccctcattttgcagagctggcagcaggtctgagaggttaagtgacttgctctcctcttctctttccgagatgaattattccgtgagtgctgggctggtggtgggcatcttcatcgggtttcagaagaaagcctacacttctccagaaaaccttcctgcccttgtggcactgctcctgctgtatgggtaagccgtttgggccattagctaatgcctctgaagagaagcctggtggtgggggtgggggatcatctcctgacagaaaacctgggctgtcctgtggtggtagcacccacaagtttagcttccggccccaggtagggtctgaagctgataaccagggatctgtctggcttctgattctgactccactgacagaggtatctctgaggcctggtcctgtcagtgacaatgagagaagtcccacatgatctgaatctcctactcaaactgaggccttgaccaaagcctgggggcagccattccccaacccctcacccagctctgactctcactcatctgtggccaatctgtccacctcagtgtccccatgtgaactggccaagagttaccgcccacagtagaagactccggccaaaaagctcctcctgagtcagggacagaggatgacacaggggttacatcagcagagttacagggcccagcatgcaactttctttcccacgtgtgtaaatttgaatgagtaattcatccatctcggcctcagtttcctcatctgtaaaagaaaatagtgatcctggtccttcctctgtgggccagtagagccttgccaaagcattgttctccacatctttctcttggaaatagagaatttgggaaccaacctgactataagctgtgaagatgagctcactgggctcatctgagatgacctcagctgggctttgctgacccaggctagagtgggaggtgttgcaggctggagaaccctcctatgaattgtacagggctttgtagtttacagagtatatacacagctagcagcccatttgctcctcacaaaaccccatgaagtggtcaaggcaggcatcattatctccatttaaagttgaggcacagagaccaacaaatggagtatctctctggtcccctgggactctggccagttcacacacatcacctcaggtgtaaggggagtgcattatatccagacgtattgtaggtggaatggaatgtggaactccatcactctgagttgtctcatttcacacagatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccgggtgagcataactttcttggcttttttgtttgattagtaggatagtagagtatgtgttggtcgagcagagccaggggcaagcatcgtacatgtagcagctgtatgcggatgagtgccactttcttcctccctacccccgaccctgcctcctttccttccttccttcctcccatccttccttcctctttccttcttctcctccctcctccctccttcccccgtccctccttccttcctttttcattgcttccttccttccttcgtccctccttcccttcctctttccttctgccctctctccctttttcctttcatcctccctccatccctccctccatccttccttctttcttccttctttccttcctataagcacctttttcatttctgtgctctgaatgaaatggttttctgtgtttattctgcaagcaaaacttgattcttgcaataaactttaagctttgcttactctttcagaaaggttttctcagggactttgggtgttgggttttacacacacacacatcaatacatttgggtaatttcaaaatctaaaaggaacaaaaaggcatacaatgaaaaaatctccttcctacccctgtttcccactcatgcagttctcttctccagaggcaaactcttacttgagtttcctgtgtgctctggagacacatcagcagatccctatacggtctttctcccgctttcttatggaaattgtaacactctgacatatactattccttgggcaagttaatcttgatgaagagactgggtgttctccatgctgaatgcctcacttttatgagctgccaagcccagttgtcccttccacctgacctccccctgtccagagacagatggccaaactgaatcataaaaagagggggaaaaaaagaaggcagtcgctgcagggctgtctttactccacactccacactcccagtccccaccgctgtgtctgagtcctggctgtggctgtccttggaacatttgcctcaccacgtgcctgtgtccccaggcgcctcaacctttcctctcctcattagctcttcccagttcagagggtgggaccggccagcacatctgcactgctgccctgccacacccacctccacctgcctctgggccccactggggaacacaggacaaatctgtgcggaggccccaccatgaaccgcccagacccgtggacccctgagactgactctttccagatcttgttagggtttcgtggctgctaggcaagtaacgaagcctcatctgtcccatgaatgataagaaattcagcatgtcagagtcagactctggaaaggcggggggataagaacacagccccagcagatggccagagcacccaggtgactgaaagtgctgctttgcagagctgtgtttgccacaggctcacagcccactaagtcttaagacagttttccttcagaataattaaatagccagcttaaagcaactcagaacattttcccctctgaggctgcacccatttagccaacatttgctaagcacccgccttcaaaaacctggtattttcatgtaaattatccgatacacagctgctatggaaacccccagtatcccacaggaagctccccagctcccagcagctgccggcccgtgtgagatcaggaggtctttaccagctgaacaccacgtgccgggtgtgtgctgatataaacaagcgtggcccactcgtcctgccctccagaggctcccgttccagtcggaaaaggacctgcccacgaagtttgcaacgatataagccacagtgtatgatcctccataatacagcgtgtgacagagcagcagaggagcgaggcagataacatgctgcaggccagaggcagcgggaagagccaggctgcaggggctgggggagccgtggtggaggaagttcaatttcagcctgtagatttctattagcccatttaataaataatgaagtgcctactctgagctaatcattgtgcaggtatttaggaaggacaaaaaaataattaggactcagtgcccaccctccaggggcccactgactagtagagaaagtaggcagatttttaaaaaattaatcatgggaatgtgataagtgctgggagagaggaatggatactttctcatgggaatcttggaaggcttgtaagggaaggcactctctgagccagctgtctaaagaagaacaggaatctttaagaaagcagaagggaaaagagcattctttcctgcttggagcaataggtaacagcctgcacatgcccaggcctagaggccaaagagcacagtgattccagaaagagtggggagaaagggtaggcagggaaggatgaggtaatgtgggcgcaggtgtggaggctggagagggaggaggttgtgggactgggaggagccagatggaatggacagcagtggcccagccaggagctatgctggcctcgtacgcctcgatgtcccttctattttctcaggggaggctctgcccaacatgccaagtccgaccacttgaaaacaagtccctggcttaacacagaccccagagagagtctccaaccctcctctccctagacaatggtagttgccctgtgaggggctgaaaagcagagctggagatggctcagggcctggtgttaacaaatgccttgagggctcctgttgtttcaaagtgagtctgcagggagagctccctaagtggacagcaggagggctgcagcttctctgcacattcctgctgtcacccccagagtcacctaggggaggggtaaggacagtaatgcaggttcctcacagttagcctcggtgcccacatggtactgagcatagtaaatgtttagaagatgctgcctggctagacaaaggggaagctcccgcccactagaaacttgcagggagccccagtccttgattggtcatttaattgattagctccttggcctggccttgaggcactgcttgtaagtacttcatgacctccattgcaaacccatgatgctctgctggacaaatccctccagtggccagtctggctgcaaggactctctgtctgcaggccttgccctgtgctgtcctgtgagagcatctgggccccacctgctgaagagagggggggtggggtttgccccgtttccaacagtcctacttctctgtttcagacgctgctcaggttcaacgccgtgctgaggaagctgctcattgtcttcccccacttctgcctgggccggggcctcattgaccttgcactgagccaggctgtgacagatgtctatgcccggtttggtgggtggtagccgaggcccatggagcatgggccctgggtccaaagctgggagggttaccgggggggctcctgcatcagactgtggcaggggctggtgctaggaggggaccttgttgggctggaggtgtcctgccagctggagaggattagggtgcctctgtttccatggctggggagccacaggagggatggagggcagcccttatgaggcgggtgtttggctcttgctcagttcccacataaggcctggtctagtgggccctgtgctgtggccaggtctgtggggtgagctggggcggctgaagtggactcaattcctgttgatgcccaggtgaggagcactctgcaaatccgttccactgggacctgattgggaagaacctgtttgccatggtggtggaaggggtggtgtacttcctcctgaccctgctggtccagcgccacttcttcctctcccaatggtacgtccatgccacaccctgggccagtgggcagctcagggcatccagaactggaccttatacccacatggtcatttctttcctcaggagccccactccacaatgttttttctacattctcaaagcctggcttttctccaataatacaagtagaggatcgggttaaaataggcacattcaaatatgtgaagagcatccactttaaaatatttaaaatgcagtgctattaatttcaattgctgatatttaatccttctcatttaattaccaaatgtgtattttgattagatgatagtattgcaaataacaatggttacagggtatccaaagtactaggaaatagactaatgtatttatgagagaaaggacacagcaggcccctttgctaattagagatttgggagcatgggagtaatatgggagccatgtggaggggtgcgggcagtgatcacgaccccccactcctggaggaaggtgggtagctgccaaccctgacttttgaccagggcttctcaaatgccaggttagctggcaattgccattcttccgcaggctcttcctgaagctgggtgggcccctgcctcactcccctctgcaatccagtcctacctttattgtcctcacccaggggcctgaattgccaagcagcagcccttcctagcaagctttccccaatagtgttttgtttcttaacttttcctcctctcaggctgagtgtggtcacctgtaaatagattccaaggacttggttttatgttttgatccacagggaattgatttattggaaatgaatctgcctttctactcacaggactgtgagaggtgaatgagatcacaggtgtcaacacacgcctgatgaaacaggatacacaagcagttctagttatgggagacagtgtcaggaattgttgtccttggcaccctcagcccctgcagaccctttctgcagccttggccataccttttagaggcttttgtgtgggagagagcaggtcaggaggttgactacccaaattgactcattagcttcaaactctgatgtcaacacatttgaatgagtcctgcctgctttagggcctaaagaggaccagagaagtacaccatagtccctggcttccagaaggtcagggagggtttcaaagaagaggctgtgtctttaagaatggggaagattccatttggtggggcaggaggaggagaacattgagggactggaaacacatgcggaggctgggagacgggaatgaccaataggactgggaaccagggggagatgccaattgctgacagaggagttagtgcaagaggtaagtgagaagggtaggtggggctggattgcagggctgtaactacagctgcagagggagggcttcaacctacagctgatggggaacaacagaaggttttgaggcatgaggtggcctgatgacaactctgttttggaaaggtggagttggcagggcagactggaggaagtgggaggctcggaggttagtaactaccccttactgagtgcttgctgtagaggaagcattttagtcctgacggtgatcccaggccctgagtctttactctgtgccaggcactgtgctgagttcatcttcagcacaatcctatgagacaggtattgttaccctcctcctcatcacatggttgaagtaggcaaggttcagagaggtccaatgcccaagatcacacatgaggaggccaggactggaacccaaggctgactctggacatgagcacctgacctctctacctaatgcctaatgcctctcctgctgggagccctttttagaatttaagtcttaaaggatggaagcccagaaggaagcagaagcaaggaagtggaagagaggtcccatggaaaggacagtgccaaggacactgtacagccagcccaatcctgaccccttttcttcatctaggattgccgagcccactaaggagcccattgttgatgaagatgatgatgtggctgaagaaagacaaagaattattactggtggaaataaaactgacatcttaaggctacatgaactaaccaaggtaagggaatgggtatgagtttggaggtgctggttagatccacagttggcatgatgttgccattttccttctatagaacaattgatatgcttatgcaagcaatttggttcccagttttatgtagggtcatcatccctgtgttataactcgtcttccaagagcatctaattccaatgtgtgttccctgctattcatctcgggcactgacacagggcctcagtgagaatcactccagctgagcatcattcccttttctgtgttctgtttctgcagagcatgggtcagcctcgagatgtctcagtactcaccacacctctgtgcctgcccatgtcaatatgtaacctcctagtgctggtagttttctcctaaaccatcctttgctctttgttccctcttcccctccttgctctcaccctgtctcagttctcagtccggtttcttcgtatcttgcagatttatccaggcacctccagcccagcagtggacaggctgtgtgtcggagttcgccctggagaggtgggtactctgcagaccacgtgtgaaaggcttccgaacatcagctcttgtgcctgcctctcctccccataaggcagagctattcaataggaacataatgccataatgcaagtcacatatgtaattttaaatcttccactagccacatgagaaaagtaaaaagaaaataggtaaaattaatttcattagtattttttattttactcaatataaccaaaatattatttcaaaatgtaattaatagaaaaccttattaatgaaatatttgacaatttctcgttgtttttaagtctttgaatctttacactcagggcccgtgtcaactgggacttagatgtgtttcaagtgcttagtagccacatatggctcgtggcctctgatggcagcccaggtctaaaattcctcccccagctcacacacacacttaccctggggcctgacattttagaccttcttgatctctagggccaggctagctctgtgttttctcctagtgctttggcctcctgggagtgaatggtgccggcaaaacaaccacattcaagatgctcactggggacaccacagtgacctcaggggatgccaccgtagcaggcaagaggtgagtatcctgctcctcctgtctcagggagtctctcacaggtcctgtgagaagaataggaagggtgatcatcagaccctatagtagggtggctctgaggccctgaaagatctgtacagagaaggaggcctcccagagagcatggcccaaaaagcccaacacatagacccaatggaaaagtgaactgaattgtgatagttaagagattcctctgttgggatggattcttggaaagacctgggaagcactaagtgtgtggttcttaatctcttagaggtcacggaaccttttaagcatctgatgaatatttgtagcctattcctataaaaatgcaccattgcttcccattacctccctccacacatttttacaaaacgtttcagggagtttactgagccccaggtcacatttatgatcctgcaggagctcttgaatcccaggttaagaacccctgtgatgaatgaagaatccttcctctgggttgagtttctagataggggctcatgcatgggcctttggggtagcctaacctgcattggctatttgtaggctgatatttggctttgccagaccaaggagcatagagggaaaactggcgtgtgcccttggattctggagggtgactgctgctctctgtaataaaatgtgtttaaacagactggtcccctatgggcaggacagagaggatgagctctcactcatctgcctctttcctggctgcaggaaaagcttgaacagtaaaacttcagcacacacaatagaggtgcccagaggaagcctctgccctggtttataagtggagttaggtgctgctgacatctgtccagcatctgcttgactggggcctcttcctctctcctgaaagccatcctcagcatggcccaatgcccagtgggcaggacgagtcctgagcacgcttcactggctcagacaggatgaatttgattctttggcctccatagccagccctactgggtttacagaaaagggacaggcaggggtgaagccaggtcatggctgagtccatctcaacagatccagcttcacctgcaagtgaccacgcaggtgacttcctcatggtgacaaaaggagtcatggcagggtagagatatcataccatggcaggggaaagatatcatagaattttccatgagcacatttatgagacatcaagttacaactgtgtccaagtgaggcacagtctgacatccagaaggtaaaactgagctggacgctagaaagaaactataggcttaagacacagaattgggattatatggtagggtagctcccactaatttggaaacgtaccctacttgcttccctgagtagttttaattggcccagccatgcctttggtggcttttgtcattgtggggaactgtaatggtctctctgtaccatcctatatcatccatcctttattcatagaccctaagctataagaagaaaaggatgagattagactaaatgtctatgtatagtttattttccatcttggcaatatattttttagtgggggtgaatatattagccaaagggagttggtggaacccaactcactctacccctgctccctgcaggcctctcgctgtgggtagttatctgactggctcctctttcattgctatctttgccaataaatacagatagagaagtttacttccatcgggacacatgcatcttttctagttacttcccaaatgtctgaaaattattgataaatcatgaatcattttcttaaacctgatcttccctctgtttttaaactcacatgtgaggtgatctgatccaaaatgaaagctgacttttggcgtaacagggattcaattaatcctagacatggaaacatggaagaatctgacaggattcagtttctaaccgaagggcccctgttttgattcccaaatatcccatgcatttctgaagccaaataggagaagagaagaagcagcttccttttcccgttggcagaagcttctccagccctagctctatggtcatccctccactccttgaaggatactcagtaattgctttttttcttgcagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggtgaaaaatgttttgttgtggccacataggagtctggttaattacaagcctgtttcatgagagtgcattctcttggagatgagaaactgaagcgtgctattcattcattcattccaacaaatgtttactatgtgtctactgtgtgccaagtactgttctagaaaccaggagtatagcagtgaacaagacagacaaaaaaaaatccccactctcatatctaacaaaatgttgtatgcatttatcctctgactcagcaatcacacgtctaagagtttatcctgaagatgcatctcccacagtgcaaaatgaatatgtataaggtgatccattgcatttgtaattgcaaaatgctggaagttacctaaatgtttagtcattgtagattggctgaataatttatggtacagacacacaataaagtcttacgcaactataaaaaagaagaagaaaagtctcagtaaactgatatggagatatttccagtaaatactgttaaatgataaaaagcaaagtggaaaacagaacatagagaacgctactttgtatgtaagaaagaaggaaaaacaagaaagtaaacgtatgtctgcttacctttgcaaatagaacgtagaaaggataaaccagaaaacaatgaatttggtgatcaacaagaagaaaatgggaagaaagaaaaatgggaggaaacagtacttctggggatatatttttgtatagttttaatttttggaagcatgttaatgttccacatattcaaaaaaaatcagtaagaatgggaagtaggcaaaaatgaaaacaaaaagaaaacctaacactgacagcaaactaaataaagtaacccaattttatttcaaataaatatcataatcttgcaaaagggggatagagctaacacaaacaactgctgaacacagtgtttgactctatatcctcattcttgggcagggtggagcgggggagaagaactacaaataatttctgagttctttttagtttgttttttatagtggtataggcaaagtgattctgaaaattttagatgtgttacaggattaaataaattaataaatgttttgatgttattgggacccagaattctcaccgtggaagaagggacttacaaatatggaaaagggaaaagcaagaaagaactgtgaggtcatggataggaaccggaggtagcactgggaattcaggaatatttatatgcttgtgtttgtgggtgcatgcagatgtgttcatgtttcatgcacataggcatgtatatatagacatatatttgcatgtgtgtatctgtcttccgaaaggctcaagaagcaaaaacaccccagtagccatgagcacacttagcactcaggcttttgtcttaataacattccccactaaaagtaaccctgattcctccaataaatgataagttccagggctggaatggcataggtataaaatgaacctggaatatcttatgccagaaagtaaggaagtgcttttaaaaaaaaaataaggggctgggcatggtggctcacacctgtaatcgcagcactttgggaggccaaggtaggaagatcgcttgagcccaggagttccagattagcctgtgcaacatagggagaccctgtctctacaaaaaattagcaaacaaattagctgggcctggtggtgcacgcctatagtcccagctactcaggtggctgaggtgggaggaatgcttgagcccaggaggttgaggctgcagtgagctgtgatcaagccactgctctccagcctgggaaacagagcaagactctgtctcttaaaataataataatataattttaaagaaataaaagtaactctgtacagattgcttattggttacatgggagaaacataataattttacaatggagaaattagacagcaccttaactgggtgatcaaaattaaccataaggggcagatggacatctcatgccccgagatgtgataccctgtgaaggacacaatttcacttatgtagaatccagattggagatatgtaacctgaatcttatcatgaggaaacatctgacaagctccaaagaaggaatattccttaaaaaaaaaaaaaggagactgtattcttcaaaaacataagagtcataaaagacaaagaaagagctatggaaatatctctgatcgcaggaggctaaacaggcataatgactgaatagcagacaatagactacatcttgtgcagaagagaaaaaaaatgatagaaggatattattggaccaactgacaaaactgaactatgaacagtagattaggtaaatgtatcataacattaagtttactgacattgataatgtactgtggttatgtaagagaagatctctattcttaggaaatatgccctgaagtatttaggagtgaagggctgtgatgagtaatttaccctcaaatgggtcacaaaaaattgtgtgtgagagagagaagggttttattagttaataattctatgaactatttttattcctatatgtttgtgtgagtttgaaactatttccaaataaaaagttaaaaatggagattacattctagtgggagggatagacgatctgtagataaataggtaaaatatccagtacattagagagtgaaaagtcctcagggaaaagtaacgcagggaggaactgctggggcagggtttgcattttgaggtagggtggcccagggagagcctgcagaggagagaacctgaatgaagaactagaggtgagagaaggagccacgtgcacacctagggaggaacattccaggcacgggggactagtatagaaggcagaagcatggtgagcttgtctccagtggcttccctagatcccctcctgcgcatgtgcacacacacctggtgtctctgtcatcgttccctcacagcactgtcacgatctgccagtattctgtttattttgactgccacctccccgcagtctgaggatagcagcaatggctgtgttcacattgttctccagtgcctggttcagtgcctggcgtatggtcagtgctccataggtatgtgtcggatgcacaaggctttgggtgtaaccctcttgacgggtgggatcaacaggtctgggactcaccatcttctcaaacagagccttcctcctccactgctagccatggtccaggacgctgggcgagacccactgtcttgctctttgtaaggctgaagtccatttcccaggcggctacacccaacagatgctgagcaggctgggccaccctgggatccaagacacagagagaaagagcccctgtctggcgcctgaagcacatgccagaggacaggagccagcaggagcctgtttcagcctagctggggatttcattctggaggcgtgagatctgggagcccaaggctttgaactgggggaggtttggggtgtttgcttgtcttctccaaatggcatttctttctcttccctaggttgcaaactggagtattaagagcctgggcctgactgtctacgccgactgcctggctggcacgtacagtgggggcaacaagcggaaactctccacagccatcgcactcattggctgcccaccgctggtgctgctggtaactgcgggcttgggccgcaccaagggcttaaaccaagtgctgggtctcttgggttggggaaataggttctgggtcggcagatttagaaactgcagcagtttggctttagtctggactgtttcctgtgttgctcattttgagcgatcagcccagtgtttggttcacacagctccggagaaaaacaagtcacggcacagccttgacttgggactgcgcacatcctgcgttcccaggatgtctcctgtggggccatcggctcacagccgggaagttcagcccactctgcggcctgtcggtgtctggtccccatacaggagcactgagctgggtcaaaggctcctgagctgagccaggccaggcctgaggccatgcccacgcagcccaaggatcatgagggcacaggacatagcgggaaccaaggaagtgacctgagtgacctccctgccttctgacaaatgtatttgcaggattttctttttttgaggagaattctgtcattgccttaatccactttaatcccctcgtgggctgaaatgggcccaggatggacgccacgcttctttactcttggatccacctcctgccttccctaccctacaccagggtacccctgtcttgctcaagtgaggggagtgactgtgtgcgccttctgtcagctcatcctccacaggggagccagcccagggggaagcagtaatcagaagggccagctcccagcctgtgcccccaaccttctctccaccccccaggatgagcccaccacagggatggacccccaggcacgccgcatgctgtggaacgtcatcgtgagcatcatcagagaagggagggctgtggtcctcacatcccacaggcaagagattcccagggctggggaaggtgggtgggaatcctctcctgctcacctcctctctcctgccccacagcatggaagaatgtgaggcactgtgtacccggctggccatcatggtaaagggcgcctttcgatgtatgggcaccattcagcatctcaagtccaagtaagcagatggtggggcgtgccccttgttgccttctgtggatccacctggatcctgtgttctccattgacacttggaagagtcctgctgctccgtcatcccctggggcagaggcaggtggtggctgggcctcattctccagcagcagatggagaaggccatcatgctgataagaaactcctctatattggcctaatttcctgtggtcgaagactcgcccaagtctctggatggggcatctgatcaggatgcatgcagagcctggctgggatgagggagggctgctaccactgcctcaatatttcaccacttatctcaacagatccgggacctgtggcctatttactaagagtccactccaatgtaggaatggttaggagaccaactgacttgaggacccatctttgtttttagaatattgtatgcttttgagtttgaaaaaagaccatatgttatatgacaaaccaacaatggcagtaatcttgaataggattatccttatcctgtacccacacattgtaaactattgtagataattccttattattaagagtttgcatgccaaagctaacagtttaagattatcagcatattgccgtgctcattcacgttctgatatgctttataacctagaaaagagcagagttacaattactcatttatttaacaaacacttattaagagctcagaatataagtcactaagctggttggtgggaggaacagcacataacccaccttatctatgctgaggtgcataatcctgatgcacccacaggagggtgttacacagaagatgtcatcctttcatatgtgtcagagcagataaataattgagagaaaggtctaatagattagctgcttgtggcaagtggacgtttgacccatgatttattgagcaactacaacttggacactgcatagatatctatagaaatagcagcatgtcaggtcaccagacctgtgtcagcaacttcctgtgtccaactgctggagaaagggaagtctcctattcctttccctccagctccttaatatctccatgatagagggggtgagaggggagtgttccctgtgtggagggatggtgagttttctggagctgaaaggtaaacagcctttctcctctgcatcttactgcagaggagaacagccctagactgtggaggaagctttggagtcagttatgactgacacaggataccagggcatagggtactgacacccgctagccgtgcacacactctctggtggaccatcactcatccaagagagggtaaccagccatcctgctgaaggagaaagaaagcaccaatggcccaagccctagcagctccattgtttcaggaagcttcctcagggaagtgctgccttcccgagcctttgctcccacctggcccatcagcccttaccaccactcagtatgcactggtccacgtgtctttatgggcagtcttgggatccccacactgggctaaaactacctttgacggccaggtgcagtggcttacacctgtaatcctatcactttgggaagctgaggcaggtggatcacttgaggtcaggagttcgagaccagcctggccaacacggtgaaaccctgtctctactaaaaatacaaaaattagatgggcatggtggtatgcacctgtaatcccacctactcgggaaactgaggcacaagaattgcttgaactcagaaggcagaggttgcagtgaatcgagatcacaccactgcactccagcctgggtgaaacagcaagactctgtctcaaaaaataaaataggctgggcgtggtggctcatgcctgtaatcccagcactttgggaggccaaggcgggcggatcacttgaggtcaggagtttaagaccagcctggccaacatagtgaaaccctgtctctactaaaaatacaaaaaaaaaaaaaaaaattagccgagtgtggtggcaggtgcctgtagttccagcctctcaggagactgaggcaggagaattgcttgaacccaggaggcggaggttgcagtgagccaagatcatgccactgtactccagcctgggcaacggtgagactgtctcaaataaaataaaataaaataaaataaaataaaataaaataaaataaataaaataaaataaataaaactacctttgacttcagcaagtacgattatcccacattaccatgcagacatttgatctctaaaaactggtatcaaatgatttctccagggactaccatggtttttctctcctagttttcagtatgtacacaggtctatggtatgggcctttaatccccagtatttctttttttgttgttcttgtttgggtttgtttcttgtttttcggtttttttgagacagggtctcactctgtcacccaggctggagtgcagtggcatgatcatggctcactgtagccttgacctcctatgctcaagtgatcctcccgcctcagcctcccaagtagctgggaccacaggcatgtgccaccatgccctgctaattttcgtagagacagggtctttcttgttgcccaggcttatcttacattcctgagctcaagtgatcctcccacctctacctcccaaattgctgggatttcaggtgtgagccaccaagctgagcttaatccccaaaatttctgatgagtctactccttattttgggattaccttaggcccaaccactaacagaggcctgtcctgcactgtgtgcatcccctagatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggcaagttggccctggggcaccgagagctgagcaaagactggtccagaacacccagtgtgggttggaattgccataagagggaggcataacattcccgatttttaacaaactcttgccctctgtttattggggtaaaagctgatatatcagaaattgttttctaacaatattttttagtcatcaggaaacttcattgattcttttttttacattttccttccctgtgatgctatggtgtgttatttcattcttgctcgtttgtggtggtggtttttccttcaaatcagctttattgatgtgtaattaacatacgatgaaacacaggttctttgggaggccaaggcaggaggatcacttgagcccaggagtttaagacaggcccatgtaacaaagtgagactttgtctctacagaaaaaaaaaaaaaaaatcagaaaattagccaggcgtggtggtgcatgcctgtggtcccatctacatgggaggttgaggaaggaagattgctggagcccaggaggtcaaggctgcaatgagctgtgttcataccactgcactctagtctgggtgacagagcaagcccctgtctcaaaaaagcaaaacaaaacaaaaacacctattttaaatgtacagtttagtgagttttgataaacgtgcattccatgtgtggtttttaaaaatgtaatcacattttttattgcggtaaaatataataacataaaattgaccatgccaaccatgtttaagtgcacagtgcagtggcactaagtacatttacattgttgtgcaaccgttaccaccatccccgatagaactctttcatcttgcttcagtgaaaatctgtgcccattaaacactaactcaccacttactgcccccctcgcccttggcaactactgttctactttctgtctctaaggctctgactactatagatacctcatataagtggaatcatacagtgtttgtccttttgtgtctggcttattatgcgaggacttagcataatgtcctcaaggttcatccgtgttgtatcatgtgccagaatttccttcctttttcaggccgaataatattcctttgtacgtatatgtgctacattttgttcatccatctattcattcattgatagacatttgggttgtttctgggttttgtgtttttatatatgtttttttaaaaataaacatctttagagacagttcagtaaagcagtggaaacagggaagtctccatttaacccctgaggatctggctcacctgcaccttctcatcagcattaagcagagggaggcacgagcaggagccacctgcacactcaatgaggagctgaacagggatcaattacctttttttttagttattaggatgctgctagctgagaatctgccttgccttgattaccccaatgtctggtgcccaagtcccttgagtcctccagcaggaactcctgtggcatcactcaggagtctagtctaagaagctagctctgaccagggcagtggtggccaggcttctgtgagtgggccagcctcccccgggtaggacacaagccataccagcagggctgtatgtgaactgtggaaaatagagagcaaagtgggtaggtgggtgtagggtgctgttttcctggaaatatctacctaatctcgctcttctcttacctctaggtgtttgtaaattttgctaaacagcagactgaaagtcatgacctccctctgcaccctcgagctgctggagccagtcgacaagcccaggtacccctgctgcttatgcagtccacagcttgaggcagttccttggctcagagcccagctggttcactgggcttgagttgctccaaggctcagatatgcctcctacagagagccccacccacaccacggtccctaccaagtccccaccacatcctcatcacatccttgctaagtccctgccactgtgtgttctgtgctgaagaacttttcattcagtagttgtaggggttcctattgtaatcaggaaaccatctggatagcatgggagagcatttttgaaaagaactttcccatgtttttgcttacagcaaaaaagcttggatttggggaataaggagcagagaaggtaatagagaatattagaatgttttgggtgcttgacatctatgtctggacatgtgtttgagtttcaagggaagggacttaactggcacatcatttcagtgtcagacacatttggttagatcaaggaatagcatctgttgtaggaagagggctctttgttctttataaaaattacaagaagatggagaaagaagcaataggaggtatgtctcctggcttgtgataactcttggaataggtgcttgtaggttcctgccctggcacagtgccccatgtaaggagcacaccacccaagaaggagagagctagagcaagtactggaggaggcaccagcatcccaatgccttggcttaagcctgggattgtagagggatgaattagccactctcttctgacttacctggagagtaaatcaaatcaaatcaagaagcaaggatatgcaaaaaccttatttccccataaagtttttattctgcccagtttctggattgcaagaaaaaccaaatacagctaatgattgaaacactgctgtctaaagcagtgcttgtgatgaattttttcccttcctcttgaccagcagagacctaatggctacttggcaaaactgactttgtcttcccaccccttacctgccagagggcccagaaatgcctaaggctcctttagttacagaaagtttgcttttactgagatcttccagccactgattcccatttatagatctggtgattgctgttgacatcagttgaaaattatttttaaaaaccacttgcagttgcaaatcctttttataactctgtaactcagaatatagaattgggtagcaaaattgtttcccagaattaccaatggtctccccacccctgcctggcatgttccctcttaaaggactaatcccaccacatcacctctgggccaggcagaacatcaggggtgctgatgttctgtgatctacagcagttaattccaaacttttctcccttattggatgagatcatttttctattgtgttttttacatttttgttcacaaagattagaaaacctgcaacacacttattggcatatttttctgataattttcatccaaaacctaattctgactttacaacatactatctttacaaaggtttgcaaaaattctttcatatagcattgtatatgtctgtcatgaaataatagtaagtatattattgtttacattataccacttcaaaataatttcctttaaagtattcttcaaacaagaaaaaggcaatttctctcaagaagttttagagagaatttacaacttgctcctaagcaaatgtgagaacttcaggaggttcatctggccattggctttacaactccaaattgtgagccaggaccacacagatatttctctagaaatcagcgtttgcttaccaagaacatttttactctccaaaggactccatcctggaaaacatgttttgggataaggtcttatgcaatcttatactctgttattaaaaccagtgagggtcaaggtgttaatagattaagtagtgacagatgatcagacaacttagaaacatcctaaataggttaataattatgtgaccatcgcatgtgcattcccaaattaggaacaactcagatcaatttctaatccttattcttacactgttccagttcccccatataactcgtatctttgtgttagtttcagaagtttctgaagtaccctcagccttgatggggatcctcgcaccacctcaaatcctgttctcagccctaagaactgtgttagtcatcctcttaagaggatgtgtgattttaaatcagataatgggataaaccacatttcgtctagactggtcaggcctttgtccagtcccctcctcgcccacactaccccagctccacagcgggcattggttcaggaattcaacccacactttataactggagacagtatctctccagttaaaaaggtcaccttggtgtccgcttctcaaggaacatggacatctttattaatcaaagcccaagctttgatctggagcctaatatcctgcactccagctctcatctctcccctcccccagtcacactttcatgcttcccagagccacccctacaggaagtggtcaagggaattctatacctcagggctgacctaaattaggatttcttggcttttaagataatggtaactttcttaagctaaaaaagccccaaaagaccctgtaagagcccttggaaacagcaccatgggtgtagcttccccccaggatgtaagcatgtatgcacacatctcgtatgtgtgtctttgtaacaaatgcctggatcttagtaccagggagacctgattcatagatttcatagagaaggagagaaagatggcccataacctgggtgatctgacagaatcacagtgccctcagctgagtgcccttcagaaattgattgacaactgtttagcttttgaaatctaaaagtagtacagcatctcagaaaaccaagatgacgcgagtccatgtgatctccttccacaggactgatctttcacaccgctcgttcctgcagccagaaaggaactctgggcagctggaggcgcaggagcctgtgcccatatggtcatccaaatggactggccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccccgggccatcccactagcagctttggcctccatattgctctcatttcaagcagatctgcttttctgcatgtttgtctgtgtgtctgcgttgtgtgtgattttcatggaaaaataaaatgcaaatgcactcatcacaaactatcctaattcacagtctccctggtgtgcaccacctagtatagttttagacattctttagatgggtgcatagctccttgtcagtcccatgcacttctgtgagtgttactgcctcaggactgctcgttctggcaagattctgcaacactaggttggaagtgaatggactagtcttaatgttccatgtcaagtctttgtagagtttgaagaaaacacccaactagtaatgcctgtaaacattatccattgtcagctgggtattactgactttaagtttcgtgtctgtttgcccagcttatttgagtgtttacctcacaagtgtaattaggacaggagacaaagaggcatgcacaggcgagagttggtccttggttggacgtgagacccgacaggactttgtaaccatttgaagaggtcaggacctcattctcatgctgctgtgccttttctgcagtgctaccatgtgcatcttctgcagtggtgttagaagggaatgaaggccgggcgcagtagctcacgcctgtaatcccagcactttgggagcctgaggtgggcagatcacaagatcagaagatcaagaccatcctgactaacacagtgagaccccgtgtctactaaaactatgaaaaattagctgagcatggtggcacatgcctgtagtcccagctactcaggaggctgaggcaggagaatcgcttgaacccaggaggcagaggttgcagtgagctgagattgtgccactgcacaccagcctggcgacagagcaagactccatctaaaaataaataaataaataaaaaggaatgaagatggacatctagttcacataaatgctcatcaagattcagaaaataataattttaaaccaaacatttccagagactgtggatgaggagaacaagtgggtttccttgtcagggcagtctcccctccatgacctagagatgggcttcgtcgtaaacatgctttcattatactgaggatagactccagagtcggggtgaggcaagaggaaaaggggagagatgctgaggcccaggaactgttgctgagaagaggatgagaaagaaagtgagcacaaggtgctaaaatttttgtttcagctgtgcttgtttagaggcagacagagggcaagggctacacaactttaagtcctgcacacctcctggcttgccactttgcaccttctagatgctaggcaaacaactgcccatagagactataaaactactttggtaaccccgcagcttcatctggtgttgactttttcttttaagttaccaaggaccaaaactgtaagaccattttagctagggctaggatagaggttgggaaagcccagcacactgcttgcatcacactgctgcaccctggctggtttcataatttaaattagcaactgcaatatcacaggaaagaagactacactcttcggggctgcttaggaaaagaaaactaaaaaaagactatgtaggggaggtggtttagcagccattctgtttggctgtgagggtttcggaaaggcatcatgaactgggaagagtcgatgcaggtaaaatctgcacacccccttaaggaaaaatctcagtttaccttttgtctccactacacctgaggtctgtgtcttttcatcctgttttttccagctcctggcacacagaaaatgttcaactaatacccaccaaactgaaaacccagcaaactatgaaaactcagcaagaaaaatagacagaaaagaagtgggtccaagaaaatgatgcctccaaaaagcagcaaagggcaagtggagcaggaggatgccgtgctttaaaaacagccacaggccgggtgtggtggctcacgtctaatcctagcactttgggaggccgaggcgggcggattgcctgagctcaggagttcgagaccagctggccaatgtggtgaaagcccgtctctattaaaatacaaaaaaaaaaaaaagaaagaaagaaaattagccaggcgtggtggtgggtgcctgt

SEQ ID NO: 2 - DNA, 20 bp, Artificial Sequence
  Description of Artificial Sequence Synthetic oligonucleotide
ataccttgtgttacatggcg

SEQ ID NO: 3 - DNA, 17 bp, Artificial Sequence
  Description of Artificial Sequence Synthetic oligonucleotide
gggaataccttgtgtta

SEQ ID NO: 4 - DNA, 21 bp, Artificial Sequence
  Description of Artificial Sequence Synthetic oligonucleotide
agaacctgggaataccttgtg

SEQ ID NO: 5 - DNA, 19 bp, Artificial Sequence
  Description of Artificial Sequence Synthetic oligonucleotide
ctaacccacagaacctggg

SEQ ID NO: 6 - DNA, 20 bp, Artificial Sequence
  Description of Artificial Sequence Synthetic oligonucleotide
ccacgtcctaacccacagaa

SEQ ID NO: 7 - DNA, 20 bp, Artificial Sequence
  Description of Artificial Sequence Synthetic oligonucleotide
gaaagacacccacgtcctaa

SEQ ID NO: 8 - DNA, 18 bp, Artificial Sequence
  Description of Artificial Sequence Synthetic oligonucleotide
taggaaagacacccacgt

SEQ ID NO: 9 - DNA, 17 bp, Artificial Sequence
  Description of Artificial Sequence Synthetic oligonucleotide
ggtaggaaagacaccca

SEQ ID NO: 10 - DNA, 19 bp, Artificial Sequence
  Description of Artificial Sequence Synthetic oligonucleotide
ccctgtggtaggaaagaca

SEQ ID NO: 11 - DNA, 18 bp, Artificial Sequence
  Description of Artificial Sequence Synthetic oligonucleotide
ctgccctgtggtaggaaa

SEQ ID NO: 12 - DNA, 17 bp, Artificial Sequence
  Description of Artificial Sequence Synthetic oligonucleotide
aactgccctgtggtagg

SEQ ID NO: 13 - DNA, 17 bp, Artificial Sequence
  Description of Artificial Sequence Synthetic oligonucleotide
gaaactgccctgtggta

SEQ ID NO: 14 - DNA, 18 bp, Artificial Sequence
  Description of Artificial Sequence Synthetic oligonucleotide
ctagaaactgccctgtgg

SEQ ID NO: 15 - DNA, 20 bp, Artificial Sequence
  Description of Artificial Sequence Synthetic oligonucleotide
ggcaacactagaaactgccc

SEQ ID NO: 16 - DNA, 21 bp, Artificial Sequence
  Description of Artificial Sequence Synthetic oligonucleotide
ggagaagaggcaacactagaa

SEQ ID NO: 17 - DNA, 18 bp, Artificial Sequence
  Description of Artificial Sequence Synthetic oligonucleotide
cagggagaagaggcaaca

SEQ ID NO: 18 - DNA, 17 bp, Artificial Sequence
  Description of Artificial Sequence Synthetic oligonucleotide
actgcagggagaagagg

SEQ ID NO: 19 - DNA, 20 bp, Artificial Sequence
  Description of Artificial Sequence Synthetic oligonucleotide
gagcgaactgcagggagaag

SEQ ID NO: 20 - DNA, 20 bp, Artificial Sequence
  Description of Artificial Sequence Synthetic oligonucleotide
tccatgagcgaactgcaggg

SEQ ID NO: 21 - DNA, 20 bp, Artificial Sequence
  Description of Artificial Sequence Synthetic oligonucleotide
gggactccatgagcgaactg

SEQ ID NO: 22 - DNA, 17 bp, Artificial Sequence
  Description of Artificial Sequence Synthetic oligonucleotide
tccgggactccatgagc

SEQ ID NO: 23 - DNA, 21 bp, Artificial Sequence
  Description of Artificial Sequence Synthetic oligonucleotide
agcgccaggtccgggactcca

SEQ ID NO: 24 - DNA, 21 bp, Artificial Sequence
  Description of Artificial Sequence Synthetic oligonucleotide
gtccttcagcgccaggtccgg

SEQ ID NO: 25 - DNA, 21 bp, Artificial Sequence
  Description of Artificial Sequence Synthetic oligonucleotide
caggcgatgtccttcagcgcc

SEQ ID NO: 26 - DNA, 19 bp, Artificial Sequence
  Description of Artificial Sequence Synthetic oligonucleotide
cctcgctgcaggcgatgtc

SEQ ID NO: 27 - DNA, 18 bp, Artificial Sequence
  Description of Artificial Sequence Synthetic oligonucleotide
ggagggcctcgctgcagg

SEQ ID NO: 28 - DNA, 18 bp, Artificial Sequence
  Description of Artificial Sequence Synthetic oligonucleotide
gctccaggagggcctcgc

SEQ ID NO: 29 - DNA, 17 bp, Artificial Sequence
  Description of Artificial Sequence Synthetic oligonucleotide
gcgctccaggagggcct

SEQ ID NO: 30 - DNA, 18 bp, Artificial Sequence
  Description of Artificial Sequence Synthetic oligonucleotide
tgaagcgctccaggaggg

SEQ ID NO: 31 - DNA, 20 bp, Artificial Sequence
  Description of Artificial Sequence Synthetic oligonucleotide
gaagatgatgaagcgctcca

SEQ ID NO: 32 - DNA, 19 bp, Artificial Sequence
  Description of Artificial Sequence Synthetic oligonucleotide
tggctgaagatgatgaagc

SEQ ID NO: 33 - DNA, 19 bp, Artificial Sequence
  Description of Artificial Sequence Synthetic oligonucleotide
tctctggctgaagatgatg

SEQ ID NO: 34 - DNA, 17 bp, Artificial Sequence
  Description of Artificial Sequence Synthetic oligonucleotide
cgtctctggctgaagat

SEQ ID NO: 35 - DNA, 20 bp, Artificial Sequence
  Description of Artificial Sequence Synthetic oligonucleotide
ttgccccgcgtctctggctg

SEQ ID NO: 36 - DNA, 18 bp, Artificial Sequence
  Description of Artificial Sequence Synthetic oligonucleotide
caccgtctttgccccgcg

SEQ ID NO: 37 - DNA, 21 bp, Artificial Sequence
  Description of Artificial Sequence Synthetic oligonucleotide
atagcgcaccgtctttgcccc

SEQ ID NO: 38 - DNA, 17 bp, Artificial Sequence
  Description of Artificial Sequence Synthetic oligonucleotide
ggcatagcgcaccgtct

SEQ ID NO: 39 - DNA, 18 bp, Artificial Sequence
  Description of Artificial Sequence Synthetic oligonucleotide
cagggcatagcgcaccgt

SEQ ID NO: 40 - DNA, 19 bp, Artificial Sequence
  Description of Artificial Sequence Synthetic oligonucleotide
gagcacagggcatagcgca

SEQ ID NO: 41 - DNA, 17 bp, Artificial Sequence
  Description of Artificial Sequence Synthetic oligonucleotide
agagggagcacagggca

SEQ ID NO: 42 - DNA, 19 bp, Artificial Sequence
  Description of Artificial Sequence Synthetic oligonucleotide
tgggagagggagcacaggg

SEQ ID NO: 43 - DNA, 21 bp, Artificial Sequence
  Description of Artificial Sequence Synthetic oligonucleotide
tagggtgccctgggagaggga

SEQ ID NO: 44 - DNA, 21 bp, Artificial Sequence
  Description of Artificial Sequence Synthetic oligonucleotide
tatccactgtagggtgccctg

SEQ ID NO: 45 - DNA, 17 bp, Artificial Sequence
  Description of Artificial Sequence Synthetic oligonucleotide
tcttctatccactgtag

SEQ ID NO: 46 - DNA, 17 bp, Artificial Sequence
  Description of Artificial Sequence Synthetic oligonucleotide
agtgtcttctatccact

SEQ ID NO: 47 - DNA, 18 bp, Artificial Sequence
  Description of Artificial Sequence Synthetic oligonucleotide
cagagtgtcttctatcca

SEQ ID NO: 48 - DNA, 20 bp, Artificial Sequence
  Description of Artificial Sequence Synthetic oligonucleotide
gttggcatacagagtgtctt

SEQ ID NO: 49 - DNA, 19 bp, Artificial Sequence
  Description of Artificial Sequence Synthetic oligonucleotide
ccacgttggcatacagagt

SEQ ID NO: 50 - DNA, 18 bp, Artificial Sequence
  Description of Artificial Sequence Synthetic oligonucleotide
aagtccacgttggcatac

SEQ ID NO: 51 - DNA, 18 bp, Artificial Sequence
  Description of Artificial Sequence Synthetic oligonucleotide
aagaagtccacgttggca

SEQ ID NO: 52 - DNA, 19 bp, Artificial Sequence
  Description of Artificial Sequence Synthetic oligonucleotide
gcttgaagaagtccacgtt

SEQ ID NO: 53 - DNA, 18 bp, Artificial Sequence
  Description of Artificial Sequence Synthetic oligonucleotide
aagagcttgaagaagtcc

SEQ ID NO: 54 - DNA, 18 bp, Artificial Sequence
  Description of Artificial Sequence Synthetic oligonucleotide
cggaagagcttgaagaag

SEQ ID NO: 55 - DNA, 17 bp, Artificial Sequence
  Description of Artificial Sequence Synthetic oligonucleotide
aacacggaagagcttga

SEQ ID NO: 56 - DNA, 19 bp, Artificial Sequence
  Description of Artificial Sequence Synthetic oligonucleotide
cttacaacacggaagagct

SEQ ID NO: 57 - DNA, 18 bp, Artificial Sequence
  Description of Artificial Sequence Synthetic oligonucleotide
ctcccttacaacacggaa

SEQ ID NO: 58 - DNA, 21 bp, Artificial Sequence
  Description of Artificial Sequence Synthetic oligonucleotide
ccaaacccctcccttacaaca

SEQ ID NO: 59 - DNA, 17 bp, Artificial Sequence
  Description of Artificial Sequence Synthetic oligonucleotide
cagccaaacccctccct

SEQ ID NO: 60 - DNA, 17 bp, Artificial Sequence
  Description of Artificial Sequence Synthetic oligonucleotide
agcagccaaacccctcc

SEQ ID NO: 61 - DNA, 17 bp, Artificial Sequence
  Description of Artificial Sequence Synthetic oligonucleotide
cgagcagccaaacccct

SEQ ID NO: 62 - DNA, 18 bp, Artificial Sequence
  Description of Artificial Sequence Synthetic oligonucleotide
tggcgagcagccaaaccc

SEQ ID NO: 63 - DNA, 20 bp, Artificial Sequence
  Description of Artificial Sequence Synthetic oligonucleotide
tgcaattggcgagcagccaa

SEQ ID NO: 64 - DNA, 17 bp, Artificial Sequence
  Description of Artificial Sequence Synthetic oligonucleotide
aattggcgagcagccaa

SEQ ID NO: 65 - DNA, 18 bp, Artificial Sequence
  Description of Artificial Sequence Synthetic oligonucleotide
caattggcgagcagccaa

SEQ ID NO: 66 - DNA, 19 bp, Artificial Sequence
  Description of Artificial Sequence Synthetic oligonucleotide
gcaattggcgagcagccaa

SEQ ID NO: 67 - DNA, 21 bp, Artificial Sequence
  Description of Artificial Sequence Synthetic oligonucleotide
ttgcaattggcgagcagccaa

SEQ ID NO: 68 - DNA, 17 bp, Artificial Sequence
  Description of Artificial Sequence Synthetic oligonucleotide
caattggcgagcagcca

SEQ ID NO: 69 - DNA, 18 bp, Artificial Sequence
  Description of Artificial Sequence Synthetic oligonucleotide
gcaattggcgagcagcca

SEQ ID NO: 70 - DNA, 19 bp, Artificial Sequence
  Description of Artificial Sequence Synthetic oligonucleotide
tgcaattggcgagcagcca

SEQ ID NO: 71 - DNA, 20 bp, Artificial Sequence
  Description of Artificial Sequence Synthetic oligonucleotide
ttgcaattggcgagcagcca

SEQ ID NO: 72 - DNA, 21 bp, Artificial Sequence
  Description of Artificial Sequence Synthetic oligonucleotide
cttgcaattggcgagcagcca

SEQ ID NO: 73 - DNA, 17 bp, Artificial Sequence
  Description of Artificial Sequence Synthetic oligonucleotide
gcaattggcgagcagcc

SEQ ID NO: 74 - DNA, 18 bp, Artificial Sequence
  Description of Artificial Sequence Synthetic oligonucleotide
tgcaattggcgagcagcc

SEQ ID NO: 75 - DNA, 19 bp, Artificial Sequence
  Description of Artificial Sequence Synthetic oligonucleotide
ttgcaattggcgagcagcc

SEQ ID NO: 76 - DNA, 20 bp, Artificial Sequence
  Description of Artificial Sequence Synthetic oligonucleotide
cttgcaattggcgagcagcc

SEQ ID NO: 77 - DNA, 21 bp, Artificial Sequence
  Description of Artificial Sequence Synthetic oligonucleotide
ccttgcaattggcgagcagcc

SEQ ID NO: 78 - DNA, 17 bp, Artificial Sequence
  Description of Artificial Sequence Synthetic oligonucleotide
tgcaattggcgagcagc

SEQ ID NO: 79 - DNA, 18 bp, Artificial Sequence
  Description of Artificial Sequence Synthetic oligonucleotide
ttgcaattggcgagcagc

SEQ ID NO: 80 - DNA, 19 bp, Artificial Sequence
  Description of Artificial Sequence Synthetic oligonucleotide
cttgcaattggcgagcagc

SEQ ID NO: 81 - DNA, 20 bp, Artificial Sequence
  Description of Artificial Sequence Synthetic oligonucleotide
ccttgcaattggcgagcagc

SEQ ID NO: 82 - DNA, 21 bp, Artificial Sequence
  Description of Artificial Sequence Synthetic oligonucleotide
accttgcaattggcgagcagc

SEQ ID NO: 83 - DNA, 17 bp, Artificial Sequence
  Description of Artificial Sequence Synthetic oligonucleotide
ttgcaattggcgagcag

SEQ ID NO: 84 - DNA, 18 bp, Artificial Sequence
  Description of Artificial Sequence Synthetic oligonucleotide
cttgcaattggcgagcag

SEQ ID NO: 85 - DNA, 19 bp, Artificial Sequence
  Description of Artificial Sequence Synthetic oligonucleotide
ccttgcaattggcgagcag

SEQ ID NO: 86 - DNA, 20 bp, Artificial Sequence
  Description of Artificial Sequence Synthetic oligonucleotide
accttgcaattggcgagcag

SEQ ID NO: 87 - DNA, 21 bp, Artificial Sequence
  Description of Artificial Sequence Synthetic oligonucleotide
caccttgcaattggcgagcag

SEQ ID NO: 88 - DNA, 17 bp, Artificial Sequence
  Description of Artificial Sequence Synthetic oligonucleotide
cttgcaattggcgagca

SEQ ID NO: 89 - DNA, 18 bp, Artificial Sequence
  Description of Artificial Sequence Synthetic oligonucleotide
ccttgcaattggcgagca

SEQ ID NO: 90 - DNA, 19 bp, Artificial Sequence
  Description of Artificial Sequence Synthetic oligonucleotide
accttgcaattggcgagca

SEQ ID NO: 91 - DNA, 20 bp, Artificial Sequence
  Description of Artificial Sequence Synthetic oligonucleotide
caccttgcaattggcgagca

SEQ ID NO: 92 - DNA, 21 bp, Artificial Sequence
  Description of Artificial Sequence Synthetic oligonucleotide
tcaccttgcaattggcgagca

SEQ ID NO: 93 - DNA, 17 bp, Artificial Sequence
  Description of Artificial Sequence Synthetic oligonucleotide
ccttgcaattggcgagc

SEQ ID NO: 94 - DNA, 18 bp, Artificial Sequence
  Description of Artificial Sequence Synthetic oligonucleotide
accttgcaattggcgagc

SEQ ID NO: 95 - DNA, 19 bp, Artificial Sequence
  Description of Artificial Sequence Synthetic oligonucleotide
caccttgcaattggcgagc

SEQ ID NO: 96 - DNA, 20 bp, Artificial Sequence
  Description of Artificial Sequence Synthetic oligonucleotide
tcaccttgcaattggcgagc

SEQ ID NO: 97 - DNA, 21 bp, Artificial Sequence
  Description of Artificial Sequence Synthetic oligonucleotide
atcaccttgcaattggcgagc

SEQ ID NO: 98 - DNA, 17 bp, Artificial Sequence
  Description of Artificial Sequence Synthetic oligonucleotide
accttgcaattggcgag

SEQ ID NO: 99 - DNA, 18 bp, Artificial Sequence
  Description of Artificial Sequence Synthetic oligonucleotide
caccttgcaattggcgag

SEQ ID NO: 100 - DNA, 19 bp, Artificial Sequence
  Description of Artificial Sequence Synthetic oligonucleotide
tcaccttgcaattggcgag

SEQ ID NO: 101 - DNA, 20 bp, Artificial Sequence
  Description of Artificial Sequence Synthetic oligonucleotide
atcaccttgcaattggcgag

SEQ ID NO: 102 - DNA, 21 bp, Artificial Sequence
  Description of Artificial Sequence Synthetic oligonucleotide
aatcaccttgcaattggcgag

SEQ ID NO: 103 - DNA, 17 bp, Artificial Sequence
  Description of Artificial Sequence Synthetic oligonucleotide
caccttgcaattggcga

SEQ ID NO: 104 - DNA, 18 bp, Artificial Sequence
  Description of Artificial Sequence Synthetic oligonucleotide
tcaccttgcaattggcga

SEQ ID NO: 105 - DNA, 19 bp, Artificial Sequence
  Description of Artificial Sequence Synthetic oligonucleotide
atcaccttgcaattggcga

SEQ ID NO: 106 - DNA, 20 bp, Artificial Sequence
  Description of Artificial Sequence Synthetic oligonucleotide
aatcaccttgcaattggcga

SEQ ID NO: 107 - DNA, 21 bp, Artificial Sequence
  Description of Artificial Sequence Synthetic oligonucleotide
gaatcaccttgcaattggcga

SEQ ID NO: 108 - DNA, 19 bp, Artificial Sequence
  Description of Artificial Sequence Synthetic oligonucleotide
aatcaccttgcaattggcg

SEQ ID NO: 109 - DNA, 17 bp, Artificial Sequence
  Description of Artificial Sequence Synthetic oligonucleotide
tcaccttgcaattggcg

SEQ ID NO: 110 - DNA, 18 bp, Artificial Sequence
  Description of Artificial Sequence Synthetic oligonucleotide
atcaccttgcaattggcg

SEQ ID NO: 111 - DNA, 20 bp, Artificial Sequence
  Description of Artificial Sequence Synthetic oligonucleotide
gaatcaccttgcaattggcg

SEQ ID NO: 112 - DNA, 21 bp, Artificial Sequence
  Description of Artificial Sequence Synthetic oligonucleotide
ggaatcaccttgcaattggcg

SEQ ID NO: 113 - DNA, 17 bp, Artificial Sequence
  Description of Artificial Sequence Synthetic oligonucleotide
atcaccttgcaattggc

SEQ ID NO: 114 - DNA, 18 bp, Artificial Sequence
  Description of Artificial Sequence Synthetic oligonucleotide
aatcaccttgcaattggc

SEQ ID NO: 115 - DNA, 19 bp, Artificial Sequence
  Description of Artificial Sequence Synthetic oligonucleotide
gaatcaccttgcaattggc

SEQ ID NO: 116 - DNA, 20 bp, Artificial Sequence
  Description of Artificial Sequence Synthetic oligonucleotide
ggaatcaccttgcaattggc

SEQ ID NO: 117 - DNA, 21 bp, Artificial Sequence
  Description of Artificial Sequence Synthetic oligonucleotide
aggaatcaccttgcaattggc

SEQ ID NO: 118 - DNA, 17 bp, Artificial Sequence
  Description of Artificial Sequence Synthetic oligonucleotide
aatcaccttgcaattgg

SEQ ID NO: 119 - DNA, 18 bp, Artificial Sequence
  Description of Artificial Sequence Synthetic oligonucleotide
gaatcaccttgcaattgg

SEQ ID NO: 120 - DNA, 19 bp, Artificial Sequence
  Description of Artificial Sequence Synthetic oligonucleotide
ggaatcaccttgcaattgg

SEQ ID NO: 121 - DNA, 20 bp, Artificial Sequence
  Description of Artificial Sequence Synthetic oligonucleotide
aggaatcaccttgcaattgg

SEQ ID NO: 122 - DNA, 21 bp, Artificial Sequence
  Description of Artificial Sequence Synthetic oligonucleotide
caggaatcaccttgcaattgg

SEQ ID NO: 123 - DNA, 17 bp, Artificial Sequence
  Description of Artificial Sequence Synthetic oligonucleotide
gaatcaccttgcaattg

SEQ ID NO: 124 - DNA, 18 bp, Artificial Sequence
  Description of Artificial Sequence Synthetic oligonucleotide
ggaatcaccttgcaattg

SEQ ID NO: 125 - DNA, 19 bp, Artificial Sequence
  Description of Artificial Sequence Synthetic oligonucleotide
aggaatcaccttgcaattg

SEQ ID NO: 126 - DNA, 20 bp, Artificial Sequence
  Description of Artificial Sequence Synthetic oligonucleotide
caggaatcaccttgcaattg

SEQ ID NO: 127 - DNA, 21 bp, Artificial Sequence
  Description of Artificial Sequence Synthetic oligonucleotide
ccaggaatcaccttgcaattg

SEQ ID NO: 128 - DNA, 17 bp, Artificial Sequence
  Description of Artificial Sequence Synthetic oligonucleotide
ggaatcaccttgcaatt

SEQ ID NO: 129 - DNA, 18 bp, Artificial Sequence
  Description of Artificial Sequence Synthetic oligonucleotide
aggaatcaccttgcaatt

SEQ ID NO: 130 - DNA, 19 bp, Artificial Sequence
  Description of Artificial Sequence Synthetic oligonucleotide
caggaatcaccttgcaatt

SEQ ID NO: 131 - DNA, 20 bp, Artificial Sequence
  Description of Artificial Sequence Synthetic oligonucleotide
ccaggaatcaccttgcaatt

SEQ ID NO: 132 - DNA, 21 bp, Artificial Sequence
  Description of Artificial Sequence Synthetic oligonucleotide
cccaggaatcaccttgcaatt

SEQ ID NO: 133 - DNA, 17 bp, Artificial Sequence
  Description of Artificial Sequence Synthetic oligonucleotide
aggaatcaccttgcaat

SEQ ID NO: 134 - DNA, 18 bp, Artificial Sequence
  Description of Artificial Sequence Synthetic oligonucleotide
caggaatcaccttgcaat

SEQ ID NO: 135 - DNA, 19 bp, Artificial Sequence
  Description of Artificial Sequence Synthetic oligonucleotide
ccaggaatcaccttgcaat

SEQ ID NO: 136 - DNA, 20 bp, Artificial Sequence
  Description of Artificial Sequence Synthetic oligonucleotide
cccaggaatcaccttgcaat

SEQ ID NO: 137 - DNA, 21 bp, Artificial Sequence
  Description of Artificial Sequence Synthetic oligonucleotide
ccccaggaatcaccttgcaat

SEQ ID NO: 138 - DNA, 19 bp, Artificial Sequence
  Description of Artificial Sequence Synthetic oligonucleotide
cccaggaatcaccttgcaa

SEQ ID NO: 139 - DNA, 17 bp, Artificial Sequence
  Description of Artificial Sequence Synthetic oligonucleotide
caggaatcaccttgcaa

SEQ ID NO: 140 - DNA, 18 bp, Artificial Sequence
  Description of Artificial Sequence Synthetic oligonucleotide
ccaggaatcaccttgcaa

SEQ ID NO: 141 - DNA, 20 bp, Artificial Sequence
  Description of Artificial Sequence Synthetic oligonucleotide
ccccaggaatcaccttgcaa

SEQ ID NO: 142 - DNA, 21 bp, Artificial Sequence
  Description of Artificial Sequence Synthetic oligonucleotide
accccaggaatcaccttgcaa

SEQ ID NO: 143 - DNA, 17 bp, Artificial Sequence
  Description of Artificial Sequence Synthetic oligonucleotide
ccaggaatcaccttgca

SEQ ID NO: 144 - DNA, 18 bp, Artificial Sequence
  Description of Artificial Sequence Synthetic oligonucleotide
cccaggaatcaccttgca

SEQ ID NO: 145 - DNA, 19 bp, Artificial Sequence
  Description of Artificial Sequence Synthetic oligonucleotide
ccccaggaatcaccttgca

SEQ ID NO: 146 - DNA, 20 bp, Artificial Sequence
  Description of Artificial Sequence Synthetic oligonucleotide
accccaggaatcaccttgca

SEQ ID NO: 147 - DNA, 21 bp, Artificial Sequence
  Description of Artificial Sequence Synthetic oligonucleotide
taccccaggaatcaccttgca

SEQ ID NO: 148 - DNA, 17 bp, Artificial Sequence
  Description of Artificial Sequence Synthetic oligonucleotide
cccaggaatcaccttgc

SEQ ID NO: 149 - DNA, 18 bp, Artificial Sequence
  Description of Artificial Sequence Synthetic oligonucleotide
ccccaggaatcaccttgc

SEQ ID NO: 150 - DNA, 19 bp, Artificial Sequence
  Description of Artificial Sequence Synthetic oligonucleotide
accccaggaatcaccttgc

SEQ ID NO: 151 - DNA, 20 bp, Artificial Sequence
  Description of Artificial Sequence Synthetic oligonucleotide
taccccaggaatcaccttgc

SEQ ID NO: 152 - DNA, 21 bp, Artificial Sequence
  Description of Artificial Sequence Synthetic oligonucleotide
ctaccccaggaatcaccttgc

SEQ ID NO: 153 - DNA, 17 bp, Artificial Sequence
  Description of Artificial Sequence Synthetic oligonucleotide
ccccaggaatcaccttg

SEQ ID NO: 154 - DNA, 18 bp, Artificial Sequence
  Description of Artificial Sequence Synthetic oligonucleotide
accccaggaatcaccttg

SEQ ID NO: 155 - DNA, 19 bp, Artificial Sequence
  Description of Artificial Sequence Synthetic oligonucleotide
taccccaggaatcaccttg

SEQ ID NO: 156 - DNA, 20 bp, Artificial Sequence
  Description of Artificial Sequence Synthetic oligonucleotide
ctaccccaggaatcaccttg

SEQ ID NO: 157 - DNA, 21 bp, Artificial Sequence
  Description of Artificial Sequence Synthetic oligonucleotide
gctaccccaggaatcaccttg

SEQ ID NO: 158 - DNA, 17 bp, Artificial Sequence
  Description of Artificial Sequence Synthetic oligonucleotide
accccaggaatcacctt

SEQ ID NO: 159 - DNA, 18 bp, Artificial Sequence
  Description of Artificial Sequence Synthetic oligonucleotide
taccccaggaatcacctt

SEQ ID NO: 160 - DNA, 20 bp, Artificial Sequence
  Description of Artificial Sequence Synthetic oligonucleotide
gctaccccaggaatcacctt

SEQ ID NO: 161 - DNA, 21 bp, Artificial Sequence
  Description of Artificial Sequence Synthetic oligonucleotide
tgctaccccaggaatcacctt

SEQ ID NO: 162 - DNA, 18 bp, Artificial Sequence
  Description of Artificial Sequence Synthetic oligonucleotide
ctaccccaggaatcacct

SEQ ID NO: 163 - DNA, 17 bp, Artificial Sequence
  Description of Artificial Sequence Synthetic oligonucleotide
taccccaggaatcacct

SEQ ID NO: 164 - DNA, 19 bp, Artificial Sequence
  Description of Artificial Sequence Synthetic oligonucleotide
gctaccccaggaatcacct

SEQ ID NO: 165 - DNA, 20 bp, Artificial Sequence
  Description of Artificial Sequence Synthetic oligonucleotide
tgctaccccaggaatcacct

SEQ ID NO: 166 - DNA, 21 bp, Artificial Sequence
  Description of Artificial Sequence Synthetic oligonucleotide
ctgctaccccaggaatcacct

SEQ ID NO: 167 - DNA, 17 bp, Artificial Sequence
  Description of Artificial Sequence Synthetic oligonucleotide
ctaccccaggaatcacc

SEQ ID NO: 168 - DNA, 18 bp, Artificial Sequence
  Description of Artificial Sequence Synthetic oligonucleotide
gctaccccaggaatcacc

SEQ ID NO: 169 - DNA, 19 bp, Artificial Sequence
  Description of Artificial Sequence Synthetic oligonucleotide
tgctaccccaggaatcacc

SEQ ID NO: 170 - DNA, 20 bp, Artificial Sequence
  Description of Artificial Sequence Synthetic oligonucleotide
ctgctaccccaggaatcacc

SEQ ID NO: 171 - DNA, 21 bp, Artificial Sequence
  Description of Artificial Sequence Synthetic oligonucleotide
tctgctaccccaggaatcacc

SEQ ID NO: 172 - DNA, 17 bp, Artificial Sequence
  Description of Artificial Sequence Synthetic oligonucleotide
gctaccccaggaatcac

SEQ ID NO: 173 - DNA, 18 bp, Artificial Sequence
  Description of Artificial Sequence Synthetic oligonucleotide
tgctaccccaggaatcac

SEQ ID NO: 174 - DNA, 19 bp, Artificial Sequence
  Description of Artificial Sequence Synthetic oligonucleotide
ctgctaccccaggaatcac

SEQ ID NO: 175 - DNA, 20 bp, Artificial Sequence
  Description of Artificial Sequence Synthetic oligonucleotide
tctgctaccccaggaatcac

SEQ ID NO: 176 - DNA, 21 bp, Artificial Sequence
  Description of Artificial Sequence Synthetic oligonucleotide
ctctgctaccccaggaatcac

SEQ ID NO: 177 - DNA, 17 bp, Artificial Sequence
  Description of Artificial Sequence Synthetic oligonucleotide
tgctaccccaggaatca

SEQ ID NO: 178 - DNA, 18 bp, Artificial Sequence
  Description of Artificial Sequence Synthetic oligonucleotide
ctgctaccccaggaatca

SEQ ID NO: 179 - DNA, 19 bp, Artificial Sequence
  Description of Artificial Sequence Synthetic oligonucleotide
tctgctaccccaggaatca

SEQ ID NO: 180 - DNA, 20 bp, Artificial Sequence
  Description of Artificial Sequence Synthetic oligonucleotide
ctctgctaccccaggaatca

SEQ ID NO: 181 - DNA, 21 bp, Artificial Sequence
  Description of Artificial Sequence Synthetic oligonucleotide
gctctgctaccccaggaatca

SEQ ID NO: 182 - DNA, 17 bp, Artificial Sequence
  Description of Artificial Sequence Synthetic oligonucleotide
ctgctaccccaggaatc

SEQ ID NO: 183 - DNA, 18 bp, Artificial Sequence
  Description of Artificial Sequence Synthetic oligonucleotide
tctgctaccccaggaatc

SEQ ID NO: 184 - DNA, 19 bp, Artificial Sequence
  Description of Artificial Sequence Synthetic oligonucleotide
ctctgctaccccaggaatc

SEQ ID NO: 185 - DNA, 20 bp, Artificial Sequence
  Description of Artificial Sequence Synthetic oligonucleotide
gctctgctaccccaggaatc

SEQ ID NO: 186 - DNA, 21 bp, Artificial Sequence
  Description of Artificial Sequence Synthetic oligonucleotide
ggctctgctaccccaggaatc

SEQ ID NO: 187 - DNA, 17 bp, Artificial Sequence
  Description of Artificial Sequence Synthetic oligonucleotide
tctgctaccccaggaat

SEQ ID NO: 188 - DNA, 18 bp, Artificial Sequence
  Description of Artificial Sequence Synthetic oligonucleotide
ctctgctaccccaggaat

SEQ ID NO: 189 - DNA, 19 bp, Artificial Sequence
  Description of Artificial Sequence Synthetic oligonucleotide
gctctgctaccccaggaat

SEQ ID NO: 190 - DNA, 20 bp, Artificial Sequence
  Description of Artificial Sequence Synthetic oligonucleotide
ggctctgctaccccaggaat

SEQ ID NO: 191 - DNA, 20 bp, Artificial Sequence
  Description of Artificial Sequence Synthetic oligonucleotide
aggctctgctaccccaggaa

SEQ ID NO: 192 - DNA, 17 bp, Artificial Sequence
  Description of Artificial Sequence Synthetic oligonucleotide
ctctgctaccccaggaa

SEQ ID NO: 193 - DNA, 18 bp, Artificial Sequence
  Description of Artificial Sequence Synthetic oligonucleotide
gctctgctaccccaggaa

SEQ ID NO: 194 - DNA, 19 bp, Artificial Sequence
  Description of Artificial Sequence Synthetic oligonucleotide
ggctctgctaccccaggaa

SEQ ID NO: 195 - DNA, 17 bp, Artificial Sequence
  Description of Artificial Sequence Synthetic oligonucleotide
gctctgctaccccagga

SEQ ID NO: 196 - DNA, 18 bp, Artificial Sequence
  Description of Artificial Sequence Synthetic oligonucleotide
ggctctgctaccccagga

SEQ ID NO: 197 - DNA, 17 bp, Artificial Sequence
  Description of Artificial Sequence Synthetic oligonucleotide
ggctctgctaccccagg

SEQ ID NO: 198 - DNA, 19 bp, Artificial Sequence
  Description of Artificial Sequence Synthetic oligonucleotide
cgtgaggctctgctacccc

SEQ ID NO: 199 - DNA, 18 bp, Artificial Sequence
  Description of Artificial Sequence Synthetic oligonucleotide
aattcgtgaggctctgct

SEQ ID NO: 200 - DNA, 19 bp, Artificial Sequence
  Description of Artificial Sequence Synthetic oligonucleotide
ggtcaattcgtgaggctct

SEQ ID NO: 201 - DNA, 17 bp, Artificial Sequence
  Description of Artificial Sequence Synthetic oligonucleotide
caaggtcaattcgtgag

SEQ ID NO: 202 - DNA, 21 bp, Artificial Sequence
  Description of Artificial Sequence Synthetic oligonucleotide
cctccccaaggtcaattcgtg

SEQ ID NO: 203 - DNA, 21 bp, Artificial Sequence
  Description of Artificial Sequence Synthetic oligonucleotide
ggctcacgccctccccaaggt

SEQ ID NO: 204 - DNA, 17 bp, Artificial Sequence
  Description of Artificial Sequence Synthetic oligonucleotide
caggctcacgccctccc

SEQ ID NO: 205 - DNA, 18 bp, Artificial Sequence
  Description of Artificial Sequence Synthetic oligonucleotide
caccaggctcacgccctc

SEQ ID NO: 206 - DNA, 20 bp, Artificial Sequence
  Description of Artificial Sequence Synthetic oligonucleotide
ccagaacaccaggctcacgc

SEQ ID NO: 207 - DNA, 19 bp, Artificial Sequence
  Description of Artificial Sequence Synthetic oligonucleotide
ctaccccaggaatcacctt

SEQ ID NO: 208 - DNA, 20 bp, Artificial Sequence
  Description of Artificial Sequence Synthetic oligonucleotide
taaaaacccaacaagtgctt

SEQ ID NO: 209 - DNA, 20 bp, Artificial Sequence
  Description of Artificial Sequence Synthetic oligonucleotide
ttaaaaacccaacaagtgct

SEQ ID NO: 210 - DNA, 20 bp, Artificial Sequence
  Description of Artificial Sequence Synthetic oligonucleotide
cttaaaaacccaacaagtgc

SEQ ID NO: 211 - DNA, 20 bp, Artificial Sequence
  Description of Artificial Sequence Synthetic oligonucleotide
gcttaaaaacccaacaagtg

SEQ ID NO: 212 - DNA, 20 bp, Artificial Sequence
  Description of Artificial Sequence Synthetic oligonucleotide
cgcttaaaaacccaacaagt

SEQ ID NO: 213 - DNA, 20 bp, Artificial Sequence
  Description of Artificial Sequence Synthetic oligonucleotide
ccccgctcacattcatgatc

SEQ ID NO: 214 - DNA, 20 bp, Artificial Sequence
  Description of Artificial Sequence Synthetic oligonucleotide
cacaccccgctcacattcat

SEQ ID NO: 215 - DNA, 20 bp, Artificial Sequence
  Description of Artificial Sequence Synthetic oligonucleotide
tgtttacacaccccgctcac

SEQ ID NO: 216 - DNA, 21 bp, Artificial Sequence
  Description of Artificial Sequence Synthetic oligonucleotide
tctccagtctgtttacacacc

SEQ ID NO: 217 - DNA, 20 bp, Artificial Sequence
  Description of Artificial Sequence Synthetic oligonucleotide
ctccagtctgtttacacacc

SEQ ID NO: 218 - DNA, 18 bp, Artificial Sequence
  Description of Artificial Sequence Synthetic oligonucleotide
ccagtctgtttacacacc

SEQ ID NO: 219 - DNA, 17 bp, Artificial Sequence
  Description of Artificial Sequence Synthetic oligonucleotide
cagtctgtttacacacc

SEQ ID NO: 220 - DNA, 21 bp, Artificial Sequence
  Description of Artificial Sequence Synthetic oligonucleotide
atctccagtctgtttacacac

SEQ ID NO: 221 - DNA, 20 bp, Artificial Sequence
  Description of Artificial Sequence Synthetic oligonucleotide
tctccagtctgtttacacac

SEQ ID NO: 222 - DNA, 19 bp, Artificial Sequence
  Description of Artificial Sequence Synthetic oligonucleotide
ctccagtctgtttacacac

SEQ ID NO: 223 - DNA, 18 bp, Artificial Sequence
  Description of Artificial Sequence Synthetic oligonucleotide
tccagtctgtttacacac

SEQ ID NO: 224 - DNA, 17 bp, Artificial Sequence
  Description of Artificial Sequence Synthetic oligonucleotide
ccagtctgtttacacac

SEQ ID NO: 225 - DNA, 21 bp, Artificial Sequence
  Description of Artificial Sequence Synthetic oligonucleotide
aatctccagtctgtttacaca

SEQ ID NO: 226 - DNA, 20 bp, Artificial Sequence
  Description of Artificial Sequence Synthetic oligonucleotide
atctccagtctgtttacaca

SEQ ID NO: 227 - DNA, 19 bp, Artificial Sequence
  Description of Artificial Sequence Synthetic oligonucleotide
tctccagtctgtttacaca

SEQ ID NO: 228 - DNA, 18 bp, Artificial Sequence
  Description of Artificial Sequence Synthetic oligonucleotide
ctccagtctgtttacaca

SEQ ID NO: 229 - DNA, 17 bp, Artificial Sequence
  Description of Artificial Sequence Synthetic oligonucleotide
tccagtctgtttacaca

SEQ ID NO: 230 - DNA, 21 bp, Artificial Sequence
  Description of Artificial Sequence Synthetic oligonucleotide
aaatctccagtctgtttacac

SEQ ID NO: 231 - DNA, 20 bp, Artificial Sequence
  Description of Artificial Sequence Synthetic oligonucleotide
aatctccagtctgtttacac

SEQ ID NO: 232 - DNA, 18 bp, Artificial Sequence
  Description of Artificial Sequence Synthetic oligonucleotide
tctccagtctgtttacac

SEQ ID NO: 233 - DNA, 17 bp, Artificial Sequence
  Description of Artificial Sequence Synthetic oligonucleotide
ctccagtctgtttacac

SEQ ID NO: 234 - DNA, 21 bp, Artificial Sequence
  Description of Artificial Sequence Synthetic oligonucleotide
caaatctccagtctgtttaca

SEQ ID NO: 235 - DNA, 20 bp, Artificial Sequence
  Description of Artificial Sequence Synthetic oligonucleotide
aaatctccagtctgtttaca

SEQ ID NO: 236 - DNA, 19 bp, Artificial Sequence
  Description of Artificial Sequence Synthetic oligonucleotide
aatctccagtctgtttaca

SEQ ID NO: 237 - DNA, 18 bp, Artificial Sequence
  Description of Artificial Sequence Synthetic oligonucleotide
atctccagtctgtttaca

SEQ ID NO: 238 - DNA, 17 bp, Artificial Sequence
  Description of Artificial Sequence Synthetic oligonucleotide
tctccagtctgtttaca

SEQ ID NO: 239 - DNA, 21 bp, Artificial Sequence
  Description of Artificial Sequence Synthetic oligonucleotide
tcaaatctccagtctgtttac

SEQ ID NO: 240 - DNA, 20 bp, Artificial Sequence
  Description of Artificial Sequence Synthetic oligonucleotide
caaatctccagtctgtttac

SEQ ID NO: 241 - DNA, 19 bp, Artificial Sequence
  Description of Artificial Sequence Synthetic oligonucleotide
aaatctccagtctgtttac

SEQ ID NO: 242 - DNA, 17 bp, Artificial Sequence
  Description of Artificial Sequence Synthetic oligonucleotide
atctccagtctgtttac

SEQ ID NO: 243 - DNA, 21 bp, Artificial Sequence
  Description of Artificial Sequence Synthetic oligonucleotide
ctcaaatctccagtctgttta

SEQ ID NO: 244 - DNA, 20 bp, Artificial Sequence
  Description of Artificial Sequence Synthetic oligonucleotide
tcaaatctccagtctgttta

SEQ ID NO: 245 - DNA, 19 bp, Artificial Sequence
  Description of Artificial Sequence Synthetic oligonucleotide
caaatctccagtctgttta

SEQ ID NO: 246 - DNA, 18 bp, Artificial Sequence
  Description of Artificial Sequence Synthetic oligonucleotide
aaatctccagtctgttta

SEQ ID NO: 247 - DNA, 17 bp, Artificial Sequence
  Description of Artificial Sequence Synthetic oligonucleotide
aatctccagtctgttta

SEQ ID NO: 248 - DNA, 20 bp, Artificial Sequence
  Description of Artificial Sequence Synthetic oligonucleotide
ctcaaatctccagtctgttt

SEQ ID NO: 249 - DNA, 19 bp, Artificial Sequence
  Description of Artificial Sequence Synthetic oligonucleotide
tcaaatctccagtctgttt

SEQ ID NO: 250 - DNA, 18 bp, Artificial Sequence
  Description of Artificial Sequence Synthetic oligonucleotide
caaatctccagtctgttt

SEQ ID NO: 251 - DNA, 17 bp, Artificial Sequence
  Description of Artificial Sequence Synthetic oligonucleotide
aaatctccagtctgttt

SEQ ID NO: 252 - DNA, 21 bp, Artificial Sequence
  Description of Artificial Sequence Synthetic oligonucleotide
tactcaaatctccagtctgtt

SEQ ID NO: 253 - DNA, 20 bp, Artificial Sequence
  Description of Artificial Sequence Synthetic oligonucleotide
actcaaatctccagtctgtt

SEQ ID NO: 254 - DNA, 18 bp, Artificial Sequence
  Description of Artificial Sequence Synthetic oligonucleotide
tcaaatctccagtctgtt

SEQ ID NO: 255 - DNA, 17 bp, Artificial Sequence
  Description of Artificial Sequence Synthetic oligonucleotide
caaatctccagtctgtt

SEQ ID NO: 256 - DNA, 21 bp, Artificial Sequence
  Description of Artificial Sequence Synthetic oligonucleotide
ctactcaaatctccagtctgt

SEQ ID NO: 257 - DNA, 19 bp, Artificial Sequence
  Description of Artificial Sequence Synthetic oligonucleotide
actcaaatctccagtctgt

SEQ ID NO: 258 - DNA, 18 bp, Artificial Sequence
  Description of Artificial Sequence Synthetic oligonucleotide
ctcaaatctccagtctgt

SEQ ID NO: 259 - DNA, 17 bp, Artificial Sequence
  Description of Artificial Sequence Synthetic oligonucleotide
tcaaatctccagtctgt

SEQ ID NO: 260 - DNA, 21 bp, Artificial Sequence
  Description of Artificial Sequence Synthetic oligonucleotide
cctactcaaatctccagtctg

SEQ ID NO: 261 - DNA, 19 bp, Artificial Sequence
  Description of Artificial Sequence Synthetic oligonucleotide
tactcaaatctccagtctg

SEQ ID NO: 262 - DNA, 18 bp, Artificial Sequence
  Description of Artificial Sequence Synthetic oligonucleotide
actcaaatctccagtctg

SEQ ID NO: 263 - DNA, 17 bp, Artificial Sequence
  Description of Artificial Sequence Synthetic oligonucleotide
ctcaaatctccagtctg

SEQ ID NO: 264 - DNA, 21 bp, Artificial Sequence
  Description of Artificial Sequence Synthetic oligonucleotide
tcctactcaaatctccagtct

SEQ ID NO: 265 - DNA, 19 bp, Artificial Sequence
  Description of Artificial Sequence Synthetic oligonucleotide
ctactcaaatctccagtct

SEQ ID NO: 266 - DNA, 18 bp, Artificial Sequence
  Description of Artificial Sequence Synthetic oligonucleotide
tactcaaatctccagtct

SEQ ID NO: 267 - DNA, 17 bp, Artificial Sequence
  Description of Artificial Sequence Synthetic oligonucleotide
actcaaatctccagtct

SEQ ID NO: 268 - DNA, 21 bp, Artificial Sequence
  Description of Artificial Sequence Synthetic oligonucleotide
atcctactcaaatctccagtc

SEQ ID NO: 269 - DNA, 20 bp, Artificial Sequence
  Description of Artificial Sequence Synthetic oligonucleotide
tcctactcaaatctccagtc

SEQ ID NO: 270 - DNA, 19 bp, Artificial Sequence
  Description of Artificial Sequence Synthetic oligonucleotide
cctactcaaatctccagtc

SEQ ID NO: 271 - DNA, 18 bp, Artificial Sequence
  Description of Artificial Sequence Synthetic oligonucleotide
ctactcaaatctccagtc

SEQ ID NO: 272 - DNA, 17 bp, Artificial Sequence
  Description of Artificial Sequence Synthetic oligonucleotide
tactcaaatctccagtc

SEQ ID NO: 273 - DNA, 21 bp, Artificial Sequence
  Description of Artificial Sequence Synthetic oligonucleotide
aatcctactcaaatctccagt

SEQ ID NO: 274 - DNA, 20 bp, Artificial Sequence
  Description of Artificial Sequence Synthetic oligonucleotide
atcctactcaaatctccagt

SEQ ID NO: 275 - DNA, 19 bp, Artificial Sequence
  Description of Artificial Sequence Synthetic oligonucleotide
tcctactcaaatctccagt

SEQ ID NO: 276 - DNA, 18 bp, Artificial Sequence
  Description of Artificial Sequence Synthetic oligonucleotide
cctactcaaatctccagt

SEQ ID NO: 277 - DNA, 21 bp, Artificial Sequence
  Description of Artificial Sequence Synthetic oligonucleotide
aaatcctactcaaatctccag

SEQ ID NO: 278 - DNA, 20 bp, Artificial Sequence
  Description of Artificial Sequence Synthetic oligonucleotide
aatcctactcaaatctccag

SEQ ID NO: 279 - DNA, 19 bp, Artificial Sequence
  Description of Artificial Sequence Synthetic oligonucleotide
atcctactcaaatctccag

SEQ ID NO: 280 - DNA, 18 bp, Artificial Sequence
  Description of Artificial Sequence Synthetic oligonucleotide
tcctactcaaatctccag

SEQ ID NO: 281 - DNA, 17 bp, Artificial Sequence
  Description of Artificial Sequence Synthetic oligonucleotide
cctactcaaatctccag

SEQ ID NO: 282 - DNA, 21 bp, Artificial Sequence
  Description of Artificial Sequence Synthetic oligonucleotide
aaaatcctactcaaatctcca

SEQ ID NO: 283 - DNA, 21 bp, Artificial Sequence
  Description of Artificial Sequence Synthetic oligonucleotide
aaaaatcctactcaaatctcc

SEQ ID NO: 284 - DNA, 20 bp, Artificial Sequence
  Description of Artificial Sequence Synthetic oligonucleotide
aaaatcctactcaaatctcc

SEQ ID NO: 285 - DNA, 19 bp, Artificial Sequence
  Description of Artificial Sequence Synthetic oligonucleotide
aaatcctactcaaatctcc

SEQ ID NO: 286 - DNA, 18 bp, Artificial Sequence
  Description of Artificial Sequence Synthetic oligonucleotide
aatcctactcaaatctcc

SEQ ID NO: 287 - DNA, 17 bp, Artificial Sequence
  Description of Artificial Sequence Synthetic oligonucleotide
atcctactcaaatctcc

SEQ ID NO: 288 - DNA, 20 bp, Artificial Sequence
  Description of Artificial Sequence Synthetic oligonucleotide
aaaaatcctactcaaatctc

SEQ ID NO: 289 - DNA, 19 bp, Artificial Sequence
  Description of Artificial Sequence Synthetic oligonucleotide
aaaatcctactcaaatctc

SEQ ID NO: 290 - DNA, 21 bp, Artificial Sequence
  Description of Artificial Sequence Synthetic oligonucleotide
tcaaaaatcctactcaaatct

SEQ ID NO: 291 - DNA, 19 bp, Artificial Sequence
  Description of Artificial Sequence Synthetic oligonucleotide
aaaaatcctactcaaatct

SEQ ID NO: 292 - DNA, 18 bp, Artificial Sequence
  Description of Artificial Sequence Synthetic oligonucleotide
aaaatcctactcaaatct

SEQ ID NO: 293 - DNA, 17 bp, Artificial Sequence
  Description of Artificial Sequence Synthetic oligonucleotide
aaatcctactcaaatct

SEQ ID NO: 294 - DNA, 19 bp, Artificial Sequence
  Description of Artificial Sequence Synthetic oligonucleotide
caaaaatcctactcaaatc

SEQ ID NO: 295 - DNA, 17 bp, Artificial Sequence
  Description of Artificial Sequence Synthetic oligonucleotide
aaaatcctactcaaatc

SEQ ID NO: 296 - DNA, 21 bp, Artificial Sequence
  Description of Artificial Sequence Synthetic oligonucleotide
agtcaaaaatcctactcaaat

SEQ ID NO: 297 - DNA, 20 bp, Artificial Sequence
  Description of Artificial Sequence Synthetic oligonucleotide
gtcaaaaatcctactcaaat

SEQ ID NO: 298 - DNA, 19 bp, Artificial Sequence
  Description of Artificial Sequence Synthetic oligonucleotide
tcaaaaatcctactcaaat

SEQ ID NO: 299 - DNA, 18 bp, Artificial Sequence
  Description of Artificial Sequence Synthetic oligonucleotide
caaaaatcctactcaaat

SEQ ID NO: 300 - DNA, 20 bp, Artificial Sequence
  Description of Artificial Sequence Synthetic oligonucleotide
agtcaaaaatcctactcaaa

SEQ ID NO: 301 - DNA, 19 bp, Artificial Sequence
  Description of Artificial Sequence Synthetic oligonucleotide
gtcaaaaatcctactcaaa

SEQ ID NO: 302 - DNA, 17 bp, Artificial Sequence
  Description of Artificial Sequence Synthetic oligonucleotide
caaaaatcctactcaaa

SEQ ID NO: 303 - DNA, 20 bp, Artificial Sequence
  Description of Artificial Sequence Synthetic oligonucleotide
aagtcaaaaatcctactcaa

SEQ ID NO: 304 - DNA, 19 bp, Artificial Sequence
  Description of Artificial Sequence Synthetic oligonucleotide
agtcaaaaatcctactcaa

SEQ ID NO: 305 - DNA, 18 bp, Artificial Sequence
  Description of Artificial Sequence Synthetic oligonucleotide
gtcaaaaatcctactcaa

SEQ ID NO: 306 - DNA, 17 bp, Artificial Sequence
  Description of Artificial Sequence Synthetic oligonucleotide
tcaaaaatcctactcaa

SEQ ID NO: 307 - DNA, 19 bp, Artificial Sequence
  Description of Artificial Sequence Synthetic oligonucleotide
aagtcaaaaatcctactca

SEQ ID NO: 308 - DNA, 18 bp, Artificial Sequence
  Description of Artificial Sequence Synthetic oligonucleotide
agtcaaaaatcctactca

SEQ ID NO: 309 - DNA, 17 bp, Artificial Sequence
  Description of Artificial Sequence Synthetic oligonucleotide
gtcaaaaatcctactca

SEQ ID NO: 310 - DNA, 18 bp, Artificial Sequence
  Description of Artificial Sequence Synthetic oligonucleotide
aagtcaaaaatcctactc

SEQ ID NO: 311 - DNA, 17 bp, Artificial Sequence
  Description of Artificial Sequence Synthetic oligonucleotide
agtcaaaaatcctactc

SEQ ID NO: 312 - DNA, 17 bp, Artificial Sequence
  Description of Artificial Sequence Synthetic oligonucleotide
aagtcaaaaatcctact

SEQ ID NO: 313 - DNA, 20 bp, Artificial Sequence
  Description of Artificial Sequence Synthetic oligonucleotide
ttaagcaagtcaaaaatcct

SEQ ID NO: 314 - DNA, 20 bp, Artificial Sequence
  Description of Artificial Sequence Synthetic oligonucleotide
tcattcatggtagttaagca

SEQ ID NO: 315 - DNA, 20 bp, Artificial Sequence
  Description of Artificial Sequence Synthetic oligonucleotide
ctcattcatggtagttaagc

SEQ ID NO: 316 - DNA, 20 bp, Artificial Sequence
  Description of Artificial Sequence Synthetic oligonucleotide
tttagttgctactgataatc

SEQ ID NO: 317 - DNA, 20 bp, Artificial Sequence
  Description of Artificial Sequence Synthetic oligonucleotide
atttagttgctactgataat

SEQ ID NO: 318 - DNA, 20 bp, Artificial Sequence
  Description of Artificial Sequence Synthetic oligonucleotide
aatttagttgctactgataa

SEQ ID NO: 319 - DNA, 20 bp, Artificial Sequence
  Description of Artificial Sequence Synthetic oligonucleotide
aataatttagttgctactga

SEQ ID NO: 320 - DNA, 20 bp, Artificial Sequence
  Description of Artificial Sequence Synthetic oligonucleotide
agagaggaaataatttagtt

SEQ ID NO: 321 - DNA, 20 bp, Artificial Sequence
  Description of Artificial Sequence Synthetic oligonucleotide
gagagaggaaataatttagt

SEQ ID NO: 322 - DNA, 20 bp, Artificial Sequence
  Description of Artificial Sequence Synthetic oligonucleotide
gaagagagaggaaataattt

SEQ ID NO: 323 - DNA, 20 bp, Artificial Sequence
  Description of Artificial Sequence Synthetic oligonucleotide
agaagagagaggaaataatt

SEQ ID NO: 324 - DNA, 20 bp, Artificial Sequence
  Description of Artificial Sequence Synthetic oligonucleotide
acagaagagagaggaaataa

SEQ ID NO: 325 - DNA, 20 bp, Artificial Sequence
  Description of Artificial Sequence Synthetic oligonucleotide
agacagaagagagaggaaat

SEQ ID NO: 326 - DNA, 20 bp, Artificial Sequence
  Description of Artificial Sequence Synthetic oligonucleotide
gtgtagacagaagagagagg

SEQ ID NO: 327 - DNA, 20 bp, Artificial Sequence
  Description of Artificial Sequence Synthetic oligonucleotide
tgtgtagacagaagagagag

SEQ ID NO: 328 - DNA, 20 bp, Artificial Sequence
  Description of Artificial Sequence Synthetic oligonucleotide
cttgtgtagacagaagagag

SEQ ID NO: 329 - DNA, 20 bp, Artificial Sequence
  Description of Artificial Sequence Synthetic oligonucleotide
tccttgtgtagacagaagag

SEQ ID NO: 330 - DNA, 20 bp, Artificial Sequence
  Description of Artificial Sequence Synthetic oligonucleotide
tttccttgtgtagacagaag

SEQ ID NO: 331 - DNA, 20 bp, Artificial Sequence
  Description of Artificial Sequence Synthetic oligonucleotide
atgagtgtttccttgtgtag

SEQ ID NO: 332 - DNA, 20 bp, Artificial Sequence
  Description of Artificial Sequence Synthetic oligonucleotide
ttatgagtgtttccttgtgt

SEQ ID NO: 333 - DNA, 20 bp, Artificial Sequence
  Description of Artificial Sequence Synthetic oligonucleotide
tgcatttatgagtgtttcct

SEQ ID NO: 334 - DNA, 20 bp, Artificial Sequence
  Description of Artificial Sequence Synthetic oligonucleotide
cgtgcatttatgagtgtttc

SEQ ID NO: 335 - DNA, 20 bp, Artificial Sequence
  Description of Artificial Sequence Synthetic oligonucleotide
ccgtgcatttatgagtgttt

SEQ ID NO: 336 - DNA, 20 bp, Artificial Sequence
  Description of Artificial Sequence Synthetic oligonucleotide
ccccgtgcatttatgagtgt

SEQ ID NO: 337 - DNA, 20 bp, Artificial Sequence
  Description of Artificial Sequence Synthetic oligonucleotide
cctccccgtgcatttatgag

SEQ ID NO: 338 - DNA, 20 bp, Artificial Sequence
  Description of Artificial Sequence Synthetic oligonucleotide
ctcctccccgtgcatttatg

SEQ ID NO: 339 - DNA, 20 bp, Artificial Sequence
  Description of Artificial Sequence Synthetic oligonucleotide
cctcctccccgtgcatttat

SEQ ID NO: 340 - DNA, 20 bp, Artificial Sequence
  Description of Artificial Sequence Synthetic oligonucleotide
ctgacctcctccccgtgcat

SEQ ID NO: 341 - DNA, 20 bp, Artificial Sequence
  Description of Artificial Sequence Synthetic oligonucleotide
ttctgacctcctccccgtgc

SEQ ID NO: 342 - DNA, 20 bp, Artificial Sequence
  Description of Artificial Sequence Synthetic oligonucleotide
gttctgacctcctccccgtg

SEQ ID NO: 343 - DNA, 20 bp, Artificial Sequence
  Description of Artificial Sequence Synthetic oligonucleotide
ggttctgacctcctccccgt

SEQ ID NO: 344 - DNA, 20 bp, Artificial Sequence
  Description of Artificial Sequence Synthetic oligonucleotide
caggttctgacctcctcccc

SEQ ID NO: 345 - DNA, 20 bp, Artificial Sequence
  Description of Artificial Sequence Synthetic oligonucleotide
tcaggttctgacctcctccc

SEQ ID NO: 346 - DNA, 20 bp, Artificial Sequence
  Description of Artificial Sequence Synthetic oligonucleotide
ttcaggttctgacctcctcc

SEQ ID NO: 347 - DNA, 20 bp, Artificial Sequence
  Description of Artificial Sequence Synthetic oligonucleotide
tttcaggttctgacctcctc

SEQ ID NO: 348 - DNA, 20 bp, Artificial Sequence
  Description of Artificial Sequence Synthetic oligonucleotide
aaaggctttcaggttctgac

SEQ ID NO: 349 - DNA, 20 bp, Artificial Sequence
  Description of Artificial Sequence Synthetic oligonucleotide
aagaaaggctttcaggttct

SEQ ID NO: 350 - DNA, 20 bp, Artificial Sequence
  Description of Artificial Sequence Synthetic oligonucleotide
caaagaaaggctttcaggtt

SEQ ID NO: 351 - DNA, 20 bp, Artificial Sequence
  Description of Artificial Sequence Synthetic oligonucleotide
ccaaagaaaggctttcaggt

SEQ ID NO: 352 - DNA, 20 bp, Artificial Sequence
  Description of Artificial Sequence Synthetic oligonucleotide
tccaaagaaaggctttcagg

SEQ ID NO: 353 - DNA, 20 bp, Artificial Sequence
  Description of Artificial Sequence Synthetic oligonucleotide
ttatccaaagaaaggctttc

SEQ ID NO: 354 - DNA, 20 bp, Artificial Sequence
  Description of Artificial Sequence Synthetic oligonucleotide
tgctcttatccaaagaaagg

SEQ ID NO: 355 - DNA, 20 bp, Artificial Sequence
  Description of Artificial Sequence Synthetic oligonucleotide
tgatgctcttatccaaagaa

SEQ ID NO: 356 - DNA, 20 bp, Artificial Sequence
  Description of Artificial Sequence Synthetic oligonucleotide
gttgatgctcttatccaaag

SEQ ID NO: 357 - DNA, 20 bp, Artificial Sequence
  Description of Artificial Sequence Synthetic oligonucleotide
gcagttgatgctcttatcca

SEQ ID NO: 358 - DNA, 20 bp, Artificial Sequence
  Description of Artificial Sequence Synthetic oligonucleotide
tgcagttgatgctcttatcc

SEQ ID NO: 359 - DNA, 20 bp, Artificial Sequence
  Description of Artificial Sequence Synthetic oligonucleotide
ctgcagttgatgctcttatc

SEQ ID NO: 360 - DNA, 20 bp, Artificial Sequence
  Description of Artificial Sequence Synthetic oligonucleotide
cctgcagttgatgctcttat

SEQ ID NO: 361 - DNA, 20 bp, Artificial Sequence
  Description of Artificial Sequence Synthetic oligonucleotide
acctgcagttgatgctctta

SEQ ID NO: 362 - DNA, 20 bp, Artificial Sequence
  Description of Artificial Sequence Synthetic oligonucleotide
tacctgcagttgatgctctt

SEQ ID NO: 363 - DNA, 20 bp, Artificial Sequence
  Description of Artificial Sequence Synthetic oligonucleotide
gtacctgcagttgatgctct

SEQ ID NO: 364 - DNA, 20 bp, Artificial Sequence
  Description of Artificial Sequence Synthetic oligonucleotide
ggtacctgcagttgatgctc

SEQ ID NO: 365 - DNA, 20 bp, Artificial Sequence
  Description of Artificial Sequence Synthetic oligonucleotide
tggtacctgcagttgatgct

SEQ ID NO: 366 - DNA, 20 bp, Artificial Sequence
  Description of Artificial Sequence Synthetic oligonucleotide
gtggtacctgcagttgatgc

SEQ ID NO: 367 - DNA, 20 bp, Artificial Sequence
  Description of Artificial Sequence Synthetic oligonucleotide
tgtggtacctgcagttgatg

SEQ ID NO: 368 - DNA, 20 bp, Artificial Sequence
  Description of Artificial Sequence Synthetic oligonucleotide
atgtggtacctgcagttgat

SEQ ID NO: 369 - DNA, 20 bp, Artificial Sequence
  Description of Artificial Sequence Synthetic oligonucleotide
aatgtggtacctgcagttga

SEQ ID NO: 370 - DNA, 20 bp, Artificial Sequence
  Description of Artificial Sequence Synthetic oligonucleotide
caatgtggtacctgcagttg

SEQ ID NO: 371 - DNA, 20 bp, Artificial Sequence
  Description of Artificial Sequence Synthetic oligonucleotide
gccaatgtggtacctgcagt

SEQ ID NO: 372 - DNA, 20 bp, Artificial Sequence
  Description of Artificial Sequence Synthetic oligonucleotide
agggccaatgtggtacctgc

SEQ ID NO: 373 - DNA, 20 bp, Artificial Sequence
  Description of Artificial Sequence Synthetic oligonucleotide
acagggccaatgtggtacct

SEQ ID NO: 374 - DNA, 20 bp, Artificial Sequence
  Description of Artificial Sequence Synthetic oligonucleotide
tcacagggccaatgtggtac

SEQ ID NO: 375 - DNA, 20 bp, Artificial Sequence
  Description of Artificial Sequence Synthetic oligonucleotide
attagcatcacagggccaat

SEQ ID NO: 376 - DNA, 20 bp, Artificial Sequence
  Description of Artificial Sequence Synthetic oligonucleotide
tattagcatcacagggccaa

SEQ ID NO: 377 - DNA, 20 bp, Artificial Sequence
  Description of Artificial Sequence Synthetic oligonucleotide
tatattagcatcacagggcc

SEQ ID NO: 378 - DNA, 20 bp, Artificial Sequence
  Description of Artificial Sequence Synthetic oligonucleotide
tttatattagcatcacaggg

SEQ ID NO: 379 - DNA, 20 bp, Artificial Sequence
  Description of Artificial Sequence Synthetic oligonucleotide
ccttttatattagcatcaca

SEQ ID NO: 380 - DNA, 20 bp, Artificial Sequence
  Description of Artificial Sequence Synthetic oligonucleotide
tccttttatattagcatcac

SEQ ID NO: 381 - DNA, 20 bp, Artificial Sequence
  Description of Artificial Sequence Synthetic oligonucleotide
gctccttttatattagcatc

SEQ ID NO: 382 - DNA, 20 bp, Artificial Sequence
  Description of Artificial Sequence Synthetic oligonucleotide
agctccttttatattagcat

SEQ ID NO: 383 - DNA, 20 bp, Artificial Sequence
  Description of Artificial Sequence Synthetic oligonucleotide
tagctccttttatattagca

SEQ ID NO: 384 - DNA, 20 bp, Artificial Sequence
  Description of Artificial Sequence Synthetic oligonucleotide
ggcctagctccttttatatt

SEQ ID NO: 385 - DNA, 20 bp, Artificial Sequence
  Description of Artificial Sequence Synthetic oligonucleotide
ccggtgggcctagctccttt

SEQ ID NO: 386 - DNA, 19 bp, Artificial Sequence
  Description of Artificial Sequence Synthetic oligonucleotide
accaggccttatgtgggaa

SEQ ID NO: 387 - DNA, 20 bp, Artificial Sequence
  Description of Artificial Sequence Synthetic oligonucleotide
actagaccaggccttatgtg

SEQ ID NO: 388 - DNA, 17 bp, Artificial Sequence
  Description of Artificial Sequence Synthetic oligonucleotide
cccactagaccaggcct

SEQ ID NO: 389 - DNA, 21 bp, Artificial Sequence
  Description of Artificial Sequence Synthetic oligonucleotide
gccacagcacagggcccacta

SEQ ID NO: 390 - DNA, 21 bp, Artificial Sequence
  Description of Artificial Sequence Synthetic oligonucleotide
agacctggccacagcacaggg

SEQ ID NO: 391 - DNA, 19 bp, Artificial Sequence
  Description of Artificial Sequence Synthetic oligonucleotide
gctcaccccacagacctgg

SEQ ID NO: 392 - DNA, 21 bp, Artificial Sequence
  Description of Artificial Sequence Synthetic oligonucleotide
gccgccccagctcaccccaca

SEQ ID NO: 393 - DNA, 21 bp, Artificial Sequence
  Description of Artificial Sequence Synthetic oligonucleotide
ccacttcagccgccccagctc

SEQ ID NO: 394 - DNA, 21 bp, Artificial Sequence
  Description of Artificial Sequence Synthetic oligonucleotide
aattgagtccacttcagccgc

SEQ ID NO: 395 - DNA, 18 bp, Artificial Sequence
  Description of Artificial Sequence Synthetic oligonucleotide
aacaggaattgagtccac

SEQ ID NO: 396 - DNA, 19 bp, Artificial Sequence
  Description of Artificial Sequence Synthetic oligonucleotide
catcaacaggaattgagtc

SEQ ID NO: 397 - DNA, 17 bp, Artificial Sequence
  Description of Artificial Sequence Synthetic oligonucleotide
ggcatcaacaggaattg

SEQ ID NO: 398 - DNA, 18 bp, Artificial Sequence
  Description of Artificial Sequence Synthetic oligonucleotide
ctgggcatcaacaggaat

SEQ ID NO: 399 - DNA, 19 bp, Artificial Sequence
  Description of Artificial Sequence Synthetic oligonucleotide
ctcacctgggcatcaacag

SEQ ID NO: 400 - DNA, 17 bp, Artificial Sequence
  Description of Artificial Sequence Synthetic oligonucleotide
ctcctcacctgggcatc

SEQ ID NO: 401 - DNA, 17 bp, Artificial Sequence
  Description of Artificial Sequence Synthetic oligonucleotide
gtgctcctcacctgggc

SEQ ID NO: 402 - DNA, 20 bp, Artificial Sequence
  Description of Artificial Sequence Synthetic oligonucleotide
gcagagtgctcctcacctgg

SEQ ID NO: 403 - DNA, 19 bp, Artificial Sequence
  Description of Artificial Sequence Synthetic oligonucleotide
ggatttgcagagtgctcct

SEQ ID NO: 404 - DNA, 20 bp, Artificial Sequence
  Description of Artificial Sequence Synthetic oligonucleotide
ccagtggaacggatttgcag

SEQ ID NO: 405 - DNA, 17 bp, Artificial Sequence
  Description of Artificial Sequence Synthetic oligonucleotide
gtcccagtggaacggat

SEQ ID NO: 406 - DNA, 17 bp, Artificial Sequence
  Description of Artificial Sequence Synthetic oligonucleotide
aggtcccagtggaacgg

SEQ ID NO: 407 - DNA, 18 bp, Artificial Sequence
  Description of Artificial Sequence Synthetic oligonucleotide
atcaggtcccagtggaac

SEQ ID NO: 408 - DNA, 18 bp, Artificial Sequence
  Description of Artificial Sequence Synthetic oligonucleotide
tcccaatcaggtcccagt

SEQ ID NO: 409 - DNA, 18 bp, Artificial Sequence
  Description of Artificial Sequence Synthetic oligonucleotide
tcttcccaatcaggtccc

SEQ ID NO: 410 - DNA, 21 bp, Artificial Sequence
  Description of Artificial Sequence Synthetic oligonucleotide
acaggttcttcccaatcaggt

SEQ ID NO: 411 - DNA, 20 bp, Artificial Sequence
  Description of Artificial Sequence Synthetic oligonucleotide
ggcaaacaggttcttcccaa

SEQ ID NO: 412 - DNA, 18 bp, Artificial Sequence
  Description of Artificial Sequence Synthetic oligonucleotide
catggcaaacaggttctt

SEQ ID NO: 413 - DNA, 17 bp, Artificial Sequence
  Description of Artificial Sequence Synthetic oligonucleotide
accatggcaaacaggtt

SEQ ID NO: 414 - DNA, 17 bp, Artificial Sequence
  Description of Artificial Sequence Synthetic oligonucleotide
ccaccatggcaaacagg

SEQ ID NO: 415 - DNA, 18 bp, Artificial Sequence
  Description of Artificial Sequence Synthetic oligonucleotide
ccaccaccatggcaaaca

SEQ ID NO: 416 - DNA, 17 bp, Artificial Sequence
  Description of Artificial Sequence Synthetic oligonucleotide
cccttccaccaccatgg

SEQ ID NO: 417 - DNA, 18 bp, Artificial Sequence
  Description of Artificial Sequence Synthetic oligonucleotide
caccccttccaccaccat

SEQ ID NO: 418 - DNA, 18 bp, Artificial Sequence
  Description of Artificial Sequence Synthetic oligonucleotide
agtacaccaccccttcca

SEQ ID NO: 419 - DNA, 20 bp, Artificial Sequence
  Description of Artificial Sequence Synthetic oligonucleotide
gaggaagtacaccacccctt

SEQ ID NO: 420 - DNA, 19 bp, Artificial Sequence
  Description of Artificial Sequence Synthetic oligonucleotide
ggtcaggaggaagtacacc

SEQ ID NO: 421 - DNA, 17 bp, Artificial Sequence
  Description of Artificial Sequence Synthetic oligonucleotide
cagggtcaggaggaagt

SEQ ID NO: 422 - DNA, 17 bp, Artificial Sequence
  Description of Artificial Sequence Synthetic oligonucleotide
ccagcagggtcaggagg

SEQ ID NO: 423 - DNA, 18 bp, Artificial Sequence
  Description of Artificial Sequence Synthetic oligonucleotide
ctggaccagcagggtcag

SEQ ID NO: 424 - DNA, 20 bp, Artificial Sequence
  Description of Artificial Sequence Synthetic oligonucleotide
gtggcgctggaccagcaggg

SEQ ID NO: 425 - DNA, 18 bp, Artificial Sequence
  Description of Artificial Sequence Synthetic oligonucleotide
gaagaagtggcgctggac

SEQ ID NO: 426 - DNA, 18 bp, Artificial Sequence
  Description of Artificial Sequence Synthetic oligonucleotide
gaggaagaagtggcgctg

SEQ ID NO: 427 - DNA, 20 bp, Artificial Sequence
  Description of Artificial Sequence Synthetic oligonucleotide
attgggagaggaagaagtgg

SEQ ID NO: 428 - DNA, 17 bp, Artificial Sequence
  Description of Artificial Sequence Synthetic oligonucleotide
ccattgggagaggaaga

SEQ ID NO: 429 - DNA, 18 bp, Artificial Sequence
  Description of Artificial Sequence Synthetic oligonucleotide
gtaccattgggagaggaa

SEQ ID NO: 430 - DNA, 21 bp, Artificial Sequence
  Description of Artificial Sequence Synthetic oligonucleotide
catggatgtaccattgggaga

SEQ ID NO: 431 - DNA, 19 bp, Artificial Sequence
  Description of Artificial Sequence Synthetic oligonucleotide
gtgtggcatggatgtacca

SEQ ID NO: 432 - DNA, 17 bp, Artificial Sequence
  Description of Artificial Sequence Synthetic oligonucleotide
agggtgtggcatggatg

SEQ ID NO: 433 - DNA, 19 bp, Artificial Sequence
  Description of Artificial Sequence Synthetic oligonucleotide
ggcccagggtgtggcatgg

SEQ ID NO: 434 - DNA, 18 bp, Artificial Sequence
  Description of Artificial Sequence Synthetic oligonucleotide
actggcccagggtgtggc

SEQ ID NO: 435 - DNA, 20 bp, Artificial Sequence
  Description of Artificial Sequence Synthetic oligonucleotide
tgagctgcccactggcccag

SEQ ID NO: 436 - DNA, 20 bp, Artificial Sequence
  Description of Artificial Sequence Synthetic oligonucleotide
tgccctgagctgcccactgg

SEQ ID NO: 437 - DNA, 20 bp, Artificial Sequence
  Description of Artificial Sequence Synthetic oligonucleotide
ctggatgccctgagctgccc

SEQ ID NO: 438 - DNA, 17 bp, Artificial Sequence
  Description of Artificial Sequence Synthetic oligonucleotide
ttctggatgccctgagc

SEQ ID NO: 439 - DNA, 18 bp, Artificial Sequence
  Description of Artificial Sequence Synthetic oligonucleotide
gtccagttctggatgccc

SEQ ID NO: 440 - DNA, 17 bp, Artificial Sequence
  Description of Artificial Sequence Synthetic oligonucleotide
taaggtccagttctgga

SEQ ID NO: 441 - DNA, 18 bp, Artificial Sequence
  Description of Artificial Sequence Synthetic oligonucleotide
gtataaggtccagttctg

SEQ ID NO: 442 - DNA, 19 bp, Artificial Sequence
  Description of Artificial Sequence Synthetic oligonucleotide
gtgggtataaggtccagtt

SEQ ID NO: 443 - DNA, 18 bp, Artificial Sequence
  Description of Artificial Sequence Synthetic oligonucleotide
gaaatgaccatgtgggta

SEQ ID NO: 444 - DNA, 21 bp, Artificial Sequence
  Description of Artificial Sequence Synthetic oligonucleotide
tgaggaaagaaatgaccatgt

SEQ ID NO: 445 - DNA, 19 bp, Artificial Sequence
  Description of Artificial Sequence Synthetic oligonucleotide
gctcctgaggaaagaaatg

SEQ ID NO: 446 - DNA, 17 bp, Artificial Sequence
  Description of Artificial Sequence Synthetic oligonucleotide
gggctcctgaggaaaga

SEQ ID NO: 447 - DNA, 18 bp, Artificial Sequence
  Description of Artificial Sequence Synthetic oligonucleotide
gtggggctcctgaggaaa

SEQ ID NO: 448 - DNA, 17 bp, Artificial Sequence
  Description of Artificial Sequence Synthetic oligonucleotide
gagtggggctcctgagg

SEQ ID NO: 449 - DNA, 17 bp, Artificial Sequence
  Description of Artificial Sequence Synthetic oligonucleotide
tggagtggggctcctga

SEQ ID NO: 450 - DNA, 24 bp, Artificial Sequence
  Description of Artificial Sequence Synthetic primer
gattacaaggatgacgacgataag

SEQ ID NO: 451 - DNA, 21 bp, Artificial Sequence
  Description of Artificial Sequence Synthetic primer
tcttcatcaacaatgggctcc

SEQ ID NO: 452 - DNA, 19 bp, Artificial Sequence
  Description of Artificial Sequence Synthetic primer
atgggcctgtctgactcag

SEQ ID NO: 453 - DNA, 23 bp, Artificial Sequence
  Description of Artificial Sequence Synthetic primer
tcattcctccccaagatctcaga

SEQ ID NO: 454 - DNA, 21 bp, Artificial Sequence
  Description of Artificial Sequence Synthetic primer
gtttatcagtggagtgagccc

SEQ ID NO: 455 - DNA, 19 bp, Artificial Sequence
  Description of Artificial Sequence Synthetic primer
gatgaagatgcccaccacc

SEQ ID NO: 456 - DNA, 21 bp, Artificial Sequence
  Description of Artificial Sequence Synthetic primer
gttctgggtcaatgaacagag

SEQ ID NO: 457 - DNA, 25 bp, Artificial Sequence
  Description of Artificial Sequence Synthetic primer
gaaatcaggtatttctttagaggcc

SEQ ID NO: 458 - DNA, 25 bp, Artificial Sequence
  Description of Artificial Sequence Synthetic primer
atgttctgggtcaatgaacagaggt

SEQ ID NO: 459 - DNA, 26 bp, Artificial Sequence
  Description of Artificial Sequence Synthetic primer
ctatcaggtatttctttagaggcctc

SEQ ID NO: 460 - DNA, 47 bp, Artificial Sequence
  Description of Artificial Sequence Synthetic primer
atcatgaatgtgagcggggtgtgtaaacagactggagatttgagtag

SEQ ID NO: 461 - DNA, 42 bp, Artificial Sequence
  Description of Artificial Sequence Synthetic primer
aaatctccagtctgtttacacaccccgctcacattcatgatc

SEQ ID NO: 462 - protein, 8 aa, Artificial Sequence
  Description of Artificial Sequence Synthetic peptide
RRRRRRRR

SEQ ID NO: 463 - DNA, 17 bp, Artificial Sequence
  Description of Artificial Sequence Synthetic oligonucleotide
tgagtgtttccttgtgt

SEQ ID NO: 464 - DNA, 18 bp, Artificial Sequence
  Description of Artificial Sequence Synthetic oligonucleotide
atgagtgtttccttgtgt

SEQ ID NO: 465 - DNA, 19 bp, Artificial Sequence
  Description of Artificial Sequence Synthetic oligonucleotide
tatgagtgtttccttgtgt

SEQ ID NO: 466 - DNA, 21 bp, Artificial Sequence
  Description of Artificial Sequence Synthetic oligonucleotide
tttatgagtgtttccttgtgt

SEQ ID NO: 467 - DNA, 17 bp, Artificial Sequence
  Description of Artificial Sequence Synthetic oligonucleotide
atgagtgtttccttgtg

SEQ ID NO: 468 - DNA, 18 bp, Artificial Sequence
  Description of Artificial Sequence Synthetic oligonucleotide
tatgagtgtttccttgtg

SEQ ID NO: 469 - DNA, 19 bp, Artificial Sequence
  Description of Artificial Sequence Synthetic oligonucleotide
ttatgagtgtttccttgtg

SEQ ID NO: 470 - DNA, 20 bp, Artificial Sequence
  Description of Artificial Sequence Synthetic oligonucleotide
tttatgagtgtttccttgtg

SEQ ID NO: 471 - DNA, 21 bp, Artificial Sequence
  Description of Artificial Sequence Synthetic oligonucleotide
atttatgagtgtttccttgtg

SEQ ID NO: 472 - DNA, 17 bp, Artificial Sequence
  Description of Artificial Sequence Synthetic oligonucleotide
tatgagtgtttccttgt

SEQ ID NO: 473 - DNA, 18 bp, Artificial Sequence
  Description of Artificial Sequence Synthetic oligonucleotide
ttatgagtgtttccttgt

SEQ ID NO: 474 - DNA, 19 bp, Artificial Sequence
  Description of Artificial Sequence Synthetic oligonucleotide
tttatgagtgtttccttgt

SEQ ID NO: 475 - DNA, 20 bp, Artificial Sequence
  Description of Artificial Sequence Synthetic oligonucleotide
atttatgagtgtttccttgt

SEQ ID NO: 476 - DNA, 21 bp, Artificial Sequence
  Description of Artificial Sequence Synthetic oligonucleotide
catttatgagtgtttccttgt

SEQ ID NO: 477 - DNA, 17 bp, Artificial Sequence
  Description of Artificial Sequence Synthetic oligonucleotide
ttatgagtgtttccttg

SEQ ID NO: 478 - DNA, 18 bp, Artificial Sequence
  Description of Artificial Sequence Synthetic oligonucleotide
tttatgagtgtttccttg

SEQ ID NO: 479 - DNA, 19 bp, Artificial Sequence
  Description of Artificial Sequence Synthetic oligonucleotide
atttatgagtgtttccttg

SEQ ID NO: 480 - DNA, 20 bp, Artificial Sequence
  Description of Artificial Sequence Synthetic oligonucleotide
catttatgagtgtttccttg

SEQ ID NO: 481 - DNA, 21 bp, Artificial Sequence
  Description of Artificial Sequence Synthetic oligonucleotide
gcatttatgagtgtttccttg

SEQ ID NO: 482 - DNA, 17 bp, Artificial Sequence
  Description of Artificial Sequence Synthetic oligonucleotide
tttatgagtgtttcctt

SEQ ID NO: 483 - DNA, 18 bp, Artificial Sequence
  Description of Artificial Sequence Synthetic oligonucleotide
atttatgagtgtttcctt

SEQ ID NO: 484 - DNA, 19 bp, Artificial Sequence
  Description of Artificial Sequence Synthetic oligonucleotide
catttatgagtgtttcctt

SEQ ID NO: 485 - DNA, 20 bp, Artificial Sequence
  Description of Artificial Sequence Synthetic oligonucleotide
gcatttatgagtgtttcctt

SEQ ID NO: 486 - DNA, 21 bp, Artificial Sequence
  Description of Artificial Sequence Synthetic oligonucleotide
tgcatttatgagtgtttcctt

SEQ ID NO: 487 - DNA, 17 bp, Artificial Sequence
  Description of Artificial Sequence Synthetic oligonucleotide
atttatgagtgtttcct

SEQ ID NO: 488 - DNA, 18 bp, Artificial Sequence
  Description of Artificial Sequence Synthetic oligonucleotide
catttatgagtgtttcct

SEQ ID NO: 489 - DNA, 19 bp, Artificial Sequence
  Description of Artificial Sequence Synthetic oligonucleotide
gcatttatgagtgtttcct

SEQ ID NO: 490 - DNA, 21 bp, Artificial Sequence
  Description of Artificial Sequence Synthetic oligonucleotide
gtgcatttatgagtgtttcct

SEQ ID NO: 491 - DNA, 17 bp, Artificial Sequence
  Description of Artificial Sequence Synthetic oligonucleotide
catttatgagtgtttcc

SEQ ID NO: 492 - DNA, 18 bp, Artificial Sequence
  Description of Artificial Sequence Synthetic oligonucleotide
gcatttatgagtgtttcc

SEQ ID NO: 493 - DNA, 19 bp, Artificial Sequence
  Description of Artificial Sequence Synthetic oligonucleotide
tgcatttatgagtgtttcc

SEQ ID NO: 494 - DNA, 20 bp, Artificial Sequence
  Description of Artificial Sequence Synthetic oligonucleotide
gtgcatttatgagtgtttcc

SEQ ID NO: 495 - DNA, 21 bp, Artificial Sequence
  Description of Artificial Sequence Synthetic oligonucleotide
cgtgcatttatgagtgtttcc

SEQ ID NO: 496 - DNA, 17 bp, Artificial Sequence
  Description of Artificial Sequence Synthetic oligonucleotide
gcatttatgagtgtttc

SEQ ID NO: 497 - DNA, 18 bp, Artificial Sequence
  Description of Artificial Sequence Synthetic oligonucleotide
tgcatttatgagtgtttc

SEQ ID NO: 498 - DNA, 19 bp, Artificial Sequence
  Description of Artificial Sequence Synthetic oligonucleotide
gtgcatttatgagtgtttc

SEQ ID NO: 499 - DNA, 21 bp, Artificial Sequence
  Description of Artificial Sequence Synthetic oligonucleotide
ccgtgcatttatgagtgtttc

SEQ ID NO: 500 - DNA, 17 bp, Artificial Sequence
  Description of Artificial Sequence Synthetic oligonucleotide
tgcatttatgagtgttt

SEQ ID NO: 501 - DNA, 18 bp, Artificial Sequence
  Description of Artificial Sequence Synthetic oligonucleotide
gtgcatttatgagtgttt

SEQ ID NO: 502 - DNA, 19 bp, Artificial Sequence
  Description of Artificial Sequence Synthetic oligonucleotide
cgtgcatttatgagtgttt

SEQ ID NO: 503 - DNA, 21 bp, Artificial Sequence
  Description of Artificial Sequence Synthetic oligonucleotide
cccgtgcatttatgagtgttt

SEQ ID NO: 504 - DNA, 17 bp, Artificial Sequence
  Description of Artificial Sequence Synthetic oligonucleotide
gtgcatttatgagtgtt

SEQ ID NO: 505 - DNA, 18 bp, Artificial Sequence
  Description of Artificial Sequence Synthetic oligonucleotide
cgtgcatttatgagtgtt

SEQ ID NO: 506 - DNA, 19 bp, Artificial Sequence
  Description of Artificial Sequence Synthetic oligonucleotide
ccgtgcatttatgagtgtt

SEQ ID NO: 507 - DNA, 20 bp, Artificial Sequence
  Description of Artificial Sequence Synthetic oligonucleotide
cccgtgcatttatgagtgtt

SEQ ID NO: 508 - DNA, 21 bp, Artificial Sequence
  Description of Artificial Sequence Synthetic oligonucleotide
ccccgtgcatttatgagtgtt

SEQ ID NO: 509 - DNA, 17 bp, Artificial Sequence
  Description of Artificial Sequence Synthetic oligonucleotide
cgtgcatttatgagtgt

SEQ ID NO: 510 - DNA, 18 bp, Artificial Sequence
  Description of Artificial Sequence Synthetic oligonucleotide
ccgtgcatttatgagtgt

SEQ ID NO: 511 - DNA, 19 bp, Artificial Sequence
  Description of Artificial Sequence Synthetic oligonucleotide
cccgtgcatttatgagtgt

SEQ ID NO: 512 - DNA, 21 bp, Artificial Sequence
  Description of Artificial Sequence Synthetic oligonucleotide
tccccgtgcatttatgagtgt

SEQ ID NO: 513 - DNA, 17 bp, Artificial Sequence
  Description of Artificial Sequence Synthetic oligonucleotide
ccgtgcatttatgagtg

SEQ ID NO: 514 - DNA, 18 bp, Artificial Sequence
  Description of Artificial Sequence Synthetic oligonucleotide
cccgtgcatttatgagtg

SEQ ID NO: 515 - DNA, 19 bp, Artificial Sequence
  Description of Artificial Sequence Synthetic oligonucleotide
ccccgtgcatttatgagtg

SEQ ID NO: 516 - DNA, 20 bp, Artificial Sequence
  Description of Artificial Sequence Synthetic oligonucleotide
tccccgtgcatttatgagtg

SEQ ID NO: 517 - DNA, 21 bp, Artificial Sequence
  Description of Artificial Sequence Synthetic oligonucleotide
ctccccgtgcatttatgagtg

SEQ ID NO: 518 - DNA, 17 bp, Artificial Sequence
  Description of Artificial Sequence Synthetic oligonucleotide
cccgtgcatttatgagt

SEQ ID NO: 519 - DNA, 18 bp, Artificial Sequence
  Description of Artificial Sequence Synthetic oligonucleotide
ccccgtgcatttatgagt

SEQ ID NO: 520 - DNA, 19 bp, Artificial Sequence
  Description of Artificial Sequence Synthetic oligonucleotide
tccccgtgcatttatgagt

SEQ ID NO: 521 - DNA, 20 bp, Artificial Sequence
  Description of Artificial Sequence Synthetic oligonucleotide
ctccccgtgcatttatgagt

SEQ ID NO: 522 - DNA, 21 bp, Artificial Sequence
  Description of Artificial Sequence Synthetic oligonucleotide
cctccccgtgcatttatgagt

SEQ ID NO: 523 - DNA, 17 bp, Artificial Sequence
  Description of Artificial Sequence Synthetic oligonucleotide
ccccgtgcatttatgag

SEQ ID NO: 524 - DNA, 18 bp, Artificial Sequence
  Description of Artificial Sequence Synthetic oligonucleotide
tccccgtgcatttatgag

SEQ ID NO: 525 - DNA, 19 bp, Artificial Sequence
  Description of Artificial Sequence Synthetic oligonucleotide
ctccccgtgcatttatgag

SEQ ID NO: 526 - DNA, 21 bp, Artificial Sequence
  Description of Artificial Sequence Synthetic oligonucleotide
tcctccccgtgcatttatgag

SEQ ID NO: 527 - DNA, 17 bp, Artificial Sequence
  Description of Artificial Sequence Synthetic oligonucleotide
tccccgtgcatttatga

SEQ ID NO: 528 - DNA, 18 bp, Artificial Sequence
  Description of Artificial Sequence Synthetic oligonucleotide
ctccccgtgcatttatga

SEQ ID NO: 529 - DNA, 19 bp, Artificial Sequence
  Description of Artificial Sequence Synthetic oligonucleotide
cctccccgtgcatttatga

SEQ ID NO: 530 - DNA, 20 bp, Artificial Sequence
  Description of Artificial Sequence Synthetic oligonucleotide
tcctccccgtgcatttatga

SEQ ID NO: 531 - DNA, 21 bp, Artificial Sequence
  Description of Artificial Sequence Synthetic oligonucleotide
ctcctccccgtgcatttatga

SEQ ID NO: 532 - DNA, 17 bp, Artificial Sequence
  Description of Artificial Sequence Synthetic oligonucleotide
ctccccgtgcatttatg

SEQ ID NO: 533 - DNA, 18 bp, Artificial Sequence
  Description of Artificial Sequence Synthetic oligonucleotide
cctccccgtgcatttatg

SEQ ID NO: 534 - DNA, 19 bp, Artificial Sequence
  Description of Artificial Sequence Synthetic oligonucleotide
tcctccccgtgcatttatg

SEQ ID NO: 535 - DNA, 21 bp, Artificial Sequence
  Description of Artificial Sequence Synthetic oligonucleotide
cctcctccccgtgcatttatg

SEQ ID NO: 536 - DNA, 17 bp, Artificial Sequence
  Description of Artificial Sequence Synthetic oligonucleotide
cctccccgtgcatttat

SEQ ID NO: 537 - DNA, 18 bp, Artificial Sequence
  Description of Artificial Sequence Synthetic oligonucleotide
tcctccccgtgcatttat

SEQ ID NO: 538 - DNA, 19 bp, Artificial Sequence
  Description of Artificial Sequence Synthetic oligonucleotide
ctcctccccgtgcatttat

SEQ ID NO: 539 - DNA, 21 bp, Artificial Sequence
  Description of Artificial Sequence Synthetic oligonucleotide
acctcctccccgtgcatttat

SEQ ID NO: 540 - DNA, 17 bp, Artificial Sequence
  Description of Artificial Sequence Synthetic oligonucleotide
tcctccccgtgcattta

SEQ ID NO: 541 - DNA, 18 bp, Artificial Sequence
  Description of Artificial Sequence Synthetic oligonucleotide
ctcctccccgtgcattta

SEQ ID NO: 542 - DNA, 19 bp, Artificial Sequence
  Description of Artificial Sequence Synthetic oligonucleotide
cctcctccccgtgcattta

SEQ ID NO: 543 - DNA, 20 bp, Artificial Sequence
  Description of Artificial Sequence Synthetic oligonucleotide
acctcctccccgtgcattta

SEQ ID NO: 544 - DNA, 21 bp, Artificial Sequence
  Description of Artificial Sequence Synthetic oligonucleotide
gacctcctccccgtgcattta

SEQ ID NO: 545 - DNA, 17 bp, Artificial Sequence
  Description of Artificial Sequence Synthetic oligonucleotide
ctcctccccgtgcattt

SEQ ID NO: 546 - DNA, 18 bp, Artificial Sequence
  Description of Artificial Sequence Synthetic oligonucleotide
cctcctccccgtgcattt

SEQ ID NO: 547 - DNA, 19 bp, Artificial Sequence
  Description of Artificial Sequence Synthetic oligonucleotide
acctcctccccgtgcattt

SEQ ID NO: 548 - DNA, 20 bp, Artificial Sequence
  Description of Artificial Sequence Synthetic oligonucleotide
gacctcctccccgtgcattt

SEQ ID NO: 549 - DNA, 21 bp, Artificial Sequence
  Description of Artificial Sequence Synthetic oligonucleotide
tgacctcctccccgtgcattt

SEQ ID NO: 550 - DNA, 17 bp, Artificial Sequence
  Description of Artificial Sequence Synthetic oligonucleotide
cctcctccccgtgcatt

SEQ ID NO: 551 - DNA, 18 bp, Artificial Sequence
  Description of Artificial Sequence Synthetic oligonucleotide
acctcctccccgtgcatt

SEQ ID NO: 552 - DNA, 19 bp, Artificial Sequence
  Description of Artificial Sequence Synthetic oligonucleotide
gacctcctccccgtgcatt

SEQ ID NO: 553 - DNA, 20 bp, Artificial Sequence
  Description of Artificial Sequence Synthetic oligonucleotide
tgacctcctccccgtgcatt

SEQ ID NO: 554 - DNA, 21 bp, Artificial Sequence
  Description of Artificial Sequence Synthetic oligonucleotide
ctgacctcctccccgtgcatt

SEQ ID NO: 555 - DNA, 17 bp, Artificial Sequence
  Description of Artificial Sequence Synthetic oligonucleotide
acctcctccccgtgcat

SEQ ID NO: 556 - DNA, 18 bp, Artificial Sequence
  Description of Artificial Sequence Synthetic oligonucleotide
gacctcctccccgtgcat

SEQ ID NO: 557 - DNA, 19 bp, Artificial Sequence
  Description of Artificial Sequence Synthetic oligonucleotide
tgacctcctccccgtgcat

SEQ ID NO: 558 - DNA, 17 bp, Artificial Sequence
  Description of Artificial Sequence Synthetic oligonucleotide
gacctcctccccgtgca

SEQ ID NO: 559 - DNA, 18 bp, Artificial Sequence
  Description of Artificial Sequence Synthetic oligonucleotide
tgacctcctccccgtgca

SEQ ID NO: 560 - DNA, 19 bp, Artificial Sequence
  Description of Artificial Sequence Synthetic oligonucleotide
ctgacctcctccccgtgca

SEQ ID NO: 561 - DNA, 20 bp, Artificial Sequence
  Description of Artificial Sequence Synthetic oligonucleotide
tctgacctcctccccgtgca

SEQ ID NO: 562 - DNA, 21 bp, Artificial Sequence
  Description of Artificial Sequence Synthetic oligonucleotide
ttctgacctcctccccgtgca

SEQ ID NO: 563 - DNA, 17 bp, Artificial Sequence
  Description of Artificial Sequence Synthetic oligonucleotide
tgacctcctccccgtgc

SEQ ID NO: 564 - DNA, 18 bp, Artificial Sequence
  Description of Artificial Sequence Synthetic oligonucleotide
ctgacctcctccccgtgc

SEQ ID NO: 565 - DNA, 19 bp, Artificial Sequence
  Description of Artificial Sequence Synthetic oligonucleotide
tctgacctcctccccgtgc

SEQ ID NO: 566 - DNA, 21 bp, Artificial Sequence
  Description of Artificial Sequence Synthetic oligonucleotide
gttctgacctcctccccgtgc

SEQ ID NO: 567 - DNA, 17 bp, Artificial Sequence
  Description of Artificial Sequence Synthetic oligonucleotide
ctgacctcctccccgtg

SEQ ID NO: 568 - DNA, 18 bp, Artificial Sequence
  Description of Artificial Sequence Synthetic oligonucleotide
tctgacctcctccccgtg

SEQ ID NO: 569 - DNA, 19 bp, Artificial Sequence
  Description of Artificial Sequence Synthetic oligonucleotide
ttctgacctcctccccgtg

SEQ ID NO: 570 - DNA, 21 bp, Artificial Sequence
  Description of Artificial Sequence Synthetic oligonucleotide
ggttctgacctcctccccgtg

SEQ ID NO: 571 - DNA, 17 bp, Artificial Sequence
  Description of Artificial Sequence Synthetic oligonucleotide
tctgacctcctccccgt

SEQ ID NO: 572 - DNA, 18 bp, Artificial Sequence
  Description of Artificial Sequence Synthetic oligonucleotide
ttctgacctcctccccgt

SEQ ID NO: 573 - DNA, 19 bp, Artificial Sequence
  Description of Artificial Sequence Synthetic oligonucleotide
gttctgacctcctccccgt

SEQ ID NO: 574 - DNA, 21 bp, Artificial Sequence
  Description of Artificial Sequence Synthetic oligonucleotide
aggttctgacctcctccccgt

SEQ ID NO: 575 - DNA, 17 bp, Artificial Sequence
  Description of Artificial Sequence Synthetic oligonucleotide
ttctgacctcctccccg

SEQ ID NO: 576 - DNA, 18 bp, Artificial Sequence
  Description of Artificial Sequence Synthetic oligonucleotide
gttctgacctcctccccg

SEQ ID NO: 577 - DNA, 19 bp, Artificial Sequence
  Description of Artificial Sequence Synthetic oligonucleotide
ggttctgacctcctccccg

SEQ ID NO: 578 - DNA, 20 bp, Artificial Sequence
  Description of Artificial Sequence Synthetic oligonucleotide
aggttctgacctcctccccg

SEQ ID NO: 579 - DNA, 21 bp, Artificial Sequence
  Description of Artificial Sequence Synthetic oligonucleotide
caggttctgacctcctccccg

SEQ ID NO: 580 - DNA, 17 bp, Artificial Sequence
  Description of Artificial Sequence Synthetic oligonucleotide
gttctgacctcctcccc

SEQ ID NO: 581 - DNA, 18 bp, Artificial Sequence
  Description of Artificial Sequence Synthetic oligonucleotide
ggttctgacctcctcccc

SEQ ID NO: 582 - DNA, 19 bp, Artificial Sequence
  Description of Artificial Sequence Synthetic oligonucleotide
aggttctgacctcctcccc

SEQ ID NO: 583 - DNA, 21 bp, Artificial Sequence
  Description of Artificial Sequence Synthetic oligonucleotide
tcaggttctgacctcctcccc

SEQ ID NO: 584 - DNA, 17 bp, Artificial Sequence
  Description of Artificial Sequence Synthetic oligonucleotide
ggttctgacctcctccc

SEQ ID NO: 585 - DNA, 18 bp, Artificial Sequence
  Description of Artificial Sequence Synthetic oligonucleotide
aggttctgacctcctccc

SEQ ID NO: 586 - DNA, 19 bp, Artificial Sequence
  Description of Artificial Sequence Synthetic oligonucleotide
caggttctgacctcctccc

SEQ ID NO: 587 - DNA, 21 bp, Artificial Sequence
  Description of Artificial Sequence Synthetic oligonucleotide
ttcaggttctgacctcctccc

SEQ ID NO: 588 - DNA, 17 bp, Artificial Sequence
  Description of Artificial Sequence Synthetic oligonucleotide
aggttctgacctcctcc

SEQ ID NO: 589 - DNA, 18 bp, Artificial Sequence
  Description of Artificial Sequence Synthetic oligonucleotide
caggttctgacctcctcc

SEQ ID NO: 590 - DNA, 19 bp, Artificial Sequence
  Description of Artificial Sequence Synthetic oligonucleotide
tcaggttctgacctcctcc

SEQ ID NO: 591 - DNA, 17 bp, Artificial Sequence
  Description of Artificial Sequence Synthetic oligonucleotide
caggttctgacctcctc

SEQ ID NO: 592 - DNA, 18 bp, Artificial Sequence
  Description of Artificial Sequence Synthetic oligonucleotide
tcaggttctgacctcctc

SEQ ID NO: 593 - DNA, 19 bp, Artificial Sequence
  Description of Artificial Sequence Synthetic oligonucleotide
ttcaggttctgacctcctc

SEQ ID NO: 594 - DNA, 17 bp, Artificial Sequence
  Description of Artificial Sequence Synthetic oligonucleotide
tcaggttctgacctcct

SEQ ID NO: 595 - DNA, 18 bp, Artificial Sequence
  Description of Artificial Sequence Synthetic oligonucleotide
ttcaggttctgacctcct

SEQ ID NO: 596 - DNA, 17 bp, Artificial Sequence
  Description of Artificial Sequence Synthetic oligonucleotide
ttcaggttctgacctcc

(19) United States
(12) Patent Application Publication
(10) Pub. No.: US 2022/0282246 A1
(43) Pub. Date: Sep. 8, 2022

(54) OLIGONUCLEOTIDE THERAPY FOR STARGARDT DISEASE

(71) Applicant: Deep Genomics Incorporated, Toronto (CA)

(72) Inventors: Daniele MERICO, Toronto (CA); Kahlin CHEUNG-ONG, Toronto (CA)

(21) Appl. No.: 17/572,321

(22) Filed: Jan. 10, 2022

Related U.S. Application Data
(63) Continuation of application No. PCT/CA2020/050954, filed on Jul. 10, 2020.
(60) Provisional application No. 62/873,792, filed on Jul. 12, 2019.

(51) Int. Cl. C12N 15/113; C12N 15/11; A61P 27/00; A61K 31/7088

(52) U.S. Cl. CPC C12N 15/113; C12N 15/111; A61P 27/00; A61K 31/7088; C12N 2310/11; C12N 2310/315; C12N 2310/321; C12N 2310/3341

(57) ABSTRACT

The present disclosure provides antisense oligonucleotides, compositions, and methods that target a ABCA4 exon or intron flanking an exon, thereby modulating splicing of ABCA4 pre-mRNA to increase the level of wild type ABCA4 mRNA molecules, e.g., to provide a therapy for retinitis pigmentosa, cone-rod dystrophy, or Stargardt disease. The present disclosure provides an antisense oligonucleotide including a nucleobase sequence at least 70% complementary to a ABCA4 pre-mRNA target sequence in an intron, 5′-flanking intron, a 3′-flanking intron, or a combination of an exon and the 5′-flanking or 3′-flanking intron.

### CROSS-REFERENCE

This application is a continuation of International Application No. PCT/CA2020/050954, filed on Jul. 10, 2020, which claims the benefit of U.S. Provisional Patent Application No. 62/873,792, filed Jul. 12, 2019, each of which is entirely incorporated herein by reference in its entirety.

### SEQUENCE LISTING

The instant application contains a Sequence Listing which has been submitted electronically in ASCII format and is hereby incorporated by reference in its entirety. Said ASCII copy, created on May 12, 2022, is named 51110-711_301_SL.txt and is 317,049 bytes in size.

### FIELD OF THE DISCLOSURE

The present disclosure relates to the field of oligonucleotides and their use for the treatment of disease. In particular, the disclosure pertains to antisense oligonucleotides that may be used in the treatment of Stargardt disease.

### BACKGROUND

ABCA4 (ATP binding cassette subfamily A member 4; entrez gene 24) is a transmembrane lipid transporter expressed in the photoreceptor outer segment, within the disc membranes. It is required to clear the reactive all-trans retinal from the photoreceptor disc lumen.

As part of the light cycle, 11-cis-retinal is generated in the retinal epithelium cells (RPE) and transported to the photoreceptor outer segment, where light triggers isomerization of rhodopsin-bound 11-cis-retinal to all-trans retinal. All-trans retinal can spontaneously flip to the photoreceptor disc membrane cytoplasm-facing side, or it can spontaneously react with phosphatidylethanolamine (PE), a phospholipid that is abundant in the photoreceptor outer segment, to form N-retinylidene-PE. N-retinylidene-PE cannot spontaneously flip, and it would accumulate without a specific transporter.

ABCA4 expression is restricted to photoreceptor cells. RefSeq contains only one curated isoform (NM_000350) comprising 50 exons, which is categorized principal by APPRIS. GENCODE contains one isoform categorized principal by APPRIS (ENST00000370225), which has the same CDS as NM_000350, and two minor isoforms (ENST00000536513, ENST00000649773). NM_000350 can be treated as the only ABCA4 functional isoform.

ABCA4 transports N-retinylidene-PE from the lumen-facing side of the membrane to the cytoplasm-facing side, where it spontaneously dissociates to all-trans retinal and PE. All-trans retinal is then reduced to all-trans retinol by the cytoplasmic enzyme RDH8 and transported back to RPE cells. In addition, ABCA4 transports PE from the lumen-facing to the cytoplasm-facing side of the photoreceptor disc membrane, maintaining the PE concentration lower.

If N-retinylidene-PE accumulates, it can form di-retinoid-pyridinium-PE (A2PE); all-trans retinal can also accumulate and form dimers. Since RPE cells recycle photoreceptor outer segments every 10 days, these compounds end up accumulating in their lysosomes. There, A2PE is hydrolyzed to di-retinoid-pyridinium-ethanolamine (A2E), which can be photoactivated and form highly reactive epoxides. This process is toxic for RPE cells and can lead to cell death. As photoreceptors lose the support of RPE, they can in turn suffer cell death.

The ABCA4 transport reaction follows three main steps: (i) binding of N-retinylidene-PE, binding of ATP, NBD domain dimerization, (ii) using the energy from ATP hydrolysis, change to a conformation that exposes N-retinylidene-PE to the cytoplasmic side and has lower affinity to it, (iii) release of N-retinylidene-PE and ADP, reversal to the original configuration.

Lack of ABCA4 function causes N-retinylidene-PE accumulation, which leads to formation of di-retinoid-pyridinium-PE (A2PE); all-trans retinal can also accumulate and form dimers. Since RPE cells recycle photoreceptor outer segments every 10 days, these compounds end up accumulating in their lysosomes. There, A2PE is hydrolyzed to di-retinoid-pyridinium-ethanolamine (A2E), which can be photoactivated and form highly reactive epoxides. This process is toxic for RPE cells and can lead to cell death. As photoreceptors lose the support of RPE, they can in turn suffer cell death. Higher levels of A2PE accumulation are directly toxic to photoreceptors, and cones are more sensitive than rods.

Pathogenic variants in ABCA4 cause a spectrum of recessive disorders, all characterized by progressive retinal degeneration; the phenotypic severity of the disorder is typically correlated to the extent of loss-of-function imparted by the variants. When both alleles are severely affected by variants severe cone-rod dystrophy may result, with a presentation similar to other forms of retinitis pigmentosa (RP). When one allele is severely affected by a variant while the other is only partially affected cone-rod dystrophy (CRD) may result. When one allele is severely affected by a variant while the other is not or only minorly affected or alternatively both alleles are only partially affected by a variant Stargardt disease (STGD1) may result.

Each disorder follows a progression with retinitis pigmentosa (RP) onset in the 1st decade of life typically progressing to blindness by the 2nd or 3d decade, cone-rod dystrophy (CRD) onset in the 1st decade of life progressing to blindness by mid-adulthood, and Stargardt disease (STGD1) with onset in the 1st or 2nd decade of life following progressive course.

No FDA-approved treatment exists.

Certain human genetic diseases (e.g., caused by genetic aberrations, such as point mutations) may be caused by aberrant splicing. As such, there is a need for a splicing modulator to treat diseases that are caused by aberrant splicing.

### SUMMARY

In general, the disclosure provides antisense oligonucleotides and methods of their use in the treatment of conditions associated with incorrect splicing of ABCA4 pre-mRNA (e.g., intron 6 or 36 inclusion, and exon 33 or 40 skipping).

In one aspect, the disclosure provides an antisense oligonucleotide including a nucleobase sequence that is at least 70% (e.g., at least 80%, at least 90%, at least 95%, or 100%) complementary to an ABCA4 pre-mRNA target sequence (e.g., g.107705G>A, g.104307A>G, g.115355G>A, or g.27356G>T mutation in SEQ ID NO: 1). The ABCA4 pre-mRNA target sequence may be disposed in, e.g., a 5′-flanking intron, a 3′-flanking intron, intron, exon, or a combination of an exon and the 5′-flanking or 3′-flanking intron.

In some embodiments, the ABCA4 pre-mRNA target sequence is in exon 6, a 5′-flanking intron adjacent to exon 6, 3′-flanking intron adjacent to exon 6, or a combination of exon 6 and the adjacent 5′-flanking or 3′-flanking intron. In certain embodiments, binding of the antisense oligonucleotide to the ABCA4 pre-mRNA target sequence reduces binding of a splicing factor to an intronic splicing enhancer in an exon, the 5′-flanking intron, the 3′-flanking intron, or a splicing enhancer.

In some embodiments, the ABCA4 pre-mRNA target sequence is in exon 33, a 5′-flanking intron adjacent to exon 33, 3′-flanking intron adjacent to exon 33, or a combination of exon 33 and the adjacent 5′-flanking or 3′-flanking intron. In certain embodiments, the ABCA4 pre-mRNA target sequence reduces the binding of a splicing factor to an intronic splicing silencer in the 5′-flanking intron or 3′-flanking intron.

In some embodiments, the ABCA4 pre-mRNA target sequence is in intron 36. In certain embodiments, the ABCA4 pre-mRNA target sequence reduces the binding of a splicing factor to an intronic splicing enhancer in an intron.

In some embodiments, the ABCA4 pre-mRNA target sequence is in exon 40, a 5′-flanking intron adjacent to exon 40, 3′-flanking intron adjacent to exon 40, or a combination of exon 40 and the adjacent 5′-flanking or 3′-flanking intron. In certain embodiments, the ABCA4 pre-mRNA target sequence reduces the binding of a splicing factor to an intronic splicing silencer in the 5′-flanking or 3′-flanking intron.

In particular embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 27362-27419 in SEQ ID NO: 1 (e.g., the ABCA4 pre-mRNA target sequence is wholly within these positions). In further embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 27372-27411 in SEQ ID NO: 1. In yet further embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 27377-27397 in SEQ ID NO: 1 (e.g., the ABCA4 pre-mRNA target sequence is wholly within these positions). In still further embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 27383-27402 in SEQ ID NO: 1 (e.g., the ABCA4 pre-mRNA target sequence is wholly within these positions). In other embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 27388-27411 in SEQ ID NO: 1 (e.g., the ABCA4 pre-mRNA target sequence is wholly within these positions). In other embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 27390-27411 in SEQ ID NO: 1 (e.g., the ABCA4 pre-mRNA target sequence is wholly within these positions). In other embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 27396-27414 in SEQ ID NO: 1 (e.g., the ABCA4 pre-mRNA target sequence is wholly within these positions). In other embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 27061-27152 in SEQ ID NO: 1 (e.g., the ABCA4 pre-mRNA target sequence is wholly within these positions).

In particular embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 104314-104336 in SEQ ID NO: 1 (e.g., the ABCA4 pre-mRNA target sequence is wholly within these positions

In particular embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 107659-107800 in SEQ ID NO: 1 (e.g., the ABCA4 pre-mRNA target sequence is wholly within these positions). In further embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 107690-107744 in SEQ ID NO: 1.

In particular embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 115149-115205 in SEQ ID NO: 1 (e.g., the ABCA4 pre-mRNA target sequence is wholly within these positions). In further embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 115306-115327 in SEQ ID NO: 1. In yet further embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 115357-115378 in SEQ ID NO: 1 (e.g., the ABCA4 pre-mRNA target sequence is wholly within these positions). In still further embodiments, the ABCA4 pre-mRNA target sequence includes at least one nucleotide (e.g., 5, 10, 11, 12, 13, 14, 15, 16, 17, 18, 19, or 20 consecutive nucleotides) located among positions 115384-115450 in SEQ ID NO: 1 (e.g., the ABCA4 pre-mRNA target sequence is wholly within these positions).

In some embodiments, the nucleobase sequence has at least 70% (e.g., at least 80%, at least 90%, at least 95%, or 100%) sequence identity to SEQ ID NO: 107, 102, 113, 129, 130,133, 134, 269, 270, 329, 333, 336, 337, 342, 343, 393, 422, 433, 438. In some embodiments, the nucleobase sequence is complementary to an aberrant ABCA4 sequence having a mutation in SEQ ID NO: 1 (e.g., a g.107705G>A, g.104307A>G, g.115355G>A, or g.27356G>T mutation in SEQ ID NO: 1).

In further embodiments, the nucleobase sequence has at least 70% (e.g., at least 80%, at least 90%, at least 95%, or 100%) sequence identity to any one of SEQ ID NOs: 60-198. In yet further embodiments, the nucleobase sequence has at least 70% (e.g., at least 80%, at least 90%, at least 95%, or 100%) sequence identity to any one of SEQ ID NOs: 73-175. In still further embodiments, the nucleobase sequence has at least 70% (e.g., at least 80%, at least 90%, at least 95%, or 100%) sequence identity to SEQ ID NO: 101-118. In some embodiments, the nucleobase sequence has at least 70% (e.g., at least 80%, at least 90%, at least 95%, or 100%) sequence identity to SEQ ID NO: 128-140.

In other embodiments, the nucleobase sequence has at least 70% (e.g., at least 80%, at least 90%, at least 95%, or 100%) sequence identity to SEQ ID NO: 157-171. In yet other embodiments, the nucleobase sequence has at least 70% (e.g., at least 80%, at least 90%, at least 95%, or 100%) sequence identity to SEQ ID NO: 157-171. In yet further embodiments, the nucleobase sequence has at least 70% (e.g., at least 80%, at least 90%, at least 95%, or 100%) sequence identity to SEQ ID NO: 165-171. In still other embodiments, the nucleobase sequence has at least 70% (e.g., at least 80%, at least 90%, at least 95%, or 100%) sequence identity to SEQ ID NO: 193-196. In some embodiments, the nucleobase sequence has at least 70% (e.g., at least 80%, at least 90%, at least 95%, or 100%) sequence identity to SEQ ID NO: 2-16. In certain embodiments, the nucleobase sequence has at least 70% (e.g., at least 80%, at least 90%, at least 95%, or 100%) sequence identity to SEQ ID NO: 260-287. In particular embodiments, the nucleobase sequence has at least 70% (e.g., at least 80%, at least 90%, at least 95%, or 100%) sequence identity to SEQ ID NO: 316-374 and 463-596. In further embodiments, the nucleobase sequence has at least 70% (e.g., at least 80%, at least 90%, at least 95%, or 100%) sequence identity to SEQ ID NO: 329-343 and 463-596. In yet further embodiments, the nucleobase sequence has at least 70% (e.g., at least 80%, at least 90%, at least 95%, or 100%) sequence identity to SEQ ID NO: 390-394. In still further embodiments, the nucleobase sequence has at least 70% (e.g., at least 80%, at least 90%, at least 95%, or 100%) sequence identity to SEQ ID NO: 422-423. In some embodiments, the nucleobase sequence has at least 70% (e.g., at least 80%, at least 90%, at least 95%, or 100%) sequence identity to SEQ ID NO: 433-434. In certain embodiments, the nucleobase sequence has at least 70% (e.g., at least 80%, at least 90%, at least 95%, or 100%) sequence identity to SEQ ID NO: 438-449.

In yet other embodiments, the antisense oligonucleotide includes at least one modified nucleobase. In still other embodiments, the antisense oligonucleotide includes at least one modified internucleoside linkage. In some embodiments, the modified internucleoside linkage is a phosphorothioate linkage. In certain embodiments, the phosphorothioate linkage is a stereochemically enriched phosphorothioate linkage. In particular embodiments, at least 50% of internucleoside linkages in the antisense oligonucleotide are modified internucleoside linkages. In further embodiments, at least 70% (e.g., at least 80%, at least 90%, at least 95%, or 100%) of internucleoside linkages in the antisense oligonucleotide are modified internucleoside linkage. In yet further embodiments, all internucleoside linkages in the antisense oligonucleotide are modified internucleoside linkages.

In still further embodiments, the antisense oligonucleotide includes at least one modified sugar nucleoside. In some embodiments, at least one modified sugar nucleoside is a 2′-modified sugar nucleoside. In certain embodiments, at least one 2′-modified sugar nucleoside includes a 2′-modification selected from the group consisting of 2′-fluoro, 2′-methoxy, and 2′-methoxyethoxy. In particular embodiments, the 2′-modified sugar nucleoside includes the 2′-methoxyethoxy modification. In further embodiments, at least one modified sugar nucleoside is a bridged nucleic acid. In yet further embodiments, the bridged nucleic acid is a locked nucleic acid (LNA), ethylene-bridged nucleic acid (ENA), or cEt nucleic acid. In still further embodiments, all nucleosides in the antisense oligonucleotide are modified sugar nucleosides. In some embodiments, the antisense oligonucleotide is a morpholino oligomer.

In certain embodiments, the antisense oligonucleotide further includes a targeting moiety. In particular embodiments, the targeting moiety is covalently conjugated at the 5′-terminus of the antisense oligonucleotide. In further embodiments, the targeting moiety is covalently conjugated at the 3′-terminus of the antisense oligonucleotide. In yet further embodiments, the targeting moiety is covalently conjugated at an internucleoside linkage of the antisense oligonucleotide. In still further embodiments, the targeting moiety is covalently conjugated through a linker (e.g., a cleavable linker). In other embodiments, the linker is a cleavable linker. In yet other embodiments, the targeting moiety includes N-acetylgalactosamine (e.g., is an N-acetylgalactosamine cluster).

In still other embodiments, the antisense oligonucleotide includes at least 12 nucleosides. In some embodiments, the antisense oligonucleotide includes at least 16 nucleosides. In certain embodiments, the antisense oligonucleotide includes a total of 50 nucleosides or fewer (e.g., 30 nucleosides or fewer, or 20 nucleosides or fewer). In particular embodiments, the antisense oligonucleotide includes a total of 16 to 20 nucleosides.

In another aspect, the disclosure provides a pharmaceutical composition including the antisense oligonucleotide of the disclosure and a pharmaceutically acceptable excipient.

In yet another aspect, the disclosure provides a method of increasing the level of exon-containing (e.g., exon 33 or 40-containing) ABCA4 mRNA molecules in a cell expressing an aberrant ABCA4 gene. The method includes contacting the cell with the antisense oligonucleotide of the disclosure.

In yet another aspect, the disclosure provides a method of decreasing the level of intron-containing (e.g., intron 6 or 36-containing) ABCA4 mRNA molecules in a cell expressing an aberrant ABCA4 gene. The method includes contacting the cell with the antisense oligonucleotide of the disclosure.

In some embodiments, the cell is in a subject.

In still another aspect, the disclosure provides a method of treating retinitis pigmentosa, cone-rod dystrophy, or Stargardt disease in a subject having an aberrant ABCA4 gene. The method includes administering a therapeutically effective amount of the antisense oligonucleotide of the disclosure or the pharmaceutical composition of the disclosure to the subject in need thereof.

In some embodiments, the administering step is performed parenterally. In certain embodiments, the method further includes administering to the subject a therapeutically effective amount of a second therapy for retinitis pigmentosa, cone-rod dystrophy, or Stargardt disease.

In yet further embodiments, the aberrant ABCA4 gene is ABCA4 having a g.107705G>A, g.104307A>G, g.115355G>A, or g.27356G>T mutation in SEQ ID NO: 1.

Recognized herein is the need for compositions and methods for treating diseases that may be caused by abnormal splicing resulting from an underlying genetic aberration. In some cases, antisense nucleic acid molecules, such as oligonucleotides, may be used to effectively modulate the splicing of targeted genes in genetic diseases, in order to alter the gene products produced. This approach can be applied in therapeutics to selectively modulate the expression and gene product composition for genes involved in genetic diseases.

The present disclosure provides compositions and methods that may advantageously use antisense oligonucleotides targeted to and hybridizable with nucleic acid molecules that encode for ABCA4. Such antisense oligonucleotides may target one or more splicing regulatory elements in one or more exons (e.g., exons 6, 33, 40) or introns (e.g., intron 36, 5′-flanking intro or 3′ flanking intron) of ABCA4. These splicing regulatory elements modulate splicing of ABCA4 ribonucleic acid (RNA).

In one aspect, the present disclosure provides an ABCA4 RNA splice-modulating antisense oligonucleotide having a sequence targeted to an exon or an intron adjacent to an exon (e.g., exon 6) of ABCA4. In some embodiments, a genetic aberration of ABCA4 includes the c.768G>T mutation. In some embodiments, the c.768G>T mutation results from ABCA4 chr1: 94564350:C:A [hg19/b37] (g.27356G>T in SEQ ID NO: 1). In some embodiments, the antisense oligonucleotide has a sequence targeted to one or more splicing regulatory elements. In some embodiments, the one or more splicing regulatory elements include an intronic splicing enhancer element. In some embodiments, the sequence is targeted to an intron adjacent to an abnormally spliced exon (e.g., a flanking intron). In some embodiments, the antisense oligonucleotide modulates variant splicing to yield an increase in intron exclusion (e.g., intron 6 inclusion). In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides.

In one aspect, the present disclosure provides an ABCA4 RNA splice-modulating antisense oligonucleotide having a sequence targeted to an exon or intron adjacent to an exon (e.g., exon 33) of ABCA4. In some embodiments, a genetic aberration of ABCA4 includes the c.4773+3A>G mutation. In some embodiments, the c.4773+3A>G mutation results from ABCA4 chr1: 94487399:T:C [hg19/b37] (g.104307A>G in SEQ ID NO: 1). In some embodiments, the antisense oligonucleotide has a sequence targeted to one or more splicing regulatory elements. In some embodiments, the one or more splicing regulatory elements include an intronic splicing silencer element. In some embodiments, the sequence is targeted to an intron adjacent to an abnormally spliced exon (e.g., a flanking intron). In some embodiments, the antisense oligonucleotide modulates variant splicing to yield an increase in exon inclusion (e.g., exon 33 inclusion). In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides.

In one aspect, the present disclosure provides an ABCA4 RNA splice-modulating antisense oligonucleotide having a sequence targeted to an intron (e.g., intron 36) of ABCA4. In some embodiments, a genetic aberration of ABCA4 includes the c.5196+1137G>A mutation. In some embodiments, the c.5196+1137G>A mutation results from ABCA4 chr1: 94484001:C:T [hg19/b37] (g.107705G>A in SEQ ID NO: 1). In some embodiments, the antisense oligonucleotide has a sequence targeted to one or more splicing regulatory elements. In some embodiments, the one or more splicing regulatory elements include an intronic splicing enhancer element. In some embodiments, the sequence is targeted to an intron containing an abnormally spliced intronic sequence (e.g., a pseudo exon). In some embodiments, the antisense oligonucleotide modulates variant splicing to yield an increase in intron exclusion (e.g., intron 36 inclusion). In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides.

In one aspect, the present disclosure provides an ABCA4 RNA splice-modulating antisense oligonucleotide having a sequence targeted to an exon or an intron adjacent to an exon (e.g., exon 40) of ABCA4. In some embodiments, a genetic aberration of ABCA4 includes the c.5714+5G>A mutation. In some embodiments, the c.5714+5G>A mutation results from ABCA4 chr1: 94476351:C:T [hg19/b37] (g.115355G>A in SEQ ID NO: 1). In some embodiments, the antisense oligonucleotide has a sequence targeted to one or more splicing regulatory elements. In some embodiments, the one or more splicing regulatory elements include an intronic splicing silencer element. In some embodiments, the sequence is targeted to an intron adjacent to an abnormally spliced exon (e.g., a flanking intron). In some embodiments, the antisense oligonucleotide modulates variant splicing to yield an increase in exon inclusion (e.g., exon 40 inclusion). In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides.

In another aspect, the present disclosure provides a method for modulating splicing of ABCA4 RNA in a cell, tissue, or organ of a subject, including bringing the cell, tissue, or organ in contact with an antisense oligonucleotide including one or more sequences targeted to an exon or intron adjacent to an exon (e.g., exon 6) of ABCA4. In some embodiments, the genetic aberration of ABCA4 includes the c.768G>T mutation. In some embodiments, the c.768G>T mutation results from ABCA4 chr1: 94564350:C:A [hg19/b37] (g.27356G>T in SEQ ID NO: 1). In some embodiments, the antisense oligonucleotide has a sequence targeted to one or more splicing regulatory elements. In some embodiments, the one or more splicing regulatory elements are an intronic splicing enhancer element. In some embodiments, the sequence is targeted to an intron adjacent to an abnormally spliced exon (e.g., a flanking intron). In some embodiments, the antisense oligonucleotide modulates variant splicing to yield an increase in intron exclusion (e.g., intron 6 inclusion), e.g., increase by at least 5%, at least 10%, at least 15%, at least 20%, at least 25%, at least 30%, at least 35%, at least 40%, at least 45%, or at least 50%; e.g., up to 100%, up to 90%, up to 80%, up to 70%, up to 60%, up to 50%, up to 40%, up to 30%, up to 20%, as compared to the ratio of intron-excluding ABCA4 transcripts (e.g., intron 6-excluding ABCA4 transcripts) to the total number of ABCA4 transcript molecules in a cell including ABCA4 gene having an intron-including mutation (e.g., an intron 6-including mutation) in the absence of a treatment with an antisense oligonucleotide. In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides. In some embodiments, the subject has or is suspected of having a disease, e.g., retinitis pigmentosa, cone-rod dystrophy, or Stargardt disease, and the subject is monitored for a progression or regression of the disease in response to bringing the cell, tissue, or organ in contact with the composition.

In another aspect, the present disclosure provides a method for modulating splicing of ABCA4 RNA in a cell, tissue, or organ of a subject, including bringing the cell, tissue, or organ in contact with an antisense oligonucleotide including one or more sequences targeted to an exon or intron adjacent to an exon (e.g., exon 33) of ABCA4. In some embodiments, the genetic aberration of ABCA4 includes the c.4773+3A>G mutation. In some embodiments, the c.4773+3A>G mutation results from ABCA4 chr1: 94487399:T:C [hg19/b37] (g.104307A>G in SEQ ID NO: 1). In some embodiments, the antisense oligonucleotide has a sequence targeted to one or more splicing regulatory elements. In some embodiments, the one or more splicing regulatory elements are an intronic splicing silencer element. In some embodiments, the sequence is targeted to an intron adjacent to an abnormally spliced exon (e.g., a flanking intron). In some embodiments, the antisense oligonucleotide modulates variant splicing to yield an increase in exon inclusion (e.g., exon 33 inclusion), e.g., increase by at least 5%, at least 10%, at least 15%, at least 20%, at least 25%, at least 30%, at least 35%, at least 40%, at least 45%, or at least 50%; e.g., up to 100%, up to 90%, up to 80%, up to 70%, up to 60%, up to 50%, up to 40%, up to 30%, up to 20%, as compared to the ratio of exon-including ABCA4 transcripts (e.g., exon 33-including ABCA4 transcripts) to the total number of ABCA4 transcript molecules in a cell including ABCA4 gene having an exon-skipping mutation (e.g., an exon 33-skipping mutation) in the absence of a treatment with an antisense oligonucleotide. In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides. In some embodiments, the subject has or is suspected of having a disease, e.g., retinitis pigmentosa, cone-rod dystrophy, or Stargardt disease, and the subject is monitored for a progression or regression of the disease in response to bringing the cell, tissue, or organ in contact with the composition.

In another aspect, the present disclosure provides a method for modulating splicing of ABCA4 RNA in a cell, tissue, or organ of a subject, including bringing the cell, tissue, or organ in contact with an antisense oligonucleotide including one or more sequences targeted to an intron (e.g., intron 36) of ABCA4. In some embodiments, the genetic aberration of ABCA4 includes the c.5196+1137G>A mutation. In some embodiments, the c.5196+1137G>A mutation results from ABCA4 chr1: 94484001:C:T [hg19/b37] (g.107705G>A in SEQ ID NO: 1). In some embodiments, the antisense oligonucleotide has a sequence targeted to one or more splicing regulatory elements. In some embodiments, the one or more splicing regulatory elements are an intronic splicing enhancer element. In some embodiments, the sequence is targeted to an intron containing an abnormally spliced intronic sequence (e.g., a pseudo exon). In some embodiments, the antisense oligonucleotide modulates variant splicing to yield an increase in intron exclusion (e.g., intron 36 exclusion), e.g., increase by at least 5%, at least 10%, at least 15%, at least 20%, at least 25%, at least 30%, at least 35%, at least 40%, at least 45%, or at least 50%; e.g., up to 100%, up to 90%, up to 80%, up to 70%, up to 60%, up to 50%, up to 40%, up to 30%, up to 20%, as compared to the ratio of intron-excluding ABCA4 transcripts (e.g., intron 36-excluding ABCA4 transcripts) to the total number of ABCA4 transcript molecules in a cell including ABCA4 gene having an intron-including mutation (e.g., an intron 36-including mutation) in the absence of a treatment with an antisense oligonucleotide. In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides. In some embodiments, the subject has or is suspected of having a disease, e.g., retinitis pigmentosa, cone-rod dystrophy, or Stargardt disease, and the subject is monitored for a progression or regression of the disease in response to bringing the cell, tissue, or organ in contact with the composition.

In another aspect, the present disclosure provides a method for modulating splicing of ABCA4 RNA in a cell, tissue, or organ of a subject, including bringing the cell, tissue, or organ in contact with an antisense oligonucleotide including one or more sequences targeted to an exon or intron adjacent to an exon (e.g., exon 40) of ABCA4. In some embodiments, the genetic aberration of ABCA4 includes the c.5714+5G>A mutation. In some embodiments, the c.5714+5G>A mutation results from ABCA4 chr1: 94476351:C:T [hg19/b37] (g.115355G>A in SEQ ID NO: 1). In some embodiments, the antisense oligonucleotide has a sequence targeted to one or more splicing regulatory elements. In some embodiments, the one or more splicing regulatory elements are an intronic splicing silencer element. In some embodiments, the sequence is targeted to an intron adjacent to an abnormally spliced exon (e.g., a flanking intron). In some embodiments, the antisense oligonucleotide modulates variant splicing to yield an increase in exon inclusion (e.g., exon 40 inclusion), e.g., increase by at least 5%, at least 10%, at least 15%, at least 20%, at least 25%, at least 30%, at least 35%, at least 40%, at least 45%, or at least 50%; e.g., up to 100%, up to 90%, up to 80%, up to 70%, up to 60%, up to 50%, up to 40%, up to 30%, up to 20%, as compared to the ratio of exon-including ABCA4 transcripts (e.g., exon 40-including ABCA4 transcripts) to the total number of ABCA4 transcript molecules in a cell including ABCA4 gene having an exon-skipping mutation (e.g., an exon 40-skipping mutation) in the absence of a treatment with an antisense oligonucleotide. In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides. In some embodiments, the subject has or is suspected of having a disease, e.g., retinitis pigmentosa, cone-rod dystrophy, or Stargardt disease, and the subject is monitored for a progression or regression of the disease in response to bringing the cell, tissue, or organ in contact with the composition.

In another aspect, the present disclosure provides a method for treating retinitis pigmentosa, cone-rod dystrophy, or Stargardt disease in a subject, including administering to the subject a therapeutically effective amount of an antisense oligonucleotide including one or more sequences targeted to an exon or intron adjacent to an exon (e.g., exon 6) of ABCA4. The antisense oligonucleotide modulates splicing of ABCA4 RNA. In some embodiments, the genetic aberration of ABCA4 includes the c.768G>T mutation. In some embodiments, the c.768G>T mutation results from ABCA4 chr1: 94564350:C:A [hg19/b37] (g.27356G>T in SEQ ID NO: 1). In some embodiments, the antisense oligonucleotide has a sequence targeted to one or more splicing regulatory elements. In some embodiments, the one or more splicing regulatory elements are an intronic splicing enhancer element. In some embodiments, the sequence is targeted to an intron adjacent to an abnormally spliced exon of the genetic aberration of ABCA4 that modulates variant splicing of ABCA4 RNA (e.g., a flanking intron). In some embodiments, the antisense oligonucleotide modulates variant splicing to yield an increase in intron exclusion (e.g., intron 6 inclusion), e.g., increase by at least 5%, at least 10%, at least 15%, at least 20%, at least 25%, at least 30%, at least 35%, at least 40%, at least 45%, or at least 50%; e.g., up to 100%, up to 90%, up to 80%, up to 70%, up to 60%, up to 50%, up to 40%, up to 30%, up to 20%, as compared to the ratio of intron-excluding ABCA4 transcripts (e.g., intron 6-excluding ABCA4 transcripts) to the total number of ABCA4 transcript molecules in a cell including ABCA4 gene having an intron-including mutation (e.g., an intron 6-including mutation) in the absence of a treatment with an antisense oligonucleotide. In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides. In some embodiments, the subject is monitored for a progression or regression of retinitis pigmentosa, cone-rod dystrophy, or Stargardt disease in response to administering to the subject the therapeutically effective amount of the antisense oligonucleotide.

In another aspect, the present disclosure provides a method for treating retinitis pigmentosa, cone-rod dystrophy, or Stargardt disease in a subject, including administering to the subject a therapeutically effective amount of an antisense oligonucleotide including one or more sequences targeted to an exon or intron adjacent to an exon (e.g., exon 33) of ABCA4. The antisense oligonucleotide modulates splicing of ABCA4 RNA. In some embodiments, the genetic aberration of ABCA4 includes the c.4773+3A>G mutation. In some embodiments, the c.4773+3A>G mutation results from ABCA4 chr1: 94487399:T:C [hg19/b37] (g.104307A>G in SEQ ID NO: 1). In some embodiments, the antisense oligonucleotide has a sequence targeted to one or more splicing regulatory elements. In some embodiments, the one or more splicing regulatory elements are an intronic splicing silencer element. In some embodiments, the sequence is targeted to an intron adjacent to an abnormally spliced exon of the genetic aberration of ABCA4 that modulates variant splicing of ABCA4 RNA (e.g., a flanking intron). In some embodiments, the antisense oligonucleotide modulates variant splicing to yield an increase in exon inclusion (e.g., exon 33 inclusion), e.g., increase by at least 5%, at least 10%, at least 15%, at least 20%, at least 25%, at least 30%, at least 35%, at least 40%, at least 45%, or at least 50%; e.g., up to 100%, up to 90%, up to 80%, up to 70%, up to 60%, up to 50%, up to 40%, up to 30%, up to 20%, as compared to the ratio of exon-including ABCA4 transcripts (e.g., exon 33-including ABCA4 transcripts) to the total number of ABCA4 transcript molecules in a cell including ABCA4 gene having an exon-skipping mutation (e.g., an exon 33-skipping mutation) in the absence of a treatment with an antisense oligonucleotide. In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides. In some embodiments, the subject is monitored for a progression or regression of retinitis pigmentosa, cone-rod dystrophy, or Stargardt disease in response to administering to the subject the therapeutically effective amount of the antisense oligonucleotide.

In another aspect, the present disclosure provides a method for treating retinitis pigmentosa, cone-rod dystrophy, or Stargardt disease in a subject, including administering to the subject a therapeutically effective amount of an antisense oligonucleotide including one or more sequences targeted to an intron (e.g., intron 36) of ABCA4. The antisense oligonucleotide modulates splicing of ABCA4 RNA. In some embodiments, the genetic aberration of ABCA4 includes the c.5196+1137G>A mutation. In some embodiments, the c.5196+1137G>A mutation results from ABCA4 chr1: 94484001:C:T [hg19/b37] (g.107705G>A in SEQ ID NO: 1). In some embodiments, the antisense oligonucleotide has a sequence targeted to one or more splicing regulatory elements. In some embodiments, the one or more splicing regulatory elements are an intronic splicing enhancer element. In some embodiments, the sequence is targeted to an intron containing an abnormally spliced intronic sequence containing the genetic aberration of ABCA4 that modulates variant splicing of ABCA4 RNA (e.g., a pseudo exon). In some embodiments, the antisense oligonucleotide modulates variant splicing to yield an increase in intron exclusion (e.g., intron 36 exclusion), e.g., increase by at least 5%, at least 10%, at least 15%, at least 20%, at least 25%, at least 30%, at least 35%, at least 40%, at least 45%, or at least 50%; e.g., up to 100%, up to 90%, up to 80%, up to 70%, up to 60%, up to 50%, up to 40%, up to 30%, up to 20%, as compared to the ratio of intron-excluding ABCA4 transcripts (e.g., intron 36-excluding ABCA4 transcripts) to the total number of ABCA4 transcript molecules in a cell including ABCA4 gene having an intron-including mutation (e.g., an intron 36-including mutation) in the absence of a treatment with an antisense oligonucleotide. In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides. In some embodiments, the subject is monitored for a progression or regression of retinitis pigmentosa, cone-rod dystrophy, or Stargardt disease in response to administering to the subject the therapeutically effective amount of the antisense oligonucleotide.

In another aspect, the present disclosure provides a method for treating retinitis pigmentosa, cone-rod dystrophy, or Stargardt disease in a subject, including administering to the subject a therapeutically effective amount of an antisense oligonucleotide including one or more sequences targeted to an exon or intron adjacent to an exon (e.g., exon 40) of ABCA4. The antisense oligonucleotide modulates splicing of ABCA4 RNA. In some embodiments, the genetic aberration of ABCA4 includes the c.5714+5G>A mutation. In some embodiments, the c.5714+5G>A mutation results from ABCA4 chr1: 94476351:C:T [hg19/b37] (g.115355G>A in SEQ ID NO: 1). In some embodiments, the antisense oligonucleotide has a sequence targeted to one or more splicing regulatory elements. In some embodiments, the one or more splicing regulatory elements are an intronic splicing silencer element. In some embodiments, the sequence is targeted to an intron adjacent to an abnormally spliced exon of the genetic aberration of ABCA4 that modulates variant splicing of ABCA4 RNA (e.g., a flanking intron). In some embodiments, the antisense oligonucleotide modulates variant splicing to yield an increase in exon inclusion (e.g., exon 40 inclusion), e.g., increase by at least 5%, at least 10%, at least 15%, at least 20%, at least 25%, at least 30%, at least 35%, at least 40%, at least 45%, or at least 50%; e.g., up to 100%, up to 90%, up to 80%, up to 70%, up to 60%, up to 50%, up to 40%, up to 30%, up to 20%, as compared to the ratio of exon-including ABCA4 transcripts (e.g., exon 40-including ABCA4 transcripts) to the total number of ABCA4 transcript molecules in a cell including ABCA4 gene having an exon-skipping mutation (e.g., an exon 40-skipping mutation) in the absence of a treatment with an antisense oligonucleotide. In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides. In some embodiments, the subject is monitored for a progression or regression of retinitis pigmentosa, cone-rod dystrophy, or Stargardt disease in response to administering to the subject the therapeutically effective amount of the antisense oligonucleotide.

In another aspect, the present disclosure provides a pharmaceutical composition for treatment of retinitis pigmentosa, cone-rod dystrophy, or Stargardt disease including an antisense oligonucleotide and a pharmaceutically acceptable carrier. The antisense oligonucleotide includes a sequence targeted to an exon or intron adjacent to the abnormally spliced exon. The antisense oligonucleotide modulates splicing of ABCA4 RNA. In some embodiments, the genetic aberration of ABCA4 includes c.768G>T. In some embodiments, the c.768G>T mutation results from ABCA4 chr1: 94564350:C:A [hg19/b37] (g.27356G>T in SEQ ID NO: 1).

In another aspect, the present disclosure provides a pharmaceutical composition for treatment of retinitis pigmentosa, cone-rod dystrophy, or Stargardt disease including an antisense oligonucleotide and a pharmaceutically acceptable carrier. The antisense oligonucleotide includes a sequence targeted to an exon or intron adjacent to the abnormally spliced exon. The antisense oligonucleotide modulates splicing of ABCA4 RNA. In some embodiments, the genetic aberration of ABCA4 includes c.4773+3A>G. In some embodiments, the c.4773+3A>G mutation results from ABCA4 chr1: 94487399:T:C [hg19/b37] (g.104307A>G in SEQ ID NO: 1).

In another aspect, the present disclosure provides a pharmaceutical composition for treatment of retinitis pigmentosa, cone-rod dystrophy, or Stargardt disease including an antisense oligonucleotide and a pharmaceutically acceptable carrier. The antisense oligonucleotide includes a sequence targeted to an intron abnormally spliced intron. The antisense oligonucleotide modulates splicing of ABCA4 RNA. In some embodiments, the genetic aberration of ABCA4 includes c.5196+1137G>A. In some embodiments, the c.5196+1137G>A mutation results from ABCA4 chr1: 94484001:C:T [hg19/b37] (g.107705G>A in SEQ ID NO: 1).

In another aspect, the present disclosure provides a pharmaceutical composition for treatment of retinitis pigmentosa, cone-rod dystrophy, or Stargardt disease including an antisense oligonucleotide and a pharmaceutically acceptable carrier. The antisense oligonucleotide includes a sequence targeted to an intron adjacent to the abnormally spliced exon. The antisense oligonucleotide modulates splicing of ABCA4 RNA. In some embodiments, the genetic aberration of ABCA4 includes c.5714+5G>A. In some embodiments, the c.5714+5G>A mutation results from ABCA4 chr1: 94476351:C:T [hg19/b37] (g.115355G>A in SEQ ID NO: 1).

### Definitions

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

The term “ABCA4” as used herein, generally represents a nucleic acid (e.g., genomic DNA, pre-mRNA, or mRNA) that is translated and, if genomic DNA, first transcribed, in vivo to ABCA4 protein. An exemplary genomic DNA sequence comprising the human ABCA4 gene is given by SEQ ID NO: 1 (NCBI Reference Sequence: NG_009073.1). SEQ ID NO: 1 provides the sequence for the antisense strand of the genomic DNA of ABCA4 (positions 5001-133313 in SEQ ID NO: 1). One of skill in the art will recognize that an RNA sequence typically includes uridines instead of thymidines. The term “ABCA4” as used herein, represents wild-type and mutant versions. An exemplary mutant nucleic acid (e.g., genomic DNA, pre-mRNA, or mRNA) results in ABCA4 protein lacking any of exon 33 or exon 40, or containing an extended exon 6 or pseudo exon.

The term “acyl,” as used herein, generally represents a chemical substituent of formula —C(O)—R, where R is alkyl, aryl, arylalkyl, cycloalkyl, heterocyclyl, heterocyclyl alkyl, heteroaryl, or heteroaryl alkyl. An optionally substituted acyl is an acyl that is optionally substituted as described herein for each group R.

The term “acyloxy,” as used herein, generally represents a chemical substituent of formula —OR, where R is acyl. An optionally substituted acyloxy is an acyloxy that is optionally substituted as described herein for acyl.

The term “alkane-tetrayl,” as used herein, generally represents a tetravalent, acyclic, straight or branched chain, saturated hydrocarbon group having from 1 to 16 carbons, unless otherwise specified. Alkane-tetrayl may be optionally substituted as described for alkyl.

The term “alkane-triyl,” as used herein, generally represents a trivalent, acyclic, straight or branched chain, saturated hydrocarbon group having from 1 to 16 carbons, unless otherwise specified. Alkane-triyl may be optionally substituted as described for alkyl.

The term “alkanoyl,” as used herein, generally represents a chemical substituent of formula —C(O)—R, where R is alkyl. An optionally substituted alkanoyl is an alkanoyl that is optionally substituted as described herein for alkyl.

The term “alkoxy,” as used herein, generally represents a chemical substituent of formula-OR, where R is a C₁₋₆ alkyl group, unless otherwise specified. An optionally substituted alkoxy is an alkoxy group that is optionally substituted as defined herein for alkyl.

The term “alkyl,” as used herein, generally refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; and ═NR′, where R′ is H, alkyl, aryl, or heterocyclyl. In some embodiments, a substituted alkyl includes two substituents (oxo and hydroxy, or oxo and alkoxy) to form a group -L-CO—R, where L is a bond or optionally substituted C₁₋₁₁ alkylene, and R is hydroxyl or alkoxy. Each of the substituents may itself be unsubstituted or, valency permitting, substituted with unsubstituted substituent(s) defined herein for each respective group.

The term “alkylene,” as used herein, generally represents a divalent substituent that is a monovalent alkyl having one hydrogen atom replaced with a valency. An optionally substituted alkylene is an alkylene that is optionally substituted as described herein for alkyl.

The term “aryl,” as used herein, generally represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; and cyano. Each of the substituents may itself be unsubstituted or substituted with unsubstituted substituent(s) defined herein for each respective group.

The term “aryl alkyl,” as used herein, generally represents an alkyl group substituted with an aryl group. The aryl and alkyl portions may be optionally substituted as the individual groups as described herein.

The term “arylene,” as used herein, generally represents a divalent substituent that is an aryl having one hydrogen atom replaced with a valency. An optionally substituted arylene is an arylene that is optionally substituted as described herein for aryl.

The term “aryloxy,” as used herein, generally represents a group —OR, where R is aryl. Aryloxy may be an optionally substituted aryloxy. An optionally substituted aryloxy is aryloxy that is optionally substituted as described herein for aryl.

The term “bicyclic sugar moiety,” as used herein, generally represents a modified sugar moiety including two fused rings. In certain embodiments, the bicyclic sugar moiety includes a furanosyl ring.

The expression “C_{x-y},” as used herein, generally indicates that the group, the name of which immediately follows the expression, when unsubstituted, contains a total of from x to y carbon atoms. If the group is a composite group (e.g., aryl alkyl), C_{x-y} indicates that the portion, the name of which immediately follows the expression, when unsubstituted, contains a total of from x to y carbon atoms. For example, (C₆₋₁₀-aryl)-C₁₋₆-alkyl is a group, in which the aryl portion, when unsubstituted, contains a total of from 6 to 10 carbon atoms, and the alkyl portion, when unsubstituted, contains a total of from 1 to 6 carbon atoms.

The term “complementary,” as used herein in reference to a nucleobase sequence, generally refers to the nucleobase sequence having a pattern of contiguous nucleobases that permits an oligonucleotide having the nucleobase sequence to hybridize to another oligonucleotide or nucleic acid to form a duplex structure under physiological conditions. Complementary sequences include Watson-Crick base pairs formed from natural and/or modified nucleobases. Complementary sequences can also include non-Watson-Crick base pairs, such as wobble base pairs (guanosine-uracil, hypoxanthine-uracil, hypoxanthine-adenine, and hypoxanthine-cytosine) and Hoogsteen base pairs.

The term “contiguous,” as used herein in the context of an oligonucleotide, generally refers to nucleosides, nucleobases, sugar moieties, or internucleoside linkages that are immediately adjacent to each other. For example, “contiguous nucleobases” means nucleobases that are immediately adjacent to each other in a sequence.

The term “cycloalkyl,” as used herein, generally refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; —NR′, where R′ is H, alkyl, aryl, or heterocyclyl. Each of the substituents may itself be unsubstituted or substituted with unsubstituted substituent(s) defined herein for each respective group.

The term “cycloalkylene,” as used herein, generally represents a divalent substituent that is a cycloalkyl having one hydrogen atom replaced with a valency. An optionally substituted cycloalkylene is a cycloalkylene that is optionally substituted as described herein for cycloalkyl.

The term “cycloalkoxy,” as used herein, generally represents a group —OR, where R is cycloalkyl. Cycloalkoxy may be an optionally substituted cycloalkoxy. An optionally substituted cycloalkoxy is cycloalkoxy that is optionally substituted as described herein for cycloalkyl.

The term “duplex,” as used herein, generally represents two oligonucleotides that are paired through hybridization of complementary nucleobases.

The term “exon 6,” as used herein, generally refers to exon 6 of ABCA4 pre-mRNA or genomic DNA which corresponds to positions 27159 to 27356 in SEQ ID NO: 1 (hg19/b37 coordinates chr1:94564350-94564547), or a mutant version thereof (e.g., g.27356G>T in SEQ ID NO: 1).

The term “exon 33,” as used herein, generally refers to exon 33 of ABCA4 pre-mRNA or genomic DNA, e.g. which corresponds to positions 104199 to 104304 in SEQ ID NO: 1 (hg19/b37 coordinates chr1:94487402-94487507), or a mutant version thereof.

The term “exon 40,” as used herein, generally refers to exon 40 of ABCA4 pre-mRNA or genomic DNA, e.g. which corresponds to positions 115221 to 115350 in SEQ ID NO: 1 (hg19/b37 coordinates chr1:94476356-94476485), or a mutant version thereof.

The term “flanking intron,” as used herein, generally refers to an intron that is adjacent to the 5′- or 3′-end of a ABCA4 exon (e.g., exon 6, 33, or 40) or a mutant thereof (e.g. NM_000350.2(ABCA4):c.5714+5G>A [g.115355G>A on SEQ ID NO: 1] or NM_000350.2(ABCA4):c.5196+1137G>A [g.107705G>A on SEQ ID NO: 1]). The flanking intron is a 5′-flanking intron or a 3′-flanking intron. The 5′-flanking intron corresponds to the flanking intron that is adjacent to the 5′-end of the exon (e.g., exon 6, 33, or 40) targeted for inclusion. In some embodiments, the 5′-flanking intron is disposed between exon 5 and exon 6, exon 32 and exon 33, and exon 39 and exon 40 in SEQ ID NO: 1. The 3′-flanking intron corresponds to the flanking intron that is adjacent to the 3′-end of the exon (e.g., exon 6, 33, or 40) targeted for inclusion. In some embodiments, the 3′-flanking intron is disposed between exon 6 and exon 7, exon 33 and exon 34, and exon 40 and exon 41 in SEQ ID NO: 1).

The term “genetic aberration,” as used herein, generally refers to a mutation or variant in a gene. Examples of genetic aberration may include, but are not limited to, a point mutation (single nucleotide variant or single base substitution), an insertion or deletion (indel), a transversion, a translocation, an inversion, or a truncation. An aberrant ABCA4 gene may include one or more mutations causing the splicing of pre-mRNA to: skip an exon in the ABCA4 gene (e.g., exon 33 or 40), include a portion of a flanking intron adjacent to an exon in the ABCA4 gene (e.g., a portion of a flanking intron adjacent to exon 6), or include a pseudo exon (e.g. a pseudo exon located in intro 36).

The term “halo,” as used herein, generally represents a halogen selected from bromine, chlorine, iodine, and fluorine.

The term “heteroalkane-tetrayl,” as used herein generally refers to an alkane-tetrayl group interrupted once by one heteroatom; twice, each time, independently, by one heteroatom; three times, each time, independently, by one heteroatom; or four times, each time, independently, by one heteroatom. Each heteroatom is, independently, O, N, or S. In some embodiments, the heteroatom is O or N. An unsubstituted C_{X-Y} heteroalkane-tetrayl contains from X to Y carbon atoms as well as the heteroatoms as defined herein. The heteroalkane-tetrayl group may be unsubstituted or substituted (e.g., optionally substituted heteroalkane-tetrayl), as described for heteroalkyl.

The term “heteroalkane-triyl,” as used herein generally refers to an alkane-triyl group interrupted once by one heteroatom; twice, each time, independently, by one heteroatom; three times, each time, independently, by one heteroatom; or four times, each time, independently, by one heteroatom. Each heteroatom is, independently, O, N, or S. In some embodiments, the heteroatom is O or N. An unsubstituted C_{X-Y} heteroalkane-triyl contains from X to Y carbon atoms as well as the heteroatoms as defined herein. The heteroalkane-triyl group may be unsubstituted or substituted (e.g., optionally substituted heteroalkane-triyl), as described for heteroalkyl.

The term “heteroalkyl,” as used herein, generally refers to an alkyl group interrupted one or more times by one or two heteroatoms each time. Each heteroatom is independently O, N, or S. None of the heteroalkyl groups includes two contiguous oxygen atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteroatom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, aryl, cycloalkyl, heterocyclyl, or cyano, where each R^{N2} is independently H, alkyl, cycloalkyl, aryl, or heterocyclyl, and each R^{N3} is independently alkyl, cycloalkyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. In some embodiments, carbon atoms are found at the termini of a heteroalkyl group. In some embodiments, heteroalkyl is PEG.

The term “heteroalkylene,” as used herein, generally represents a divalent substituent that is a heteroalkyl having one hydrogen atom replaced with a valency. An optionally substituted heteroalkylene is a heteroalkylene that is optionally substituted as described herein for heteroalkyl.

The term “heteroaryl,” as used herein, generally represents a monocyclic 5-, 6-, 7-, or 8-membered ring system, or a fused or bridging bicyclic, tricyclic, or tetracyclic ring system; the ring system contains one, two, three, or four heteroatoms independently selected from the group consisting of nitrogen, oxygen, and sulfur; and at least one of the rings is an aromatic ring. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, dihydroindolyl, tetrahydroquinolyl, tetrahydroisoquinolyl, etc. The term bicyclic, tricyclic, and tetracyclic heteroaryls include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heteroaryls include 1,2,3,5,8,8a-hexahydroindolizine; 2,3-dihydrobenzofuran; 2,3-dihydroindole; and 2,3-dihydrobenzothiophene. Heteroaryl may be optionally substituted with one, two, three, four, or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl. Each of the substituents may itself be unsubstituted or substituted with unsubstituted substituent(s) defined herein for each respective group.

The term “heteroarylene,” as used herein, generally represents a divalent substituent that is a heteroaryl having one hydrogen atom replaced with a valency. An optionally substituted heteroarylene is a heteroarylene that is optionally substituted as described herein for heteroaryl.

The term “heteroaryloxy,” as used herein, generally refers to a structure —OR, in which R is heteroaryl. Heteroaryloxy can be optionally substituted as defined for heteroaryl.

The term “heterocyclyl,” as used herein, generally represents a monocyclic, bicyclic, tricyclic, or tetracyclic ring system having fused or bridging 4-, 5-, 6-, 7-, or 8-membered rings, unless otherwise specified, the ring system containing one, two, three, or four heteroatoms independently selected from the group consisting of nitrogen, oxygen, and sulfur. Heterocyclyl may be aromatic or non-aromatic. An aromatic heterocyclyl is heteroaryl as described herein. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups have a carbon count of 1 to 16 carbon atoms unless otherwise specified. Certain heterocyclyl groups may have a carbon count up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, pyranyl, dihydropyranyl, dithiazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., a cyclohexane ring, a cyclohexene ring, a cyclopentane ring, a cyclopentene ring, or another heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; ═S; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl.

The term “heterocyclyl alkyl,” as used herein, generally represents an alkyl group substituted with a heterocyclyl group. The heterocyclyl and alkyl portions of an optionally substituted heterocyclyl alkyl are optionally substituted as described for heterocyclyl and alkyl, respectively.

The term “heterocyclylene,” as used herein, generally represents a divalent substituent that is a heterocyclyl having one hydrogen atom replaced with a valency. An optionally substituted heterocyclylene is a heterocyclylene that is optionally substituted as described herein for heterocyclyl.

The term “heterocyclyloxy,” as used herein, generally refers to a structure —OR, in which R is heterocyclyl. Heterocyclyloxy can be optionally substituted as described for heterocyclyl.

The term “heteroorganic,” as used herein, generally refers to (i) an acyclic hydrocarbon interrupted one or more times by one or two heteroatoms each time, or (ii) a cyclic hydrocarbon including one or more (e.g., one, two, three, or four) endocyclic heteroatoms. Each heteroatom is independently O, N, or S. None of the heteroorganic groups includes two contiguous oxygen atoms. An optionally substituted heteroorganic group is a heteroorganic group that is optionally substituted as described herein for alkyl.

The term “hydrocarbon,” as used herein, generally refers to an acyclic, branched or acyclic, linear compound or group, or a monocyclic, bicyclic, tricyclic, or tetracyclic compound or group. The hydrocarbon, when unsubstituted, consists of carbon and hydrogen atoms. Unless specified otherwise, an unsubstituted hydrocarbon includes a total of 1 to 60 carbon atoms (e.g., 1 to 16, 1 to 12, or 1 to 6 carbon atoms). An optionally substituted hydrocarbon is an optionally substituted acyclic hydrocarbon or an optionally substituted cyclic hydrocarbon. An optionally substituted acyclic hydrocarbon is optionally substituted as described herein for alkyl. An optionally substituted cyclic hydrocarbon is an optionally substituted aromatic hydrocarbon or an optionally substituted non-aromatic hydrocarbon. An optionally substituted aromatic hydrocarbon is optionally substituted as described herein for aryl. An optionally substituted non-aromatic cyclic hydrocarbon is optionally substituted as described herein for cycloalkyl. In some embodiments, an acyclic hydrocarbon is alkyl, alkylene, alkane-triyl, or alkane-tetrayl. In certain embodiments, a cyclic hydrocarbon is aryl or arylene. In particular embodiments, a cyclic hydrocarbon is cycloalkyl or cycloalkylene.

The terms “hydroxyl” and “hydroxy,” as used interchangeably herein, generally represent —OH.

The term “hydrophobic moiety,” as used herein, generally represents a monovalent group covalently linked to an oligonucleotide backbone, where the monovalent group is a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen. Non-limiting examples of the monovalent group include ergosterol, stigmasterol, β-sitosterol, campesterol, fucosterol, saringosterol, avenasterol, coprostanol, cholesterol, vitamin A, vitamin D, vitamin E, cardiolipin, and carotenoids. The linker connecting the monovalent group to the oligonucleotide may be an optionally substituted C₁₋₆₀ hydrocarbon (e.g., optionally substituted C₁₋₆₀ alkylene) or an optionally substituted C₂₋₆₀ heteroorganic (e.g., optionally substituted C₂₋₆₀ heteroalkylene), where the linker may be optionally interrupted with one, two, or three instances independently selected from the group consisting of an optionally substituted arylene, optionally substituted heterocyclylene, and optionally substituted cycloalkylene. The linker may be bonded to an oligonucleotide through, e.g., an oxygen atom attached to a 5′-terminal carbon atom, a 3′-terminal carbon atom, a 5′-terminal phosphate or phosphorothioate, a 3′-terminal phosphate or phosphorothioate, or an internucleoside linkage.

The term “internucleoside linkage,” as used herein, generally represents a divalent group or covalent bond that forms a covalent linkage between adjacent nucleosides in an oligonucleotide. An internucleoside linkage is an unmodified internucleoside linkage or a modified internucleoside linkage. An “unmodified internucleoside linkage” is a phosphate (—O—P(O)(OH)—O—) internucleoside linkage (“phosphate phosphodiester”). A “modified internucleoside linkage” is an internucleoside linkage other than a phosphate phosphodiester. The two main classes of modified internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, phosphorodithioate linkages, boranophosphonate linkages, morpholino internucleoside linkages, methylphosphonates, and phosphoramidate. Non-limiting examples of non-phosphorus internucleoside linkages include methylenemethylimino (—CH₂—N(CH₃)—O—CH₂—), thiodiester (—O—C(O)—S—), thionocarbamate (—O—C(O)(NH)—S—), siloxane (—O—Si(H)₂—O—), and N,N′-dimethylhydrazine (—CH₂—N(CH₃)—N(CH₃)—). Phosphorothioate linkages are phosphodiester linkages and phosphotriester linkages in which one of the non-bridging oxygen atoms is replaced with a sulfur atom. In some embodiments, an internucleoside linkage is a group of the following structure:

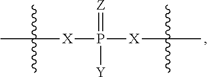

where

### Z is O, S, B, or Se;

### Y is —X-L-R1;

each X is independently —O—, —S—, —N(-L-R1)-, or L;
each L is independently a covalent bond or a linker (e.g., optionally substituted C₁₋₆₀ hydrocarbon linker or optionally substituted C₂₋₆₀ heteroorganic linker);
each R1 is independently hydrogen, —S—S—R2, —O—CO—R2, —S—CO—R2, optionally substituted C₁₋₉ heterocyclyl, a hydrophobic moiety, or a targeting moiety; and
each R2 is independently optionally substituted C₁₋₁₀ alkyl, optionally substituted C₂₋₁₀ heteroalkyl, optionally substituted C₆₋₁₀ aryl, optionally substituted C₆₋₁₀ aryl C₁₋₆ alkyl, optionally substituted C₁₋₉ heterocyclyl, or optionally substituted C₁₋₉ heterocyclyl C₁₋₆ alkyl. When L is a covalent bond, R1 is hydrogen, Z is oxygen, and all X groups are —O—, the internucleoside group is known as a phosphate phosphodiester. When L is a covalent bond, R1 is hydrogen, Z is sulfur, and all X groups are —O—, the internucleoside group is known as a phosphorothioate diester. When Z is oxygen, all X groups are —O—, and either (1) L is a linker or (2) R1 is not a hydrogen, the internucleoside group is known as a phosphotriester. When Z is sulfur, all X groups are —O—, and either (1) L is a linker or (2) R1 is not a hydrogen, the internucleoside group is known as a phosphorothioate triester. Non-limiting examples of phosphorothioate triester linkages and phosphotriester linkages are described in US 2017/0037399, the disclosure of which is incorporated herein by reference.

The term “intron 36,” as used herein, generally refers to intron 36 of ABCA4 pre-mRNA or genomic DNA, which corresponds to positions 106569 to 110295 in SEQ ID NO: 1 (hg19/b37 coordinates chr1:94481411-94485137), or a mutant version thereof (e.g., g.34393G>A in SEQ ID NO: 1).

The term “morpholino,” as used herein in reference to a class of oligonucleotides, generally represents an oligomer of at least 10 morpholino monomer units interconnected by morpholino internucleoside linkages. A morpholino includes a 5′ group and a 3′ group. For example, a morpholino may be of the following structure:

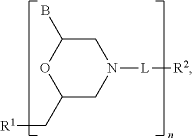

where
n is an integer of at least 10 (e.g., 12 to 50) indicating the number of morpholino units; each B is independently a nucleobase;
R¹ is a 5′ group;
R2 is a 3′ group; and
L is (i) a morpholino internucleoside linkage or, (ii) if L is attached to R², a covalent bond. A 5′ group in morpholino may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer. A 3′ group in morpholino may be, e.g., hydrogen, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer.

The term “morpholino internucleoside linkage,” as used herein, generally represents a divalent group of the following structure:

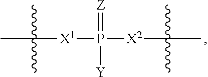

where

### Z is O or S;

X¹ is a bond, —CH₂—, or —O—;
X² is a bond, —CH₂—O—, or —O—; and
Y is —NR₂, where each R is independently C₁₋₆ alkyl (e.g., methyl), or both R combine together with the nitrogen atom to which they are attached to form a C₂₋₉ heterocyclyl (e.g., N-piperazinyl);
provided that both X¹ and X² are not simultaneously a bond.

The term “nucleobase,” as used herein, generally represents a nitrogen-containing heterocyclic ring found at the 1′ position of the ribofuranose/2′-deoxyribofuranose of a nucleoside. Nucleobases are unmodified or modified. As used herein, “unmodified” or “natural” nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and 0-6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e.g., 5-substituted pyrimidines; 6-azapyrimidines; N2-, N6-, and/or O6-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example, 7-deazaadenine, 7-deazaguanine, 2-aminopyridine, or 2-pyridone. Further nucleobases include those disclosed in U.S. Pat. No. 3,687,808; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and in Chapters 6 and 15, Antisense Drug Technology, Crooke S. T., Ed., CRC Press, 2008, 163-166 and 442-443.

The term “nucleoside,” as used herein, generally represents sugar-nucleobase compounds and groups known in the art (e.g., modified or unmodified ribofuranose-nucleobase and 2′-deoxyribofuranose-nucleobase compounds and groups known in the art). The sugar may be ribofuranose. The sugar may be modified or unmodified. An unmodified sugar nucleoside is ribofuranose or 2′-deoxyribofuranose having an anomeric carbon bonded to a nucleobase. An unmodified nucleoside is ribofuranose or 2′-deoxyribofuranose having an anomeric carbon bonded to an unmodified nucleobase. Non-limiting examples of unmodified nucleosides include adenosine, cytidine, guanosine, uridine, 2′-deoxyadenosine, 2′-deoxycytidine, 2′-deoxyguanosine, and thymidine. The modified compounds and groups include one or more modifications selected from the group consisting of nucleobase modifications and sugar modifications described herein. A nucleobase modification is a replacement of an unmodified nucleobase with a modified nucleobase. A sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. A locking modification is an incorporation of a bridge between 4′-carbon atom and 2′-carbon atom of ribofuranose. Nucleosides having a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides.

The term “nucleotide,” as used herein, generally represents a nucleoside bonded to an internucleoside linkage or a monovalent group of the following structure —X¹—P(X²)(R¹)₂, where X¹ is O, S, or NH, and X² is absent, ═O, or ═S, and each R¹ is independently —OH, —N(R²)₂, or —O—CH₂CH₂CN, where each R² is independently an optionally substituted alkyl, or both R² groups, together with the nitrogen atom to which they are attached, combine to form an optionally substituted heterocyclyl.

The term “oligonucleotide,” as used herein, generally represents a structure containing 10 or more (e.g., 10 to 50) contiguous nucleosides covalently bound together by internucleoside linkages. An oligonucleotide includes a 5′ end and a 3′ end. The 5′ end of an oligonucleotide may be, e.g., hydroxyl, a targeting moiety, a hydrophobic moiety, 5′ cap, phosphate, diphosphate, triphosphate, phosphorothioate, diphosphorothioate, triphosphorothioate, phosphorodithioate, diphosphrodithioate, triphosphorodithioate, phosphonate, phosphoramidate, a cell penetrating peptide, an endosomal escape moiety, or a neutral organic polymer. The 3′ end of an oligonucleotide may be, e.g., hydroxyl, a targeting moiety, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer (e.g., polyethylene glycol). An oligonucleotide having a 5′-hydroxyl or 5′-phosphate has an unmodified 5′ terminus. An oligonucleotide having a 5′ terminus other than 5′-hydroxyl or 5′-phosphate has a modified 5′ terminus. An oligonucleotide having a 3′-hydroxyl or 3′-phosphate has an unmodified 3′ terminus. An oligonucleotide having a 3′ terminus other than 3′-hydroxyl or 3′-phosphate has a modified 3′ terminus.

The term “oxo,” as used herein, generally represents a divalent oxygen atom (e.g., the structure of oxo may be shown as ═O).

The term “pharmaceutically acceptable,” as used herein, generally refers to those compounds, materials, compositions, and/or dosage forms, which are suitable for contact with the tissues of an individual (e.g., a human), without excessive toxicity, irritation, allergic response and other problem complications commensurate with a reasonable benefit/risk ratio.

The term “protecting group,” as used herein, generally represents a group intended to protect a functional group (e.g., a hydroxyl, an amino, or a carbonyl) from participating in one or more undesirable reactions during chemical synthesis. The term “O-protecting group,” as used herein, represents a group intended to protect an oxygen containing (e.g., phenol, hydroxyl or carbonyl) group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Wuts, “Greene's Protective Groups in Organic Synthesis,” 4^{th} Edition (John Wiley & Sons, New York, 2006), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and arylalkyl ethers (e.g., trityl; methylthiomethyl; methoxymethyl; benzyloxymethyl; siloxymethyl; 2,2,2-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydroxy carbonyl, t-butyloxycarbonyl, diisopropylmethoxycarbonyl, isopropoxycarbonyl, ethoxycarbonyl, methoxycarbonyl, allyloxycarbonyl, 2,2,2-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, arylalkyl groups such as benzyl, triphenylmethyl, benzyloxymethyl, and the like and silyl groups such as trimethylsilyl, and the like.

The term “pyrid-2-yl hydrazone,” as used herein, generally represents a group of the structure:

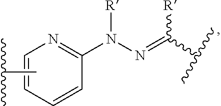

where each R′ is independently H or optionally substituted C₁₋₆ alkyl. Pyrid-2-yl hydrazone may be unsubstituted (i.e., each R′ is H).

The term “splice site,” as used herein, generally refers to a site in a genome corresponding to an end of an intron that may be involved in a splicing procedure. A splice site may be a 5′ splice site (e.g., a 5′ end of an intron) or a 3′ splice site (e.g., a 3′ end of an intron). A given 5′ splice site may be associated with one or more candidate 3′ splice sites, each of which may be coupled to its corresponding 5′ splice site in a splicing operation.

The term “splicing enhancer,” as used herein, generally refers to motifs with positive effects (e.g., causing an increase) on exon or intron inclusion.

The term “splicing regulatory element,” as used herein, generally refers to an exonic splicing silencer element, an exonic splicing enhancer element, an intronic splicing silencer element, and an intronic splicing enhancer element. An exonic splicing silencer element is a portion of the target pre-mRNA exon that reduces the ratio of transcripts including this exon relative to the total number of the gene transcripts. An intronic splicing silencer element is a portion of the target pre-mRNA intron that reduces the ratio of transcripts including the exon adjacent to the target intron relative to the total number of the gene transcripts. An exonic splicing enhancer element is a portion of the target pre-mRNA exon that increases the ratio of transcripts including this exon relative to the total number of the gene transcripts. An intronic splicing enhancer element is a portion of the target pre-mRNA intron that increases the ratio of transcripts including the exon adjacent to the target intron relative to the total number of the gene transcripts.

The term “splicing silencer,” as used herein, generally refers to motifs with negative effects (e.g., causing a decrease) on exon inclusion.

The term “stereochemically enriched,” as used herein, generally refers to a local stereochemical preference for one enantiomer of the recited group over the opposite enantiomer of the same group. Thus, an oligonucleotide containing a stereochemically enriched internucleoside linkage is an oligonucleotide in which a stereogenic internucleoside linkage (e.g., phosphorothioate) of predetermined stereochemistry is present in preference to a stereogenic internucleoside linkage (e.g., phosphorothioate) of stereochemistry that is opposite of the predetermined stereochemistry. This preference can be expressed numerically using a diastereomeric ratio for the stereogenic internucleoside linkage (e.g., phosphorothioate) of the predetermined stereochemistry. The diastereomeric ratio for the stereogenic internucleoside linkage (e.g., phosphorothioate) of the predetermined stereochemistry is the molar ratio of the diastereomers having the identified stereogenic internucleoside linkage (e.g., phosphorothioate) with the predetermined stereochemistry relative to the diastereomers having the identified stereogenic internucleoside linkage (e.g., phosphorothioate) with the stereochemistry that is opposite of the predetermined stereochemistry. The diastereomeric ratio for the phosphorothioate of the predetermined stereochemistry may be greater than or equal to 1.1 (e.g., greater than or equal to 4, greater than or equal to 9, greater than or equal to 19, or greater than or equal to 39).

The term “subject,” as used herein, generally represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject. A non-limiting example of a disease, disorder, or condition includes retinitis pigmentosa (RP), cone-rod dystrophy (CRD), and Stargardt disease (STGD1) (e.g., retinitis pigmentosa, cone-rod dystrophy, and Stargardt disease associated with skipping an exon in the ABCA4 gene (e.g., exon 33 or 40), the inclusion of a portion of a flanking intron adjacent to an exon in the ABCA4 gene (e.g., a portion of a flanking intron adjacent to exon 6), or the inclusion of a pseudo exon (e.g. a pseudo exon exon located in intro 36).

A “sugar” or “sugar moiety,” includes naturally occurring sugars having a furanose ring or a structure that is capable of replacing the furanose ring of a nucleoside. Sugars included in the nucleosides of the disclosure may be non-furanose (or 4′-substituted furanose) rings or ring systems or open systems. Such structures include simple changes relative to the natural furanose ring (e.g., a six-membered ring). Alternative sugars may also include sugar surrogates wherein the furanose ring has been replaced with another ring system such as, e.g., a morpholino or hexitol ring system. Non-limiting examples of sugar moieties useful that may be included in the oligonucleotides of the disclosure include β-D-ribose, β-D-2′-deoxyribose, substituted sugars (e.g., 2′, 5′, and bis substituted sugars), 4′-S-sugars (e.g., 4′-S-ribose, 4′-S-2′-deoxyribose, and 4′-S-2′-substituted ribose), bicyclic sugar moieties (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

The term “targeting moiety,” as used herein, generally represents a moiety (e.g., N-acetylgalactosamine or a cluster thereof) that specifically binds or reactively associates or complexes with a receptor or other receptive moiety associated with a given target cell population. An antisense oligonucleotide may contain a targeting moiety. An antisense oligonucleotide including a targeting moiety is also referred to herein as a conjugate. A targeting moiety may include one or more ligands (e.g., 1 to 6 ligands, 1 to 3 ligands, or 1 ligand). The ligand can be an antibody or an antigen-binding fragment or an engineered derivative thereof (e.g., Fcab or a fusion protein (e.g., scFv)). Alternatively, the ligand may be a small molecule (e.g., N-acetylgalactosamine).

The term “therapeutically effective amount,” as used herein, generally represents the quantity of an antisense oligonucleotide of the disclosure necessary to ameliorate, treat, or at least partially arrest the symptoms of a disease or disorder (e.g., to increase the level of ABCA4 mRNA molecules including the otherwise skipped exon (e.g., exon 33 or 40) or to increase the level of ABCA4 mRNA molecules excluding otherwise included intronic mRNA (e.g. flanking intronic sequence of exon 6 or a pseudo exon located within intron 36). Amounts effective for this use may depend, e.g., on the severity of the disease and the weight and general state of the subject. Typically, dosages used in vitro may provide useful guidance in the amounts useful for in vivo administration of the pharmaceutical composition, and animal models may be used to determine effective dosages for treatment of particular disorders. In some embodiments, a therapeutically effective amount of an antisense oligonucleotide of the disclosure reduces the plasma triglycerides level, e.g., at least 5%, at least 10%, at least 15%, at least 20%, at least 25%, at least 30%, at least 35%, at least 40%, at least 45%, or at least 50%; e.g., up to 80%, up to 70%, up to 60%, up to 50%, up to 40%, up to 30%, or up to 20%, as compared to the plasma triglycerides level prior to the administration of an antisense oligonucleotide. In some embodiments, a therapeutically effective amount of an antisense oligonucleotide of the disclosure reduces or maintains the plasma triglyceride levels in the subject to 300 mg/dL or less, 250 mg/dL or less, 200 mg/dL or less, or to 150 mg/dL or less. In some embodiments, a therapeutically effective amount of an antisense oligonucleotide of the disclosure reduces the plasma low density lipoprotein (LDL-C) level, e.g., at least 5%, at least 10%, at least 15%, at least 20%, at least 25%, at least 30%, at least 35%, at least 40%, at least 45%, or at least 50%; e.g., up to 80%, up to 70%, up to 60%, up to 50%, up to 40%, up to 30%, or up to 20%, as compared to the LDL-C level prior to the administration of an antisense oligonucleotide. In some embodiments, a therapeutically effective amount of an antisense oligonucleotide of the disclosure reduces or maintains the plasma LDL-C levels in the subject to less than 300 mg/dL, less than 250 mg/dL, less than 200 mg/dL, less than 190 mg/dL, less than 160 mg/dL, less than 150 mg/dL, less than 130 mg/dL, or less than 100 mg/dL. Lipid levels can be assessed using plasma lipid analyses or tissue lipid analysis. In plasma lipid analysis, blood plasma can be collected, and total plasma free cholesterol levels can be measured using, for example colorimetric assays with a COD-PAP kit (Wako Chemicals), total plasma triglycerides can be measured using, for example, a Triglycerides/GB kit (Boehringer Mannheim), and/or total plasma cholesterol can be determined using a Cholesterol/HP kit (Boehringer Mannheim). In tissue lipid analysis, lipids can be extracted, for example, from liver, spleen, and/or small intestine samples (e.g., using the method in Folch et al. J Biol. Chem 226: 497-505 (1957)). Total tissue cholesterol concentrations can be measured, for example, using O-phthalaldehyde.

The term “thiocarbonyl,” as used herein, generally represents a C(═S) group. Non-limiting example of functional groups containing a “thiocarbonyl” includes thioesters, thioketones, thioaldehydes, thioanhydrides, thioacyl chlorides, thioamides, thiocarboxylic acids, and thiocarboxylates.

The term “thioheterocyclylene,” as used herein, generally represents a divalent group —S—R′—, where R′ is a heterocyclylene as defined herein.

The term “thiol,” as used herein, generally represents an —SH group.

The term “triazolocycloalkenylene,” as used herein, generally refers to the heterocyclylenes containing a 1,2,3-triazole ring fused to an 8-membered ring, all of the endocyclic atoms of which are carbon atoms, and bridgehead atoms are sp²-hybridized carbon atoms. Triazocycloalkenylenes can be optionally substituted in a manner described for heterocyclyl.

The term “triazoloheterocyclylene,” as used herein, generally refers to the heterocyclylenes containing a 1,2,3-triazole ring fused to an 8-membered ring containing at least one heteroatom. The bridgehead atoms in triazoloheterocyclylene are carbon atoms. Triazoloheterocyclylenes can be optionally substituted in a manner described for heterocyclyl.

Enumeration of positions within oligonucleotides and nucleic acids, as used herein and unless specified otherwise, starts with the 5′-terminal nucleoside as 1 and proceeds in the 3′-direction.

The compounds described herein, unless otherwise noted, encompass isotopically enriched compounds (e.g., deuterated compounds), tautomers, and all stereoisomers and conformers (e.g. enantiomers, diastereomers, E/Z isomers, atropisomers, etc.), as well as racemates thereof and mixtures of different proportions of enantiomers or diastereomers, or mixtures of any of the foregoing forms as well as salts (e.g., pharmaceutically acceptable salts).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIGS. 1A-1B shows the c.768G>T variant leads to exon 6 extension in ABCA4 c.768G>T mutant minigene. FIG. TA is a schematic of the ABCA4 c.768G>T mutant minigene. FIG. 1B shows RT-PCR analysis of HEK293T and ARPE19 cells transfected with ABCA4 wild-type and c.768G>T mutant minigenes. Exon 6 inclusion (337 bp) and extension (371 bp) fragments are indicated by solid arrowheads for both wildtype minigene (WT) and c.768G>T (Mut) variant minigenes. 50 bp DNA ladder is shown for size reference.

FIGS. 2A-2B shows the c.4773+3A>G variant leads to exon 33 skipping in ABCA4 c.4773+3A>G mutant minigene. FIG. 2A is a schematic of the ABCA4 c.4773+3A>G mutant minigene. FIG. 2B shows RT-PCR analysis of HEK293T and ARPE19 cells transfected with ABCA4 wild-type and c.4773+3A>G mutant minigenes. Exon 33 inclusion (169 bp) and exclusion (69 bp) fragments are indicated by solid arrowheads for both wildtype minigene (WT) and c.4773+3A>G (Mut) variant minigenes. 50 bp DNA ladder is shown for size reference.

FIGS. 3A-3B shows the c.5196+1137G>A variant leads to intron 36 pseudo exon (36.1) inclusion in ABCA4 c.5196+1137G>A mutant minigene. FIG. 3A is a schematic of the ABCA4 c.5196+1137G>A mutant minigene. FIG. 3B shows RT-PCR analysis of HEK293T and ARPE19 cells transfected with ABCA4 wild-type and c.5196+1137G>A mutant minigenes. Pseudo exon 36.1 inclusion (173 bp) and exclusion (103 bp) fragments are indicated by solid arrowheads for both wildtype minigene (WT) and c.5196+1137G>A (Mut) variant minigenes. 50 bp DNA ladder is shown for size reference.

FIGS. 4A-4B shows the c.5714+5G>A variant leads to exon 40 skipping in ABCA4 c.5714+5G>A mutant minigene. FIG. 4A is a schematic of the ABCA4 c.5714+5G>A mutant minigene. FIG. 4B shows RT-PCR analysis of HEK293T and ARPE19 cells transfected with ABCA4 wild-type and c.5714+5G>A mutant minigenes. Exon 40 inclusion (318 bp) and exclusion (188 bp) fragments are indicated by solid arrowheads for both wildtype minigene (WT) and c.5714+5G>A (Mut) variant minigenes. 50 bp DNA ladder is shown for size reference.

### DETAILED DESCRIPTION

In general, the present disclosure provides antisense oligonucleotides, compositions, and methods that target an ABCA4 exon (e.g., exon 6, 33, or 40) or a flanking intron (e.g. intron 36). Surprisingly, the inventors have found that altering ABCA4 gene splicing to promote inclusion of an otherwise skipped exon (e.g., exon 33, or 40) or the exclusion of otherwise included intronic RNA (e.g. intronic RNA in a flanking intron adjacent to exon 6 or intronic RNA associated with a pseudo exon in intron 36) in the transcript of splice variants may be used to treat retinitis pigmentosa, cone-rod dystrophy, or Stargardt disease, and antisense oligonucleotides may be used to alter splicing of the ABCA4 gene to include the otherwise skipped exon (e.g., exon 33, or 40) or the exclusion of otherwise included intronic RNA (e.g. intronic RNA in a flanking intron adjacent to exon 6 or intronic RNA associated with a pseudo exon in intron 36). The antisense oligonucleotides of the disclosure may modulate splicing of ABCA4 pre-mRNA to increase the level of ABCA4 mRNA molecules having the otherwise skipped exon (e.g., exon 33, or 40) or ABCA4 mRNA molecules excluding otherwise included intronic RNA (e.g. intronic RNA in a flanking intron adjacent to exon 6 or intronic RNA associated with a pseudo exon in intron 36). Accordingly, the antisense oligonucleotides may be used to treat retinitis pigmentosa, cone-rod dystrophy, or Stargardt disease in a subject in need of a treatment therefor. Typically, an antisense oligonucleotide includes a nucleobase sequence at least 70% (e.g., at least 80%, at least 90%, at least 95%, or 100%) complementary to a ABCA4 pre-mRNA sequence in a 5′-flanking intron, a 3′-flanking intron, a combination of an exon (e.g., exon 6, 33, 40) and a 5′-flanking or 3′-flanking intron (e.g., a 5′-flanking or 3′-flanking intron adjacent to exon 6, 33, 40), or an intron (e.g. intron 36).

Genetic variants may correspond to changes or modifications in transcription and/or splicing. RNA is initially transcribed from DNA as pre-mRNA, with protein-coding and 5′UTR/3′UTR exons separated by introns. Splicing generally refers to the molecular process, carried out by the spliceosome complexes that may remove introns and adjoins exons, producing a mature mRNA sequence, which is then scanned and translated to protein by the ribosome. The molecular reaction catalyzed by the spliceosome may comprise (i) nucleophilic attack of the branch site adenosine 2′OH onto the outmost base of the intronic donor dinucleotide, with consequent release of the outmost exonic donor base 3′OH; and (ii) nucleophilic attack of the exonic donor 3′OH onto the outmost exonic acceptor base, with consequent release of the intron lariat and the spliced exons.

Splicing sequence changes can include the following categories: (a) alteration of a splice site (denominated canonical splice site) or exon recognition sequence required for the proper composition of a gene product, and (b) activation and utilization of an incorrect splice site (denominated cryptic splice site), or incorrect recognition of intronic sequence as an exon (denominated pseudo exon). Both (a) and (b) may result in the improper composition of a gene product. The splice site recognition signal may be required for spliceosome assembly and can comprise the following structures: (i) highly conserved intronic dinucleotide (AG, GT) immediately adjacent to the exon-intron boundary, and (ii) consensus sequence surrounding the intronic dinucleotide (often delimited to 3 exonic and 6 intronic nucleotides for the donor site, 3 exonic and 20 intronic nucleotides for the acceptor site) and branch site (variable position on the intronic acceptor side), both with lower conservation and more sequence variety.

In addition to splice site recognition, the exon recognition signal may comprise a plethora of motifs recognized by splicing factors and other RNA binding proteins, some of which may be ubiquitously expressed and some of which may be tissue specific. These motifs may be distributed over the exon body and in the proximal intronic sequence. The term “splicing enhancer” refers to motifs with positive effects (e.g., causing an increase) on exon inclusion, and the term “splicing silencer” refers to motifs with negative effects (e.g., causing a decrease) on exon inclusion. The exon recognition signal may be particularly important for correct splicing in the presence of weak consensus sequence. When a variant weakens the splice site recognition, the exon can be skipped and/or a nearby cryptic splice site which is already fairly strong can be used. In the presence of short introns, full intron retention is also a possible outcome. In particular, alteration of the intronic dinucleotide often results in splicing alteration, whereas consensus sequence alteration may be, on average, less impactful and more context-dependent. When the exon recognition signal is weakened, exon skipping may be a more likely outcome, but cryptic splice site use is also possible, especially in the presence of a very weak consensus sequence. Variants can also strengthen a weak cryptic splice site in proximity of the canonical splice site, and significantly increase its usage resulting in improper splicing and incorrect gene product (with effects including amino acid insertion/deletion, frameshift, and stop-gain).

Antisense oligonucleotides can be used to modulate gene splicing (e.g., by targeting splicing regulatory elements of the gene).

Antisense oligonucleotides may comprise splice-switching oligonucleotides (SSOs), which may modulate splicing by steric blockage, preventing the spliceosome assembly or the binding of splicing factors and RNA binding proteins. Blocking binding of specific splicing factors or RNA binding proteins that have an inhibitory effect may be used to produce increased exon inclusion (e.g. exon 33, or 40 inclusion). Blocking binding of specific splicing factors or RNA binding proteins that enhance cryptic splice site utilization may be used to decrease intron inclusion (e.g., the inclusion intronic RNA in a flanking intron adjacent to exon 6 or intronic RNA associated with a pseudo exon in intron 36). Specific steric blocker antisense oligonucleotide chemistries may include the modified RNA chemistry with phosphorothioate backbone (PS) with a sugar modification (e.g., 2′-modification) and phosphorodiamidate morpholino (PMO). Exemplary PS backbone sugar modifications may include 2′-O-methyl (2′OMe) and 2′-O-methoxyethyl (2′-MOE), which is also known as 2′-methoxyethoxy. Other nucleotide modifications may be used, for example, for the full length of the oligonucleotide or for specific bases. The oligonucleotides can be covalently conjugated to a targeting moiety (e.g., a GalNAc cluster), or to a peptide (e.g., a cell penetrating peptide), or to another molecular or multimolecular group (e.g., a hydrophobic moiety or neutral polymer) different from the rest of the oligonucleotide. Antisense oligonucleotides may be used as a single stereoisomer or a combination of stereoisomers.

The ABCA4 gene (ATP binding cassette subfamily A member 4; entrez gene 24) may play an important role in the pathogenicity of retinitis pigmentosa, cone-rod dystrophy, and Stargardt disease. ABCA4 is a transmembrane lipid transporter expressed in the photoreceptor outer segment, within the disc membranes. It is required to clear the reactive all-trans retinal from the photoreceptor disc lumen. Lack of ABCA4 function causes N-retinylidene-PE accumulation, which leads to formation of di-retinoid-pyridinium-PE (A2PE); all-trans retinal can also accumulate and form dimers. Since RPE cells recycle photoreceptor outer segments every 10 days, these compounds end up accumulating in their lysosomes. There, A2PE is hydrolyzed to di-retinoid-pyridinium-ethanolamine (A2E), which can be photoactivated and form highly reactive epoxides. This process is toxic for RPE cells and can lead to cell death. As photoreceptors lose the support of RPE, they can in turn suffer cell death. Higher levels of A2PE accumulation are directly toxic to photoreceptors, and cones are more sensitive than rods.

Recognizing a need for effective splicing modulation therapies for diseases such as retinitis pigmentosa, cone-rod dystrophy, or Stargardt disease, the present disclosure provides ABCA4 splice-modulating antisense oligonucleotides comprising sequences targeted to an intron adjacent to an abnormally spliced exon (e.g., exon 6, 33, or 40) of ABCA4 or an abnormally spliced intron (e.g. intron 36). In some embodiments, the antisense oligonucleotide has a sequence targeted to one or more splicing regulatory elements which may be located in an intron adjacent to an abnormally spliced exon (e.g., exon 6, 33, or 40) of ABCA4 or alternatively splicing regulatory elements which may be located in an intron next to a pseudo exon (e.g. intron 36). The present disclosure also provides methods for modulating splicing of ABCA4 RNA in a cell, tissue, or organ of a subject by bringing the cell, tissue, or organ in contact with an antisense oligonucleotide of the disclosure. An ABCA4 splice-modulating antisense oligonucleotide may comprise a nucleobase sequence targeted to a splicing regulatory element of an intron adjacent to an abnormally spliced exon (e.g., exon 6, 33, or 40) of ABCA4 or alternatively splicing regulatory elements which may be located in an intron next to a pseudo exon (e.g. intron 36). In addition, the present disclosure provides a method for treating retinitis pigmentosa, cone-rod dystrophy, or Stargardt disease in a subject by administering to the subject a therapeutically effective amount of an oligonucleotide of the disclosure. An ABCA4 splice-modulating antisense oligonucleotide may comprise a sequence targeted to a splicing regulatory element of or an intron adjacent to an abnormally spliced exon (e.g., exon 6, 33, or 40) of ABCA4 or alternatively splicing regulatory elements which may be located in an intron next to a pseudo exon (e.g. intron 36).

Splicing regulatory elements may include, for example, exonic splicing silencer elements or intronic splicing silencer elements. The antisense oligonucleotides may comprise sequences targeted to an intron adjacent to the exon (e.g., 33, or 40) of ABCA4 which modulates variant splicing of ABCA4 RNA. The modulation of splicing may result in an increase in exon inclusion (e.g. exon 33, or 40 inclusion). Antisense oligonucleotides may comprise a total of 8 to 50 nucleotides (e.g. 8 to 16 nucleotides, 8 to 20 nucleotides, 12 to 20 nucleotides, 12 to 30 nucleotides, or 12 to 50 nucleotides).

Additional splicing regulatory elements may include, for example, cryptic splice sites which are intronic mRNA sequences that have the potential to interact with the spliceosome. Cryptic splice sites may be activated by a variant and lead to the inclusion of a pseudo exon in the fully processed mRNA (e.g. the inclusion of a pseudo exon located in intron 36) or the elongation of an exon to include flanking intronic sequence in the fully processed (e.g. the inclusion of flanking intronic sequence in exon 6). The antisense oligonucleotides may comprise sequences targeted to an intron containing a pseudo exon (e.g. intron 36), or an exon or an intron adjacent to the exon which is mispliced (e.g. exon 6) of ABCA4 which modulates variant splicing of ABCA4 RNA. The modulation of splicing may result in a decrease in intronic sequence inclusion (e.g., partial intron 36 or 6 inclusion). Antisense oligonucleotides may comprise a total of 8 to 50 nucleotides (e.g., 8 to 16 nucleotides, 8 to 20 nucleotides, 12 to 20 nucleotides, 12 to 30 nucleotides, or 12 to 50 nucleotides).

Genetic aberrations of the ABCA4 gene may play an important role in pathogenicity. In particular, ABCA4 chr1:94484001:C:T [hg19/b37], chr1:94487399:T:C [hg19/b37], chr1:94476351:C:T [hg19/b37], and chr1:94564350:C:A [hg19/b37] genetic aberrations (g.107705G>A, g.104307A>G, g.115355G>A, g.27356G>T mutants of SEQ ID NO: 1, respectively), may result in NM_000350.2 (ABCA4) mRNA changes c.5196+1137G>A, c.4773+3A>G, c.5714+5G>A, and cDNA change c.768G>T respectively. Intronic variants c.5196+1137G>A, c.4773+3A>G, c.5714+5G>A are non-coding and c.768G>T results in no change in the protein sequence at amino acid position 256 (Val) in exon 6. Genome coordinates may be expressed, for example, with respect to human genome reference hg19/b37. For example, these variants have been reported as pathogenic in patients with retinitis pigmentosa, cone-rod dystrophy, or Stargardt disease. Exemplary variants which have been reported or predicted to be pathogenic in patients with retinitis pigmentosa, cone-rod dystrophy, or Stargardt disease variants are listed in Table 1.

TABLE 1

| | Genomic_ | mRNA coordinate |
|---|---|---|
| Genomic_coordinate | coordinate | (protein sequence |
| [hg19/b37] | [SEQ ID NO: 1] | change) [NM_000350.2] |
| chr1:94466425:C:A | g.125281G > T | c.6446G > T (p.Arg2149Leu) |
| chr1:94466602:C:T | g.125104G > A | c.6342G > A (p.Val2114=) |
| chr1:94526295:C:T | g.65411G > A | c.1958G > A (p.Arg653His) |
| chr1:94528683:T:C | g.63023A > G | c.1745A > G (p. Asn582Ser) |
| chr1:94476378:G:A | g.115328C > T | c.5692C > T (p.Arg1898Cys) |
| chr1:94480241:G:A | g.111465C > T | c.5318C > T (p.Ala1773Val) |
| chr1:94487443:C:T | g.104263G > A | c.4732G > A (p.Gly1578Arg) |
| chr1:94496008:C:T | g.95698G > A | c.4328G > A (p.Arg1443His) |
| chr1:94496610:C:T | g.95096G > A | c.4195G > A (p.Glu1399Lys) |
| chr1:94528819:G:A | g.62887C > T | c.1609C > T (p.Arg537Cys) |
| chr1:94473791:C:T | g.117915G > A | c.5898G > A (p.Glu1966=) |
| chr1:94476351:C:T | g.115355G > A | c.5714 + 5G > A |
| chr1:94487269:C:T | g.104437G > A | c.4775G > A (p.Gly1592Asp) |
| chr1:94487399:T:C | g.104307A > G | c.4773 + 3A > G |
| chr1:94496547:C:T | g.95159G > A | c.4253 + 5G > A |
| chr1:94496548:G:A | g.95158C > T | c.4253 + 4C > T |
| chr1:94510164:C:T | g.81542G > A | c.3050 + 5G > A |
| chr1:94543248:C:T | g.48458G > A | c.1552G > A (p.Glu518Lys) |
| chr1:94564350:C:A | g.27356G > T | c.768G > T (p.Val256=) |
| chr1:94586533:T:G | g.5173A > C | c.66 + 3A > C |
| chr1:94484001:C:T | g.107705G > A | c.5196 + 1137G > A |
| chr1:94566773:T:C | g.24933A > G | c.570 + 1798A > G |

These exemplary genetic aberrations may be targeted with antisense oligonucleotides to increase levels of exon inclusion (e.g., exon 33, or 40 inclusion) or decrease intronic sequence inclusion (e.g., partial intron 36 or 6 inclusion) of ABCA4.

Different antisense oligonucleotides can be combined for increasing an exon inclusion (e.g., exon 33, or 40 inclusion), or decreasing intronic sequence inclusion (e.g., partial intron 36 or 6 inclusion) of ABCA4. A combination of two antisense oligonucleotides may be used in a method of the disclosure, such as two antisense oligonucleotides, three antisense oligonucleotides, four different antisense oligonucleotides, or five different antisense oligonucleotides targeting the same or different regions or “hotspots.”

An antisense oligonucleotide according to the disclosure may be indirectly administered using suitable techniques and methods known in the art. It may for example be provided to an individual or a cell, tissue or organ of the individual in the form of an expression vector wherein the expression vector encodes a transcript comprising said oligonucleotide. The expression vector is preferably introduced into a cell, tissue, organ or individual via a gene delivery vehicle. In an embodiment, there is provided a viral based expression vector comprising an expression cassette or a transcription cassette that drives expression or transcription of an antisense oligonucleotide as identified herein. Accordingly, the present disclosure provides a viral vector expressing an antisense oligonucleotide according to the disclosure.

An antisense oligonucleotide according to the disclosure may be directly administered using suitable techniques and methods known in the art, e.g., using conjugates described herein.

### Conjugates

Oligonucleotides of the disclosure may include an auxiliary moiety, e.g., a targeting moiety, hydrophobic moiety, cell penetrating peptide, or a polymer. An auxiliary moiety may be present as a 5′ terminal modification (e.g., covalently bonded to a 5′-terminal nucleoside), a 3′ terminal modification (e.g., covalently bonded to a 3′-terminal nucleoside), or an internucleoside linkage (e.g., covalently bonded to phosphate or phosphorothioate in an internucleoside linkage).

### Targeting Moieties

An oligonucleotide of the disclosure may include a targeting moiety.

A targeting moiety is selected based on its ability to target oligonucleotides of the disclosure to a desired or selected cell population that expresses the corresponding binding partner (e.g., either the corresponding receptor or ligand) for the selected targeting moiety. For example, an oligonucleotide of the disclosure could be targeted to hepatocytes expressing asialoglycoprotein receptor (ASGP-R) by selecting a targeting moiety containing N-acetylgalactosamine (GalNAc).

A targeting moiety may include one or more ligands (e.g., 1 to 9 ligands, 1 to 6 ligands, 1 to 3 ligands, 3 ligands, or 1 ligand). The ligand may target a cell expressing asialoglycoprotein receptor (ASGP-R), IgA receptor, HDL receptor, LDL receptor, or transferrin receptor. Non-limiting examples of the ligands include N-acetylgalactosamine, glycyrrhetinic acid, glycyrrhizin, lactobionic acid, lactoferrin, IgA, or a bile acid (e.g., lithocholyltaurine or taurocholic acid).

The ligand may be a small molecule, e.g., a small molecules targeting a cell expressing asialoglycoprotein receptor (ASGP-R). A non-limiting example of a small molecule targeting an asialoglycoprotein receptor is N-acetylgalactosamine. Alternatively, the ligand can be an antibody or an antigen-binding fragment or an engineered derivative thereof (e.g., Fcab or a fusion protein (e.g., scFv)).

A targeting moiety may be -LinkA(-T)ₚ, where LinkA is a multivalent linker, each T is a ligand (e.g., asialoglycoprotein receptor-targeting ligand (e.g., N-acetylgalactosamine)), and p is an integer from 1 to 9. When each T is N-acetylgalactosamine, the targeting moiety is referred to as a galactosamine cluster. Galactosamine clusters that may be used in oligonucleotides of the disclosure are known in the art. Non-limiting examples of the galactosamine clusters that may be included in the oligonucleotides of the disclosure are provided in U.S. Pat. Nos. 5,994,517; 7,491,805; 9,714,421; 9,867,882; 9,127,276; US 2018/0326070; US 2016/0257961; WO 2017/100461; and in Sliedregt et al., J. Med. Chem., 42:609-618, 1999. Ligands other than GalNAc may also be used in clusters, as described herein for galactosamine clusters.

Targeting moiety -LinkA(-T)ₚ may be a group of formula (I):

-Q¹-Q²([-Q³-Q⁴-Q⁵]ₛ-Q⁶-T)ₚ, (I)

where
each s is independently an integer from 0 to 20 (e.g., from 0 to 10), where the repeating units are the same or different;
Q¹ is a conjugation linker (e.g., [-Q³-Q⁴-Q⁵]ₛ-Q^{C}- where Q^{C} is optionally substituted C₂₋₁₂ heteroalkylene (e.g., a heteroalkylene containing —C(O)—N(H)—, —N(H)—C(O)—, —S(O)₂—N(H)—, —N(H)—S(O)₂—, or —S—S—), optionally substituted C₁₋₁₂ thioheterocyclylene

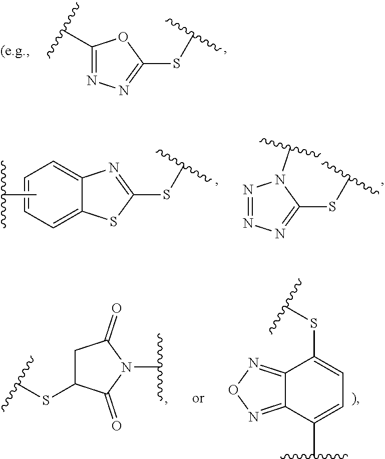

optionally substituted C₁₋₁₂ heterocyclylene (e.g., 1,2,3-triazole-1,4-diyl or

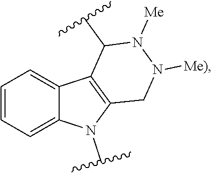

cyclobut-3-ene-1,2-dione-3,4-diyl, pyrid-2-yl hydrazone, optionally substituted C₆₋₁₆ triazoloheterocyclylene (e.g.,

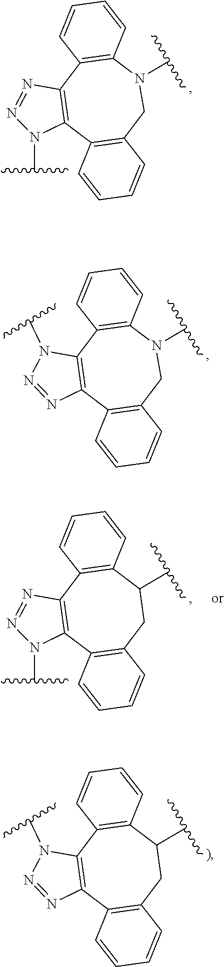

optionally substituted C₈₋₁₆ triazolocycloalkenylene

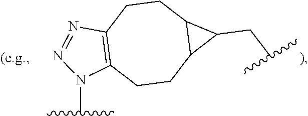 [SMILES: *CC1C2CCc3nnn(*)c3CCC12]

or a dihydropyridazine group (e.g., trans-

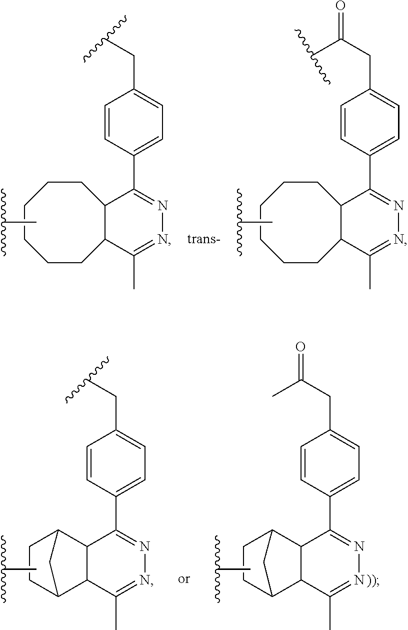

Q² is a linear group (e.g., [-Q³-Q⁴-Q⁵]ₛ-), if p is 1, or a branched group (e.g., [-Q³-Q⁴-Q⁵]ₛ-Q⁷([-Q³-Q⁴-Q⁵]ₛ-(Q⁷)ₚ₁)ₚ₂, where p1 is 0, 1, or 2, and p2 is 0, 1, 2, or 3), if p is an integer from 2 to 9;
each Q³ and each Q⁶ is independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—; each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆-10 arylene, optionally substituted C₁₋₉ heteroarylene, or optionally substituted C₁₋₉ heterocyclylene;
each Q⁵ is independently absent, —CO—, —NH—, —O—, —S—, —SO₂—, —CH₂—, —C(O)O—, —OC(O)—, —C(O)NH—, —NH—C(O)—, —NH—CH(R^{a})—C(O)—, —C(O)—CH(R^{a})—NH—, —OP(O)(OH)O—, or —OP(S)(OH)O—;
each Q⁷ is independently optionally substituted hydrocarbon or optionally substituted heteroorganic (e.g., C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl); and
each R^{a} is independently H or an amino acid side chain;
provided that at least one of Q³, Q⁴, and Q⁵ is present.

In some instances, for each occurrence of [-Q³-Q⁴-Q⁵]ₛ-, at least one of Q³, Q⁴, and Q⁵ is present.

In some instances, Q⁷ may be a structure selected from the group consisting of:

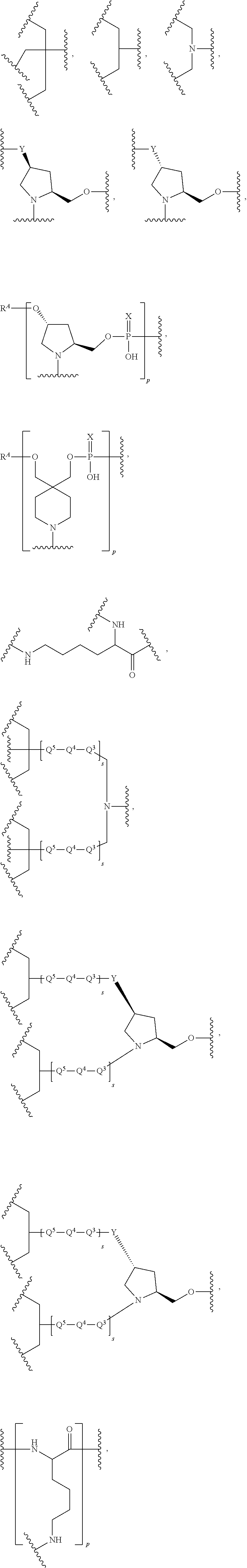

where R^{A} is H or oligonucleotide, X is O or S, Y is O or NH, and the remaining variables are as described for formula (I).

Group -LinkA- may include a poly(alkylene oxide) (e.g., polyethylene oxide, polypropylene oxide, poly(trimethylene oxide), polybutylene oxide, poly(tetramethylene oxide), and diblock or triblock co-polymers thereof). In some embodiments, -LinkA- includes polyethylene oxide (e.g., poly(ethylene oxide) having a molecular weight of less than 1 kDa).

### Hydrophobic Moieties

Advantageously, an oligonucleotide including a hydrophobic moiety may exhibit superior cellular uptake, as compared to an oligonucleotide lacking the hydrophobic moiety. Oligonucleotides including a hydrophobic moiety may therefore be used in compositions that are substantially free of transfecting agents. A hydrophobic moiety is a monovalent group (e.g., a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen) covalently linked to the oligonucleotide backbone (e.g., 5′-terminus). Non-limiting examples of the monovalent group include ergosterol, stigmasterol, β-sitosterol, campesterol, fucosterol, saringosterol, avenasterol, coprostanol, cholesterol, vitamin A, vitamin D, vitamin E, cardiolipin, and carotenoids. The linker connecting the monovalent group to the oligonucleotide may be an optionally substituted C₁₋₆₀ hydrocarbon (e.g., optionally substituted C₁₋₆₀ alkylene) or an optionally substituted C₂₋₆₀ heteroorganic (e.g., optionally substituted C₂₋₆₀ heteroalkylene), where the linker may be optionally interrupted with one, two, or three instances independently selected from the group consisting of an optionally substituted arylene, optionally substituted heterocyclylene, and optionally substituted cycloalkylene. The linker may be bonded to an oligonucleotide through, e.g., an oxygen atom attached to a 5′-terminal carbon atom, a 3′-terminal carbon atom, a 5′-terminal phosphate or phosphorothioate, a 3′-terminal phosphate or phosphorothioate, or an internucleoside linkage.

### Cell Penetrating Peptides

One or more cell penetrating peptides (e.g., from 1 to 6 or from 1 to 3) can be attached to an oligonucleotide disclosed herein as an auxiliary moiety. The CPP can be linked to the oligonucleotide through a disulfide linkage, as disclosed herein. Thus, upon delivery to a cell, the CPP can be cleaved intracellularly, e.g., by an intracellular enzyme (e.g., protein disulfide isomerase, thioredoxin, or a thioesterase) and thereby release the polynucleotide.

CPPs are known in the art (e.g., TAT or Args (SEQ ID NO: 462)) (Snyder and Dowdy, 2005, Expert Opin. Drug Deliv. 2, 43-51). Specific examples of CPPs including moieties suitable for conjugation to the oligonucleotides disclosed herein are provided, e.g., in WO 2015/188197; the disclosure of these CPPs is incorporated by reference herein.

CPPs are positively charged peptides that are capable of facilitating the delivery of biological cargo to a cell. It is believed that the cationic charge of the CPPs is essential for their function. Moreover, the transduction of these proteins does not appear to be affected by cell type, and these proteins can efficiently transduce nearly all cells in culture with no apparent toxicity. In addition to full-length proteins, CPPs have also been used successfully to induce the intracellular uptake of DNA, antisense polynucleotides, small molecules, and even inorganic 40 nm iron particles suggesting that there is considerable flexibility in particle size in this process.

In one embodiment, a CPP useful in the methods and compositions of the disclosure includes a peptide featuring substantial alpha-helicity. It has been discovered that transfection is optimized when the CPP exhibits significant alpha-helicity. In another embodiment, the CPP includes a sequence containing basic amino acid residues that are substantially aligned along at least one face of the peptide. A CPP useful in the disclosure may be a naturally occurring peptide or a synthetic peptide.

### Polymers

An oligonucleotide of the disclosure may include covalently attached neutral polymer-based auxiliary moieties. Neutral polymers include poly(C₁₋₆ alkylene oxide), e.g., poly(ethylene glycol) and poly(propylene glycol) and copolymers thereof, e.g., di- and triblock copolymers. Other examples of polymers include esterified poly(acrylic acid), esterified poly(glutamic acid), esterified poly(aspartic acid), poly(vinyl alcohol), poly(ethylene-co-vinyl alcohol), poly(N-vinyl pyrrolidone), poly(ethyloxazoline), poly(alkylacrylates), poly(acrylamide), poly(N-alkylacrylamides), poly(N-acryloylmorpholine), poly(lactic acid), poly(glycolic acid), poly(dioxanone), poly(caprolactone), styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyurethane, N-isopropylacrylamide polymers, and poly(N,N-dialkylacrylamides). Exemplary polymer auxiliary moieties may have molecular weights of less than 100, 300, 500, 1000, or 5000 Da (e.g., greater than 100 Da). Other polymers are known in the art.

### Nucleobase Modifications

Oligonucleotides of the disclosure may include one or more modified nucleobases. Unmodified nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and 0-6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e.g., 5-substituted pyrimidines; 6-azapyrimidines; N2-, N6-, and/or O6-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deazaadenine, 7-deazaguanine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

The replacement of cytidine with 5-methylcytidine can reduce immunogenicity of oligonucleotides, e.g., those oligonucleotides having CpG units.

The replacement of one or more guanosines with, e.g., 7-deazaguanosine or 6-thioguanosine, may inhibit the antisense activity reducing G tetraplex formation within antisense oligonucleotides.

### Sugar Modifications

Oligonucleotides of the disclosure may include one or more sugar modifications in nucleosides. Nucleosides having an unmodified sugar include a sugar moiety that is a furanose ring as found in ribonucleosides and 2′-deoxyribonucleosides.

Sugars included in the nucleosides of the disclosure may be non-furanose (or 4′-substituted furanose) rings or ring systems or open systems. Such structures include simple changes relative to the natural furanose ring (e.g., a six-membered ring). Alternative sugars may also include sugar surrogates wherein the furanose ring has been replaced with another ring system such as, e.g., a morpholino or hexitol ring system. Non-limiting examples of sugar moieties useful that may be included in the oligonucleotides of the disclosure include β-D-ribose, β-D-2′-deoxyribose, substituted sugars (e.g., 2′, 5′, and bis substituted sugars), 4′-S-sugars (e.g., 4′-S-ribose, 4′-S-2′-deoxyribose, and 4′-S-2′-substituted ribose), bridged sugars (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

Typically, a sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. A locking modification is an incorporation of a bridge between 4′-carbon atom and 2′-carbon atom of ribofuranose. Nucleosides having a sugar with a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides.

### Internucleoside Linkage Modifications

Oligonucleotides of the disclosure may include one or more internucleoside linkage modifications. The two main classes of internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, morpholino internucleoside linkages, methylphosphonates, and phosphoramidate. Non-limiting examples of non-phosphorus internucleoside linkages include methylenemethylimino (—CH₂—N(CH₃)—O—CH₂—), thiodiester (—O—C(O)—S—), thionocarbamate (—O—C(O)(NH)—S—), siloxane (—O—Si(H)₂—O—), and N,N′-dimethylhydrazine (—CH2-N(CH₃)—N(CH₃)—). Modified linkages, compared to natural phosphodiester linkages, can be used to alter, typically increase, nuclease resistance of the oligonucleotide. Methods of preparation of phosphorous-containing and non-phosphorous-containing internucleoside linkages are known in the art.

Internucleoside linkages may be stereochemically enriched. For example, phosphorothioate-based internucleoside linkages (e.g., phosphorothioate diester or phosphorothioate triester) may be stereochemically enriched. The stereochemically enriched internucleoside linkages including a stereogenic phosphorus are typically designated S_{P} or R_{P} to identify the absolute stereochemistry of the phosphorus atom. Within an oligonucleotide, S_{P} phosphorothioate indicates the following structure:

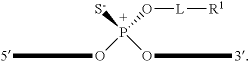

Within an oligonucleotide, R_{P} phosphorothioate indicates the following structure:

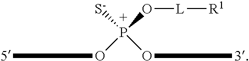

The oligonucleotides of the disclosure may include one or more neutral internucleoside linkages. Non-limiting examples of neutral internucleoside linkages include phosphotriesters, phosphorothioate triesters, methylphosphonates, methylenemethylimino (5′-CH₂—N(CH₃)—O-3′), amide-3 (5′-CH₂—C(═O)—N(H)-3′), amide-4 (5′-CH₂—N(H)—C(═O)-3′), formacetal (5′-O—CH₂—O-3′), and thioformacetal (5′-S—CH₂—O-3′). Further neutral internucleoside linkages include nonionic linkages including siloxane (dialkylsiloxane), carboxylate ester, carboxamide, sulfide, sulfonate ester, and amides (See for example: Carbohydrate Modifications in Antisense Research; Y. S. Sanghvi and P. D. Cook, Eds., ACS Symposium Series 580; Chapters 3 and 4, 40-65).

### Terminal Modifications

Oligonucleotides of the disclosure may include a terminal modification, e.g., a 5′-terminal modification or a 3′-terminal modification.

The 5′ end of an oligonucleotide may be, e.g., hydroxyl, a hydrophobic moiety, a targeting moiety, 5′ cap, phosphate, diphosphate, triphosphate, phosphorothioate, diphosphorothioate, triphosphorothioate, phosphorodithioate, diphosphrodithioate, triphosphorodithioate, phosphonate, phosphoramidate, a cell penetrating peptide, an endosomal escape moiety, or a neutral organic polymer. An unmodified 5′-terminus is hydroxyl or phosphate. An oligonucleotide having a 5′ terminus other than 5′-hydroxyl or 5′-phosphate has a modified 5′ terminus.

The 3′ end of an oligonucleotide may be, e.g., hydroxyl, a targeting moiety, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer (e.g., polyethylene glycol). An unmodified 3′-terminus is hydroxyl or phosphate. An oligonucleotide having a 3′ terminus other than 3′-hydroxyl or 3′-phosphate has a modified 3′ terminus.

The terminal modification (e.g., 5′-terminal modification) may be, e.g., a targeting moiety as described herein.

The terminal modification (e.g., 5′-terminal modification) may be, e.g., a hydrophobic moiety as described herein.

### Complementarity

In some embodiments, oligonucleotides of the disclosure are complementary to an ABCA4 target sequence over the entire length of the oligonucleotide. In other embodiments, oligonucleotides are at least 99%, 95%, 90%, 85%, 80%, or 70% complementary to the ABCA4 target sequence. In further embodiments, oligonucleotides are at least 80% (e.g., at least 90% or at least 95%) complementary to the ABCA4 target sequence over the entire length of the oligonucleotide and include a nucleobase sequence that is fully complementary to a ABCA4 target sequence. The nucleobase sequence that is fully complementary may be, e.g., 6 to 20, 10 to 18, or 18 to 20 contiguous nucleobases in length.

An oligonucleotide of the disclosure may include one or more (e.g., 1, 2, 3, or 4) mismatched nucleobases relative to the target nucleic acid. In certain embodiments, a splice-switching activity against the target is reduced by such mismatch, but activity against a non-target is reduced by a greater amount. Thus, the off-target selectivity of the oligonucleotides may be improved.

### Methods for Preparing Compositions

The present disclosure provides methods for preparing or generating compositions provided herein. A nucleic acid molecule, such as an oligonucleotide, comprising a targeted sequence may be generated, for example, by various nucleic acid synthesis approaches. For example, a nucleic acid molecule comprising a sequence targeted to a splice site may be generated by oligomerization of modified and/or unmodified nucleosides, thereby producing DNA or RNA oligonucleotides. Antisense oligonucleotides can be prepared, for example, by solid phase synthesis. Such solid phase synthesis can be performed, for example, in multi-well plates using equipment available from vendors such as Applied Biosystems (Foster City, Calif.). It is well known to use similar techniques to prepare oligonucleotides such as the phosphorothioates and alkylated derivatives. Oligonucleotides may be subjected to purification and/or analysis using methods known to those skilled in the art. For example, analysis methods may include capillary electrophoresis (CE) and electrospray-mass spectroscopy.

### Pharmaceutical Compositions

An oligonucleotide of the disclosure may be included in a pharmaceutical composition. A pharmaceutical composition typically includes a pharmaceutically acceptable diluent or carrier. A pharmaceutical composition may include (e.g., consist of), e.g., a sterile saline solution and an oligonucleotide of the disclosure. The sterile saline is typically a pharmaceutical grade saline. A pharmaceutical composition may include (e.g., consist of), e.g., sterile water and an oligonucleotide of the disclosure. The sterile water is typically a pharmaceutical grade water. A pharmaceutical composition may include (e.g., consist of), e.g., phosphate-buffered saline (PBS) and an oligonucleotide of the disclosure. The sterile PBS is typically a pharmaceutical grade PBS.

Pharmaceutical compositions may include one or more oligonucleotides and one or more excipients. Excipients may be selected from water, salt solutions, alcohol, polyethylene glycols, gelatin, lactose, amylase, magnesium stearate, talc, silicic acid, viscous paraffin, hydroxymethylcellulose and polyvinylpyrrolidone.

Pharmaceutical compositions including an oligonucleotide encompass any pharmaceutically acceptable salts of the oligonucleotide. Pharmaceutical compositions including an oligonucleotide, upon administration to a subject (e.g., a human), are capable of providing (directly or indirectly) the biologically active metabolite or residue thereof. Accordingly, for example, the disclosure is also drawn to pharmaceutically acceptable salts of oligonucleotides. Suitable pharmaceutically acceptable salts include, but are not limited to, sodium and potassium salts. In certain embodiments, prodrugs include one or more conjugate group(s) attached to an oligonucleotide, wherein the one or more conjugate group(s) is cleaved by endogenous enzymes within the body.

Lipid moieties have been used in nucleic acid therapies in a variety of methods. In certain such methods, the nucleic acid, such as an oligonucleotide, is introduced into preformed liposomes or lipoplexes made of mixtures of cationic lipids and neutral lipids. DNA complexes with mono- or poly-cationic lipids may form, e.g., without the presence of a neutral lipid. A lipid moiety may be, e.g., selected to increase distribution of a pharmaceutical agent to a particular cell or tissue. A lipid moiety may be, e.g., selected to increase distribution of a pharmaceutical agent to fat tissue. A lipid moiety may be, e.g., selected to increase distribution of a pharmaceutical agent to muscle tissue.

Pharmaceutical compositions may include a delivery system. Examples of delivery systems include, but are not limited to, liposomes and emulsions. Certain delivery systems are useful for preparing certain pharmaceutical compositions including those including hydrophobic compounds. Certain organic solvents such as dimethylsulfoxide may be used.

Pharmaceutical compositions may include one or more tissue-specific delivery molecules designed to deliver the one or more pharmaceutical agents of the present disclosure to specific tissues or cell types. For example, pharmaceutical compositions may include liposomes coated with a targeting moiety as described herein.

Pharmaceutical compositions may include a co-solvent system. Certain co-solvent systems include, e.g., benzyl alcohol, a nonpolar surfactant, a water-miscible organic polymer, and an aqueous phase. Such co-solvent systems may be used, e.g., for hydrophobic compounds. A non-limiting example of a co-solvent system is the VPD co-solvent system, which is a solution of absolute ethanol including 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

Pharmaceutical compositions may be prepared for administration by injection or infusion (e.g., intravenous, subcutaneous, intramuscular, intrathecal, intracerebroventricular, intravitreal etc.). A pharmaceutical composition may include, e.g., a carrier and may be formulated, e.g., in aqueous solution, e.g., water or physiologically compatible buffers, e.g., Hanks's solution, Ringer's solution, or physiological saline buffer. Other ingredients may also be included (e.g., ingredients that aid in solubility or serve as preservatives). Injectable suspensions may be prepared, e.g., using appropriate liquid carriers, suspending agents and the like. Certain pharmaceutical compositions for injection are presented in unit dosage form, e.g., in ampoules or in multi-dose containers. Certain pharmaceutical compositions for injection may be, e.g., suspensions, solutions, or emulsions in oily or aqueous vehicles, and may contain excipients (e.g., suspending, stabilizing and/or dispersing agents). Certain solvents suitable for use in pharmaceutical compositions for injection include, but are not limited to, lipophilic solvents and fatty oils, e.g., sesame oil, synthetic fatty acid esters (e.g., ethyl oleate or triglycerides), and liposomes.

### Methods of the Disclosure

The disclosure provides methods of using oligonucleotides of the disclosure.

A method of the disclosure may be a method of increasing the level of an exon-containing (e.g., exon 33 or 40-containing) ABCA4 mRNA molecules in a cell expressing an aberrant ABCA4 gene by contacting the cell with an antisense oligonucleotide of the disclosure.

A method of the disclosure may be a method of decreasing the level of an intron-containing (e.g., partial intron 6 or 36-containing) ABCA4 mRNA molecules in a cell expressing an aberrant ABCA4 gene by contacting the cell with an antisense oligonucleotide of the disclosure.

A method of the disclosure may be a method of treating retinitis pigmentosa, cone-rod dystrophy, or Stargardt disease in a subject having an aberrant ABCA4 gene by administering a therapeutically effective amount of an antisense oligonucleotide of the disclosure or a pharmaceutical composition of the disclosure to the subject in need thereof.

The oligonucleotide of the disclosure or the pharmaceutical composition of the disclosure may be administered to the subject using methods known in the art. For example, the oligonucleotide of the disclosure or the pharmaceutical composition of the disclosure may be administered parenterally (e.g., intravenously, intramuscularly, subcutaneously, transdermally, intranasally, intravitreally, or intrapulmonarily) to the subject.

Dosing is typically dependent on a variety of factors including, e.g., severity and responsiveness of the disease state to be treated. The treatment course may last, e.g., from several days to several years, or until a cure is effected or a diminution of the disease state is achieved. Optimal dosing schedules can be calculated from measurements of drug accumulation in the body of the patient. Thus, optimum dosages, dosing methodologies and repetition rates can be established as needed. Optimum dosages may vary depending on the relative potency of individual oligonucleotides, and can generally be estimated based on EC₅₀s found to be effective in in vitro and in vivo animal models. In general, dosage may be from 0.01 μg to 1 g per kg of body weight, and may be given once or more daily, weekly, monthly, bimonthly, trimonthly, every six months, annually, or biannually. Frequency of dosage may vary. Repetition rates for dosing may be established, for example, based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 1 g per kg of body weight, e.g., once daily, twice daily, three times daily, every other day, weekly, biweekly, monthly, bimonthly, trimonthly, every six months, annually or biannually.

### EXAMPLES

The following materials, methods, and examples are illustrative only and not intended to be limiting.

### Materials and Methods

In general, the practice of the present disclosure employs, unless otherwise indicated, conventional techniques of chemistry, molecular biology, and recombinant DNA technology. See, e.g., Sambrook, Fritsch and Maniatis, Molecular Cloning: Cold Spring Harbor Laboratory Press (1989) and Current Protocols in Molecular Biology, eds. Ausubel et al., John Wiley & Sons (1992).

Oligonucleotides. All antisense oligonucleotides used were obtained from Integrated DNA Technologies Inc. (USA). All bases in the antisense oligonucleotides were 2′-O-methoxyethyl-modified (MOE) with a full phosphorothioate backbone.

Cell culture. HEK293T cells were grown in Iscove's Modified Dulbecco's Medium (Gibco) supplemented with 10% (v/v) Cosmic Calf Serum (HyClone), 2 mM L-Glutamine (Gibco) and 1% antibiotics (100-U/ml penicillin G and 100-ug/ml streptomycin, Gibco) in a humidified incubator at 37° C. with 5% CO2. Upon reaching confluency the HEK293T cells were passaged by washing with Phosphate-Buffered Saline followed by Trypsin (Gibco) dissociation and plated in 10 to 20-fold dilution. ARPE19 cells were grown in Dulbecco's Modified Eagle Medium: Nutrient Mixture F-12 (DMEM/F-12; Gibco) with 10% (v/v) Fetal Bovine Serum (Gibco) and 1% antibiotics (100-U/ml penicillin G and 100-ug/ml streptomycin, Gibco). Upon reaching confluency the ARPE19 cells were passaged by washing with Phosphate-Buffered Saline followed by TrypLE (Gibco) dissociation and plated in a culture flask in 2 to 4-fold dilution.

Transfection of cells with minigene plasmids. HEK293T cells were seeded at 75000 cells per well in 24 well plates using Iscove's Modified Dulbecco's Medium (IMDM; Gibco) supplemented with 10% (v/v) Cosmic Calf Serum (HyClone) and 2 mM L-glutamine (Gibco) and incubated at 37° C. and 5% CO2 overnight. ARPE19 cells were seeded at 100,000 cells per well in 24 well plates using DMEM/F-12 (Gibco) with 10% Fetal Bovine Serum (Gibco). Plasmid transfection mixes were made by combining 250 ng of plasmid diluted in 25 μl Opti-MEM (Gibco) with 1 of P3000 reagent (Invitrogen). 25 μl of Opti-MEM along with 1.5 μl Lipofectamine 3000 reagent was added to the diluted DNA mix and incubated at room temperature for 10-15 minutes. 50 μl of the transfection mix was added to the cells and incubated at 37° C. and 5% CO2 overnight.

Co-transfection of cells with minigene plasmids and antisense oligonucleotides. Minigene plasmids were transfected into HEK293T cells or ARPE19 cells. HEK293T cells were seeded at 75000 cells per well in 24 well plates using IMDM supplemented with 10% Cosmic Calf Serum and 2 mM L-glutamine and incubated at 37° C. and 5% CO2 overnight. ARPE19 cells were seeded at 100,000 cells per well in 24 well plates using DMEM/F-12 (Gibco) with 10% Fetal Bovine Serum (Gibco). Plasmid transfection mixes were made by combining 250 ng of plasmid diluted in 25 μl Opti-MEM with 1 of P3000 reagent (Invitrogen). 25 μl of Opti-MEM along with 1.5 μl Lipofectamine 3000 reagent was added to the diluted DNA mix and incubated at room temperature for 10-15 minutes. 50 μl of the transfection mix was added to the cells and incubated at 37° C. and 5% CO2 overnight. 24 hours after plasmid transfection, cells were transfected with antisense oligonucleotides at absolute amounts of 150 pmol per well. For this, 150 pmol antisense oligonucleotide was mixed with 25 μl Opti-MEM and 1 μl P3000 mix to make the DNA mix. 25 μl Opti-MEM and 1.5 μl Lipofectamine 3000 was added to the DNA mix and incubated for 10-15 minutes at room temperature. Next, media was removed for the transfected cells and 500 μl of fresh IMDM (Gibco) with 10% Cosmic Calf Serum and 2 mM L-glutamine was added to each well. Subsequently, 50 μl of the antisense oligo mix was added to each well and incubated for 48 hrs hours at 37° C. and 5% CO2.

RNA isolation. RNA was isolated using ZymoResearch Magnetic Bead Kit or QIAGEN RNeasy kit, according to manufacturer's instructions.

RT-PCR analysis. First-strand cDNA synthesis was performed using the High-Capacity cDNA Reverse Transcription Kit (Thermo Fisher), according to manufacturer's instructions. Target-specific fragments were amplified by PCR using the primers listed in Table 2. PCR reactions contained 5 μl first-strand cDNA product, 0.4 μM forward primer, 0.4 μM reverse primer, 300 μM of each dNTP, 25 mM Tricine, 7.0% Glycerol (m/v), 1.6% DMSO (m/v), 2 mM MgCl2, 85 mM NH4-acetate (pH8.7), and 1 unit Taq DNA polymerase (FroggaBio) in a total volume of 25 μL. Fragments were amplified by a touchdown PCR program (95° C. for 120 sec; 10 cycles of 95° C. for 20 sec, 68° C. for 30 sec with a decrement of 1° C. per cycle, and 72° C. for 60 sec; followed by 20 cycles of 95° C. for 20 sec, 58° C. for 30 sec, and 72° C. for 60 sec; 72° C. for 180 sec).

TABLE 2

| | | | | SEQ |
|---|---|---|---|---|
| | | | Sequence | ID |
| Exon | Variant | Primers | (5′>3′) | NO: |
| 40 | c.5714+5G>A | P1009 | GATTACAAGGAT | 450 |
| | | | GACGACGATAAG | |
| | | P1986 | TCTTCATCAACA | 451 |
| | | | ATGGGCTCC | |
| 6 | c.768G>T | P863 | ATGGGCCTGTCT | 452 |
| | | | GACTCAG | |
| | | P868 | TCATTCCTCCCC | 453 |
| | | | AAGATCTCAGA | |
| 36.1 | c.5196+1137G>A | P1979 | GTTTATCAGTGG | 454 |
| | | | AGTGAGCCC | |
| | | P1980 | GATGAAGATGCC | 455 |
| | | | CACCACC | |
| 33 | c.4773+3A>G | P995 | GTTCTGGGTCAA | 456 |
| | | | TGAACAGAG | |
| | | P1978 | GAAATCAGGTAT | 457 |
| | | | TTCTTTAGAGGCC | |

Capillary electrophoresis. Samples were analyzed using a LabChip GX Touch Nucleic Acid Analyzer using a DNA 1K Hi Sensitivity LabChip and associated reagents according to manufacturer's recommendations (GE).

Minigene plasmids. Minigene plasmids for variants c.5714+5G>A, c.768G>T, and c.5196+1137G>A were synthesized by Genscript (NJ, USA). For variant c.4773+3A>G, PCR amplification was used to obtain the sequences from ARPE19 genomic DNA. To generate the ABCA4 exon 33 wildtype minigene, PCR reactions were performed with primers ATGTTCTGGGTCAATGAACAGAGGT (SEQ ID NO: 458) and CTATCAGGTATTTCTTTAGAGGCCTC (SEQ ID NO: 459) using the Q5 High-Fidelity DNA Polymerase (NEB), according to manufacturer's protocol. To generate the ABCA4 c.4773+3A>G mutant minigene, the ABCA4 exon 33 wildtype minigene PCR product was used as a template for overlap PCR. For this, PCR was performed using with the primers ATCATGAATGTGAGCGGGgtGtgtaaacagactggagatttgagtag (SEQ ID NO: 460) and aaatctccagtctgtttacaCacCCCGCTCACATTCATGATC (SEQ ID NO: 461) using the Q5 High-Fidelity DNA Polymerase (NEB), according to manufacturer's protocol to create two fragments. Overlap PCR was performed to create the minigene insert using the Phusion High-Fidelity DNA Polymerase (NEB) under the following cycling conditions: (98° C. for 30 sec; 15 cycles of 98° C. for 10 sec, 60° C. for 30 sec and 72° C. for 120 sec; followed by 20 cycles of cycles of 98° C. for 10 sec, 72° C. for 150 sec; 72° C. for 120 sec). PCR fragments were cloned into CMV containing expression vector.

### Example 1 the Splicing of ABCA4 is Disrupted in the c.768G>T Variant and can be Partially Rescued Through the Use of Antisense Oligonucleotides

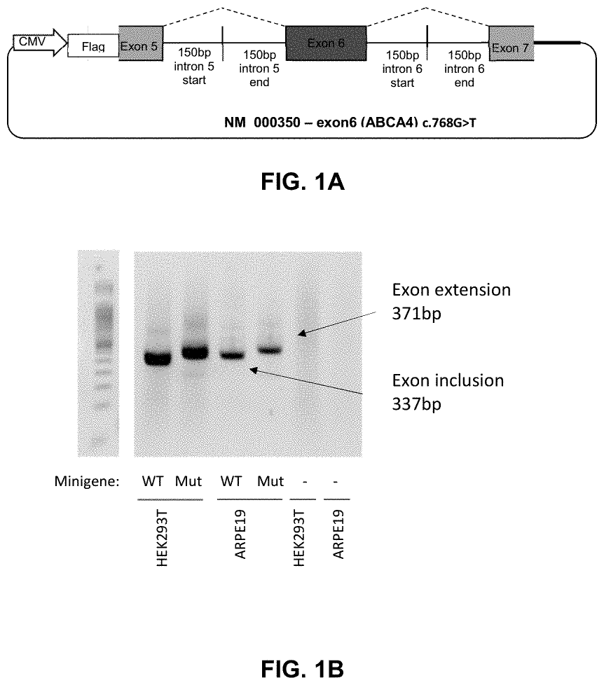

To confirm partial intron 6 inclusion (i.e. exon 6 extension) in the chr1: 94564350:C:A [hg19/b37] (c.768G>T) variant, wild type and variant containing minigenes were constructed containing exons 5-7 and the corresponding introns, 5 and 6 (FIG. 1A). Minigenes were then transfected into HEK293T and ARPE19 cells to examine the effect of the c.768G>T variant on splicing. As seen in FIG. 1B, wildtype minigenes showed intron 6 exclusion, represented by the 337 bp band. C.768G>T mutants, however, showed partial intron 6 inclusion (i.e. exon 6 extension) indicating the chr1: 94564350:C:A [hg19/b37] mutation induces partial intron 6 inclusion.

To examine the ability of antisense oligonucleotides to promote intron 6 exclusion in the c.768G>T variant the minigenes above were co-transfected with antisense oligonucleotides having sequences set forth in SEQ ID Nos: 2-207 (see Tables 3 and 4). Antisense oligonucleotides were tiled along exon 6 and the surrounding introns. Antisense oligonucleotides were cotransfected with the mutant minigene containing the c.768G>T variant in ARPE19 (Table 3) and HEK293T (Table 4) cells. RT-PCR was conducted to analyze the effect on the splicing of the minigene. Samples were measured by capillary electrophoresis. These results were quantified and are set forth in Tables 3 and 4. Observing Table 3 and 4 it is clear that targeting the intronic regions surrounding exon 6 reduces intron 6 inclusion in c.768G>T variant minigenes (high percent spliced in/correctly (PSI) and change in PSI as compared to mutant PSI (dPSI)). These observations also suggest antisense oligonucleotides targeting certain regions or “hotspots” in intron 6 (positions 27362-27419 in SEQ ID NO: 1; chr1: 94564287-94564344), e.g., those complementary to a nucleobase sequence in SEQ ID Nos: 60-198 and 207, may be particularly useful in the treatment of retinal disease associated with partial intron 6 inclusion (i.e. exon 6 extension) (e.g., retinitis pigmentosa, cone-rod dystrophy, or Stargardt disease caused by the c.768G>T mutation).

TABLE 3

| | | | | | | Start | | | |
|---|---|---|---|---|---|---|---|---|---|
| | | | | | | on | | | |
| SEQ | | | | Start | | SEQ | Stop on | | |
| ID | DG | | | Chr1 | End Chr1 | ID | SEQ ID | | |
| NO: | ID | PSI | Sequence | [hg19/b37] | [hg19/b37] | NO: 1 | NO: 1 | length | dPSI |
| 2 | 4128 | 0.02431697 | ATACCT | 94564626 | 94564645 | 27061 | 27080 | 20 | 0.00628733 |
| | | | TGTGTT | | | | | | |
| | | | ACATGG | | | | | | |
| | | | CG | | | | | | |
| 3 | 4073 | 0.11405178 | GGGAAT | 94564622 | 94564638 | 27068 | 27084 | 17 | 0.09602214 |
| | | | ACCTTG | | | | | | |
| | | | TGTTA | | | | | | |
| 4 | 4141 | 0.15732851 | AGAACC | 94564615 | 94564635 | 27071 | 27091 | 21 | 0.13929887 |
| | | | TGGGAA | | | | | | |
| | | | TACCTT | | | | | | |
| | | | GTG | | | | | | |
| 5 | 4114 | 0.01903612 | CTAACC | 94564606 | 94564624 | 27082 | 27100 | 19 | 0.00100648 |
| | | | CACAGA | | | | | | |
| | | | ACCTGG | | | | | | |
| | | | G | | | | | | |
| 6 | 4129 | 0.00522592 | CCACGT | 94564599 | 94564618 | 27088 | 27107 | 20 | −0.0128037 |
| | | | CCTAAC | | | | | | |
| | | | CCACAG | | | | | | |
| | | | AA | | | | | | |
| 7 | 4130 | 0.02776844 | GAAAGA | 94564590 | 94564609 | 27097 | 27116 | 20 | 0.0097388 |
| | | | CACCCA | | | | | | |
| | | | CGTCCT | | | | | | |
| | | | AA | | | | | | |
| 8 | 4095 | 0.02402144 | TAGGAA | 94564587 | 94564604 | 27102 | 27119 | 18 | 0.00599179 |
| | | | AGACAC | | | | | | |
| | | | CCACGT | | | | | | |
| 9 | 4074 | 0.02229321 | GGTAGG | 94564585 | 94564601 | 27105 | 27121 | 17 | 0.00426357 |
| | | | AAAGAC | | | | | | |
| | | | ACCCA | | | | | | |
| 10 | 4115 | 0.0201467 | CCCTGT | 94564579 | 94564597 | 27109 | 27127 | 19 | 0.00211706 |
| | | | GGTAGG | | | | | | |
| | | | AAAGAC | | | | | | |
| | | | A | | | | | | |
| 11 | 4096 | 0.01513791 | CTGCCC | 94564576 | 94564593 | 27113 | 27130 | 18 | −0.0028917 |
| | | | TGTGGT | | | | | | |
| | | | AGGAAA | | | | | | |
| 12 | 4075 | 0.01842179 | AACTGC | 94564574 | 94564590 | 27116 | 27132 | 17 | 0.00039215 |
| | | | CCTGTG | | | | | | |
| | | | GTAGG | | | | | | |
| 13 | 4076 | 0.01700658 | GAAACT | 94564572 | 94564588 | 27118 | 27134 | 17 | -0.0010231 |
| | | | GCCCTG | | | | | | |
| | | | TGGTA | | | | | | |
| 14 | 4097 | 0.02153469 | CTAGAA | 94564569 | 94564586 | 27120 | 27137 | 18 | 0.00350505 |
| | | | ACTGCC | | | | | | |
| | | | CTGTGG | | | | | | |
| 15 | 4131 | 0.01829548 | GGCAAC | 94564562 | 94564581 | 27125 | 27144 | 20 | 0.00026584 |
| | | | ACTAGA | | | | | | |
| | | | AACTGC | | | | | | |
| | | | CC | | | | | | |
| 16 | 4142 | 0.0197163 | GGAGAA | 94564554 | 94564574 | 27132 | 27152 | 21 | 0.00168666 |
| | | | GAGGCA | | | | | | |
| | | | ACACTA | | | | | | |
| | | | GAA | | | | | | |
| 17 | 4098 | 0.01724193 | CAGGGA | 94564551 | 94564568 | 27138 | 27155 | 18 | −0.0007877 |
| | | | GAAGAG | | | | | | |
| | | | GCAACA | | | | | | |
| 18 | 4077 | 0.02141061 | ACTGCA | 94564547 | 94564563 | 27143 | 27159 | 17 | 0.00338097 |
| | | | GGGAGA | | | | | | |
| | | | AGAGG | | | | | | |
| 19 | 4132 | 0.0033317 | GAGCGA | 94564541 | 94564560 | 27146 | 27165 | 20 | −0.0146979 |
| | | | ACTGCA | | | | | | |
| | | | GGGAGA | | | | | | |
| | | | AG | | | | | | |
| 20 | 4133 | 0.01705203 | TCCATG | 94564536 | 94564555 | 27151 | 27170 | 20 | −0.0009776 |
| | | | AGCGAA | | | | | | |
| | | | CTGCAG | | | | | | |
| | | | GG | | | | | | |
| 21 | 4134 | 0.01805005 | GGGACT | 94564531 | 94564550 | 27156 | 27175 | 20 | 2.0406E-05 |
| | | | CCATGA | | | | | | |
| | | | GCGAAC | | | | | | |
| | | | TG | | | | | | |
| 22 | 4078 | 0.01914936 | TCCGGG | 94564528 | 94564544 | 27162 | 27178 | 17 | 0.00111972 |
| | | | ACTCCA | | | | | | |
| | | | TGAGC | | | | | | |
| 23 | 4143 | 0.01964017 | AGCGCC | 94564519 | 94564539 | 27167 | 27187 | 21 | 0.00161053 |
| | | | AGGTCC | | | | | | |
| | | | GGGACT | | | | | | |
| | | | CCA | | | | | | |
| 24 | 4144 | 0.01859193 | GTCCTTC | 94564512 | 94564532 | 27174 | 27194 | 21 | 0.00056229 |
| | | | AGCGCC | | | | | | |
| | | | AGGTCC | | | | | | |
| | | | GG | | | | | | |
| 25 | 4145 | 0.02145499 | CAGGCG | 94564504 | 94564524 | 27182 | 27202 | 21 | 0.00342535 |
| | | | ATGTCC | | | | | | |
| | | | TTCAGC | | | | | | |
| | | | GCC | | | | | | |
| 26 | 4116 | 0.02012965 | CCTCGC | 94564496 | 94564514 | 27192 | 27210 | 19 | 0.00210001 |
| | | | TGCAGG | | | | | | |
| | | | CGATGT | | | | | | |
| | | | C | | | | | | |
| 27 | 4099 | 0.02319291 | GGAGGG | 94564490 | 94564507 | 27199 | 27216 | 18 | 0.00516327 |
| | | | CCTCGC | | | | | | |
| | | | TGCAGG | | | | | | |
| 28 | 4100 | 0.03543829 | GCTCCA | 94564484 | 94564501 | 27205 | 27222 | 18 | 0.01740865 |
| | | | GGAGGG | | | | | | |
| | | | CCTCGC | | | | | | |
| 29 | 4079 | 0.02046444 | GCGCTC | 94564482 | 94564498 | 27208 | 27224 | 17 | 0.0024348 |
| | | | CAGGAG | | | | | | |
| | | | GGCCT | | | | | | |
| 30 | 4101 | 0.01793776 | TGAAGC | 94564478 | 94564495 | 27211 | 27228 | 18 | −9.188E-05 |
| | | | GCTCCA | | | | | | |
| | | | GGAGGG | | | | | | |
| 31 | 4135 | 0.01365784 | GAAGAT | 94564470 | 94564489 | 27217 | 27236 | 20 | −0.0043718 |
| | | | GATGAA | | | | | | |
| | | | GCGCTC | | | | | | |
| | | | CA | | | | | | |
| 32 | 4117 | 0.01708284 | TGGCTG | 94564465 | 94564483 | 27223 | 27241 | 19 | −0.0009468 |
| | | | AAGATG | | | | | | |
| | | | ATGAAG | | | | | | |
| | | | C | | | | | | |
| 33 | 4118 | 0.02096426 | TCTCTG | 94564461 | 94564479 | 27227 | 27245 | 19 | 0.00293462 |
| | | | GCTGAA | | | | | | |
| | | | GATGAT | | | | | | |
| | | | G | | | | | | |
| 34 | 4080 | 0.01739695 | CGTCTCT | 94564459 | 94564475 | 27231 | 27247 | 17 | −0.0006327 |
| | | | GGCTGA | | | | | | |
| | | | AGAT | | | | | | |
| 35 | 4136 | 0.02244261 | TTGCCC | 94564451 | 94564470 | 27236 | 27255 | 20 | 0.00441297 |
| | | | CGCGTC | | | | | | |
| | | | TCTGGC | | | | | | |
| | | | TG | | | | | | |
| 36 | 4102 | 0.02139803 | CACCGT | 94564443 | 94564460 | 27246 | 27263 | 18 | 0.00336839 |
| | | | CTTTGCC | | | | | | |
| | | | CCGCG | | | | | | |
| 37 | 4146 | 0.0177441 | ATAGCG | 94564437 | 94564457 | 27249 | 27269 | 21 | −0.0002855 |
| | | | CACCGT | | | | | | |
| | | | CTTTGCC | | | | | | |
| | | | CC | | | | | | |
| 38 | 4081 | 0.01717889 | GGCATA | 94564434 | 94564450 | 27256 | 27272 | 17 | −0.0008508 |
| | | | GCGCAC | | | | | | |
| | | | CGTCT | | | | | | |
| 39 | 4103 | 0.01417047 | CAGGGC | 94564431 | 94564448 | 27258 | 27275 | 18 | −0.0038592 |
| | | | ATAGCG | | | | | | |
| | | | CACCGT | | | | | | |
| 40 | 4119 | 0.01682693 | GAGCAC | 94564426 | 94564444 | 27262 | 27280 | 19 | −0.0012027 |
| | | | AGGGCA | | | | | | |
| | | | TAGCGC | | | | | | |
| | | | A | | | | | | |
| 41 | 4082 | 0.00630405 | AGAGGG | 94564421 | 94564437 | 27269 | 27285 | 17 | −0.0117256 |
| | | | AGCACA | | | | | | |
| | | | GGGCA | | | | | | |
| 42 | 4120 | 0.00571713 | TGGGAG | 94564417 | 94564435 | 27271 | 27289 | 19 | −0.0123125 |
| | | | AGGGAG | | | | | | |
| | | | CACAGG | | | | | | |
| | | | G | | | | | | |
| 43 | 4147 | 0.00235213 | TAGGGT | 94564407 | 94564427 | 27279 | 27299 | 21 | −0.0156775 |
| | | | GCCCTG | | | | | | |
| | | | GGAGAG | | | | | | |
| | | | GGA | | | | | | |
| 44 | 4148 | 0.01718938 | TATCCA | 94564398 | 94564418 | 27288 | 27308 | 21 | −0.0008403 |
| | | | CTGTAG | | | | | | |
| | | | GGTGCC | | | | | | |
| | | | CTG | | | | | | |
| 45 | 4083 | 0.00387113 | TCTTCTA | 94564393 | 94564409 | 27297 | 27313 | 17 | −0.0141585 |
| | | | TCCACT | | | | | | |
| | | | GTAG | | | | | | |
| 46 | 4084 | 0.00368482 | AGTGTC | 94564389 | 94564405 | 27301 | 27317 | 17 | −0.0143448 |
| | | | TTCTATC | | | | | | |
| | | | CACT | | | | | | |
| 47 | 4104 | 0.00409522 | CAGAGT | 94564386 | 94564403 | 27303 | 27320 | 18 | −0.0139344 |
| | | | GTCTTCT | | | | | | |
| | | | ATCCA | | | | | | |
| 48 | 4137 | 0.00445977 | GTTGGC | 94564377 | 94564396 | 27310 | 27329 | 20 | −0.0135699 |
| | | | ATACAG | | | | | | |
| | | | AGTGTC | | | | | | |
| | | | TT | | | | | | |
| 49 | 4121 | 0.00750785 | CCACGT | 94564373 | 94564391 | 27315 | 27333 | 19 | −0.0105218 |
| | | | TGGCAT | | | | | | |
| | | | ACAGAG | | | | | | |
| | | | T | | | | | | |
| 50 | 4105 | 0.00641796 | AAGTCC | 94564369 | 94564386 | 27320 | 27337 | 18 | −0.0116117 |
| | | | ACGTTG | | | | | | |
| | | | GCATAC | | | | | | |
| 51 | 4106 | 0.00402175 | AAGAAG | 94564366 | 94564383 | 27323 | 27340 | 18 | −0.0140079 |
| | | | TCCACG | | | | | | |
| | | | TTGGCA | | | | | | |
| 52 | 4122 | 0.00421162 | GCTTGA | 94564361 | 94564379 | 27327 | 27345 | 19 | −0.013818 |
| | | | AGAAGT | | | | | | |
| | | | CCACGT | | | | | | |
| | | | T | | | | | | |
| 53 | 4107 | 0.00378806 | AAGAGC | 94564357 | 94564374 | 27332 | 27349 | 18 | −0.0142416 |
| | | | TTGAAG | | | | | | |
| | | | AAGTCC | | | | | | |
| 54 | 4108 | 0.00324747 | CGGAAG | 94564354 | 94564371 | 27335 | 27352 | 18 | −0.0147822 |
| | | | AGCTTG | | | | | | |
| | | | AAGAAG | | | | | | |
| 55 | 4085 | 0.00334551 | AACACG | 94564350 | 94564366 | 27340 | 27356 | 17 | −0.0146841 |
| | | | GAAGAG | | | | | | |
| | | | CTTGA | | | | | | |
| 56 | 4123 | 0.01473282 | CTTACA | 94564345 | 94564363 | 27343 | 27361 | 19 | −0.0032968 |
| | | | ACACGG | | | | | | |
| | | | AAGAGC | | | | | | |
| | | | T | | | | | | |
| 57 | 4109 | 0.02021009 | CTCCCTT | 94564341 | 94564358 | 27348 | 27365 | 18 | 0.00218045 |
| | | | ACAACA | | | | | | |
| | | | CGGAA | | | | | | |
| 58 | 4149 | 0.01481722 | CCAAAC | 94564333 | 94564353 | 27353 | 27373 | 21 | −0.0032124 |
| | | | CCCTCC | | | | | | |
| | | | CTTACA | | | | | | |
| | | | ACA | | | | | | |
| 59 | 4086 | 0.01443598 | CAGCCA | 94564330 | 94564346 | 27360 | 27376 | 17 | −0.0035937 |
| | | | AACCCC | | | | | | |
| | | | TCCCT | | | | | | |
| 60 | 4087 | 0.02024966 | AGCAGC | 94564328 | 94564344 | 27362 | 27378 | 17 | 0.00222002 |
| | | | CAAACC | | | | | | |
| | | | CCTCC | | | | | | |
| 61 | 4088 | 0.0372636 | CGAGCA | 94564326 | 94564342 | 27364 | 27380 | 17 | 0.01923396 |
| | | | GCCAAA | | | | | | |
| | | | CCCCT | | | | | | |
| 62 | 4110 | 0.07618036 | TGGCGA | 94564323 | 94564340 | 27366 | 27383 | 18 | 0.05815072 |
| | | | GCAGCC | | | | | | |
| | | | AAACCC | | | | | | |
| 63 | 4138 | 0.17283501 | TGCAAT | 94564317 | 94564336 | 27370 | 27389 | 20 | 0.15480537 |
| | | | TGGCGA | | | | | | |
| | | | GCAGCC | | | | | | |
| | | | AA | | | | | | |
| 64 | 4597 | 0.10930086 | AATTGG | 94564320 | 94564336 | 27370 | 27386 | 17 | 0.09127122 |
| | | | CGAGCA | | | | | | |
| | | | GCCAA | | | | | | |
| 65 | 4598 | 0.09078839 | CAATTG | 94564319 | 94564336 | 27370 | 27387 | 18 | 0.07275875 |
| | | | GCGAGC | | | | | | |
| | | | AGCCAA | | | | | | |
| 66 | 4599 | 0.16039823 | GCAATT | 94564318 | 94564336 | 27370 | 27388 | 19 | 0.14236859 |
| | | | GGCGAG | | | | | | |
| | | | CAGCCA | | | | | | |
| | | | A | | | | | | |
| 67 | 4600 | 0.16117871 | TTGCAA | 94564316 | 94564336 | 27370 | 27390 | 21 | 0.14314907 |
| | | | TTGGCG | | | | | | |
| | | | AGCAGC | | | | | | |
| | | | CAA | | | | | | |
| 68 | 4601 | 0.11209091 | CAATTG | 94564319 | 94564335 | 27371 | 27387 | 17 | 0.09406127 |
| | | | GCGAGC | | | | | | |
| | | | AGCCA | | | | | | |
| 69 | 4602 | 0.23648176 | GCAATT | 94564318 | 94564335 | 27371 | 27388 | 18 | 0.21845211 |
| | | | GGCGAG | | | | | | |
| | | | CAGCCA | | | | | | |
| 70 | 4603 | 0.20595156 | TGCAAT | 94564317 | 94564335 | 27371 | 27389 | 19 | 0.18792192 |
| | | | TGGCGA | | | | | | |
| | | | GCAGCC | | | | | | |
| | | | A | | | | | | |
| 71 | 4604 | 0.17100969 | TTGCAA | 94564316 | 94564335 | 27371 | 27390 | 20 | 0.15298005 |
| | | | TTGGCG | | | | | | |
| | | | AGCAGC | | | | | | |
| | | | CA | | | | | | |
| 72 | 4605 | 0.14927085 | CTTGCA | 94564315 | 94564335 | 27371 | 27391 | 21 | 0.13124121 |
| | | | ATTGGC | | | | | | |
| | | | GAGCAG | | | | | | |
| | | | CCA | | | | | | |
| 73 | 4606 | 0.26777524 | GCAATT | 94564318 | 94564334 | 27372 | 27388 | 17 | 0.2497456 |
| | | | GGCGAG | | | | | | |
| | | | CAGCC | | | | | | |
| 74 | 4607 | 0.29621478 | TGCAAT | 94564317 | 94564334 | 27372 | 27389 | 18 | 0.27818514 |
| | | | TGGCGA | | | | | | |
| | | | GCAGCC | | | | | | |
| 75 | 4608 | 0.31043846 | TTGCAA | 94564316 | 94564334 | 27372 | 27390 | 19 | 0.29240882 |
| | | | TTGGCG | | | | | | |
| | | | AGCAGC | | | | | | |
| | | | C | | | | | | |
| 76 | 4609 | 0.26478391 | CTTGCA | 94564315 | 94564334 | 27372 | 27391 | 20 | 0.24675427 |
| | | | ATTGGC | | | | | | |
| | | | GAGCAG | | | | | | |
| | | | CC | | | | | | |
| 77 | 4610 | 0.25010219 | CCTTGC | 94564314 | 94564334 | 27372 | 27392 | 21 | 0.23207255 |
| | | | AATTGG | | | | | | |
| | | | CGAGCA | | | | | | |
| | | | GCC | | | | | | |
| 78 | 4611 | 0.26743515 | TGCAAT | 94564317 | 94564333 | 27373 | 27389 | 17 | 0.24940551 |
| | | | TGGCGA | | | | | | |
| | | | GCAGC | | | | | | |
| 79 | 4612 | 0.20968878 | TTGCAA | 94564316 | 94564333 | 27373 | 27390 | 18 | 0.19165914 |
| | | | TTGGCG | | | | | | |
| | | | AGCAGC | | | | | | |
| 80 | 4613 | 0.24661075 | CTTGCA | 94564315 | 94564333 | 27373 | 27391 | 19 | 0.22858111 |
| | | | ATTGGC | | | | | | |
| | | | GAGCAG | | | | | | |
| | | | C | | | | | | |
| 81 | 4614 | 0.23289843 | CCTTGC | 94564314 | 94564333 | 27373 | 27392 | 20 | 0.21486879 |
| | | | AATTGG | | | | | | |
| | | | CGAGCA | | | | | | |
| | | | GC | | | | | | |
| 82 | 4615 | 0.29501713 | ACCTTG | 94564313 | 94564333 | 27373 | 27393 | 21 | 0.27698749 |
| | | | CAATTG | | | | | | |
| | | | GCGAGC | | | | | | |
| | | | AGC | | | | | | |
| 83 | 4616 | 0.27962315 | TTGCAA | 94564316 | 94564332 | 27374 | 27390 | 17 | 0.26159351 |
| | | | TTGGCG | | | | | | |
| | | | AGCAG | | | | | | |
| 84 | 4617 | 0.22421363 | CTTGCA | 94564315 | 94564332 | 27374 | 27391 | 18 | 0.20618399 |
| | | | ATTGGC | | | | | | |
| | | | GAGCAG | | | | | | |
| 85 | 4618 | 0.26986428 | CCTTGC | 94564314 | 94564332 | 27374 | 27392 | 19 | 0.25183464 |
| | | | AATTGG | | | | | | |
| | | | CGAGCA | | | | | | |
| | | | G | | | | | | |
| 86 | 4619 | 0.29570147 | ACCTTG | 94564313 | 94564332 | 27374 | 27393 | 20 | 0.27767183 |
| | | | CAATTG | | | | | | |
| | | | GCGAGC | | | | | | |
| | | | AG | | | | | | |
| 87 | 4620 | 0.26279915 | CACCTT | 94564312 | 94564332 | 27374 | 27394 | 21 | 0.24476951 |
| | | | GCAATT | | | | | | |
| | | | GGCGAG | | | | | | |
| | | | CAG | | | | | | |
| 88 | 4089 | 0.17943073 | CTTGCA | 94564315 | 94564331 | 27375 | 27391 | 17 | 0.16140109 |
| | | | ATTGGC | | | | | | |
| | | | GAGCA | | | | | | |
| 89 | 4621 | 0.26260696 | CCTTGC | 94564314 | 94564331 | 27375 | 27392 | 18 | 0.24457732 |
| | | | AATTGG | | | | | | |
| | | | CGAGCA | | | | | | |
| 90 | 4622 | 0.31982099 | ACCTTG | 94564313 | 94564331 | 27375 | 27393 | 19 | 0.30179135 |
| | | | CAATTG | | | | | | |
| | | | GCGAGC | | | | | | |
| | | | A | | | | | | |
| 91 | 4623 | 0.2558288 | CACCTT | 94564312 | 94564331 | 27375 | 27394 | 20 | 0.23779916 |
| | | | GCAATT | | | | | | |
| | | | GGCGAG | | | | | | |
| | | | CA | | | | | | |
| 92 | 4624 | 0.23800896 | TCACCTT | 94564311 | 94564331 | 27375 | 27395 | 21 | 0.21997932 |
| | | | GCAATT | | | | | | |
| | | | GGCGAG | | | | | | |
| | | | CA | | | | | | |
| 93 | 4625 | 0.25760784 | CCTTGC | 94564314 | 94564330 | 27376 | 27392 | 17 | 0.2395782 |
| | | | AATTGG | | | | | | |
| | | | CGAGC | | | | | | |
| 94 | 4626 | 0.29734234 | ACCTTG | 94564313 | 94564330 | 27376 | 27393 | 18 | 0.2793127 |
| | | | CAATTG | | | | | | |
| | | | GCGAGC | | | | | | |
| 95 | 4627 | 0.26139422 | CACCTT | 94564312 | 94564330 | 27376 | 27394 | 19 | 0.24336458 |
| | | | GCAATT | | | | | | |
| | | | GGCGAG | | | | | | |
| | | | C | | | | | | |
| 96 | 4628 | 0.18097064 | TCACCTT | 94564311 | 94564330 | 27376 | 27395 | 20 | 0.162941 |
| | | | GCAATT | | | | | | |
| | | | GGCGAG | | | | | | |
| | | | C | | | | | | |
| 97 | 4629 | 0.27847245 | ATCACC | 94564310 | 94564330 | 27376 | 27396 | 21 | 0.26044281 |
| | | | TTGCAA | | | | | | |
| | | | TTGGCG | | | | | | |
| | | | AGC | | | | | | |
| 98 | 4090 | 0.26236346 | ACCTTG | 94564313 | 94564329 | 27377 | 27393 | 17 | 0.24433382 |
| | | | CAATTG | | | | | | |
| | | | GCGAG | | | | | | |
| 99 | 4630 | 0.31917424 | CACCTT | 94564312 | 94564329 | 27377 | 27394 | 18 | 0.3011446 |
| | | | GCAATT | | | | | | |
| | | | GGCGAG | | | | | | |
| 100 | 4631 | 0.76759466 | TCACCTT | 94564311 | 94564329 | 27377 | 27395 | 19 | 0.74956501 |
| | | | GCAATT | | | | | | |
| | | | GGCGAG | | | | | | |
| 101 | 4632 | 0.81860163 | ATCACC | 94564310 | 94564329 | 27377 | 27396 | 20 | 0.80057199 |
| | | | TTGCAA | | | | | | |
| | | | TTGGCG | | | | | | |
| | | | AG | | | | | | |
| 102 | 4633 | 0.89239232 | AATCAC | 94564309 | 94564329 | 27377 | 27397 | 21 | 0.87436268 |
| | | | CTTGCA | | | | | | |
| | | | ATTGGC | | | | | | |
| | | | GAG | | | | | | |
| 103 | 4634 | 0.84651316 | CACCTT | 94564312 | 94564328 | 27378 | 27394 | 17 | 0.82848352 |
| | | | GCAATT | | | | | | |
| | | | GGCGA | | | | | | |
| 104 | 4635 | 0.8390091 | TCACCTT | 94564311 | 94564328 | 27378 | 27395 | 18 | 0.82097946 |
| | | | GCAATT | | | | | | |
| | | | GGCGA | | | | | | |
| 105 | 4636 | 0.87739626 | ATCACC | 94564310 | 94564328 | 27378 | 27396 | 19 | 0.85936662 |
| | | | TTGCAA | | | | | | |
| | | | TTGGCG | | | | | | |
| | | | A | | | | | | |
| 106 | 4637 | 0.87346315 | AATCAC | 94564309 | 94564328 | 27378 | 27397 | 20 | 0.85543351 |
| | | | CTTGCA | | | | | | |
| | | | ATTGGC | | | | | | |
| | | | GA | | | | | | |
| 107 | 4638 | 0.90143132 | GAATCA | 94564308 | 94564328 | 27378 | 27398 | 21 | 0.88340168 |
| | | | CCTTGC | | | | | | |
| | | | AATTGG | | | | | | |
| | | | CGA | | | | | | |
| 108 | 4124 | 0.44721392 | AATCAC | 94564309 | 94564327 | 27379 | 27397 | 19 | 0.42918427 |
| | | | CTTGCA | | | | | | |
| | | | ATTGGC | | | | | | |
| | | | G | | | | | | |
| 109 | 4639 | 0.79968337 | TCACCTT | 94564311 | 94564327 | 27379 | 27395 | 17 | 0.78165373 |
| | | | GCAATT | | | | | | |
| | | | GGCG | | | | | | |
| 110 | 4640 | 0.80763727 | ATCACC | 94564310 | 94564327 | 27379 | 27396 | 18 | 0.78960763 |
| | | | TTGCAA | | | | | | |
| | | | TTGGCG | | | | | | |
| 111 | 4641 | 0.87411122 | GAATCA | 94564308 | 94564327 | 27379 | 27398 | 20 | 0.85608158 |
| | | | CCTTGC | | | | | | |
| | | | AATTGG | | | | | | |
| | | | CG | | | | | | |
| 112 | 4642 | 0.80500233 | GGAATC | 94564307 | 94564327 | 27379 | 27399 | 21 | 0.78697268 |
| | | | ACCTTG | | | | | | |
| | | | CAATTG | | | | | | |
| | | | GCG | | | | | | |
| 113 | 4643 | 0.88269558 | ATCACC | 94564310 | 94564326 | 27380 | 27396 | 17 | 0.86466594 |
| | | | TTGCAA | | | | | | |
| | | | TTGGC | | | | | | |
| 114 | 4644 | 0.87044459 | AATCAC | 94564309 | 94564326 | 27380 | 27397 | 18 | 0.85241495 |
| | | | CTTGCA | | | | | | |
| | | | ATTGGC | | | | | | |
| 115 | 4645 | 0.73199713 | GAATCA | 94564308 | 94564326 | 27380 | 27398 | 19 | 0.71396749 |
| | | | CCTTGC | | | | | | |
| | | | AATTGG | | | | | | |
| | | | C | | | | | | |
| 116 | 4646 | 0.68348265 | GGAATC | 94564307 | 94564326 | 27380 | 27399 | 20 | 0.66545301 |
| | | | ACCTTG | | | | | | |
| | | | CAATTG | | | | | | |
| | | | GC | | | | | | |
| 117 | 4647 | 0.82294769 | AGGAAT | 94564306 | 94564326 | 27380 | 27400 | 21 | 0.80491805 |
| | | | CACCTT | | | | | | |
| | | | GCAATT | | | | | | |
| | | | GGC | | | | | | |
| 118 | 4648 | 0.84365284 | AATCAC | 94564309 | 94564325 | 27381 | 27397 | 17 | 0.8256232 |
| | | | CTTGCA | | | | | | |
| | | | ATTGG | | | | | | |
| 119 | 4649 | 0.78266251 | GAATCA | 94564308 | 94564325 | 27381 | 27398 | 18 | 0.76463287 |
| | | | CCTTGC | | | | | | |
| | | | AATTGG | | | | | | |
| 120 | 4650 | 0.67659075 | GGAATC | 94564307 | 94564325 | 27381 | 27399 | 19 | 0.65856111 |
| | | | ACCTTG | | | | | | |
| | | | CAATTG | | | | | | |
| | | | G | | | | | | |
| 121 | 4651 | 0.67533495 | AGGAAT | 94564306 | 94564325 | 27381 | 27400 | 20 | 0.65730531 |
| | | | CACCTT | | | | | | |
| | | | GCAATT | | | | | | |
| | | | GG | | | | | | |
| 122 | 4652 | 0.70200627 | CAGGAA | 94564305 | 94564325 | 27381 | 27401 | 21 | 0.68397663 |
| | | | TCACCTT | | | | | | |
| | | | GCAATT | | | | | | |
| | | | GG | | | | | | |
| 123 | 4653 | 0.7782903 | GAATCA | 94564308 | 94564324 | 27382 | 27398 | 17 | 0.76026066 |
| | | | CCTTGC | | | | | | |
| | | | AATTG | | | | | | |
| 124 | 4654 | 0.78731012 | GGAATC | 94564307 | 94564324 | 27382 | 27399 | 18 | 0.76928048 |
| | | | ACCTTG | | | | | | |
| | | | CAATTG | | | | | | |
| 125 | 4655 | 0.78132802 | AGGAAT | 94564306 | 94564324 | 27382 | 27400 | 19 | 0.76329838 |
| | | | CACCTT | | | | | | |
| | | | GCAATT | | | | | | |
| | | | G | | | | | | |
| 126 | 4656 | 0.3388734 | CAGGAA | 94564305 | 94564324 | 27382 | 27401 | 20 | 0.32084376 |
| | | | TCACCTT | | | | | | |
| | | | GCAATT | | | | | | |
| | | | G | | | | | | |
| 127 | 4657 | 0.25626616 | CCAGGA | 94564304 | 94564324 | 27382 | 27402 | 21 | 0.23823652 |
| | | | ATCACC | | | | | | |
| | | | TTGCAA | | | | | | |
| | | | TTG | | | | | | |
| 128 | 4091 | 0.60563805 | GGAATC | 94564307 | 94564323 | 27383 | 27399 | 17 | 0.58760841 |
| | | | ACCTTG | | | | | | |
| | | | CAATT | | | | | | |
| 129 | 4658 | 0.88473952 | AGGAAT | 94564306 | 94564323 | 27383 | 27400 | 18 | 0.86670988 |
| | | | CACCTT | | | | | | |
| | | | GCAATT | | | | | | |
| 130 | 4659 | 0.88226254 | CAGGAA | 94564305 | 94564323 | 27383 | 27401 | 19 | 0.8642329 |
| | | | TCACCTT | | | | | | |
| | | | GCAATT | | | | | | |
| 131 | 4660 | 0.85095103 | CCAGGA | 94564304 | 94564323 | 27383 | 27402 | 20 | 0.83292139 |
| | | | ATCACC | | | | | | |
| | | | TTGCAA | | | | | | |
| | | | TT | | | | | | |
| 132 | 4661 | 0.83219493 | CCCAGG | 94564303 | 94564323 | 27383 | 27403 | 21 | 0.81416529 |
| | | | AATCAC | | | | | | |
| | | | CTTGCA | | | | | | |
| | | | ATT | | | | | | |
| 133 | 4662 | 0.88970276 | AGGAAT | 94564306 | 94564322 | 27384 | 27400 | 17 | 0.87167312 |
| | | | CACCTT | | | | | | |
| | | | GCAAT | | | | | | |
| 134 | 4663 | 0.87956906 | CAGGAA | 94564305 | 94564322 | 27384 | 27401 | 18 | 0.86153942 |
| | | | TCACCTT | | | | | | |
| | | | GCAAT | | | | | | |
| 135 | 4664 | 0.81659418 | CCAGGA | 94564304 | 94564322 | 27384 | 27402 | 19 | 0.79856454 |
| | | | ATCACC | | | | | | |
| | | | TTGCAA | | | | | | |
| | | | T | | | | | | |
| 136 | 4665 | 0.85952746 | CCCAGG | 94564303 | 94564322 | 27384 | 27403 | 20 | 0.84149781 |
| | | | AATCAC | | | | | | |
| | | | CTTGCA | | | | | | |
| | | | AT | | | | | | |
| 137 | 4666 | 0.69318589 | CCCCAG | 94564302 | 94564322 | 27384 | 27404 | 21 | 0.67515625 |
| | | | GAATCA | | | | | | |
| | | | CCTTGC | | | | | | |
| | | | AAT | | | | | | |
| 138 | 4125 | 0.29460087 | CCCAGG | 94564303 | 94564321 | 27385 | 27403 | 19 | 0.27657123 |
| | | | AATCAC | | | | | | |
| | | | CTTGCA | | | | | | |
| | | | A | | | | | | |
| 139 | 4667 | 0.36645782 | CAGGAA | 94564305 | 94564321 | 27385 | 27401 | 17 | 0.34842818 |
| | | | TCACCTT | | | | | | |
| | | | GCAA | | | | | | |
| 140 | 4668 | 0.83743902 | CCAGGA | 94564304 | 94564321 | 27385 | 27402 | 18 | 0.81940938 |
| | | | ATCACC | | | | | | |
| | | | TTGCAA | | | | | | |
| 141 | 4669 | 0.29444226 | CCCCAG | 94564302 | 94564321 | 27385 | 27404 | 20 | 0.27641262 |
| | | | GAATCA | | | | | | |
| | | | CCTTGC | | | | | | |
| | | | AA | | | | | | |
| 142 | 4670 | 0.23897641 | ACCCCA | 94564301 | 94564321 | 27385 | 27405 | 21 | 0.22094677 |
| | | | GGAATC | | | | | | |
| | | | ACCTTG | | | | | | |
| | | | CAA | | | | | | |
| 143 | 4671 | 0.22377272 | CCAGGA | 94564304 | 94564320 | 27386 | 27402 | 17 | 0.20574308 |
| | | | ATCACC | | | | | | |
| | | | TTGCA | | | | | | |
| 144 | 4672 | 0.27703321 | CCCAGG | 94564303 | 94564320 | 27386 | 27403 | 18 | 0.25900356 |
| | | | AATCAC | | | | | | |
| | | | CTTGCA | | | | | | |
| 145 | 4673 | 0.22181682 | CCCCAG | 94564302 | 94564320 | 27386 | 27404 | 19 | 0.20378717 |
| | | | GAATCA | | | | | | |
| | | | CCTTGC | | | | | | |
| | | | A | | | | | | |
| 146 | 4674 | 0.73692266 | ACCCCA | 94564301 | 94564320 | 27386 | 27405 | 20 | 0.71889302 |
| | | | GGAATC | | | | | | |
| | | | ACCTTG | | | | | | |
| | | | CA | | | | | | |
| 147 | 4675 | 0.16174868 | TACCCC | 94564300 | 94564320 | 27386 | 27406 | 21 | 0.14371904 |
| | | | AGGAAT | | | | | | |
| | | | CACCTT | | | | | | |
| | | | GCA | | | | | | |
| 148 | 4676 | 0.2452912 | CCCAGG | 94564303 | 94564319 | 27387 | 27403 | 17 | 0.22726156 |
| | | | AATCAC | | | | | | |
| | | | CTTGC | | | | | | |
| 149 | 4677 | 0.23007754 | CCCCAG | 94564302 | 94564319 | 27387 | 27404 | 18 | 0.2120479 |
| | | | GAATCA | | | | | | |
| | | | CCTTGC | | | | | | |
| 150 | 4678 | 0.20199157 | ACCCCA | 94564301 | 94564319 | 27387 | 27405 | 19 | 0.18396193 |
| | | | GGAATC | | | | | | |
| | | | ACCTTG | | | | | | |
| | | | C | | | | | | |
| 151 | 4679 | 0.22664884 | TACCCC | 94564300 | 94564319 | 27387 | 27406 | 20 | 0.2086192 |
| | | | AGGAAT | | | | | | |
| | | | CACCTT | | | | | | |
| | | | GC | | | | | | |
| 152 | 4680 | 0.24065276 | CTACCC | 94564299 | 94564319 | 27387 | 27407 | 21 | 0.22262312 |
| | | | CAGGAA | | | | | | |
| | | | TCACCTT | | | | | | |
| | | | GC | | | | | | |
| 153 | 4681 | 0.31432345 | CCCCAG | 94564302 | 94564318 | 27388 | 27404 | 17 | 0.29629381 |
| | | | GAATCA | | | | | | |
| | | | CCTTG | | | | | | |
| 154 | 4682 | 0.27533803 | ACCCCA | 94564301 | 94564318 | 27388 | 27405 | 18 | 0.25730839 |
| | | | GGAATC | | | | | | |
| | | | ACCTTG | | | | | | |
| 155 | 4683 | 0.35359545 | TACCCC | 94564300 | 94564318 | 27388 | 27406 | 19 | 0.33556581 |
| | | | AGGAAT | | | | | | |
| | | | CACCTT | | | | | | |
| | | | G | | | | | | |
| 156 | 4684 | 0.29786175 | CTACCC | 94564299 | 94564318 | 27388 | 27407 | 20 | 0.27983211 |
| | | | CAGGAA | | | | | | |
| | | | TCACCTT | | | | | | |
| | | | G | | | | | | |
| 157 | 4685 | 0.84163308 | GCTACC | 94564298 | 94564318 | 27388 | 27408 | 21 | 0.82360344 |
| | | | CCAGGA | | | | | | |
| | | | ATCACC | | | | | | |
| | | | TTG | | | | | | |
| 158 | 4686 | 0.28817154 | ACCCCA | 94564301 | 94564317 | 27389 | 27405 | 17 | 0.2701419 |
| | | | GGAATC | | | | | | |
| | | | ACCTT | | | | | | |
| 159 | 4687 | 0.25414838 | TACCCC | 94564300 | 94564317 | 27389 | 27406 | 18 | 0.23611874 |
| | | | AGGAAT | | | | | | |
| | | | CACCTT | | | | | | |
| 160 | 4689 | 0.87305965 | GCTACC | 94564298 | 94564317 | 27389 | 27408 | 20 | 0.85503 |
| | | | CCAGGA | | | | | | |
| | | | ATCACC | | | | | | |
| | | | TT | | | | | | |
| 161 | 4690 | 0.82648716 | TGCTAC | 94564297 | 94564317 | 27389 | 27409 | 21 | 0.80845752 |
| | | | CCCAGG | | | | | | |
| | | | AATCAC | | | | | | |
| | | | CTT | | | | | | |
| 162 | 4111 | 0.14924213 | CTACCC | 94564299 | 94564316 | 27390 | 27407 | 18 | 0.13121249 |
| | | | CAGGAA | | | | | | |
| | | | TCACCT | | | | | | |
| 163 | 4691 | 0.19736827 | TACCCC | 94564300 | 94564316 | 27390 | 27406 | 17 | 0.17933863 |
| | | | AGGAAT | | | | | | |
| | | | CACCT | | | | | | |
| 164 | 4692 | 0.3686295 | GCTACC | 94564298 | 94564316 | 27390 | 27408 | 19 | 0.35059986 |
| | | | CCAGGA | | | | | | |
| | | | ATCACC | | | | | | |
| | | | T | | | | | | |
| 165 | 4693 | 0.79136767 | TGCTAC | 94564297 | 94564316 | 27390 | 27409 | 20 | 0.77333803 |
| | | | CCCAGG | | | | | | |
| | | | AATCAC | | | | | | |
| | | | CT | | | | | | |
| 166 | 4694 | 0.82715435 | CTGCTA | 94564296 | 94564316 | 27390 | 27410 | 21 | 0.80912471 |
| | | | CCCCAG | | | | | | |
| | | | GAATCA | | | | | | |
| | | | CCT | | | | | | |
| 167 | 4695 | 0.19457674 | CTACCC | 94564299 | 94564315 | 27391 | 27407 | 17 | 0.1765471 |
| | | | CAGGAA | | | | | | |
| | | | TCACC | | | | | | |
| 168 | 4696 | 0.81253152 | GCTACC | 94564298 | 94564315 | 27391 | 27408 | 18 | 0.79450188 |
| | | | CCAGGA | | | | | | |
| | | | ATCACC | | | | | | |
| 169 | 4697 | 0.77605781 | TGCTAC | 94564297 | 94564315 | 27391 | 27409 | 19 | 0.75802817 |
| | | | CCCAGG | | | | | | |
| | | | AATCAC | | | | | | |
| | | | C | | | | | | |
| 170 | 4698 | 0.8033507 | CTGCTA | 94564296 | 94564315 | 27391 | 27410 | 20 | 0.78532106 |
| | | | CCCCAG | | | | | | |
| | | | GAATCA | | | | | | |
| | | | CC | | | | | | |
| 171 | 4699 | 0.76580739 | TCTGCT | 94564295 | 94564315 | 27391 | 27411 | 21 | 0.74777775 |
| | | | ACCCCA | | | | | | |
| | | | GGAATC | | | | | | |
| | | | ACC | | | | | | |
| 172 | 4700 | 0.20463344 | GCTACC | 94564298 | 94564314 | 27392 | 27408 | 17 | 0.1866038 |
| | | | CCAGGA | | | | | | |
| | | | ATCAC | | | | | | |
| 173 | 4701 | 0.19263715 | TGCTAC | 94564297 | 94564314 | 27392 | 27409 | 18 | 0.17460751 |
| | | | CCCAGG | | | | | | |
| | | | AATCAC | | | | | | |
| 174 | 4702 | 0.25031864 | CTGCTA | 94564296 | 94564314 | 27392 | 27410 | 19 | 0.232289 |
| | | | CCCCAG | | | | | | |
| | | | GAATCA | | | | | | |
| | | | C | | | | | | |
| 175 | 4703 | 0.22951121 | TCTGCT | 94564295 | 94564314 | 27392 | 27411 | 20 | 0.21148157 |
| | | | ACCCCA | | | | | | |
| | | | GGAATC | | | | | | |
| | | | AC | | | | | | |
| 176 | 4704 | 0.1954459 | CTCTGCT | 94564294 | 94564314 | 27392 | 27412 | 21 | 0.17741626 |
| | | | ACCCCA | | | | | | |
| | | | GGAATC | | | | | | |
| | | | AC | | | | | | |
| 177 | 4092 | 0.13500456 | TGCTAC | 94564297 | 94564313 | 27393 | 27409 | 17 | 0.11697492 |
| | | | CCCAGG | | | | | | |
| | | | AATCA | | | | | | |
| 178 | 4705 | 0.16096575 | CTGCTA | 94564296 | 94564313 | 27393 | 27410 | 18 | 0.1429361 |
| | | | CCCCAG | | | | | | |
| | | | GAATCA | | | | | | |
| 179 | 4706 | 0.158593 | TCTGCT | 94564295 | 94564313 | 27393 | 27411 | 19 | 0.14056336 |
| | | | ACCCCA | | | | | | |
| | | | GGAATC | | | | | | |
| | | | A | | | | | | |
| 180 | 4707 | 0.13411114 | CTCTGCT | 94564294 | 94564313 | 27393 | 27412 | 20 | 0.1160815 |
| | | | ACCCCA | | | | | | |
| | | | GGAATC | | | | | | |
| | | | A | | | | | | |
| 181 | 4708 | 0.20781816 | GCTCTG | 94564293 | 94564313 | 27393 | 27413 | 21 | 0.18978852 |
| | | | CTACCC | | | | | | |
| | | | CAGGAA | | | | | | |
| | | | TCA | | | | | | |
| 182 | 4709 | 0.0784893 | CTGCTA | 94564296 | 94564312 | 27394 | 27410 | 17 | 0.06045966 |
| | | | CCCCAG | | | | | | |
| | | | GAATC | | | | | | |
| 183 | 4710 | 0.0891908 | TCTGCT | 94564295 | 94564312 | 27394 | 27411 | 18 | 0.07116116 |
| | | | ACCCCA | | | | | | |
| | | | GGAATC | | | | | | |
| 184 | 4711 | 0.05290537 | CTCTGCT | 94564294 | 94564312 | 27394 | 27412 | 19 | 0.03487573 |
| | | | ACCCCA | | | | | | |
| | | | GGAATC | | | | | | |
| 185 | 4712 | 0.15401065 | GCTCTG | 94564293 | 94564312 | 27394 | 27413 | 20 | 0.13598101 |
| | | | CTACCC | | | | | | |
| | | | CAGGAA | | | | | | |
| | | | TC | | | | | | |
| 186 | 4713 | 0.09604376 | GGCTCT | 94564292 | 94564312 | 27394 | 27414 | 21 | 0.07801412 |
| | | | GCTACC | | | | | | |
| | | | CCAGGA | | | | | | |
| | | | ATC | | | | | | |
| 187 | 4714 | 0.13741142 | TCTGCT | 94564295 | 94564311 | 27395 | 27411 | 17 | 0.11938178 |
| | | | ACCCCA | | | | | | |
| | | | GGAAT | | | | | | |
| 188 | 4715 | 0.1047728 | CTCTGCT | 94564294 | 94564311 | 27395 | 27412 | 18 | 0.08674316 |
| | | | ACCCCA | | | | | | |
| | | | GGAAT | | | | | | |
| 189 | 4716 | 0.23153099 | GCTCTG | 94564293 | 94564311 | 27395 | 27413 | 19 | 0.21350135 |
| | | | CTACCC | | | | | | |
| | | | CAGGAA | | | | | | |
| | | | T | | | | | | |
| 190 | 4717 | 0.27661374 | GGCTCT | 94564292 | 94564311 | 27395 | 27414 | 20 | 0.2585841 |
| | | | GCTACC | | | | | | |
| | | | CCAGGA | | | | | | |
| | | | AT | | | | | | |
| 191 | 4139 | 0.15666069 | AGGCTC | 94564291 | 94564310 | 27396 | 27415 | 20 | 0.13863105 |
| | | | TGCTAC | | | | | | |
| | | | CCCAGG | | | | | | |
| | | | AA | | | | | | |
| 192 | 4718 | 0.13584046 | CTCTGCT | 94564294 | 94564310 | 27396 | 27412 | 17 | 0.11781081 |
| | | | ACCCCA | | | | | | |
| | | | GGAA | | | | | | |
| 193 | 4719 | 0.48672796 | GCTCTG | 94564293 | 94564310 | 27396 | 27413 | 18 | 0.46869832 |
| | | | CTACCC | | | | | | |
| | | | CAGGAA | | | | | | |
| 194 | 4720 | 0.37749689 | GGCTCT | 94564292 | 94564310 | 27396 | 27414 | 19 | 0.35946725 |
| | | | GCTACC | | | | | | |
| | | | CCAGGA | | | | | | |
| | | | A | | | | | | |
| 195 | 4721 | 0.50288272 | GCTCTG | 94564293 | 94564309 | 27397 | 27413 | 17 | 0.48485308 |
| | | | CTACCC | | | | | | |
| | | | CAGGA | | | | | | |
| 196 | 4722 | 0.43230889 | GGCTCT | 94564292 | 94564309 | 27397 | 27414 | 18 | 0.41427924 |
| | | | GCTACC | | | | | | |
| | | | CCAGGA | | | | | | |
| 197 | 4723 | 0.19564733 | GGCTCT | 94564292 | 94564308 | 27398 | 27414 | 17 | 0.17761769 |
| | | | GCTACC | | | | | | |
| | | | CCAGG | | | | | | |
| 198 | 4126 | 0.04292774 | CGTGAG | 94564287 | 94564305 | 27401 | 27419 | 19 | 0.02489809 |
| | | | GCTCTG | | | | | | |
| | | | CTACCC | | | | | | |
| | | | C | | | | | | |
| 199 | 4112 | 0.00596452 | AATTCG | 94564283 | 94564300 | 27406 | 27423 | 18 | −0.0120651 |
| | | | TGAGGC | | | | | | |
| | | | TCTGCT | | | | | | |
| 200 | 4127 | 0.01072732 | GGTCAA | 94564279 | 94564297 | 27409 | 27427 | 19 | −0.0073023 |
| | | | TTCGTG | | | | | | |
| | | | AGGCTC | | | | | | |
| | | | T | | | | | | |
| 201 | 4093 | 0.01129358 | CAAGGT | 94564276 | 94564292 | 27414 | 27430 | 17 | −0.0067361 |
| | | | CAATTC | | | | | | |
| | | | GTGAG | | | | | | |
| 202 | 4150 | 0.00813254 | CCTCCC | 94564270 | 94564290 | 27416 | 27436 | 21 | −0.0098971 |
| | | | CAAGGT | | | | | | |
| | | | CAATTC | | | | | | |
| | | | GTG | | | | | | |
| 203 | 4151 | 0.01433631 | GGCTCA | 94564261 | 94564281 | 27425 | 27445 | 21 | −0.0036933 |
| | | | CGCCCT | | | | | | |
| | | | CCCCAA | | | | | | |
| | | | GGT | | | | | | |
| 204 | 4094 | 0.02260101 | CAGGCT | 94564259 | 94564275 | 27431 | 27447 | 17 | 0.00457137 |
| | | | CACGCC | | | | | | |
| | | | CTCCC | | | | | | |
| 205 | 4113 | 0.01461124 | CACCAG | 94564256 | 94564273 | 27433 | 27450 | 18 | −0.0034184 |
| | | | GCTCAC | | | | | | |
| | | | GCCCTC | | | | | | |
| 206 | 4140 | 0.02414921 | CCAGAA | 94564250 | 94564269 | 27437 | 27456 | 20 | 0.00611957 |
| | | | CACCAG | | | | | | |
| | | | GCTCAC | | | | | | |
| | | | GC | | | | | | |

TABLE 4

| | | | | | | Start | Stop | | |
|---|---|---|---|---|---|---|---|---|---|
| | | | | | | on | on | | |
| SEQ | | | | Start | | SEQ | SEQ | | |
| ID | DG | | | Chr1 | End Chrl | ID | ID | | |
| NO: | ID | PSI | Sequence | [hg19/b37] | [hg19/b37] | NO: 1 | NO: 1 | length | dPSI |
| 2 | 4128 | 0.00852007 | ATACCT | 94564626 | 94564645 | 27061 | 27080 | 20 | -0.0095096 |
| | | | TGTGTT | | | | | | |
| | | | ACATGG | | | | | | |
| | | | CG | | | | | | |
| 3 | 4073 | 0.02900731 | GGGAAT | 94564622 | 94564638 | 27068 | 27084 | 17 | 0.01097767 |
| | | | ACCTTG | | | | | | |
| | | | TGTTA | | | | | | |
| 4 | 4141 | 0.07391646 | AGAACC | 94564615 | 94564635 | 27071 | 27091 | 21 | 0.05588682 |
| | | | TGGGAA | | | | | | |
| | | | TACCTT | | | | | | |
| | | | GTG | | | | | | |
| 5 | 4114 | 0.01283934 | CTAACC | 94564606 | 94564624 | 27082 | 27100 | 19 | -0.0051903 |
| | | | CACAGA | | | | | | |
| | | | ACCTGG | | | | | | |
| | | | G | | | | | | |
| 6 | 4129 | 0.01265609 | CCACGT | 94564599 | 94564618 | 27088 | 27107 | 20 | -0.0053736 |
| | | | CCTAAC | | | | | | |
| | | | CCACAG | | | | | | |
| | | | AA | | | | | | |
| 7 | 4130 | 0.01522623 | GAAAGA | 94564590 | 94564609 | 27097 | 27116 | 20 | -0.0028034 |
| | | | CACCCA | | | | | | |
| | | | CGTCCT | | | | | | |
| | | | AA | | | | | | |
| 8 | 4095 | 0.00990721 | TAGGAA | 94564587 | 94564604 | 27102 | 27119 | 18 | -0.0081224 |
| | | | AGACAC | | | | | | |
| | | | CCACGT | | | | | | |
| 9 | 4074 | 0.02108604 | GGTAGG | 94564585 | 94564601 | 27105 | 27121 | 17 | 0.0030564 |
| | | | AAAGAC | | | | | | |
| | | | ACCCA | | | | | | |
| 10 | 4115 | 0.02587134 | CCCTGT | 94564579 | 94564597 | 27109 | 27127 | 19 | 0.0078417 |
| | | | GGTAGG | | | | | | |
| | | | AAAGAC | | | | | | |
| | | | A | | | | | | |
| 11 | 4096 | 0.01078192 | CTGCCC | 94564576 | 94564593 | 27113 | 27130 | 18 | -0.0072477 |
| | | | TGTGGT | | | | | | |
| | | | AGGAAA | | | | | | |
| 12 | 4075 | 0.01630967 | AACTGC | 94564574 | 94564590 | 27116 | 27132 | 17 | -0.00172 |
| | | | CCTGTG | | | | | | |
| | | | GTAGG | | | | | | |
| 13 | 4076 | 0.01604054 | GAAACT | 94564572 | 94564588 | 27118 | 27134 | 17 | -0.0019891 |
| | | | GCCCTG | | | | | | |
| | | | TGGTA | | | | | | |
| 14 | 4097 | 0.00826475 | CTAGAA | 94564569 | 94564586 | 27120 | 27137 | 18 | -0.0097649 |
| | | | ACTGCC | | | | | | |
| | | | CTGTGG | | | | | | |
| 15 | 4131 | 0.01220225 | GGCAAC | 94564562 | 94564581 | 27125 | 27144 | 20 | -0.0058274 |
| | | | ACTAGA | | | | | | |
| | | | AACTGC | | | | | | |
| | | | CC | | | | | | |
| 16 | 4142 | 0.01869085 | GGAGAA | 94564554 | 94564574 | 27132 | 27152 | 21 | 0.00066121 |
| | | | GAGGCA | | | | | | |
| | | | ACACTA | | | | | | |
| | | | GAA | | | | | | |
| 17 | 4098 | 0.0129716 | CAGGGA | 94564551 | 94564568 | 27138 | 27155 | 18 | -0.005058 |
| | | | GAAGAG | | | | | | |
| | | | GCAACA | | | | | | |
| 18 | 4077 | 0.01036923 | ACTGCA | 94564547 | 94564563 | 27143 | 27159 | 17 | -0.0076604 |
| | | | GGGAGA | | | | | | |
| | | | AGAGG | | | | | | |
| 19 | 4132 | 0.01542554 | GAGCGA | 94564541 | 94564560 | 27146 | 27165 | 20 | -0.0026041 |
| | | | ACTGCA | | | | | | |
| | | | GGGAGA | | | | | | |
| | | | AG | | | | | | |
| 20 | 4133 | 0.0144133 | TCCATG | 94564536 | 94564555 | 27151 | 27170 | 20 | -0.0036163 |
| | | | AGCGAA | | | | | | |
| | | | CTGCAG | | | | | | |
| | | | GG | | | | | | |
| 21 | 4134 | 0.01992633 | GGGACT | 94564531 | 94564550 | 27156 | 27175 | 20 | 0.00189669 |
| | | | CCATGA | | | | | | |
| | | | GCGAAC | | | | | | |
| | | | TG | | | | | | |
| 22 | 4078 | 0.01516713 | TCCGGG | 94564528 | 94564544 | 27162 | 27178 | 17 | -0.0028625 |
| | | | ACTCCA | | | | | | |
| | | | TGAGC | | | | | | |
| 23 | 4143 | 0.01312488 | AGCGCC | 94564519 | 94564539 | 27167 | 27187 | 21 | -0.0049048 |
| | | | AGGTCC | | | | | | |
| | | | GGGACT | | | | | | |
| | | | CCA | | | | | | |
| 24 | 4144 | 0.01627758 | GTCCTTC | 94564512 | 94564532 | 27174 | 27194 | 21 | -0.0017521 |
| | | | AGCGCC | | | | | | |
| | | | AGGTCC | | | | | | |
| | | | GG | | | | | | |
| 25 | 4145 | 0.01750626 | CAGGCG | 94564504 | 94564524 | 27182 | 27202 | 21 | -0.0005234 |
| | | | ATGTCC | | | | | | |
| | | | TTCAGC | | | | | | |
| | | | GCC | | | | | | |
| 26 | 4116 | 0.01152383 | CCTCGC | 94564496 | 94564514 | 27192 | 27210 | 19 | -0.0065058 |
| | | | TGCAGG | | | | | | |
| | | | CGATGT | | | | | | |
| | | | C | | | | | | |
| 27 | 4099 | 0.03132164 | GGAGGG | 94564490 | 94564507 | 27199 | 27216 | 18 | 0.013292 |
| | | | CCTCGC | | | | | | |
| | | | TGCAGG | | | | | | |
| 28 | 4100 | 0.04411962 | GCTCCA | 94564484 | 94564501 | 27205 | 27222 | 18 | 0.02608998 |
| | | | GGAGGG | | | | | | |
| | | | CCTCGC | | | | | | |
| 29 | 4079 | 0.02378016 | GCGCTC | 94564482 | 94564498 | 27208 | 27224 | 17 | 0.00575051 |
| | | | CAGGAG | | | | | | |
| | | | GGCCT | | | | | | |
| 30 | 4101 | 0.01407391 | TGAAGC | 94564478 | 94564495 | 27211 | 27228 | 18 | -0.0039557 |
| | | | GCTCCA | | | | | | |
| | | | GGAGGG | | | | | | |
| 31 | 4135 | 0.0122176 | GAAGAT | 94564470 | 94564489 | 27217 | 27236 | 20 | -0.005812 |
| | | | GATGAA | | | | | | |
| | | | GCGCTC | | | | | | |
| | | | CA | | | | | | |
| 32 | 4117 | 0.00913255 | TGGCTG | 94564465 | 94564483 | 27223 | 27241 | 19 | -0.0088971 |
| | | | AAGATG | | | | | | |
| | | | ATGAAG | | | | | | |
| | | | C | | | | | | |
| 33 | 4118 | 0.01154571 | TCTCTG | 94564461 | 94564479 | 27227 | 27245 | 19 | -0.0064839 |
| | | | GCTGAA | | | | | | |
| | | | GATGAT | | | | | | |
| | | | G | | | | | | |
| 34 | 4080 | 0.01103206 | CGTCTCT | 94564459 | 94564475 | 27231 | 27247 | 17 | -0.0069976 |
| | | | GGCTGA | | | | | | |
| | | | AGAT | | | | | | |
| 35 | 4136 | 0.01414565 | TTGCCC | 94564451 | 94564470 | 27236 | 27255 | 20 | -0.003884 |
| | | | CGCGTC | | | | | | |
| | | | TCTGGC | | | | | | |
| | | | TG | | | | | | |
| 36 | 4102 | 0.01511915 | CACCGT | 94564443 | 94564460 | 27246 | 27263 | 18 | -0.0029105 |
| | | | CTTTGCC | | | | | | |
| | | | CCGCG | | | | | | |
| 37 | 4146 | 0.01070549 | ATAGCG | 94564437 | 94564457 | 27249 | 27269 | 21 | -0.0073241 |
| | | | CACCGT | | | | | | |
| | | | CTTTGCC | | | | | | |
| | | | CC | | | | | | |
| 38 | 4081 | 0.01051709 | GGCATA | 94564434 | 94564450 | 27256 | 27272 | 17 | -0.0075125 |
| | | | GCGCAC | | | | | | |
| | | | CGTCT | | | | | | |
| 39 | 4103 | 0.01277919 | CAGGGC | 94564431 | 94564448 | 27258 | 27275 | 18 | -0.0052504 |
| | | | ATAGCG | | | | | | |
| | | | CACCGT | | | | | | |
| 40 | 4119 | 0.01240376 | GAGCAC | 94564426 | 94564444 | 27262 | 27280 | 19 | -0.0056259 |
| | | | AGGGCA | | | | | | |
| | | | TAGCGC | | | | | | |
| | | | A | | | | | | |
| 41 | 4082 | 0.01090273 | AGAGGG | 94564421 | 94564437 | 27269 | 27285 | 17 | -0.0071269 |
| | | | AGCACA | | | | | | |
| | | | GGGCA | | | | | | |
| 42 | 4120 | 0.01957139 | TGGGAG | 94564417 | 94564435 | 27271 | 27289 | 19 | 0.00154175 |
| | | | AGGGAG | | | | | | |
| | | | CACAGG | | | | | | |
| | | | G | | | | | | |
| 43 | 4147 | 0.00065793 | TAGGGT | 94564407 | 94564427 | 27279 | 27299 | 21 | -0.0173717 |
| | | | GCCCTG | | | | | | |
| | | | GGAGAG | | | | | | |
| | | | GGA | | | | | | |
| 44 | 4148 | 0.00875718 | TATCCA | 94564398 | 94564418 | 27288 | 27308 | 21 | -0.0092725 |
| | | | CTGTAG | | | | | | |
| | | | GGTGCC | | | | | | |
| | | | CTG | | | | | | |
| 45 | 4083 | 0.01195793 | TCTTCTA | 94564393 | 94564409 | 27297 | 27313 | 17 | -0.0060717 |
| | | | TCCACT | | | | | | |
| | | | GTAG | | | | | | |
| 46 | 4084 | 0.00608376 | AGTGTC | 94564389 | 94564405 | 27301 | 27317 | 17 | -0.0119459 |
| | | | TTCTATC | | | | | | |
| | | | CACT | | | | | | |
| 47 | 4104 | 0.00557296 | CAGAGT | 94564386 | 94564403 | 27303 | 27320 | 18 | -0.0124567 |
| | | | GTCTTCT | | | | | | |
| | | | ATCCA | | | | | | |
| 48 | 4137 | 0.01727846 | GTTGGC | 94564377 | 94564396 | 27310 | 27329 | 20 | -0.0007512 |
| | | | ATACAG | | | | | | |
| | | | AGTGTC | | | | | | |
| | | | TT | | | | | | |
| 49 | 4121 | 0.00523364 | CCACGT | 94564373 | 94564391 | 27315 | 27333 | 19 | -0.012796 |
| | | | TGGCAT | | | | | | |
| | | | ACAGAG | | | | | | |
| | | | T | | | | | | |
| 50 | 4105 | 0.0132362 | AAGTCC | 94564369 | 94564386 | 27320 | 27337 | 18 | -0.0047934 |
| | | | ACGTTG | | | | | | |
| | | | GCATAC | | | | | | |
| 51 | 4106 | 0.01811265 | AAGAAG | 94564366 | 94564383 | 27323 | 27340 | 18 | 8.3006E-05 |
| | | | TCCACG | | | | | | |
| | | | TTGGCA | | | | | | |
| 52 | 4122 | 0.00735466 | GCTTGA | 94564361 | 94564379 | 27327 | 27345 | 19 | -0.010675 |
| | | | AGAAGT | | | | | | |
| | | | CCACGT | | | | | | |
| | | | T | | | | | | |
| 53 | 4107 | 0.00854169 | AAGAGC | 94564357 | 94564374 | 27332 | 27349 | 18 | -0.009488 |
| | | | TTGAAG | | | | | | |
| | | | AAGTCC | | | | | | |
| 54 | 4108 | 0.00238904 | CGGAAG | 94564354 | 94564371 | 27335 | 27352 | 18 | -0.0156406 |
| | | | AGCTTG | | | | | | |
| | | | AAGAAG | | | | | | |
| 55 | 4085 | 0.00493693 | AACACG | 94564350 | 94564366 | 27340 | 27356 | 17 | -0.0130927 |
| | | | GAAGAG | | | | | | |
| | | | CTTGA | | | | | | |
| 56 | 4123 | 0.00374432 | CTTACA | 94564345 | 94564363 | 27343 | 27361 | 19 | -0.0142853 |
| | | | ACACGG | | | | | | |
| | | | AAGAGC | | | | | | |
| | | | T | | | | | | |
| 57 | 4109 | 0.01006963 | CTCCCTT | 94564341 | 94564358 | 27348 | 27365 | 18 | -0.00796 |
| | | | ACAACA | | | | | | |
| | | | CGGAA | | | | | | |
| 58 | 4149 | 0.01178247 | CCAAAC | 94564333 | 94564353 | 27353 | 27373 | 21 | -0.0062472 |
| | | | CCCTCC | | | | | | |
| | | | CTTACA | | | | | | |
| | | | ACA | | | | | | |
| 59 | 4086 | 0.00939203 | CAGCCA | 94564330 | 94564346 | 27360 | 27376 | 17 | -0.0086376 |
| | | | AACCCC | | | | | | |
| | | | TCCCT | | | | | | |
| 60 | 4087 | 0.03079641 | AGCAGC | 94564328 | 94564344 | 27362 | 27378 | 17 | 0.01276677 |
| | | | CAAACC | | | | | | |
| | | | CCTCC | | | | | | |
| 61 | 4088 | 0.29179785 | CGAGCA | 94564326 | 94564342 | 27364 | 27380 | 17 | 0.27376821 |
| | | | GCCAAA | | | | | | |
| | | | CCCCT | | | | | | |
| 62 | 4110 | 0.08947937 | TGGCGA | 94564323 | 94564340 | 27366 | 27383 | 18 | 0.07144973 |
| | | | GCAGCC | | | | | | |
| | | | AAACCC | | | | | | |
| 63 | 4138 | 0.22120365 | TGCAAT | 94564317 | 94564336 | 27370 | 27389 | 20 | 0.20317401 |
| | | | TGGCGA | | | | | | |
| | | | GCAGCC | | | | | | |
| | | | AA | | | | | | |
| 64 | 4597 | 0.47513581 | AATTGG | 94564320 | 94564336 | 27370 | 27386 | 17 | 0.45710617 |
| | | | CGAGCA | | | | | | |
| | | | GCCAA | | | | | | |
| 65 | 4598 | 0.72634299 | CAATTG | 94564319 | 94564336 | 27370 | 27387 | 18 | 0.70831335 |
| | | | GCGAGC | | | | | | |
| | | | AGCCAA | | | | | | |
| 66 | 4599 | 0.51076267 | GCAATT | 94564318 | 94564336 | 27370 | 27388 | 19 | 0.49273303 |
| | | | GGCGAG | | | | | | |
| | | | CAGCCA | | | | | | |
| | | | A | | | | | | |
| 67 | 4600 | 0.23376829 | TTGCAA | 94564316 | 94564336 | 27370 | 27390 | 21 | 0.21573864 |
| | | | TTGGCG | | | | | | |
| | | | AGCAGC | | | | | | |
| | | | CAA | | | | | | |
| 68 | 4601 | 0.74320192 | CAATTG | 94564319 | 94564335 | 27371 | 27387 | 17 | 0.72517227 |
| | | | GCGAGC | | | | | | |
| | | | AGCCA | | | | | | |
| 69 | 4602 | 0.59473771 | GCAATT | 94564318 | 94564335 | 27371 | 27388 | 18 | 0.57670806 |
| | | | GGCGAG | | | | | | |
| | | | CAGCCA | | | | | | |
| 70 | 4603 | 0.66762071 | TGCAAT | 94564317 | 94564335 | 27371 | 27389 | 19 | 0.64959107 |
| | | | TGGCGA | | | | | | |
| | | | GCAGCC | | | | | | |
| | | | A | | | | | | |
| 71 | 4604 | 0.58471501 | TTGCAA | 94564316 | 94564335 | 27371 | 27390 | 20 | 0.56668537 |
| | | | TTGGCG | | | | | | |
| | | | AGCAGC | | | | | | |
| | | | CA | | | | | | |
| 72 | 4605 | 0.65609249 | CTTGCA | 94564315 | 94564335 | 27371 | 27391 | 21 | 0.63806285 |
| | | | ATTGGC | | | | | | |
| | | | GAGCAG | | | | | | |
| | | | CCA | | | | | | |
| 73 | 4606 | 0.72313482 | GCAATT | 94564318 | 94564334 | 27372 | 27388 | 17 | 0.70510518 |
| | | | GGCGAG | | | | | | |
| | | | CAGCC | | | | | | |
| 74 | 4607 | 0.8716546 | TGCAAT | 94564317 | 94564334 | 27372 | 27389 | 18 | 0.85362496 |
| | | | TGGCGA | | | | | | |
| | | | GCAGCC | | | | | | |
| 75 | 4608 | 0.74564326 | TTGCAA | 94564316 | 94564334 | 27372 | 27390 | 19 | 0.72761362 |
| | | | TTGGCG | | | | | | |
| | | | AGCAGC | | | | | | |
| | | | C | | | | | | |
| 76 | 4609 | 0.78299129 | CTTGCA | 94564315 | 94564334 | 27372 | 27391 | 20 | 0.76496165 |
| | | | ATTGGC | | | | | | |
| | | | GAGCAG | | | | | | |
| | | | CC | | | | | | |
| 77 | 4610 | 0.67006409 | CCTTGC | 94564314 | 94564334 | 27372 | 27392 | 21 | 0.65203445 |
| | | | AATTGG | | | | | | |
| | | | CGAGCA | | | | | | |
| | | | GCC | | | | | | |
| 78 | 4611 | 0.85497825 | TGCAAT | 94564317 | 94564333 | 27373 | 27389 | 17 | 0.83694861 |
| | | | TGGCGA | | | | | | |
| | | | GCAGC | | | | | | |
| 79 | 4612 | 0.52063801 | TTGCAA | 94564316 | 94564333 | 27373 | 27390 | 18 | 0.50260837 |
| | | | TTGGCG | | | | | | |
| | | | AGCAGC | | | | | | |
| 80 | 4613 | 0.68203054 | CTTGCA | 94564315 | 94564333 | 27373 | 27391 | 19 | 0.6640009 |
| | | | ATTGGC | | | | | | |
| | | | GAGCAG | | | | | | |
| | | | C | | | | | | |
| 81 | 4614 | 0.37065258 | CCTTGC | 94564314 | 94564333 | 27373 | 27392 | 20 | 0.35262294 |
| | | | AATTGG | | | | | | |
| | | | CGAGCA | | | | | | |
| | | | GC | | | | | | |
| 82 | 4615 | 0.4217697 | ACCTTG | 94564313 | 94564333 | 27373 | 27393 | 21 | 0.40374006 |
| | | | CAATTG | | | | | | |
| | | | GCGAGC | | | | | | |
| | | | AGC | | | | | | |
| 83 | 4616 | 0.71775973 | TTGCAA | 94564316 | 94564332 | 27374 | 27390 | 17 | 0.69973009 |
| | | | TTGGCG | | | | | | |
| | | | AGCAG | | | | | | |
| 84 | 4617 | 0.7403724 | CTTGCA | 94564315 | 94564332 | 27374 | 27391 | 18 | 0.72234275 |
| | | | ATTGGC | | | | | | |
| | | | GAGCAG | | | | | | |
| 85 | 4618 | 0.55691816 | CCTTGC | 94564314 | 94564332 | 27374 | 27392 | 19 | 0.53888852 |
| | | | AATTGG | | | | | | |
| | | | CGAGCA | | | | | | |
| | | | G | | | | | | |
| 86 | 4619 | 0.81497515 | ACCTTG | 94564313 | 94564332 | 27374 | 27393 | 20 | 0.79694551 |
| | | | CAATTG | | | | | | |
| | | | GCGAGC | | | | | | |
| | | | AG | | | | | | |
| 87 | 4620 | 0.72321098 | CACCTT | 94564312 | 94564332 | 27374 | 27394 | 21 | 0.70518134 |
| | | | GCAATT | | | | | | |
| | | | GGCGAG | | | | | | |
| | | | CAG | | | | | | |
| 88 | 4089 | 0.82127394 | CTTGCA | 94564315 | 94564331 | 27375 | 27391 | 17 | 0.8032443 |
| | | | ATTGGC | | | | | | |
| | | | GAGCA | | | | | | |
| 89 | 4621 | 0.88664722 | CCTTGC | 94564314 | 94564331 | 27375 | 27392 | 18 | 0.86861758 |
| | | | AATTGG | | | | | | |
| | | | CGAGCA | | | | | | |
| 90 | 4622 | 0.87451707 | ACCTTG | 94564313 | 94564331 | 27375 | 27393 | 19 | 0.85648742 |
| | | | CAATTG | | | | | | |
| | | | GCGAGC | | | | | | |
| | | | A | | | | | | |
| 91 | 4623 | 0.89267292 | CACCTT | 94564312 | 94564331 | 27375 | 27394 | 20 | 0.87464328 |
| | | | GCAATT | | | | | | |
| | | | GGCGAG | | | | | | |
| | | | CA | | | | | | |
| 92 | 4624 | 0.56133913 | TCACCTT | 94564311 | 94564331 | 27375 | 27395 | 21 | 0.54330949 |
| | | | GCAATT | | | | | | |
| | | | GGCGAG | | | | | | |
| | | | CA | | | | | | |
| 93 | 4625 | 0.73532055 | CCTTGC | 94564314 | 94564330 | 27376 | 27392 | 17 | 0.71729091 |
| | | | AATTGG | | | | | | |
| | | | CGAGC | | | | | | |
| 94 | 4626 | 0.82730273 | ACCTTG | 94564313 | 94564330 | 27376 | 27393 | 18 | 0.80927309 |
| | | | CAATTG | | | | | | |
| | | | GCGAGC | | | | | | |
| 95 | 4627 | 0.8159207 | CACCTT | 94564312 | 94564330 | 27376 | 27394 | 19 | 0.79789106 |
| | | | GCAATT | | | | | | |
| | | | GGCGAG | | | | | | |
| | | | C | | | | | | |
| 96 | 4628 | 0.59808349 | TCACCTT | 94564311 | 94564330 | 27376 | 27395 | 20 | 0.58005385 |
| | | | GCAATT | | | | | | |
| | | | GGCGAG | | | | | | |
| | | | C | | | | | | |
| 97 | 4629 | 0.67216645 | ATCACC | 94564310 | 94564330 | 27376 | 27396 | 21 | 0.65413681 |
| | | | TTGCAA | | | | | | |
| | | | TTGGCG | | | | | | |
| | | | AGC | | | | | | |
| 98 | 4090 | 0.88361284 | ACCTTG | 94564313 | 94564329 | 27377 | 27393 | 17 | 0.8655832 |
| | | | CAATTG | | | | | | |
| | | | GCGAG | | | | | | |
| 99 | 4630 | 0.86571736 | CACCTT | 94564312 | 94564329 | 27377 | 27394 | 18 | 0.84768772 |
| | | | GCAATT | | | | | | |
| | | | GGCGAG | | | | | | |
| 100 | 4631 | 0.92856185 | TCACCTT | 94564311 | 94564329 | 27377 | 27395 | 19 | 0.91053221 |
| | | | GCAATT | | | | | | |
| | | | GGCGAG | | | | | | |
| 101 | 4632 | 0.88361444 | ATCACC | 94564310 | 94564329 | 27377 | 27396 | 20 | 0.8655848 |
| | | | TTGCAA | | | | | | |
| | | | TTGGCG | | | | | | |
| | | | AG | | | | | | |
| 102 | 4633 | 0.92078171 | AATCAC | 94564309 | 94564329 | 27377 | 27397 | 21 | 0.90275207 |
| | | | CTTGCA | | | | | | |
| | | | ATTGGC | | | | | | |
| | | | GAG | | | | | | |
| 103 | 4634 | 0.92540904 | CACCTT | 94564312 | 94564328 | 27378 | 27394 | 17 | 0.9073794 |
| | | | GCAATT | | | | | | |
| | | | GGCGA | | | | | | |
| 104 | 4635 | 0.8837001 | TCACCTT | 94564311 | 94564328 | 27378 | 27395 | 18 | 0.86567046 |
| | | | GCAATT | | | | | | |
| | | | GGCGA | | | | | | |
| 105 | 4636 | 0.84273478 | ATCACC | 94564310 | 94564328 | 27378 | 27396 | 19 | 0.82470514 |
| | | | TTGCAA | | | | | | |
| | | | TTGGCG | | | | | | |
| | | | A | | | | | | |
| 106 | 4637 | 0.90290584 | AATCAC | 94564309 | 94564328 | 27378 | 27397 | 20 | 0.8848762 |
| | | | CTTGCA | | | | | | |
| | | | ATTGGC | | | | | | |
| | | | GA | | | | | | |
| 107 | 4638 | 0.77352068 | GAATCA | 94564308 | 94564328 | 27378 | 27398 | 21 | 0.75549104 |
| | | | CCTTGC | | | | | | |
| | | | AATTGG | | | | | | |
| | | | CGA | | | | | | |
| 108 | 4124 | 0.87866651 | AATCAC | 94564309 | 94564327 | 27379 | 27397 | 19 | 0.86063687 |
| | | | CTTGCA | | | | | | |
| | | | ATTGGC | | | | | | |
| | | | G | | | | | | |
| 109 | 4639 | 0.91849987 | TCACCTT | 94564311 | 94564327 | 27379 | 27395 | 17 | 0.90047023 |
| | | | GCAATT | | | | | | |
| | | | GGCG | | | | | | |
| 110 | 4640 | 0.79921991 | ATCACC | 94564310 | 94564327 | 27379 | 27396 | 18 | 0.78119027 |
| | | | TTGCAA | | | | | | |
| | | | TTGGCG | | | | | | |
| 111 | 4641 | 0.84375916 | GAATCA | 94564308 | 94564327 | 27379 | 27398 | 20 | 0.82572952 |
| | | | CCTTGC | | | | | | |
| | | | AATTGG | | | | | | |
| | | | CG | | | | | | |
| 112 | 4642 | 0.89609416 | GGAATC | 94564307 | 94564327 | 27379 | 27399 | 21 | 0.87806452 |
| | | | ACCTTG | | | | | | |
| | | | CAATTG | | | | | | |
| | | | GCG | | | | | | |
| 113 | 4643 | 0.9454494 | ATCACC | 94564310 | 94564326 | 27380 | 27396 | 17 | 0.92741976 |
| | | | TTGCAA | | | | | | |
| | | | TTGGC | | | | | | |
| 114 | 4644 | 0.92651139 | AATCAC | 94564309 | 94564326 | 27380 | 27397 | 18 | 0.90848175 |
| | | | CTTGCA | | | | | | |
| | | | ATTGGC | | | | | | |
| 115 | 4645 | 0.85076613 | GAATCA | 94564308 | 94564326 | 27380 | 27398 | 19 | 0.83273649 |
| | | | CCTTGC | | | | | | |
| | | | AATTGG | | | | | | |
| | | | C | | | | | | |
| 116 | 4646 | 0.8129502 | GGAATC | 94564307 | 94564326 | 27380 | 27399 | 20 | 0.79492056 |
| | | | ACCTTG | | | | | | |
| | | | CAATTG | | | | | | |
| | | | GC | | | | | | |
| 117 | 4647 | 0.79016891 | AGGAAT | 94564306 | 94564326 | 27380 | 27400 | 21 | 0.77213927 |
| | | | CACCTT | | | | | | |
| | | | GCAATT | | | | | | |
| | | | GGC | | | | | | |
| 118 | 4648 | 0.90098533 | AATCAC | 94564309 | 94564325 | 27381 | 27397 | 17 | 0.88295569 |
| | | | CTTGCA | | | | | | |
| | | | ATTGG | | | | | | |
| 119 | 4649 | 0.72815081 | GAATCA | 94564308 | 94564325 | 27381 | 27398 | 18 | 0.71012116 |
| | | | CCTTGC | | | | | | |
| | | | AATTGG | | | | | | |
| 120 | 4650 | 0.64728201 | GGAATC | 94564307 | 94564325 | 27381 | 27399 | 19 | 0.62925237 |
| | | | ACCTTG | | | | | | |
| | | | CAATTG | | | | | | |
| | | | G | | | | | | |
| 121 | 4651 | 0.76330538 | AGGAAT | 94564306 | 94564325 | 27381 | 27400 | 20 | 0.74527574 |
| | | | CACCTT | | | | | | |
| | | | GCAATT | | | | | | |
| | | | GG | | | | | | |
| 122 | 4652 | 0.62727959 | CAGGAA | 94564305 | 94564325 | 27381 | 27401 | 21 | 0.60924995 |
| | | | TCACCTT | | | | | | |
| | | | GCAATT | | | | | | |
| | | | GG | | | | | | |
| 123 | 4653 | 0.78546741 | GAATCA | 94564308 | 94564324 | 27382 | 27398 | 17 | 0.76743777 |
| | | | CCTTGC | | | | | | |
| | | | AATTG | | | | | | |
| 124 | 4654 | 0.8267452 | GGAATC | 94564307 | 94564324 | 27382 | 27399 | 18 | 0.80871556 |
| | | | ACCTTG | | | | | | |
| | | | CAATTG | | | | | | |
| 125 | 4655 | 0.82641003 | AGGAAT | 94564306 | 94564324 | 27382 | 27400 | 19 | 0.80838039 |
| | | | CACCTT | | | | | | |
| | | | GCAATT | | | | | | |
| | | | G | | | | | | |
| 126 | 4656 | 0.7584858 | CAGGAA | 94564305 | 94564324 | 27382 | 27401 | 20 | 0.74045616 |
| | | | TCACCTT | | | | | | |
| | | | GCAATT | | | | | | |
| | | | G | | | | | | |
| 127 | 4657 | 0.70433919 | CCAGGA | 94564304 | 94564324 | 27382 | 27402 | 21 | 0.68630955 |
| | | | ATCACC | | | | | | |
| | | | TTGCAA | | | | | | |
| | | | TTG | | | | | | |
| 128 | 4091 | 0.96455353 | GGAATC | 94564307 | 94564323 | 27383 | 27399 | 17 | 0.94652389 |
| | | | ACCTTG | | | | | | |
| | | | CAATT | | | | | | |
| 129 | 4658 | 0.89000659 | AGGAAT | 94564306 | 94564323 | 27383 | 27400 | 18 | 0.87197695 |
| | | | CACCTT | | | | | | |
| | | | GCAATT | | | | | | |
| 130 | 4659 | 0.74886526 | CAGGAA | 94564305 | 94564323 | 27383 | 27401 | 19 | 0.73083562 |
| | | | TCACCTT | | | | | | |
| | | | GCAATT | | | | | | |
| 131 | 4660 | 0.8928542 | CCAGGA | 94564304 | 94564323 | 27383 | 27402 | 20 | 0.87482456 |
| | | | ATCACC | | | | | | |
| | | | TTGCAA | | | | | | |
| | | | TT | | | | | | |
| 132 | 4661 | 0.8040571 | CCCAGG | 94564303 | 94564323 | 27383 | 27403 | 21 | 0.78602745 |
| | | | AATCAC | | | | | | |
| | | | CTTGCA | | | | | | |
| | | | ATT | | | | | | |
| 133 | 4662 | 0.88681006 | AGGAAT | 94564306 | 94564322 | 27384 | 27400 | 17 | 0.86878042 |
| | | | CACCTT | | | | | | |
| | | | GCAAT | | | | | | |
| 134 | 4663 | 0.80587159 | CAGGAA | 94564305 | 94564322 | 27384 | 27401 | 18 | 0.78784195 |
| | | | TCACCTT | | | | | | |
| | | | GCAAT | | | | | | |
| 135 | 4664 | 0.7487059 | CCAGGA | 94564304 | 94564322 | 27384 | 27402 | 19 | 0.73067626 |
| | | | ATCACC | | | | | | |
| | | | TTGCAA | | | | | | |
| | | | T | | | | | | |
| 136 | 4665 | 0.85609438 | CCCAGG | 94564303 | 94564322 | 27384 | 27403 | 20 | 0.83806474 |
| | | | AATCAC | | | | | | |
| | | | CTTGCA | | | | | | |
| | | | AT | | | | | | |
| 137 | 4666 | 0.64796081 | CCCCAG | 94564302 | 94564322 | 27384 | 27404 | 21 | 0.62993117 |
| | | | GAATCA | | | | | | |
| | | | CCTTGC | | | | | | |
| | | | AAT | | | | | | |
| 138 | 4125 | 0.91268401 | CCCAGG | 94564303 | 94564321 | 27385 | 27403 | 19 | 0.89465437 |
| | | | AATCAC | | | | | | |
| | | | CTTGCA | | | | | | |
| | | | A | | | | | | |
| 139 | 4667 | 0.82019394 | CAGGAA | 94564305 | 94564321 | 27385 | 27401 | 17 | 0.8021643 |
| | | | TCACCTT | | | | | | |
| | | | GCAA | | | | | | |
| 140 | 4668 | 0.78970497 | CCAGGA | 94564304 | 94564321 | 27385 | 27402 | 18 | 0.77167533 |
| | | | ATCACC | | | | | | |
| | | | TTGCAA | | | | | | |
| 141 | 4669 | 0.80707813 | CCCCAG | 94564302 | 94564321 | 27385 | 27404 | 20 | 0.78904849 |
| | | | GAATCA | | | | | | |
| | | | CCTTGC | | | | | | |
| | | | AA | | | | | | |
| 142 | 4670 | 0.61545569 | ACCCCA | 94564301 | 94564321 | 27385 | 27405 | 21 | 0.59742605 |
| | | | GGAATC | | | | | | |
| | | | ACCTTG | | | | | | |
| | | | CAA | | | | | | |
| 143 | 4671 | 0.80883562 | CCAGGA | 94564304 | 94564320 | 27386 | 27402 | 17 | 0.79080598 |
| | | | ATCACC | | | | | | |
| | | | TTGCA | | | | | | |
| 144 | 4672 | 0.83456855 | CCCAGG | 94564303 | 94564320 | 27386 | 27403 | 18 | 0.81653891 |
| | | | AATCAC | | | | | | |
| | | | CTTGCA | | | | | | |
| 145 | 4673 | 0.69793978 | CCCCAG | 94564302 | 94564320 | 27386 | 27404 | 19 | 0.67991014 |
| | | | GAATCA | | | | | | |
| | | | CCTTGC | | | | | | |
| | | | A | | | | | | |
| 146 | 4674 | 0.63673921 | ACCCCA | 94564301 | 94564320 | 27386 | 27405 | 20 | 0.61870957 |
| | | | GGAATC | | | | | | |
| | | | ACCTTG | | | | | | |
| | | | CA | | | | | | |
| 147 | 4675 | 0.64104813 | TACCCC | 94564300 | 94564320 | 27386 | 27406 | 21 | 0.62301849 |
| | | | AGGAAT | | | | | | |
| | | | CACCTT | | | | | | |
| | | | GCA | | | | | | |
| 148 | 4676 | 0.87014332 | CCCAGG | 94564303 | 94564319 | 27387 | 27403 | 17 | 0.85211368 |
| | | | AATCAC | | | | | | |
| | | | CTTGC | | | | | | |
| 149 | 4677 | 0.77803887 | CCCCAG | 94564302 | 94564319 | 27387 | 27404 | 18 | 0.76000923 |
| | | | GAATCA | | | | | | |
| | | | CCTTGC | | | | | | |
| 150 | 4678 | 0.84159721 | ACCCCA | 94564301 | 94564319 | 27387 | 27405 | 19 | 0.82356757 |
| | | | GGAATC | | | | | | |
| | | | ACCTTG | | | | | | |
| | | | C | | | | | | |
| 151 | 4679 | 0.81830134 | TACCCC | 94564300 | 94564319 | 27387 | 27406 | 20 | 0.8002717 |
| | | | AGGAAT | | | | | | |
| | | | CACCTT | | | | | | |
| | | | GC | | | | | | |
| 152 | 4680 | 0.87797865 | CTACCC | 94564299 | 94564319 | 27387 | 27407 | 21 | 0.85994901 |
| | | | CAGGAA | | | | | | |
| | | | TCACCTT | | | | | | |
| | | | GC | | | | | | |
| 153 | 4681 | 0.86670248 | CCCCAG | 94564302 | 94564318 | 27388 | 27404 | 17 | 0.84867284 |
| | | | GAATCA | | | | | | |
| | | | CCTTG | | | | | | |
| 154 | 4682 | 0.87625691 | ACCCCA | 94564301 | 94564318 | 27388 | 27405 | 18 | 0.85822727 |
| | | | GGAATC | | | | | | |
| | | | ACCTTG | | | | | | |
| 155 | 4683 | 0.84275371 | TACCCC | 94564300 | 94564318 | 27388 | 27406 | 19 | 0.82472406 |
| | | | AGGAAT | | | | | | |
| | | | CACCTT | | | | | | |
| | | | G | | | | | | |
| 156 | 4684 | 0.84487036 | CTACCC | 94564299 | 94564318 | 27388 | 27407 | 20 | 0.82684072 |
| | | | CAGGAA | | | | | | |
| | | | TCACCTT | | | | | | |
| | | | G | | | | | | |
| 157 | 4685 | 0.70957679 | GCTACC | 94564298 | 94564318 | 27388 | 27408 | 21 | 0.69154715 |
| | | | CCAGGA | | | | | | |
| | | | ATCACC | | | | | | |
| | | | TTG | | | | | | |
| 158 | 4686 | 0.84873383 | ACCCCA | 94564301 | 94564317 | 27389 | 27405 | 17 | 0.83070419 |
| | | | GGAATC | | | | | | |
| | | | ACCTT | | | | | | |
| 159 | 4687 | 0.81850076 | TACCCC | 94564300 | 94564317 | 27389 | 27406 | 18 | 0.80047112 |
| | | | AGGAAT | | | | | | |
| | | | CACCTT | | | | | | |
| 207 | 4688 | 0.85763794 | CTACCC | 94564299 | 94564317 | 27389 | 27407 | 19 | 0.8396083 |
| | | | CAGGAA | | | | | | |
| | | | TCACCTT | | | | | | |
| 160 | 4689 | 0.77144079 | GCTACC | 94564298 | 94564317 | 27389 | 27408 | 20 | 0.75341115 |
| | | | CCAGGA | | | | | | |
| | | | ATCACC | | | | | | |
| | | | TT | | | | | | |
| 161 | 4690 | 0.80045646 | TGCTAC | 94564297 | 94564317 | 27389 | 27409 | 21 | 0.78242682 |
| | | | CCCAGG | | | | | | |
| | | | AATCAC | | | | | | |
| | | | CTT | | | | | | |
| 162 | 4111 | 0.3795993 | CTACCC | 94564299 | 94564316 | 27390 | 27407 | 18 | 0.36156966 |
| | | | CAGGAA | | | | | | |
| | | | TCACCT | | | | | | |
| 163 | 4691 | 0.82615894 | TACCCC | 94564300 | 94564316 | 27390 | 27406 | 17 | 0.80812929 |
| | | | AGGAAT | | | | | | |
| | | | CACCT | | | | | | |
| 164 | 4692 | 0.83877867 | GCTACC | 94564298 | 94564316 | 27390 | 27408 | 19 | 0.82074903 |
| | | | CCAGGA | | | | | | |
| | | | ATCACC | | | | | | |
| | | | T | | | | | | |
| 165 | 4693 | 0.84312158 | TGCTAC | 94564297 | 94564316 | 27390 | 27409 | 20 | 0.82509194 |
| | | | CCCAGG | | | | | | |
| | | | AATCAC | | | | | | |
| | | | CT | | | | | | |
| 166 | 4694 | 0.75358321 | CTGCTA | 94564296 | 94564316 | 27390 | 27410 | 21 | 0.73555356 |
| | | | CCCCAG | | | | | | |
| | | | GAATCA | | | | | | |
| | | | CCT | | | | | | |
| 167 | 4695 | 0.71573819 | CTACCC | 94564299 | 94564315 | 27391 | 27407 | 17 | 0.69770855 |
| | | | CAGGAA | | | | | | |
| | | | TCACC | | | | | | |
| 168 | 4696 | 0.775299 | GCTACC | 94564298 | 94564315 | 27391 | 27408 | 18 | 0.75726936 |
| | | | CCAGGA | | | | | | |
| | | | ATCACC | | | | | | |
| 169 | 4697 | 0.78009723 | TGCTAC | 94564297 | 94564315 | 27391 | 27409 | 19 | 0.76206759 |
| | | | CCCAGG | | | | | | |
| | | | AATCAC | | | | | | |
| | | | C | | | | | | |
| 170 | 4698 | 0.67240676 | CTGCTA | 94564296 | 94564315 | 27391 | 27410 | 20 | 0.65437712 |
| | | | CCCCAG | | | | | | |
| | | | GAATCA | | | | | | |
| | | | CC | | | | | | |
| 171 | 4699 | 0.73032379 | TCTGCT | 94564295 | 94564315 | 27391 | 27411 | 21 | 0.71229414 |
| | | | ACCCCA | | | | | | |
| | | | GGAATC | | | | | | |
| | | | ACC | | | | | | |
| 172 | 4700 | 0.61028686 | GCTACC | 94564298 | 94564314 | 27392 | 27408 | 17 | 0.59225721 |
| | | | CCAGGA | | | | | | |
| | | | ATCAC | | | | | | |
| 173 | 4701 | 0.69254508 | TGCTAC | 94564297 | 94564314 | 27392 | 27409 | 18 | 0.67451543 |
| | | | CCCAGG | | | | | | |
| | | | AATCAC | | | | | | |
| 174 | 4702 | 0.70030276 | CTGCTA | 94564296 | 94564314 | 27392 | 27410 | 19 | 0.68227312 |
| | | | CCCCAG | | | | | | |
| | | | GAATCA | | | | | | |
| | | | C | | | | | | |
| 175 | 4703 | 0.55123289 | TCTGCT | 94564295 | 94564314 | 27392 | 27411 | 20 | 0.53320325 |
| | | | ACCCCA | | | | | | |
| | | | GGAATC | | | | | | |
| | | | AC | | | | | | |
| 176 | 4704 | 0.44734228 | CTCTGCT | 94564294 | 94564314 | 27392 | 27412 | 21 | 0.42931264 |
| | | | ACCCCA | | | | | | |
| | | | GGAATC | | | | | | |
| | | | AC | | | | | | |
| 177 | 4092 | 0.78761999 | TGCTAC | 94564297 | 94564313 | 27393 | 27409 | 17 | 0.76959035 |
| | | | CCCAGG | | | | | | |
| | | | AATCA | | | | | | |
| 178 | 4705 | 0.83351676 | CTGCTA | 94564296 | 94564313 | 27393 | 27410 | 18 | 0.81548712 |
| | | | CCCCAG | | | | | | |
| | | | GAATCA | | | | | | |
| 179 | 4706 | 0.61126527 | TCTGCT | 94564295 | 94564313 | 27393 | 27411 | 19 | 0.59323563 |
| | | | ACCCCA | | | | | | |
| | | | GGAATC | | | | | | |
| | | | A | | | | | | |
| 180 | 4707 | 0.34441052 | CTCTGCT | 94564294 | 94564313 | 27393 | 27412 | 20 | 0.32638087 |
| | | | ACCCCA | | | | | | |
| | | | GGAATC | | | | | | |
| | | | A | | | | | | |
| 181 | 4708 | 0.57416296 | GCTCTG | 94564293 | 94564313 | 27393 | 27413 | 21 | 0.55613332 |
| | | | CTACCC | | | | | | |
| | | | CAGGAA | | | | | | |
| | | | TCA | | | | | | |
| 182 | 4709 | 0.20688401 | CTGCTA | 94564296 | 94564312 | 27394 | 27410 | 17 | 0.18885437 |
| | | | CCCCAG | | | | | | |
| | | | GAATC | | | | | | |
| 183 | 4710 | 0.37699084 | TCTGCT | 94564295 | 94564312 | 27394 | 27411 | 18 | 0.3589612 |
| | | | ACCCCA | | | | | | |
| | | | GGAATC | | | | | | |
| 184 | 4711 | 0.16262582 | CTCTGCT | 94564294 | 94564312 | 27394 | 27412 | 19 | 0.14459618 |
| | | | ACCCCA | | | | | | |
| | | | GGAATC | | | | | | |
| 185 | 4712 | 0.39432372 | GCTCTG | 94564293 | 94564312 | 27394 | 27413 | 20 | 0.37629408 |
| | | | CTACCC | | | | | | |
| | | | CAGGAA | | | | | | |
| | | | TC | | | | | | |
| 186 | 4713 | 0.30527196 | GGCTCT | 94564292 | 94564312 | 27394 | 27414 | 21 | 0.28724232 |
| | | | GCTACC | | | | | | |
| | | | CCAGGA | | | | | | |
| | | | ATC | | | | | | |
| 187 | 4714 | 0.66369416 | TCTGCT | 94564295 | 94564311 | 27395 | 27411 | 17 | 0.64566452 |
| | | | ACCCCA | | | | | | |
| | | | GGAAT | | | | | | |
| 188 | 4715 | 0.49201464 | CTCTGCT | 94564294 | 94564311 | 27395 | 27412 | 18 | 0.473985 |
| | | | ACCCCA | | | | | | |
| | | | GGAAT | | | | | | |
| 189 | 4716 | 0.65363111 | GCTCTG | 94564293 | 94564311 | 27395 | 27413 | 19 | 0.63560147 |
| | | | CTACCC | | | | | | |
| | | | CAGGAA | | | | | | |
| | | | T | | | | | | |
| 190 | 4717 | 0.70829044 | GGCTCT | 94564292 | 94564311 | 27395 | 27414 | 20 | 0.6902608 |
| | | | GCTACC | | | | | | |
| | | | CCAGGA | | | | | | |
| | | | AT | | | | | | |
| 191 | 4139 | 0.33884001 | AGGCTC | 94564291 | 94564310 | 27396 | 27415 | 20 | 0.32081037 |
| | | | TGCTAC | | | | | | |
| | | | CCCAGG | | | | | | |
| | | | AA | | | | | | |
| 192 | 4718 | 0.46989482 | CTCTGCT | 94564294 | 94564310 | 27396 | 27412 | 17 | 0.45186518 |
| | | | ACCCCA | | | | | | |
| | | | GGAA | | | | | | |
| 193 | 4719 | 0.51069562 | GCTCTG | 94564293 | 94564310 | 27396 | 27413 | 18 | 0.49266597 |
| | | | CTACCC | | | | | | |
| | | | CAGGAA | | | | | | |
| 194 | 4720 | 0.39270541 | GGCTCT | 94564292 | 94564310 | 27396 | 27414 | 19 | 0.37467577 |
| | | | GCTACC | | | | | | |
| | | | CCAGGA | | | | | | |
| | | | A | | | | | | |
| 195 | 4721 | 0.38953287 | GCTCTG | 94564293 | 94564309 | 27397 | 27413 | 17 | 0.37150323 |
| | | | CTACCC | | | | | | |
| | | | CAGGA | | | | | | |
| 196 | 4722 | 0.27990987 | GGCTCT | 94564292 | 94564309 | 27397 | 27414 | 18 | 0.26188022 |
| | | | GCTACC | | | | | | |
| | | | CCAGGA | | | | | | |
| 197 | 4723 | 0.0791666 | GGCTCT | 94564292 | 94564308 | 27398 | 27414 | 17 | 0.06113696 |
| | | | GCTACC | | | | | | |
| | | | CCAGG | | | | | | |
| 198 | 4126 | 0.01690878 | CGTGAG | 94564287 | 94564305 | 27401 | 27419 | 19 | -0.0011209 |
| | | | GCTCTG | | | | | | |
| | | | CTACCC | | | | | | |
| | | | C | | | | | | |
| 199 | 4112 | 0.0039981 | AATTCG | 94564283 | 94564300 | 27406 | 27423 | 18 | -0.0140315 |
| | | | TGAGGC | | | | | | |
| | | | TCTGCT | | | | | | |
| 200 | 4127 | 0 | GGTCAA | 94564279 | 94564297 | 27409 | 27427 | 19 | -0.0180296 |
| | | | TTCGTG | | | | | | |
| | | | AGGCTC | | | | | | |
| | | | T | | | | | | |
| 201 | 4093 | 0.00230947 | CAAGGT | 94564276 | 94564292 | 27414 | 27430 | 17 | -0.0157202 |
| | | | CAATTC | | | | | | |
| | | | GTGAG | | | | | | |
| 202 | 4150 | 0.00677073 | CCTCCC | 94564270 | 94564290 | 27416 | 27436 | 21 | -0.0112589 |
| | | | CAAGGT | | | | | | |
| | | | CAATTC | | | | | | |
| | | | GTG | | | | | | |
| 203 | 4151 | 0.00776482 | GGCTCA | 94564261 | 94564281 | 27425 | 27445 | 21 | -0.0102648 |
| | | | CGCCCT | | | | | | |
| | | | CCCCAA | | | | | | |
| | | | GGT | | | | | | |
| 204 | 4094 | 0.01458947 | CAGGCT | 94564259 | 94564275 | 27431 | 27447 | 17 | -0.0034402 |
| | | | CACGCC | | | | | | |
| | | | CTCCC | | | | | | |
| 205 | 4113 | 0.01159775 | CACCAG | 94564256 | 94564273 | 27433 | 27450 | 18 | -0.0064319 |
| | | | GCTCAC | | | | | | |
| | | | GCCCTC | | | | | | |
| 206 | 4140 | 0.01532544 | CCAGAA | 94564250 | 94564269 | 27437 | 27456 | 20 | -0.0027042 |
| | | | CACCAG | | | | | | |
| | | | GCTCAC | | | | | | |
| | | | GC | | | | | | |

### Example 2 the Splicing of ABCA4 is Disrupted in the c.4773+3A>G Variant and can be Partially Rescued Through the Use of Antisense Oligonucleotides

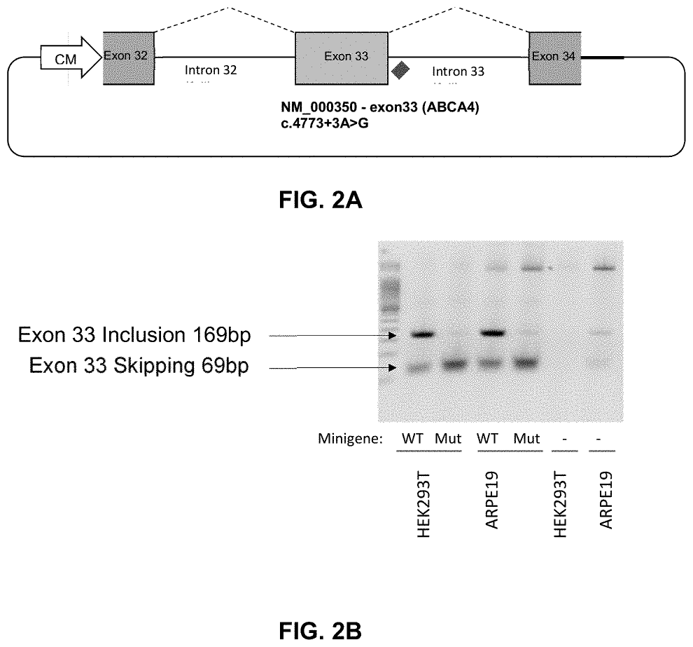

To confirm exon 33 skipping in the chr1: 94487399:T:C [hg19/b37] (c.4773+3A>G) variant, wild type and variant containing minigenes were constructed containing exons 32-34 and the corresponding introns, 32 and 33 (FIG. 2A). Minigenes were then transfected into HEK293T and ARPE19 cells to examine the effect of the c.4773+3A>G variant on splicing. As seen in FIG. 2B, wildtype minigenes showed both exon 33 inclusion, represented by the upper band, and exclusion. c.4773+3A>G mutants, however, showed little exon 33 inclusion indicating the chr1: 94487399:T:C [hg9/b37] mutation induces exon 33 skipping.

To examine the ability of antisense oligonucleotides to promote exon 33 inclusion in the c.4773+3A>G variant the minigenes above were co-transfected with antisense oligonucleotides having sequences set forth in SEQ ID NOs: 208-315 (see Table 5). Antisense oligonucleotides were tiled along exon 33 and intron 33 Antisense oligonucleotides were cotransfected with the mutant minigene containing the c.4773+3A>G variant in HEK293T cells. RT-PCR was conducted to analyze the effect on the splicing of the minigene. Samples were measured by capillary electrophoresis. These results were quantified and are set forth in Table 5. Observing Table 5 it is clear that targeting the intronic regions surrounding exon 33 induces exon 33 inclusion in c.4773+3A>G variant minigenes (high percent spliced in/correctly (PSI) and change in PSI as compared to mutant PSI (dPSI). These observations also suggest antisense oligonucleotides targeting certain regions or “hotspots” in intron 33 (positions 104314-104336 in SEQ ID NO: 1; chr1: 94487370-94487392), e.g., those complementary to a nucleobase sequence in SEQ ID NOs: 260-287, may be particularly useful in the treatment of retinal disease associated with exon 33 skipping (e.g., retinitis pigmentosa, cone-rod dystrophy, or Stargardt disease caused by the c.4773+3A>G mutation).

TABLE 5

| SEQ | | | | Start | End | Start on | Stop on | | |
|---|---|---|---|---|---|---|---|---|---|
| ID | DG | | | Chr1 | Chr1 | SEQ ID | SEQ ID | | |
| NO: | ID | PSI | Sequence | [hg19/b37] | [hg19/b37] | NO: 1 | NO: 1 | length | dPSI |
| 208 | 2870 | 0 | TAAAA | 94487435 | 94487454 | 104252 | 104271 | 20 | −0.0213675 |
| | | | ACCCA | | | | | | |
| | | | ACAAG | | | | | | |
| | | | TGCTT | | | | | | |
| 209 | 2868 | 0 | TTAAA | 94487434 | 94487453 | 104253 | 104272 | 20 | −0.0213675 |
| | | | AACCC | | | | | | |
| | | | AACAA | | | | | | |
| | | | GTGCT | | | | | | |
| 210 | 2869 | 0 | CTTAA | 94487433 | 94487452 | 104254 | 104273 | 20 | −0.0213675 |
| | | | AAACC | | | | | | |
| | | | CAACA | | | | | | |
| | | | AGTGC | | | | | | |
| 211 | 2872 | 0 | GCTTA | 94487432 | 94487451 | 104255 | 104274 | 20 | −0.0213675 |
| | | | AAAAC | | | | | | |
| | | | CCAAC | | | | | | |
| | | | AAGTG | | | | | | |
| 212 | 2871 | 0 | CGCTT | 94487431 | 94487450 | 104256 | 104275 | 20 | −0.0213675 |
| | | | AAAAA | | | | | | |
| | | | CCCAA | | | | | | |
| | | | CAAGT | | | | | | |
| 213 | 2862 | 0.00052457 | CCCCG | 94487401 | 94487420 | 104286 | 104305 | 20 | −0.020843 |
| | | | CTCAC | | | | | | |
| | | | ATTCA | | | | | | |
| | | | TGATC | | | | | | |
| 214 | 2874 | 0.00080667 | CACAC | 94487397 | 94487416 | 104290 | 104309 | 20 | −0.0205609 |
| | | | CCCGC | | | | | | |
| | | | TCACA | | | | | | |
| | | | TTCAT | | | | | | |
| 215 | 2875 | 0.00474358 | TGTTT | 94487391 | 94487410 | 104296 | 104315 | 20 | −0.0166239 |
| | | | ACACA | | | | | | |
| | | | CCCCG | | | | | | |
| | | | CTCAC | | | | | | |
| 216 | 4284 | 0.04784349 | TCTCC | 94487382 | 94487402 | 104304 | 104324 | 21 | 0.02647596 |
| | | | AGTCT | | | | | | |
| | | | GTTTA | | | | | | |
| | | | CACAC | | | | | | |
| | | | C | | | | | | |
| 217 | 2876 | 0.08451165 | CTCCA | 94487383 | 94487402 | 104304 | 104323 | 20 | 0.06314412 |
| | | | GTCTG | | | | | | |
| | | | TTTAC | | | | | | |
| | | | ACACC | | | | | | |
| 218 | 4290 | 0.02633169 | CCAGT | 94487385 | 94487402 | 104304 | 104321 | 18 | 0.00496416 |
| | | | CTGTTT | | | | | | |
| | | | ACACA | | | | | | |
| | | | CC | | | | | | |
| 219 | 4359 | 0.0160642 | CAGTC | 94487386 | 94487402 | 104304 | 104320 | 17 | −0.0053033 |
| | | | TGTTT | | | | | | |
| | | | ACACA | | | | | | |
| | | | CC | | | | | | |
| 220 | 4320 | 0.04684946 | ATCTC | 94487381 | 94487401 | 104305 | 104325 | 21 | 0.02548193 |
| | | | CAGTC | | | | | | |
| | | | TGTTT | | | | | | |
| | | | ACACA | | | | | | |
| | | | C | | | | | | |
| 221 | 4304 | 0.03986191 | TCTCC | 94487382 | 94487401 | 104305 | 104324 | 20 | 0.01849438 |
| | | | AGTCT | | | | | | |
| | | | GTTTA | | | | | | |
| | | | CACAC | | | | | | |
| 222 | 4317 | 0.05247774 | CTCCA | 94487383 | 94487401 | 104305 | 104323 | 19 | 0.03111022 |
| | | | GTCTG | | | | | | |
| | | | TTTAC | | | | | | |
| | | | ACAC | | | | | | |
| 223 | 4288 | 0.02440678 | TCCAG | 94487384 | 94487401 | 104305 | 104322 | 18 | 0.00303925 |
| | | | TCTGTT | | | | | | |
| | | | TACAC | | | | | | |
| | | | AC | | | | | | |
| 224 | 4345 | 0.0152116 | CCAGT | 94487385 | 94487401 | 104305 | 104321 | 17 | −0.0061559 |
| | | | CTGTTT | | | | | | |
| | | | ACACA | | | | | | |
| | | | C | | | | | | |
| 225 | 4338 | 0.02968089 | AATCT | 94487380 | 94487400 | 104306 | 104326 | 21 | 0.00831336 |
| | | | CCAGT | | | | | | |
| | | | CTGTTT | | | | | | |
| | | | ACACA | | | | | | |
| 226 | 4297 | 0.02919964 | ATCTC | 94487381 | 94487400 | 104306 | 104325 | 20 | 0.00783211 |
| | | | CAGTC | | | | | | |
| | | | TGTTT | | | | | | |
| | | | ACACA | | | | | | |
| 227 | 4295 | 0.02665574 | TCTCC | 94487382 | 94487400 | 104306 | 104324 | 19 | 0.00528821 |
| | | | AGTCT | | | | | | |
| | | | GTTTA | | | | | | |
| | | | CACA | | | | | | |
| 228 | 4300 | 0.02227967 | CTCCA | 94487383 | 94487400 | 104306 | 104323 | 18 | 0.00091214 |
| | | | GTCTG | | | | | | |
| | | | TTTAC | | | | | | |
| | | | ACA | | | | | | |
| 229 | 4307 | 0.01566261 | TCCAG | 94487384 | 94487400 | 104306 | 104322 | 17 | −0.0057049 |
| | | | TCTGTT | | | | | | |
| | | | TACAC | | | | | | |
| | | | A | | | | | | |
| 230 | 4348 | 0.02854314 | AAATC | 94487379 | 94487399 | 104307 | 104327 | 21 | 0.00717561 |
| | | | TCCAG | | | | | | |
| | | | TCTGTT | | | | | | |
| | | | TACAC | | | | | | |
| 231 | 4331 | 0.01222792 | AATCT | 94487380 | 94487399 | 104307 | 104326 | 20 | −0.0091396 |
| | | | CCAGT | | | | | | |
| | | | CTGTTT | | | | | | |
| | | | ACAC | | | | | | |
| 232 | 4357 | 0.01851217 | TCTCC | 94487382 | 94487399 | 104307 | 104324 | 18 | −0.0028554 |
| | | | AGTCT | | | | | | |
| | | | GTTTA | | | | | | |
| | | | CAC | | | | | | |
| 233 | 4339 | 0.01564375 | CTCCA | 94487383 | 94487399 | 104307 | 104323 | 17 | −0.0057238 |
| | | | GTCTG | | | | | | |
| | | | TTTAC | | | | | | |
| | | | AC | | | | | | |
| 234 | 4347 | 0.01732577 | CAAAT | 94487378 | 94487398 | 104308 | 104328 | 21 | −0.0040418 |
| | | | CTCCA | | | | | | |
| | | | GTCTG | | | | | | |
| | | | TTTAC | | | | | | |
| | | | A | | | | | | |
| 235 | 4319 | 0.02028748 | AAATC | 94487379 | 94487398 | 104308 | 104327 | 20 | −0.00108 |
| | | | TCCAG | | | | | | |
| | | | TCTGTT | | | | | | |
| | | | TACA | | | | | | |
| 236 | 4316 | 0.02157724 | AATCT | 94487380 | 94487398 | 104308 | 104326 | 19 | 0.00020972 |
| | | | CCAGT | | | | | | |
| | | | CTGTTT | | | | | | |
| | | | ACA | | | | | | |
| 237 | 4308 | 0.01404085 | ATCTC | 94487381 | 94487398 | 104308 | 104325 | 18 | −0.0073267 |
| | | | CAGTC | | | | | | |
| | | | TGTTT | | | | | | |
| | | | ACA | | | | | | |
| 238 | 4299 | 0.01686652 | TCTCC | 94487382 | 94487398 | 104308 | 104324 | 17 | −0.004501 |
| | | | AGTCT | | | | | | |
| | | | GTTTA | | | | | | |
| | | | CA | | | | | | |
| 239 | 4318 | 0.02311438 | TCAAA | 94487377 | 94487397 | 104309 | 104329 | 21 | 0.00174686 |
| | | | TCTCC | | | | | | |
| | | | AGTCT | | | | | | |
| | | | GTTTA | | | | | | |
| | | | C | | | | | | |
| 240 | 2877 | 0.0159866 | CAAAT | 94487378 | 94487397 | 104309 | 104328 | 20 | −0.0053809 |
| | | | CTCCA | | | | | | |
| | | | GTCTG | | | | | | |
| | | | TTTAC | | | | | | |
| 241 | 4315 | 0.02033591 | AAATC | 94487379 | 94487397 | 104309 | 104327 | 19 | −0.0010316 |
| | | | TCCAG | | | | | | |
| | | | TCTGTT | | | | | | |
| | | | TAC | | | | | | |
| 242 | 4324 | 0.01464558 | ATCTC | 94487381 | 94487397 | 104309 | 104325 | 17 | −0.0067219 |
| | | | CAGTC | | | | | | |
| | | | TGTTT | | | | | | |
| | | | AC | | | | | | |
| 243 | 4311 | 0.0241704 | CTCAA | 94487376 | 94487396 | 104310 | 104330 | 21 | 0.00280287 |
| | | | ATCTC | | | | | | |
| | | | CAGTC | | | | | | |
| | | | TGTTT | | | | | | |
| | | | A | | | | | | |
| 244 | 2878 | 0.01586952 | TCAAA | 94487377 | 94487396 | 104310 | 104329 | 20 | −0.005498 |
| | | | TCTCC | | | | | | |
| | | | AGTCT | | | | | | |
| | | | GTTTA | | | | | | |
| 245 | 4334 | 0.01096985 | CAAAT | 94487378 | 94487396 | 104310 | 104328 | 19 | −0.0103977 |
| | | | CTCCA | | | | | | |
| | | | GTCTG | | | | | | |
| | | | TTTA | | | | | | |
| 246 | 4306 | 0.0082054 | AAATC | 94487379 | 94487396 | 104310 | 104327 | 18 | −0.0131621 |
| | | | TCCAG | | | | | | |
| | | | TCTGTT | | | | | | |
| | | | TA | | | | | | |
| 247 | 4336 | 0.00893915 | AATCT | 94487380 | 94487396 | 104310 | 104326 | 17 | −0.0124284 |
| | | | CCAGT | | | | | | |
| | | | CTGTTT | | | | | | |
| | | | A | | | | | | |
| 248 | 4332 | 0.01779842 | CTCAA | 94487376 | 94487395 | 104311 | 104330 | 20 | −0.0035691 |
| | | | ATCTC | | | | | | |
| | | | CAGTC | | | | | | |
| | | | TGTTT | | | | | | |
| 249 | 4314 | 0.02020412 | TCAAA | 94487377 | 94487395 | 104311 | 104329 | 19 | −0.0011634 |
| | | | TCTCC | | | | | | |
| | | | AGTCT | | | | | | |
| | | | GTTT | | | | | | |
| 250 | 4352 | 0.02273897 | CAAAT | 94487378 | 94487395 | 104311 | 104328 | 18 | 0.00137144 |
| | | | CTCCA | | | | | | |
| | | | GTCTG | | | | | | |
| | | | TTT | | | | | | |
| 251 | 4303 | 0.01092555 | AAATC | 94487379 | 94487395 | 104311 | 104327 | 17 | −0.010442 |
| | | | TCCAG | | | | | | |
| | | | TCTGTT | | | | | | |
| | | | T | | | | | | |
| 252 | 4342 | 0.03608537 | TACTC | 94487374 | 94487394 | 104312 | 104332 | 21 | 0.01471785 |
| | | | AAATC | | | | | | |
| | | | TCCAG | | | | | | |
| | | | TCTGTT | | | | | | |
| 253 | 4346 | 0.03163721 | ACTCA | 94487375 | 94487394 | 104312 | 104331 | 20 | 0.01026968 |
| | | | AATCT | | | | | | |
| | | | CCAGT | | | | | | |
| | | | CTGTT | | | | | | |
| 254 | 4277 | 0.02538751 | TCAAA | 94487377 | 94487394 | 104312 | 104329 | 18 | 0.00401999 |
| | | | TCTCC | | | | | | |
| | | | AGTCT | | | | | | |
| | | | GTT | | | | | | |
| 255 | 4341 | 0.0133478 | CAAAT | 94487378 | 94487394 | 104312 | 104328 | 17 | −0.0080197 |
| | | | CTCCA | | | | | | |
| | | | GTCTG | | | | | | |
| | | | TT | | | | | | |
| 256 | 4361 | 0.03839499 | CTACT | 94487373 | 94487393 | 104313 | 104333 | 21 | 0.01702747 |
| | | | CAAAT | | | | | | |
| | | | CTCCA | | | | | | |
| | | | GTCTG | | | | | | |
| | | | T | | | | | | |
| 257 | 4328 | 0.02221052 | ACTCA | 94487375 | 94487393 | 104313 | 104331 | 19 | 0.00084299 |
| | | | AATCT | | | | | | |
| | | | CCAGT | | | | | | |
| | | | CTGT | | | | | | |
| 258 | 4358 | 0.01898736 | CTCAA | 94487376 | 94487393 | 104313 | 104330 | 18 | −0.0023802 |
| | | | ATCTC | | | | | | |
| | | | CAGTC | | | | | | |
| | | | TGT | | | | | | |
| 259 | 4343 | 0.01753224 | TCAAA | 94487377 | 94487393 | 104313 | 104329 | 17 | −0.0038353 |
| | | | TCTCC | | | | | | |
| | | | AGTCT | | | | | | |
| | | | GT | | | | | | |
| 260 | 4298 | 0.07456743 | CCTAC | 94487372 | 94487392 | 104314 | 104334 | 21 | 0.05319991 |
| | | | TCAAA | | | | | | |
| | | | TCTCC | | | | | | |
| | | | AGTCT | | | | | | |
| | | | G | | | | | | |
| 261 | 4289 | 0.05263352 | TACTC | 94487374 | 94487392 | 104314 | 104332 | 19 | 0.031266 |
| | | | AAATC | | | | | | |
| | | | TCCAG | | | | | | |
| | | | TCTG | | | | | | |
| 262 | 4355 | 0.05632484 | ACTCA | 94487375 | 94487392 | 104314 | 104331 | 18 | 0.03495732 |
| | | | AATCT | | | | | | |
| | | | CCAGT | | | | | | |
| | | | CTG | | | | | | |
| 263 | 4312 | 0.04068388 | CTCAA | 94487376 | 94487392 | 104314 | 104330 | 17 | 0.01931635 |
| | | | ATCTC | | | | | | |
| | | | CAGTC | | | | | | |
| | | | TG | | | | | | |
| 264 | 4285 | 0.10321842 | TCCTA | 94487371 | 94487391 | 104315 | 104335 | 21 | 0.0818509 |
| | | | CTCAA | | | | | | |
| | | | ATCTC | | | | | | |
| | | | CAGTC | | | | | | |
| | | | T | | | | | | |
| 265 | 4329 | 0.06474209 | CTACT | 94487373 | 94487391 | 104315 | 104333 | 19 | 0.04337457 |
| | | | CAAAT | | | | | | |
| | | | CTCCA | | | | | | |
| | | | GTCT | | | | | | |
| 266 | 4349 | 0.07991069 | TACTC | 94487374 | 94487391 | 104315 | 104332 | 18 | 0.05854316 |
| | | | AAATC | | | | | | |
| | | | TCCAG | | | | | | |
| | | | TCT | | | | | | |
| 267 | 4282 | 0.05279718 | ACTCA | 94487375 | 94487391 | 104315 | 104331 | 17 | 0.03142965 |
| | | | AATCT | | | | | | |
| | | | CCAGT | | | | | | |
| | | | CT | | | | | | |
| 268 | 4305 | 0.10192797 | ATCCT | 94487370 | 94487390 | 104316 | 104336 | 21 | 0.08056044 |
| | | | ACTCA | | | | | | |
| | | | AATCT | | | | | | |
| | | | CCAGT | | | | | | |
| | | | C | | | | | | |
| 269 | 2863 | 0.12769861 | TCCTA | 94487371 | 94487390 | 104316 | 104335 | 20 | 0.10633108 |
| | | | CTCAA | | | | | | |
| | | | ATCTC | | | | | | |
| | | | CAGTC | | | | | | |
| 270 | 4340 | 0.10554271 | CCTAC | 94487372 | 94487390 | 104316 | 104334 | 19 | 0.08417518 |
| | | | TCAAA | | | | | | |
| | | | TCTCC | | | | | | |
| | | | AGTC | | | | | | |
| 271 | 4309 | 0.07190236 | CTACT | 94487373 | 94487390 | 104316 | 104333 | 18 | 0.05053484 |
| | | | CAAAT | | | | | | |
| | | | CTCCA | | | | | | |
| | | | GTC | | | | | | |
| 272 | 4322 | 0.06185338 | TACTC | 94487374 | 94487390 | 104316 | 104332 | 17 | 0.04048585 |
| | | | AAATC | | | | | | |
| | | | TCCAG | | | | | | |
| | | | TC | | | | | | |
| 273 | 4354 | 0.09178354 | AATCC | 94487369 | 94487389 | 104317 | 104337 | 21 | 0.07041601 |
| | | | TACTC | | | | | | |
| | | | AAATC | | | | | | |
| | | | TCCAG | | | | | | |
| | | | T | | | | | | |
| 274 | 4286 | 0.07464417 | ATCCT | 94487370 | 94487389 | 104317 | 104336 | 20 | 0.05327664 |
| | | | ACTCA | | | | | | |
| | | | AATCT | | | | | | |
| | | | CCAGT | | | | | | |
| 275 | 4323 | 0.05544928 | TCCTA | 94487371 | 94487389 | 104317 | 104335 | 19 | 0.03408175 |
| | | | CTCAA | | | | | | |
| | | | ATCTC | | | | | | |
| | | | CAGT | | | | | | |
| 276 | 4313 | 0.0777456 | CCTAC | 94487372 | 94487389 | 104317 | 104334 | 18 | 0.05637807 |
| | | | TCAAA | | | | | | |
| | | | TCTCC | | | | | | |
| | | | AGT | | | | | | |
| 277 | 4296 | 0.06060062 | AAATC | 94487368 | 94487388 | 104318 | 104338 | 21 | 0.0392331 |
| | | | CTACT | | | | | | |
| | | | CAAAT | | | | | | |
| | | | CTCCA | | | | | | |
| | | | G | | | | | | |
| 278 | 2867 | 0.11830793 | AATCC | 94487369 | 94487388 | 104318 | 104337 | 20 | 0.0969404 |
| | | | TACTC | | | | | | |
| | | | AAATC | | | | | | |
| | | | TCCAG | | | | | | |
| 279 | 4294 | 0.05698576 | ATCCT | 94487370 | 94487388 | 104318 | 104336 | 19 | 0.03561823 |
| | | | ACTCA | | | | | | |
| | | | AATCT | | | | | | |
| | | | CCAG | | | | | | |
| 280 | 4364 | 0.05505851 | TCCTA | 94487371 | 94487388 | 104318 | 104335 | 18 | 0.03369098 |
| | | | CTCAA | | | | | | |
| | | | ATCTC | | | | | | |
| | | | CAG | | | | | | |
| 281 | 4350 | 0.06485799 | CCTAC | 94487372 | 94487388 | 104318 | 104334 | 17 | 0.04349046 |
| | | | TCAAA | | | | | | |
| | | | TCTCC | | | | | | |
| | | | AG | | | | | | |
| 282 | 4287 | 0.04057979 | AAAAT | 94487367 | 94487387 | 104319 | 104339 | 21 | 0.01921226 |
| | | | CCTAC | | | | | | |
| | | | TCAAA | | | | | | |
| | | | TCTCC | | | | | | |
| | | | A | | | | | | |
| 283 | 4330 | 0.03754774 | AAAAA | 94487366 | 94487386 | 104320 | 104340 | 21 | 0.01618022 |
| | | | TCCTA | | | | | | |
| | | | CTCAA | | | | | | |
| | | | ATCTC | | | | | | |
| | | | C | | | | | | |
| 284 | 4326 | 0.03679981 | AAAAT | 94487367 | 94487386 | 104320 | 104339 | 20 | 0.01543229 |
| | | | CCTAC | | | | | | |
| | | | TCAAA | | | | | | |
| | | | TCTCC | | | | | | |
| 285 | 4356 | 0.03101451 | AAATC | 94487368 | 94487386 | 104320 | 104338 | 19 | 0.00964698 |
| | | | CTACT | | | | | | |
| | | | CAAAT | | | | | | |
| | | | CTCC | | | | | | |
| 286 | 4335 | 0.02140241 | AATCC | 94487369 | 94487386 | 104320 | 104337 | 18 | 3.4885E−05 |
| | | | TACTC | | | | | | |
| | | | AAATC | | | | | | |
| | | | TCC | | | | | | |
| 287 | 4344 | 0.02608654 | ATCCT | 94487370 | 94487386 | 104320 | 104336 | 17 | 0.00471901 |
| | | | ACTCA | | | | | | |
| | | | AATCT | | | | | | |
| | | | CC | | | | | | |
| 288 | 4337 | 0.01612763 | AAAAA | 94487366 | 94487385 | 104321 | 104340 | 20 | −0.0052399 |
| | | | TCCTA | | | | | | |
| | | | CTCAA | | | | | | |
| | | | ATCTC | | | | | | |
| 289 | 4283 | 0.01625135 | AAAAT | 94487367 | 94487385 | 104321 | 104339 | 19 | −0.0051162 |
| | | | CCTAC | | | | | | |
| | | | TCAAA | | | | | | |
| | | | TCTC | | | | | | |
| 290 | 4310 | 0.00703731 | TCAAA | 94487364 | 94487384 | 104322 | 104342 | 21 | −0.0143302 |
| | | | AATCC | | | | | | |
| | | | TACTC | | | | | | |
| | | | AAATC | | | | | | |
| | | | T | | | | | | |
| 291 | 4360 | 0.01312168 | AAAAA | 94487366 | 94487384 | 104322 | 104340 | 19 | −0.0082458 |
| | | | TCCTA | | | | | | |
| | | | CTCAA | | | | | | |
| | | | ATCT | | | | | | |
| 292 | 4302 | 0.00716491 | AAAAT | 94487367 | 94487384 | 104322 | 104339 | 18 | −0.0142026 |
| | | | CCTAC | | | | | | |
| | | | TCAAA | | | | | | |
| | | | TCT | | | | | | |
| 293 | 4333 | 0.00594284 | AAATC | 94487368 | 94487384 | 104322 | 104338 | 17 | −0.0154247 |
| | | | CTACT | | | | | | |
| | | | CAAAT | | | | | | |
| | | | CT | | | | | | |
| 294 | 4327 | 0.00735476 | CAAAA | 94487365 | 94487383 | 104323 | 104341 | 19 | −0.0140128 |
| | | | ATCCT | | | | | | |
| | | | ACTCA | | | | | | |
| | | | AATC | | | | | | |
| 295 | 4293 | 0.0062991 | AAAAT | 94487367 | 94487383 | 104323 | 104339 | 17 | −0.0150684 |
| | | | CCTAC | | | | | | |
| | | | TCAAA | | | | | | |
| | | | TC | | | | | | |
| 296 | 4301 | 0.00766725 | AGTCA | 94487362 | 94487382 | 104324 | 104344 | 21 | −0.0137003 |
| | | | AAAAT | | | | | | |
| | | | CCTAC | | | | | | |
| | | | TCAAA | | | | | | |
| | | | T | | | | | | |
| 297 | 2879 | 0.0306372 | GTCAA | 94487363 | 94487382 | 104324 | 104343 | 20 | 0.00926968 |
| | | | AAATC | | | | | | |
| | | | CTACT | | | | | | |
| | | | CAAAT | | | | | | |
| 298 | 4325 | 0.00521359 | TCAAA | 94487364 | 94487382 | 104324 | 104342 | 19 | −0.0161539 |
| | | | AATCC | | | | | | |
| | | | TACTC | | | | | | |
| | | | AAAT | | | | | | |
| 299 | 4281 | 0.00556784 | CAAAA | 94487365 | 94487382 | 104324 | 104341 | 18 | −0.0157997 |
| | | | ATCCT | | | | | | |
| | | | ACTCA | | | | | | |
| | | | AAT | | | | | | |
| 300 | 4278 | 0.00674261 | AGTCA | 94487362 | 94487381 | 104325 | 104344 | 20 | −0.0146249 |
| | | | AAAAT | | | | | | |
| | | | CCTAC | | | | | | |
| | | | TCAAA | | | | | | |
| 301 | 4363 | 0.01433914 | GTCAA | 94487363 | 94487381 | 104325 | 104343 | 19 | −0.0070284 |
| | | | AAATC | | | | | | |
| | | | CTACT | | | | | | |
| | | | CAAA | | | | | | |
| 302 | 4321 | 0.0030924 | CAAAA | 94487365 | 94487381 | 104325 | 104341 | 17 | −0.0182751 |
| | | | ATCCT | | | | | | |
| | | | ACTCA | | | | | | |
| | | | AA | | | | | | |
| 303 | 2880 | 0.03800592 | AAGTC | 94487361 | 94487380 | 104326 | 104345 | 20 | 0.0166384 |
| | | | AAAAA | | | | | | |
| | | | TCCTA | | | | | | |
| | | | CTCAA | | | | | | |
| 304 | 4353 | 0.00893723 | AGTCA | 94487362 | 94487380 | 104326 | 104344 | 19 | −0.0124303 |
| | | | AAAAT | | | | | | |
| | | | CCTAC | | | | | | |
| | | | TCAA | | | | | | |
| 305 | 4280 | 0.00531292 | GTCAA | 94487363 | 94487380 | 104326 | 104343 | 18 | −0.0160546 |
| | | | AAATC | | | | | | |
| | | | CTACT | | | | | | |
| | | | CAA | | | | | | |
| 306 | 4291 | 0.00374818 | TCAAA | 94487364 | 94487380 | 104326 | 104342 | 17 | −0.0176193 |
| | | | AATCC | | | | | | |
| | | | TACTC | | | | | | |
| | | | AA | | | | | | |
| 307 | 4279 | 0.00686827 | AAGTC | 94487361 | 94487379 | 104327 | 104345 | 19 | −0.0144993 |
| | | | AAAAA | | | | | | |
| | | | TCCTA | | | | | | |
| | | | CTCA | | | | | | |
| 308 | 4275 | 0.00534896 | AGTCA | 94487362 | 94487379 | 104327 | 104344 | 18 | −0.0160186 |
| | | | AAAAT | | | | | | |
| | | | CCTAC | | | | | | |
| | | | TCA | | | | | | |
| 309 | 4276 | 0.00592412 | GTCAA | 94487363 | 94487379 | 104327 | 104343 | 17 | −0.0154434 |
| | | | AAATC | | | | | | |
| | | | CTACT | | | | | | |
| | | | CA | | | | | | |
| 310 | 4351 | 0.00988739 | AAGTC | 94487361 | 94487378 | 104328 | 104345 | 18 | −0.0114801 |
| | | | AAAAA | | | | | | |
| | | | TCCTA | | | | | | |
| | | | CTC | | | | | | |
| 311 | 4292 | 0.00570931 | AGTCA | 94487362 | 94487378 | 104328 | 104344 | 17 | −0.0156582 |
| | | | AAAAT | | | | | | |
| | | | CCTAC | | | | | | |
| | | | TC | | | | | | |
| 312 | 4362 | 0.00618523 | AAGTC | 94487361 | 94487377 | 104329 | 104345 | 17 | −0.0151823 |
| | | | AAAAA | | | | | | |
| | | | TCCTA | | | | | | |
| | | | CT | | | | | | |
| 313 | 2881 | 0.0253028 | TTAAG | 94487355 | 94487374 | 104332 | 104351 | 20 | 0.00393528 |
| | | | CAAGT | | | | | | |
| | | | CAAAA | | | | | | |
| | | | ATCCT | | | | | | |
| 314 | 2864 | 0.00584037 | TCATT | 94487342 | 94487361 | 104345 | 104364 | 20 | −0.0155272 |
| | | | CATGG | | | | | | |
| | | | TAGTT | | | | | | |
| | | | AAGCA | | | | | | |
| 315 | 2865 | 0.00560728 | CTCAT | 94487341 | 94487360 | 104346 | 104365 | 20 | −0.0157602 |
| | | | TCATG | | | | | | |
| | | | GTAGT | | | | | | |
| | | | TAAGC | | | | | | |

### Example 3 the Splicing of ABCA4 is Disrupted in the c.5196+1137G>A Variant and can be Partially Rescued Through the Use of Antisense Oligonucleotides

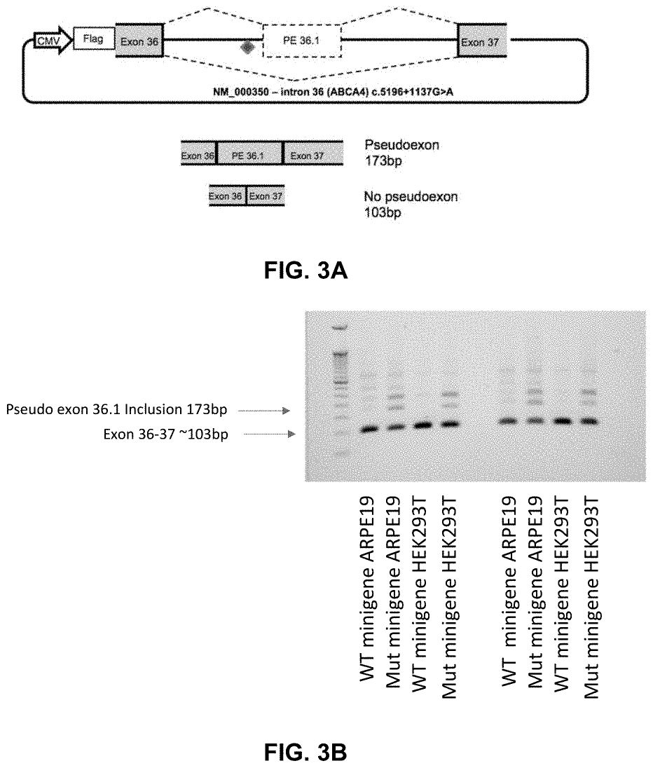

To confirm partial intron 36 inclusion (i.e. pseudo exon inclusion) in the chr1: 94484001:C:T [hg19/b37] (c.5196+1137G>A) variant, wild type and variant containing minigenes were constructed containing exons 36-37 and the corresponding intron 36 (FIG. 3A). Minigenes were then transfected into HEK293T and ARPE19 cells to examine the effect of the c.5196+1137G>A variant on splicing. As seen in FIG. 3B, wildtype minigenes showed little to no intron 36 inclusion, represented by the upper band. c.5196+1137G>A mutants, however, showed no partial intron 36 inclusion (i.e. pseudo exon 36.1 inclusion) indicating the chr1:94484001:C:T [hg19/b37] mutation induces intron 36 inclusion.

To examine the ability of antisense oligonucleotides to promote intron 36 exclusion in the c.5196+1137G>A variant the minigenes above were co-transfected with antisense oligonucleotides having sequences set forth in SEQ ID NOs: 316-385 and 463-596 (see Table 6). Antisense oligonucleotides were tiled along intron 36. Antisense oligonucleotides were cotransfected with the mutant minigene containing the c.5196+1137G>A variant in HEK293T cells. RT-PCR was conducted to analyze the effect on the splicing of the minigene. Samples were measured by capillary electrophoresis. These results were quantified and are set forth in Table 6. Observing Table 6 it is clear that targeting intron 36 promotes intron 36 exclusion in c.5196+1137G>A variant minigenes (high percent spliced in/correctly (PSI) and change in PSI as compared to mutant PSI (dPSI). These observations suggest antisense oligonucleotides targeting this region or “hotspot” (positions 107659-107800 in SEQ ID NO: 1; chr1: 94483906-94484047), e.g., those complementary to a nucleobase sequence in SEQ ID NOs: 316-374 and 463-596, may be particularly useful in the treatment of retinal disease associated with intron 36 inclusion (e.g., retinitis pigmentosa, cone-rod dystrophy, or Stargardt disease caused by the c.5196+1137G>A mutation).

TABLE 6

| SEQ | | | | Start | End | Start on | Stop on | | |
|---|---|---|---|---|---|---|---|---|---|
| ID | DG | | | Chr1 | Chr1 | SEQ ID | SEQ ID | | |
| NO: | ID | PSI | Sequence | [hg19/b37] | [hg19/b37] | NO: 1 | NO: 1 | length | dPSI |
| 316 | 3892 | 0.92360722 | TTTAGTT | 94484028 | 94484047 | 107659 | 107678 | 20 | 0.06408227 |
| | | | GCTACT | | | | | | |
| | | | GATAAT | | | | | | |
| | | | C | | | | | | |
| 317 | 3877 | 0.94385808 | ATTTAG | 94484027 | 94484046 | 107660 | 107679 | 20 | 0.08433313 |
| | | | TTGCTA | | | | | | |
| | | | CTGATA | | | | | | |
| | | | AT | | | | | | |
| 318 | 3891 | 0.92758041 | AATTTA | 94484026 | 94484045 | 107661 | 107680 | 20 | 0.06805546 |
| | | | GTTGCT | | | | | | |
| | | | ACTGAT | | | | | | |
| | | | AA | | | | | | |
| 319 | 3893 | 0.94758288 | AATAAT | 94484023 | 94484042 | 107664 | 107683 | 20 | 0.08805793 |
| | | | TTAGTT | | | | | | |
| | | | GCTACT | | | | | | |
| | | | GA | | | | | | |
| 320 | 3883 | 0.98602336 | AGAGAG | 94484015 | 94484034 | 107672 | 107691 | 20 | 0.12649841 |
| | | | GAAATA | | | | | | |
| | | | ATTTAG | | | | | | |
| | | | TT | | | | | | |
| 321 | 3910 | 0.98162144 | GAGAGA | 94484014 | 94484033 | 107673 | 107692 | 20 | 0.12209649 |
| | | | GGAAAT | | | | | | |
| | | | AATTTA | | | | | | |
| | | | GT | | | | | | |
| 322 | 3902 | 0.97208233 | GAAGAG | 94484011 | 94484030 | 107676 | 107695 | 20 | 0.11255738 |
| | | | AGAGGA | | | | | | |
| | | | AATAAT | | | | | | |
| | | | TT | | | | | | |
| 323 | 3846 | 0.98452774 | AGAAGA | 94484010 | 94484029 | 107677 | 107696 | 20 | 0.12500279 |
| | | | GAGAGG | | | | | | |
| | | | AAATAA | | | | | | |
| | | | TT | | | | | | |
| 324 | 3899 | 0.97837164 | ACAGAA | 94484008 | 94484027 | 107679 | 107698 | 20 | 0.11884669 |
| | | | GAGAGA | | | | | | |
| | | | GGAAAT | | | | | | |
| | | | AA | | | | | | |
| 325 | 3905 | 0.97124617 | AGACAG | 94484006 | 94484025 | 107681 | 107700 | 20 | 0.11172122 |
| | | | AAGAGA | | | | | | |
| | | | GAGGAA | | | | | | |
| | | | AT | | | | | | |
| 326 | 3876 | 0.96455012 | GTGTAG | 94484002 | 94484021 | 107685 | 107704 | 20 | 0.10502517 |
| | | | ACAGAA | | | | | | |
| | | | GAGAGA | | | | | | |
| | | | GG | | | | | | |
| 327 | 3881 | 0.96973536 | TGTGTA | 94484001 | 94484020 | 107686 | 107705 | 20 | 0.1102104 |
| | | | GACAGA | | | | | | |
| | | | AGAGAG | | | | | | |
| | | | AG | | | | | | |
| 328 | 3867 | 0.97895873 | CTTGTGT | 94483999 | 94484018 | 107688 | 107707 | 20 | 0.11943377 |
| | | | AGACAG | | | | | | |
| | | | AAGAGA | | | | | | |
| | | | G | | | | | | |
| 329 | 3865 | 0.98220319 | TCCTTGT | 94483997 | 94484016 | 107690 | 107709 | 20 | 0.12267824 |
| | | | GTAGAC | | | | | | |
| | | | AGAAGA | | | | | | |
| | | | G | | | | | | |
| 330 | 3872 | 0.98844395 | TTTCCTT | 94483995 | 94484014 | 107692 | 107711 | 20 | 0.128919 |
| | | | GTGTAG | | | | | | |
| | | | ACAGAA | | | | | | |
| | | | G | | | | | | |
| 331 | 3869 | 0.9720131 | ATGAGT | 94483988 | 94484007 | 107699 | 107718 | 20 | 0.11248815 |
| | | | GTTTCCT | | | | | | |
| | | | TGTGTA | | | | | | |
| | | | G | | | | | | |
| 332 | 3873 | 0.96449385 | TTATGA | 94483986 | 94484005 | 107701 | 107720 | 20 | 0.1049689 |
| | | | GTGTTTC | | | | | | |
| | | | CTTGTGT | | | | | | |
| 333 | 3871 | 0.99006429 | TGCATTT | 94483981 | 94484000 | 107706 | 107725 | 20 | 0.13053934 |
| | | | ATGAGT | | | | | | |
| | | | GTTTCCT | | | | | | |
| 334 | 3870 | 0.98567671 | CGTGCA | 94483979 | 94483998 | 107708 | 107727 | 20 | 0.12615175 |
| | | | TTTATG | | | | | | |
| | | | AGTGTT | | | | | | |
| | | | TC | | | | | | |
| 335 | 3882 | 0.96968677 | CCGTGC | 94483978 | 94483997 | 107709 | 107728 | 20 | 0.11016182 |
| | | | ATTTAT | | | | | | |
| | | | GAGTGT | | | | | | |
| | | | TT | | | | | | |
| 336 | 3901 | 0.99148614 | CCCCGT | 94483976 | 94483995 | 107711 | 107730 | 20 | 0.13196119 |
| | | | GCATTT | | | | | | |
| | | | ATGAGT | | | | | | |
| | | | GT | | | | | | |
| 337 | 3843 | 0.99443585 | CCTCCC | 94483973 | 94483992 | 107714 | 107733 | 20 | 0.1349109 |
| | | | CGTGCA | | | | | | |
| | | | TTTATG | | | | | | |
| | | | AG | | | | | | |
| 338 | 3851 | 0.97596017 | CTCCTCC | 94483971 | 94483990 | 107716 | 107735 | 20 | 0.11643522 |
| | | | CCGTGC | | | | | | |
| | | | ATTTAT | | | | | | |
| | | | G | | | | | | |
| 339 | 3907 | 0.96166339 | CCTCCTC | 94483970 | 94483989 | 107717 | 107736 | 20 | 0.10213844 |
| | | | CCCGTG | | | | | | |
| | | | CATTTAT | | | | | | |
| 340 | 3878 | 0.98624354 | CTGACC | 94483966 | 94483985 | 107721 | 107740 | 20 | 0.12671859 |
| | | | TCCTCCC | | | | | | |
| | | | CGTGCA | | | | | | |
| | | | T | | | | | | |
| 341 | 3844 | 0.98651575 | TTCTGA | 94483964 | 94483983 | 107723 | 107742 | 20 | 0.1269908 |
| | | | CCTCCTC | | | | | | |
| | | | CCCGTG | | | | | | |
| | | | C | | | | | | |
| 342 | 3847 | 0.99420524 | GTTCTG | 94483963 | 94483982 | 107724 | 107743 | 20 | 0.13468029 |
| | | | ACCTCC | | | | | | |
| | | | TCCCCG | | | | | | |
| | | | TG | | | | | | |
| 343 | 3906 | 0.98854692 | GGTTCT | 94483962 | 94483981 | 107725 | 107744 | 20 | 0.12902197 |
| | | | GACCTC | | | | | | |
| | | | CTCCCC | | | | | | |
| | | | GT | | | | | | |
| 344 | 3845 | 0.95483698 | CAGGTT | 94483960 | 94483979 | 107727 | 107746 | 20 | 0.09531203 |
| | | | CTGACC | | | | | | |
| | | | TCCTCCC | | | | | | |
| | | | C | | | | | | |
| 345 | 3888 | 0.95721508 | TCAGGT | 94483959 | 94483978 | 107728 | 107747 | 20 | 0.09769013 |
| | | | TCTGAC | | | | | | |
| | | | CTCCTCC | | | | | | |
| | | | C | | | | | | |
| 346 | 3890 | 0.96554722 | TTCAGG | 94483958 | 94483977 | 107729 | 107748 | 20 | 0.10602227 |
| | | | TTCTGA | | | | | | |
| | | | CCTCCTC | | | | | | |
| | | | C | | | | | | |
| 347 | 3880 | 0.95891421 | TTTCAG | 94483957 | 94483976 | 107730 | 107749 | 20 | 0.09938926 |
| | | | GTTCTG | | | | | | |
| | | | ACCTCC | | | | | | |
| | | | TC | | | | | | |
| 348 | 3884 | 0.94176661 | AAAGGC | 94483951 | 94483970 | 107736 | 107755 | 20 | 0.08224166 |
| | | | TTTCAG | | | | | | |
| | | | GTTCTG | | | | | | |
| | | | AC | | | | | | |
| 349 | 3894 | 0.9538534 | AAGAAA | 94483948 | 94483967 | 107739 | 107758 | 20 | 0.09432845 |
| | | | GGCTTT | | | | | | |
| | | | CAGGTT | | | | | | |
| | | | CT | | | | | | |
| 350 | 3849 | 0.96705708 | CAAAGA | 94483946 | 94483965 | 107741 | 107760 | 20 | 0.10753213 |
| | | | AAGGCT | | | | | | |
| | | | TTCAGG | | | | | | |
| | | | TT | | | | | | |
| 351 | 3850 | 0.95722885 | CCAAAG | 94483945 | 94483964 | 107742 | 107761 | 20 | 0.0977039 |
| | | | AAAGGC | | | | | | |
| | | | TTTCAG | | | | | | |
| | | | GT | | | | | | |
| 352 | 3889 | 0.95791095 | TCCAAA | 94483944 | 94483963 | 107743 | 107762 | 20 | 0.098386 |
| | | | GAAAGG | | | | | | |
| | | | CTTTCA | | | | | | |
| | | | GG | | | | | | |
| 353 | 3911 | 0.9793179 | TTATCC | 94483941 | 94483960 | 107746 | 107765 | 20 | 0.11979295 |
| | | | AAAGAA | | | | | | |
| | | | AGGCTT | | | | | | |
| | | | TC | | | | | | |
| 354 | 3895 | 0.98777759 | TGCTCTT | 94483936 | 94483955 | 107751 | 107770 | 20 | 0.12825264 |
| | | | ATCCAA | | | | | | |
| | | | AGAAAG | | | | | | |
| | | | G | | | | | | |
| 355 | 3879 | 0.98118092 | TGATGC | 94483933 | 94483952 | 107754 | 107773 | 20 | 0.12165597 |
| | | | TCTTATC | | | | | | |
| | | | CAAAGA | | | | | | |
| | | | A | | | | | | |
| 356 | 3868 | 0.97667072 | GTTGAT | 94483931 | 94483950 | 107756 | 107775 | 20 | 0.11714577 |
| | | | GCTCTT | | | | | | |
| | | | ATCCAA | | | | | | |
| | | | AG | | | | | | |
| 357 | 3848 | 0.97718318 | GCAGTT | 94483928 | 94483947 | 107759 | 107778 | 20 | 0.11765823 |
| | | | GATGCT | | | | | | |
| | | | CTTATCC | | | | | | |
| | | | A | | | | | | |
| 358 | 3842 | 0.98372299 | TGCAGT | 94483927 | 94483946 | 107760 | 107779 | 20 | 0.12419804 |
| | | | TGATGC | | | | | | |
| | | | TCTTATC | | | | | | |
| | | | C | | | | | | |
| 359 | 3866 | 0.97507399 | CTGCAG | 94483926 | 94483945 | 107761 | 107780 | 20 | 0.11554904 |
| | | | TTGATG | | | | | | |
| | | | CTCTTAT | | | | | | |
| | | | C | | | | | | |
| 360 | 3857 | 0.97729685 | CCTGCA | 94483925 | 94483944 | 107762 | 107781 | 20 | 0.1177719 |
| | | | GTTGAT | | | | | | |
| | | | GCTCTT | | | | | | |
| | | | AT | | | | | | |
| 361 | 3864 | 0.98073104 | ACCTGC | 94483924 | 94483943 | 107763 | 107782 | 20 | 0.12120609 |
| | | | AGTTGA | | | | | | |
| | | | TGCTCTT | | | | | | |
| | | | A | | | | | | |
| 362 | 3859 | 0.96952222 | TACCTG | 94483923 | 94483942 | 107764 | 107783 | 20 | 0.10999727 |
| | | | CAGTTG | | | | | | |
| | | | ATGCTC | | | | | | |
| | | | TT | | | | | | |
| 363 | 3852 | 0.97693621 | GTACCT | 94483922 | 94483941 | 107765 | 107784 | 20 | 0.11741126 |
| | | | GCAGTT | | | | | | |
| | | | GATGCT | | | | | | |
| | | | CT | | | | | | |
| 364 | 3856 | 0.96942457 | GGTACC | 94483921 | 94483940 | 107766 | 107785 | 20 | 0.10989962 |
| | | | TGCAGT | | | | | | |
| | | | TGATGC | | | | | | |
| | | | TC | | | | | | |
| 365 | 3855 | 0.95768882 | TGGTAC | 94483920 | 94483939 | 107767 | 107786 | 20 | 0.09816386 |
| | | | CTGCAG | | | | | | |
| | | | TTGATG | | | | | | |
| | | | CT | | | | | | |
| 366 | 3858 | 0.98094362 | GTGGTA | 94483919 | 94483938 | 107768 | 107787 | 20 | 0.12141866 |
| | | | CCTGCA | | | | | | |
| | | | GTTGAT | | | | | | |
| | | | GC | | | | | | |
| 367 | 3861 | 0.97641827 | TGTGGT | 94483918 | 94483937 | 107769 | 107788 | 20 | 0.11689331 |
| | | | ACCTGC | | | | | | |
| | | | AGTTGA | | | | | | |
| | | | TG | | | | | | |
| 368 | 3853 | 0.98023491 | ATGTGG | 94483917 | 94483936 | 107770 | 107789 | 20 | 0.12070996 |
| | | | TACCTG | | | | | | |
| | | | CAGTTG | | | | | | |
| | | | AT | | | | | | |
| 369 | 3854 | 0.9297235 | AATGTG | 94483916 | 94483935 | 107771 | 107790 | 20 | 0.07019855 |
| | | | GTACCT | | | | | | |
| | | | GCAGTT | | | | | | |
| | | | GA | | | | | | |
| 370 | 3860 | 0.97283359 | CAATGT | 94483915 | 94483934 | 107772 | 107791 | 20 | 0.11330864 |
| | | | GGTACC | | | | | | |
| | | | TGCAGT | | | | | | |
| | | | TG | | | | | | |
| 371 | 3875 | 0.96553215 | GCCAAT | 94483913 | 94483932 | 107774 | 107793 | 20 | 0.1060072 |
| | | | GTGGTA | | | | | | |
| | | | CCTGCA | | | | | | |
| | | | GT | | | | | | |
| 372 | 3862 | 0.97278364 | AGGGCC | 94483910 | 94483929 | 107777 | 107796 | 20 | 0.11325868 |
| | | | AATGTG | | | | | | |
| | | | GTACCT | | | | | | |
| | | | GC | | | | | | |
| 373 | 3863 | 0.97702638 | ACAGGG | 94483908 | 94483927 | 107779 | 107798 | 20 | 0.11750143 |
| | | | CCAATG | | | | | | |
| | | | TGGTAC | | | | | | |
| | | | CT | | | | | | |
| 374 | 3874 | 0.9517307 | TCACAG | 94483906 | 94483925 | 107781 | 107800 | 20 | 0.09220574 |
| | | | GGCCAA | | | | | | |
| | | | TGTGGT | | | | | | |
| | | | AC | | | | | | |
| 375 | 3897 | 0.37628761 | ATTAGC | 94483899 | 94483918 | 107788 | 107807 | 20 | −0.4832373 |
| | | | ATCACA | | | | | | |
| | | | GGGCCA | | | | | | |
| | | | AT | | | | | | |
| 376 | 3903 | 0.17207263 | TATTAG | 94483898 | 94483917 | 107789 | 107808 | 20 | −0.6874523 |
| | | | CATCAC | | | | | | |
| | | | AGGGCC | | | | | | |
| | | | AA | | | | | | |
| 377 | 3900 | 0.21244089 | TATATT | 94483896 | 94483915 | 107791 | 107810 | 20 | −0.6470841 |
| | | | AGCATC | | | | | | |
| | | | ACAGGG | | | | | | |
| | | | CC | | | | | | |
| 378 | 3908 | 0.14872555 | TTTATAT | 94483894 | 94483913 | 107793 | 107812 | 20 | −0.7107994 |
| | | | TAGCAT | | | | | | |
| | | | CACAGG | | | | | | |
| | | | G | | | | | | |
| 379 | 3887 | 0.25938883 | CCTTTTA | 94483891 | 94483910 | 107796 | 107815 | 20 | −0.6001361 |
| | | | TATTAG | | | | | | |
| | | | CATCAC | | | | | | |
| | | | A | | | | | | |
| 380 | 3896 | 0.26261845 | TCCTTTT | 94483890 | 94483909 | 107797 | 107816 | 20 | −0.5969065 |
| | | | ATATTA | | | | | | |
| | | | GCATCA | | | | | | |
| | | | C | | | | | | |
| 381 | 3904 | 0.45104715 | GCTCCTT | 94483888 | 94483907 | 107799 | 107818 | 20 | −0.4084778 |
| | | | TTATATT | | | | | | |
| | | | AGCATC | | | | | | |
| 382 | 3886 | 0.53710195 | AGCTCC | 94483887 | 94483906 | 107800 | 107819 | 20 | −0.322423 |
| | | | TTTTATA | | | | | | |
| | | | TTAGCA | | | | | | |
| | | | T | | | | | | |
| 383 | 3898 | 0.39262608 | TAGCTC | 94483886 | 94483905 | 107801 | 107820 | 20 | −0.4668989 |
| | | | CTTTTAT | | | | | | |
| | | | ATTAGC | | | | | | |
| | | | A | | | | | | |
| 384 | 3909 | 0.81437018 | GGCCTA | 94483882 | 94483901 | 107805 | 107824 | 20 | −0.0451548 |
| | | | GCTCCTT | | | | | | |
| | | | TTATATT | | | | | | |
| 385 | 3885 | 0.78147426 | CCGGTG | 94483876 | 94483895 | 107811 | 107830 | 20 | −0.0780507 |
| | | | GGCCTA | | | | | | |
| | | | GCTCCTT | | | | | | |
| | | | T | | | | | | |
| 463 | 6033 | 0.99708567 | TGAGTG | 94483989 | 94484005 | 107701 | 107717 | 17 | 0.13756072 |
| | | | TTTCCTT | | | | | | |
| | | | GTGT | | | | | | |
| 464 | 6034 | 0.99700831 | ATGAGT | 94483988 | 94484005 | 107701 | 107718 | 18 | 0.13748336 |
| | | | GTTTCCT | | | | | | |
| | | | TGTGT | | | | | | |
| 465 | 6035 | 0.99528582 | TATGAG | 94483987 | 94484005 | 107701 | 107719 | 19 | 0.13576086 |
| | | | TGTTTCC | | | | | | |
| | | | TTGTGT | | | | | | |
| 466 | 6036 | 0.98994658 | TTTATG | 94483985 | 94484005 | 107701 | 107721 | 21 | 0.13042163 |
| | | | AGTGTT | | | | | | |
| | | | TCCTTGT | | | | | | |
| | | | GT | | | | | | |
| 467 | 6037 | 0.99670278 | ATGAGT | 94483988 | 94484004 | 107702 | 107718 | 17 | 0.13717783 |
| | | | GTTTCCT | | | | | | |
| | | | TGTG | | | | | | |
| 468 | 6038 | 0.99552504 | TATGAG | 94483987 | 94484004 | 107702 | 107719 | 18 | 0.13600009 |
| | | | TGTTTCC | | | | | | |
| | | | TTGTG | | | | | | |
| 469 | 6039 | 0.99370127 | TTATGA | 94483986 | 94484004 | 107702 | 107720 | 19 | 0.13417632 |
| | | | GTGTTTC | | | | | | |
| | | | CTTGTG | | | | | | |
| 470 | 6040 | 0.99364496 | TTTATG | 94483985 | 94484004 | 107702 | 107721 | 20 | 0.13412001 |
| | | | AGTGTT | | | | | | |
| | | | TCCTTGT | | | | | | |
| | | | G | | | | | | |
| 471 | 6041 | 0.99742833 | ATTTAT | 94483984 | 94484004 | 107702 | 107722 | 21 | 0.13790338 |
| | | | GAGTGT | | | | | | |
| | | | TTCCTTG | | | | | | |
| | | | TG | | | | | | |
| 472 | 6042 | 0.99386028 | TATGAG | 94483987 | 94484003 | 107703 | 107719 | 17 | 0.13433532 |
| | | | TGTTTCC | | | | | | |
| | | | TTGT | | | | | | |
| 473 | 6043 | 0.9948824 | TTATGA | 94483986 | 94484003 | 107703 | 107720 | 18 | 0.13535745 |
| | | | GTGTTTC | | | | | | |
| | | | CTTGT | | | | | | |
| 474 | 6044 | 0.99560869 | TTTATG | 94483985 | 94484003 | 107703 | 107721 | 19 | 0.13608373 |
| | | | AGTGTT | | | | | | |
| | | | TCCTTGT | | | | | | |
| 475 | 6045 | 0.98836088 | ATTTAT | 94483984 | 94484003 | 107703 | 107722 | 20 | 0.12883593 |
| | | | GAGTGT | | | | | | |
| | | | TTCCTTG | | | | | | |
| | | | T | | | | | | |
| 476 | 6046 | 0.99812564 | CATTTAT | 94483983 | 94484003 | 107703 | 107723 | 21 | 0.13860069 |
| | | | GAGTGT | | | | | | |
| | | | TTCCTTG | | | | | | |
| | | | T | | | | | | |
| 477 | 6047 | 0.99661461 | TTATGA | 94483986 | 94484002 | 107704 | 107720 | 17 | 0.13708966 |
| | | | GTGTTTC | | | | | | |
| | | | CTTG | | | | | | |
| 478 | 6048 | 0.98365619 | TTTATG | 94483985 | 94484002 | 107704 | 107721 | 18 | 0.12413124 |
| | | | AGTGTT | | | | | | |
| | | | TCCTTG | | | | | | |
| 479 | 6049 | 0.99452638 | ATTTAT | 94483984 | 94484002 | 107704 | 107722 | 19 | 0.13500143 |
| | | | GAGTGT | | | | | | |
| | | | TTCCTTG | | | | | | |
| 480 | 6050 | 0.97742354 | CATTTAT | 94483983 | 94484002 | 107704 | 107723 | 20 | 0.11789859 |
| | | | GAGTGT | | | | | | |
| | | | TTCCTTG | | | | | | |
| 481 | 6051 | 0.99790655 | GCATTT | 94483982 | 94484002 | 107704 | 107724 | 21 | 0.1383816 |
| | | | ATGAGT | | | | | | |
| | | | GTTTCCT | | | | | | |
| | | | TG | | | | | | |
| 482 | 6052 | 0.99011281 | TTTATG | 94483985 | 94484001 | 107705 | 107721 | 17 | 0.13058786 |
| | | | AGTGTT | | | | | | |
| | | | TCCTT | | | | | | |
| 483 | 6053 | 0.99628751 | ATTTAT | 94483984 | 94484001 | 107705 | 107722 | 18 | 0.13676256 |
| | | | GAGTGT | | | | | | |
| | | | TTCCTT | | | | | | |
| 484 | 6054 | 0.99774963 | CATTTAT | 94483983 | 94484001 | 107705 | 107723 | 19 | 0.13822468 |
| | | | GAGTGT | | | | | | |
| | | | TTCCTT | | | | | | |
| 485 | 6055 | 0.99672063 | GCATTT | 94483982 | 94484001 | 107705 | 107724 | 20 | 0.13719568 |
| | | | ATGAGT | | | | | | |
| | | | GTTTCCT | | | | | | |
| | | | T | | | | | | |
| 486 | 6056 | 0.99696414 | TGCATTT | 94483981 | 94484001 | 107705 | 107725 | 21 | 0.13743919 |
| | | | ATGAGT | | | | | | |
| | | | GTTTCCT | | | | | | |
| | | | T | | | | | | |
| 487 | 6057 | 0.998537 | ATTTAT | 94483984 | 94484000 | 107706 | 107722 | 17 | 0.13901204 |
| | | | GAGTGT | | | | | | |
| | | | TTCCT | | | | | | |
| 488 | 6058 | 0.99733283 | CATTTAT | 94483983 | 94484000 | 107706 | 107723 | 18 | 0.13780788 |
| | | | GAGTGT | | | | | | |
| | | | TTCCT | | | | | | |
| 489 | 6059 | 0.99794292 | GCATTT | 94483982 | 94484000 | 107706 | 107724 | 19 | 0.13841796 |
| | | | ATGAGT | | | | | | |
| | | | GTTTCCT | | | | | | |
| 490 | 6060 | 0.99779486 | GTGCAT | 94483980 | 94484000 | 107706 | 107726 | 21 | 0.13826991 |
| | | | TTATGA | | | | | | |
| | | | GTGTTTC | | | | | | |
| | | | CT | | | | | | |
| 491 | 6061 | 0.99868652 | CATTTAT | 94483983 | 94483999 | 107707 | 107723 | 17 | 0.13916157 |
| | | | GAGTGT | | | | | | |
| | | | TTCC | | | | | | |
| 492 | 6062 | 0.99832234 | GCATTT | 94483982 | 94483999 | 107707 | 107724 | 18 | 0.13879739 |
| | | | ATGAGT | | | | | | |
| | | | GTTTCC | | | | | | |
| 493 | 6063 | 0.99765297 | TGCATTT | 94483981 | 94483999 | 107707 | 107725 | 19 | 0.13812802 |
| | | | ATGAGT | | | | | | |
| | | | GTTTCC | | | | | | |
| 494 | 6064 | 0.98029775 | GTGCAT | 94483980 | 94483999 | 107707 | 107726 | 20 | 0.1207728 |
| | | | TTATGA | | | | | | |
| | | | GTGTTTC | | | | | | |
| | | | C | | | | | | |
| 495 | 6065 | 0.99754751 | CGTGCA | 94483979 | 94483999 | 107707 | 107727 | 21 | 0.13802256 |
| | | | TTTATG | | | | | | |
| | | | AGTGTT | | | | | | |
| | | | TCC | | | | | | |
| 496 | 6066 | 0.9946547 | GCATTT | 94483982 | 94483998 | 107708 | 107724 | 17 | 0.13512975 |
| | | | ATGAGT | | | | | | |
| | | | GTTTC | | | | | | |
| 497 | 6067 | 0.99593138 | TGCATTT | 94483981 | 94483998 | 107708 | 107725 | 18 | 0.13640642 |
| | | | ATGAGT | | | | | | |
| | | | GTTTC | | | | | | |
| 498 | 6068 | 0.9909698 | GTGCAT | 94483980 | 94483998 | 107708 | 107726 | 19 | 0.13144485 |
| | | | TTATGA | | | | | | |
| | | | GTGTTTC | | | | | | |
| 499 | 6069 | 0.99372888 | CCGTGC | 94483978 | 94483998 | 107708 | 107728 | 21 | 0.13420393 |
| | | | ATTTAT | | | | | | |
| | | | GAGTGT | | | | | | |
| | | | TTC | | | | | | |
| 500 | 6070 | 0.99159647 | TGCATTT | 94483981 | 94483997 | 107709 | 107725 | 17 | 0.13207151 |
| | | | ATGAGT | | | | | | |
| | | | GTTT | | | | | | |
| 501 | 6071 | 0.99707014 | GTGCAT | 94483980 | 94483997 | 107709 | 107726 | 18 | 0.13754519 |
| | | | TTATGA | | | | | | |
| | | | GTGTTT | | | | | | |
| 502 | 6072 | 0.99356046 | CGTGCA | 94483979 | 94483997 | 107709 | 107727 | 19 | 0.13403551 |
| | | | TTTATG | | | | | | |
| | | | AGTGTT | | | | | | |
| | | | T | | | | | | |
| 503 | 6073 | 0.99731285 | CCCGTG | 94483977 | 94483997 | 107709 | 107729 | 21 | 0.1377879 |
| | | | CATTTAT | | | | | | |
| | | | GAGTGT | | | | | | |
| | | | TT | | | | | | |
| 504 | 6074 | 0.99667542 | GTGCAT | 94483980 | 94483996 | 107710 | 107726 | 17 | 0.13715047 |
| | | | TTATGA | | | | | | |
| | | | GTGTT | | | | | | |
| 505 | 6075 | 0.99654701 | CGTGCA | 94483979 | 94483996 | 107710 | 107727 | 18 | 0.13702206 |
| | | | TTTATG | | | | | | |
| | | | AGTGTT | | | | | | |
| 506 | 6076 | 0.99430514 | CCGTGC | 94483978 | 94483996 | 107710 | 107728 | 19 | 0.13478019 |
| | | | ATTTAT | | | | | | |
| | | | GAGTGT | | | | | | |
| | | | T | | | | | | |
| 507 | 6077 | 0.99864031 | CCCGTG | 94483977 | 94483996 | 107710 | 107729 | 20 | 0.13911536 |
| | | | CATTTAT | | | | | | |
| | | | GAGTGT | | | | | | |
| | | | T | | | | | | |
| 508 | 6078 | 0.99513775 | CCCCGT | 94483976 | 94483996 | 107710 | 107730 | 21 | 0.1356128 |
| | | | GCATTT | | | | | | |
| | | | ATGAGT | | | | | | |
| | | | GTT | | | | | | |
| 509 | 6079 | 0.98996838 | CGTGCA | 94483979 | 94483995 | 107711 | 107727 | 17 | 0.13044343 |
| | | | TTTATG | | | | | | |
| | | | AGTGT | | | | | | |
| 510 | 6080 | 0.99932461 | CCGTGC | 94483978 | 94483995 | 107711 | 107728 | 18 | 0.13979966 |
| | | | ATTTAT | | | | | | |
| | | | GAGTGT | | | | | | |
| 511 | 6081 | 0.98981026 | CCCGTG | 94483977 | 94483995 | 107711 | 107729 | 19 | 0.13028531 |
| | | | CATTTAT | | | | | | |
| | | | GAGTGT | | | | | | |
| 512 | 6082 | 0.99093164 | TCCCCG | 94483975 | 94483995 | 107711 | 107731 | 21 | 0.13140669 |
| | | | TGCATTT | | | | | | |
| | | | ATGAGT | | | | | | |
| | | | GT | | | | | | |
| 513 | 6083 | 0.99524727 | CCGTGC | 94483978 | 94483994 | 107712 | 107728 | 17 | 0.13572232 |
| | | | ATTTAT | | | | | | |
| | | | GAGTG | | | | | | |
| 514 | 6084 | 0.99255254 | CCCGTG | 94483977 | 94483994 | 107712 | 107729 | 18 | 0.13302759 |
| | | | CATTTAT | | | | | | |
| | | | GAGTG | | | | | | |
| 515 | 6085 | 0.99366018 | CCCCGT | 94483976 | 94483994 | 107712 | 107730 | 19 | 0.13413523 |
| | | | GCATTT | | | | | | |
| | | | ATGAGT | | | | | | |
| | | | G | | | | | | |
| 516 | 6086 | 0.99911074 | TCCCCG | 94483975 | 94483994 | 107712 | 107731 | 20 | 0.13958579 |
| | | | TGCATTT | | | | | | |
| | | | ATGAGT | | | | | | |
| | | | G | | | | | | |
| 517 | 6087 | 0.99968834 | CTCCCC | 94483974 | 94483994 | 107712 | 107732 | 21 | 0.14016339 |
| | | | GTGCAT | | | | | | |
| | | | TTATGA | | | | | | |
| | | | GTG | | | | | | |
| 518 | 6088 | 1 | CCCGTG | 94483977 | 94483993 | 107713 | 107729 | 17 | 0.14047505 |
| | | | CATTTAT | | | | | | |
| | | | GAGT | | | | | | |
| 519 | 6089 | 0.9965087 | CCCCGT | 94483976 | 94483993 | 107713 | 107730 | 18 | 0.13698375 |
| | | | GCATTT | | | | | | |
| | | | ATGAGT | | | | | | |
| 520 | 6090 | 0.99896379 | TCCCCG | 94483975 | 94483993 | 107713 | 107731 | 19 | 0.13943884 |
| | | | TGCATTT | | | | | | |
| | | | ATGAGT | | | | | | |
| 521 | 6091 | 0.99920439 | CTCCCC | 94483974 | 94483993 | 107713 | 107732 | 20 | 0.13967944 |
| | | | GTGCAT | | | | | | |
| | | | TTATGA | | | | | | |
| | | | GT | | | | | | |
| 522 | 6092 | 0.99359014 | CCTCCC | 94483973 | 94483993 | 107713 | 107733 | 21 | 0.13406519 |
| | | | CGTGCA | | | | | | |
| | | | TTTATG | | | | | | |
| | | | AGT | | | | | | |
| 523 | 6093 | 1 | CCCCGT | 94483976 | 94483992 | 107714 | 107730 | 17 | 0.14047505 |
| | | | GCATTT | | | | | | |
| | | | ATGAG | | | | | | |
| 524 | 6094 | 0.99679413 | TCCCCG | 94483975 | 94483992 | 107714 | 107731 | 18 | 0.13726918 |
| | | | TGCATTT | | | | | | |
| | | | ATGAG | | | | | | |
| 525 | 6095 | 0.995319 | CTCCCC | 94483974 | 94483992 | 107714 | 107732 | 19 | 0.13579405 |
| | | | GTGCAT | | | | | | |
| | | | TTATGA | | | | | | |
| | | | G | | | | | | |
| 526 | 6096 | 1 | TCCTCCC | 94483972 | 94483992 | 107714 | 107734 | 21 | 0.14047505 |
| | | | CGTGCA | | | | | | |
| | | | TTTATG | | | | | | |
| | | | AG | | | | | | |
| 527 | 6097 | 0.98989575 | TCCCCG | 94483975 | 94483991 | 107715 | 107731 | 17 | 0.1303708 |
| | | | TGCATTT | | | | | | |
| | | | ATGA | | | | | | |
| 528 | 6098 | 0.99149171 | CTCCCC | 94483974 | 94483991 | 107715 | 107732 | 18 | 0.13196676 |
| | | | GTGCAT | | | | | | |
| | | | TTATGA | | | | | | |
| 529 | 6099 | 0.99354399 | CCTCCC | 94483973 | 94483991 | 107715 | 107733 | 19 | 0.13401904 |
| | | | CGTGCA | | | | | | |
| | | | TTTATG | | | | | | |
| | | | A | | | | | | |
| 530 | 6100 | 0.99448301 | TCCTCCC | 94483972 | 94483991 | 107715 | 107734 | 20 | 0.13495806 |
| | | | CGTGCA | | | | | | |
| | | | TTTATG | | | | | | |
| | | | A | | | | | | |
| 531 | 6101 | 0.99703138 | CTCCTCC | 94483971 | 94483991 | 107715 | 107735 | 21 | 0.13750643 |
| | | | CCGTGC | | | | | | |
| | | | ATTTAT | | | | | | |
| | | | GA | | | | | | |
| 532 | 6102 | 0.99558543 | CTCCCC | 94483974 | 94483990 | 107716 | 107732 | 17 | 0.13606047 |
| | | | GTGCAT | | | | | | |
| | | | TTATG | | | | | | |
| 533 | 6103 | 0.99912813 | CCTCCC | 94483973 | 94483990 | 107716 | 107733 | 18 | 0.13960318 |
| | | | CGTGCA | | | | | | |
| | | | TTTATG | | | | | | |
| 534 | 6104 | 0.99498711 | TCCTCCC | 94483972 | 94483990 | 107716 | 107734 | 19 | 0.13546216 |
| | | | CGTGCA | | | | | | |
| | | | TTTATG | | | | | | |
| 535 | 6105 | 0.99606456 | CCTCCTC | 94483970 | 94483990 | 107716 | 107736 | 21 | 0.1365396 |
| | | | CCCGTG | | | | | | |
| | | | CATTTAT | | | | | | |
| | | | G | | | | | | |
| 536 | 6106 | 0.99538394 | CCTCCC | 94483973 | 94483989 | 107717 | 107733 | 17 | 0.13585899 |
| | | | CGTGCA | | | | | | |
| | | | TTTAT | | | | | | |
| 537 | 6107 | 0.99116241 | TCCTCCC | 94483972 | 94483989 | 107717 | 107734 | 18 | 0.13163746 |
| | | | CGTGCA | | | | | | |
| | | | TTTAT | | | | | | |
| 538 | 6108 | 0.98809019 | CTCCTCC | 94483971 | 94483989 | 107717 | 107735 | 19 | 0.12856524 |
| | | | CCGTGC | | | | | | |
| | | | ATTTAT | | | | | | |
| 539 | 6109 | 0.99708577 | ACCTCC | 94483969 | 94483989 | 107717 | 107737 | 21 | 0.13756082 |
| | | | TCCCCG | | | | | | |
| | | | TGCATTT | | | | | | |
| | | | AT | | | | | | |
| 540 | 6110 | 0.99257134 | TCCTCCC | 94483972 | 94483988 | 107718 | 107734 | 17 | 0.13304639 |
| | | | CGTGCA | | | | | | |
| | | | TTTA | | | | | | |
| 541 | 6111 | 0.9921426 | CTCCTCC | 94483971 | 94483988 | 107718 | 107735 | 18 | 0.13261765 |
| | | | CCGTGC | | | | | | |
| | | | ATTTA | | | | | | |
| 542 | 6112 | 0.99077156 | CCTCCTC | 94483970 | 94483988 | 107718 | 107736 | 19 | 0.13124661 |
| | | | CCCGTG | | | | | | |
| | | | CATTTA | | | | | | |
| 543 | 6113 | 0.92250391 | ACCTCC | 94483969 | 94483988 | 107718 | 107737 | 20 | 0.06297896 |
| | | | TCCCCG | | | | | | |
| | | | TGCATTT | | | | | | |
| | | | A | | | | | | |
| 544 | 6114 | 0.99325004 | GACCTC | 94483968 | 94483988 | 107718 | 107738 | 21 | 0.13372509 |
| | | | CTCCCC | | | | | | |
| | | | GTGCAT | | | | | | |
| | | | TTA | | | | | | |
| 545 | 6115 | 0.99636481 | CTCCTCC | 94483971 | 94483987 | 107719 | 107735 | 17 | 0.13683986 |
| | | | CCGTGC | | | | | | |
| | | | ATTT | | | | | | |
| 546 | 6116 | 0.99413994 | CCTCCTC | 94483970 | 94483987 | 107719 | 107736 | 18 | 0.13461499 |
| | | | CCCGTG | | | | | | |
| | | | CATTT | | | | | | |
| 547 | 6117 | 0.99570644 | ACCTCC | 94483969 | 94483987 | 107719 | 107737 | 19 | 0.13618149 |
| | | | TCCCCG | | | | | | |
| | | | TGCATTT | | | | | | |
| 548 | 6118 | 0.99405885 | GACCTC | 94483968 | 94483987 | 107719 | 107738 | 20 | 0.1345339 |
| | | | CTCCCC | | | | | | |
| | | | GTGCAT | | | | | | |
| | | | TT | | | | | | |
| 549 | 6119 | 0.99754622 | TGACCT | 94483967 | 94483987 | 107719 | 107739 | 21 | 0.13802127 |
| | | | CCTCCC | | | | | | |
| | | | CGTGCA | | | | | | |
| | | | TTT | | | | | | |
| 550 | 6120 | 0.97369837 | CCTCCTC | 94483970 | 94483986 | 107720 | 107736 | 17 | 0.11417342 |
| | | | CCCGTG | | | | | | |
| | | | CATT | | | | | | |
| 551 | 6121 | 0.95975907 | ACCTCC | 94483969 | 94483986 | 107720 | 107737 | 18 | 0.10023411 |
| | | | TCCCCG | | | | | | |
| | | | TGCATT | | | | | | |
| 552 | 6122 | 0.9985255 | GACCTC | 94483968 | 94483986 | 107720 | 107738 | 19 | 0.13900055 |
| | | | CTCCCC | | | | | | |
| | | | GTGCAT | | | | | | |
| | | | T | | | | | | |
| 553 | 6123 | 0.9904905 | TGACCT | 94483967 | 94483986 | 107720 | 107739 | 20 | 0.13096555 |
| | | | CCTCCC | | | | | | |
| | | | CGTGCA | | | | | | |
| | | | TT | | | | | | |
| 554 | 6124 | 0.99407828 | CTGACC | 94483966 | 94483986 | 107720 | 107740 | 21 | 0.13455333 |
| | | | TCCTCCC | | | | | | |
| | | | CGTGCA | | | | | | |
| | | | TT | | | | | | |
| 555 | 6125 | 0.99485913 | ACCTCC | 94483969 | 94483985 | 107721 | 107737 | 17 | 0.13533418 |
| | | | TCCCCG | | | | | | |
| | | | TGCAT | | | | | | |
| 556 | 6126 | 0.99153982 | GACCTC | 94483968 | 94483985 | 107721 | 107738 | 18 | 0.13201487 |
| | | | CTCCCC | | | | | | |
| | | | GTGCAT | | | | | | |
| 557 | 6127 | 0.99438632 | TGACCT | 94483967 | 94483985 | 107721 | 107739 | 19 | 0.13486137 |
| | | | CCTCCC | | | | | | |
| | | | CGTGCA | | | | | | |
| | | | T | | | | | | |
| 558 | 6129 | 0.99675885 | GACCTC | 94483968 | 94483984 | 107722 | 107738 | 17 | 0.13723389 |
| | | | CTCCCC | | | | | | |
| | | | GTGCA | | | | | | |
| 559 | 6130 | 0.99704147 | TGACCT | 94483967 | 94483984 | 107722 | 107739 | 18 | 0.13751652 |
| | | | CCTCCC | | | | | | |
| | | | CGTGCA | | | | | | |
| 560 | 6131 | 0.99707416 | CTGACC | 94483966 | 94483984 | 107722 | 107740 | 19 | 0.13754921 |
| | | | TCCTCCC | | | | | | |
| | | | CGTGCA | | | | | | |
| 561 | 6132 | 0.9970857 | TCTGAC | 94483965 | 94483984 | 107722 | 107741 | 20 | 0.13756075 |
| | | | CTCCTCC | | | | | | |
| | | | CCGTGC | | | | | | |
| | | | A | | | | | | |
| 562 | 6133 | 0.99736692 | TTCTGA | 94483964 | 94483984 | 107722 | 107742 | 21 | 0.13784197 |
| | | | CCTCCTC | | | | | | |
| | | | CCCGTG | | | | | | |
| | | | CA | | | | | | |
| 563 | 6134 | 0.9916746 | TGACCT | 94483967 | 94483983 | 107723 | 107739 | 17 | 0.13214965 |
| | | | CCTCCC | | | | | | |
| | | | CGTGC | | | | | | |
| 564 | 6135 | 0.99740995 | CTGACC | 94483966 | 94483983 | 107723 | 107740 | 18 | 0.137885 |
| | | | TCCTCCC | | | | | | |
| | | | CGTGC | | | | | | |
| 565 | 6136 | 1 | TCTGAC | 94483965 | 94483983 | 107723 | 107741 | 19 | 0.14047505 |
| | | | CTCCTCC | | | | | | |
| | | | CCGTGC | | | | | | |
| 566 | 6137 | 0.98683302 | GTTCTG | 94483963 | 94483983 | 107723 | 107743 | 21 | 0.12730807 |
| | | | ACCTCC | | | | | | |
| | | | TCCCCG | | | | | | |
| | | | TGC | | | | | | |
| 567 | 6138 | 0.99762799 | CTGACC | 94483966 | 94483982 | 107724 | 107740 | 17 | 0.13810304 |
| | | | TCCTCCC | | | | | | |
| | | | CGTG | | | | | | |
| 568 | 6139 | 0.98803138 | TCTGAC | 94483965 | 94483982 | 107724 | 107741 | 18 | 0.12850643 |
| | | | CTCCTCC | | | | | | |
| | | | CCGTG | | | | | | |
| 569 | 6140 | 0.99322322 | TTCTGA | 94483964 | 94483982 | 107724 | 107742 | 19 | 0.13369827 |
| | | | CCTCCTC | | | | | | |
| | | | CCCGTG | | | | | | |
| 570 | 6141 | 0.99086404 | GGTTCT | 94483962 | 94483982 | 107724 | 107744 | 21 | 0.13133909 |
| | | | GACCTC | | | | | | |
| | | | CTCCCC | | | | | | |
| | | | GTG | | | | | | |
| 571 | 6142 | 0.99460361 | TCTGAC | 94483965 | 94483981 | 107725 | 107741 | 17 | 0.13507865 |
| | | | CTCCTCC | | | | | | |
| | | | CCGT | | | | | | |
| 572 | 6143 | 0.9978076 | TTCTGA | 94483964 | 94483981 | 107725 | 107742 | 18 | 0.13828264 |
| | | | CCTCCTC | | | | | | |
| | | | CCCGT | | | | | | |
| 573 | 6144 | 0.99947537 | GTTCTG | 94483963 | 94483981 | 107725 | 107743 | 19 | 0.13995042 |
| | | | ACCTCC | | | | | | |
| | | | TCCCCG | | | | | | |
| | | | T | | | | | | |
| 574 | 6145 | 0.99781033 | AGGTTC | 94483961 | 94483981 | 107725 | 107745 | 21 | 0.13828538 |
| | | | TGACCT | | | | | | |
| | | | CCTCCC | | | | | | |
| | | | CGT | | | | | | |
| 575 | 6146 | 0.99578042 | TTCTGA | 94483964 | 94483980 | 107726 | 107742 | 17 | 0.13625547 |
| | | | CCTCCTC | | | | | | |
| | | | CCCG | | | | | | |
| 576 | 6147 | 0.99733058 | GTTCTG | 94483963 | 94483980 | 107726 | 107743 | 18 | 0.13780562 |
| | | | ACCTCC | | | | | | |
| | | | TCCCCG | | | | | | |
| 577 | 6148 | 1 | GGTTCT | 94483962 | 94483980 | 107726 | 107744 | 19 | 0.14047505 |
| | | | GACCTC | | | | | | |
| | | | CTCCCC | | | | | | |
| | | | G | | | | | | |
| 578 | 6149 | 0.99758052 | AGGTTC | 94483961 | 94483980 | 107726 | 107745 | 20 | 0.13805557 |
| | | | TGACCT | | | | | | |
| | | | CCTCCC | | | | | | |
| | | | CG | | | | | | |
| 579 | 6150 | 0.99711125 | CAGGTT | 94483960 | 94483980 | 107726 | 107746 | 21 | 0.1375863 |
| | | | CTGACC | | | | | | |
| | | | TCCTCCC | | | | | | |
| | | | CG | | | | | | |
| 580 | 6151 | 0.99860493 | GTTCTG | 94483963 | 94483979 | 107727 | 107743 | 17 | 0.13907998 |
| | | | ACCTCC | | | | | | |
| | | | TCCCC | | | | | | |
| 581 | 6152 | 0.99723212 | GGTTCT | 94483962 | 94483979 | 107727 | 107744 | 18 | 0.13770717 |
| | | | GACCTC | | | | | | |
| | | | CTCCCC | | | | | | |
| 582 | 6153 | 0.99282364 | AGGTTC | 94483961 | 94483979 | 107727 | 107745 | 19 | 0.13329869 |
| | | | TGACCT | | | | | | |
| | | | CCTCCC | | | | | | |
| | | | C | | | | | | |
| 583 | 6154 | 0.99716907 | TCAGGT | 94483959 | 94483979 | 107727 | 107747 | 21 | 0.13764412 |
| | | | TCTGAC | | | | | | |
| | | | CTCCTCC | | | | | | |
| | | | CC | | | | | | |
| 584 | 6155 | 0.99847681 | GGTTCT | 94483962 | 94483978 | 107728 | 107744 | 17 | 0.13895186 |
| | | | GACCTC | | | | | | |
| | | | CTCCC | | | | | | |
| 585 | 6156 | 0.99567493 | AGGTTC | 94483961 | 94483978 | 107728 | 107745 | 18 | 0.13614998 |
| | | | TGACCT | | | | | | |
| | | | CCTCCC | | | | | | |
| 586 | 6157 | 0.99506277 | CAGGTT | 94483960 | 94483978 | 107728 | 107746 | 19 | 0.13553782 |
| | | | CTGACC | | | | | | |
| | | | TCCTCCC | | | | | | |
| 587 | 6158 | 0.99636379 | TTCAGG | 94483958 | 94483978 | 107728 | 107748 | 21 | 0.13683884 |
| | | | TTCTGA | | | | | | |
| | | | CCTCCTC | | | | | | |
| | | | CC | | | | | | |
| 588 | 6159 | 0.99109538 | AGGTTC | 94483961 | 94483977 | 107729 | 107745 | 17 | 0.13157043 |
| | | | TGACCT | | | | | | |
| | | | CCTCC | | | | | | |
| 589 | 6160 | 0.98907762 | CAGGTT | 94483960 | 94483977 | 107729 | 107746 | 18 | 0.12955267 |
| | | | CTGACC | | | | | | |
| | | | TCCTCC | | | | | | |
| 590 | 6161 | 0.98093795 | TCAGGT | 94483959 | 94483977 | 107729 | 107747 | 19 | 0.121413 |
| | | | TCTGAC | | | | | | |
| | | | CTCCTCC | | | | | | |
| 591 | 6162 | 0.99262906 | CAGGTT | 94483960 | 94483976 | 107730 | 107746 | 17 | 0.13310411 |
| | | | CTGACC | | | | | | |
| | | | TCCTC | | | | | | |
| 592 | 6163 | 0.99141297 | TCAGGT | 94483959 | 94483976 | 107730 | 107747 | 18 | 0.13188801 |
| | | | TCTGAC | | | | | | |
| | | | CTCCTC | | | | | | |
| 593 | 6164 | 0.95402775 | TTCAGG | 94483958 | 94483976 | 107730 | 107748 | 19 | 0.0945028 |
| | | | TTCTGA | | | | | | |
| | | | CCTCCTC | | | | | | |
| 594 | 6165 | 0.99038866 | TCAGGT | 94483959 | 94483975 | 107731 | 107747 | 17 | 0.1308637 |
| | | | TCTGAC | | | | | | |
| | | | CTCCT | | | | | | |
| 595 | 6166 | 0.98818288 | TTCAGG | 94483958 | 94483975 | 107731 | 107748 | 18 | 0.12865793 |
| | | | TTCTGA | | | | | | |
| | | | CCTCCT | | | | | | |
| 596 | 6167 | 0.96431084 | TTCAGG | 94483958 | 94483974 | 107732 | 107748 | 17 | 0.10478589 |
| | | | TTCTGA | | | | | | |
| | | | CCTCC | | | | | | |

### Example 4 the Splicing of ABCA4 is Disrupted in the c.5714+5G>A Variant and can be Partially Rescued Through the Use of Antisense Oligonucleotides

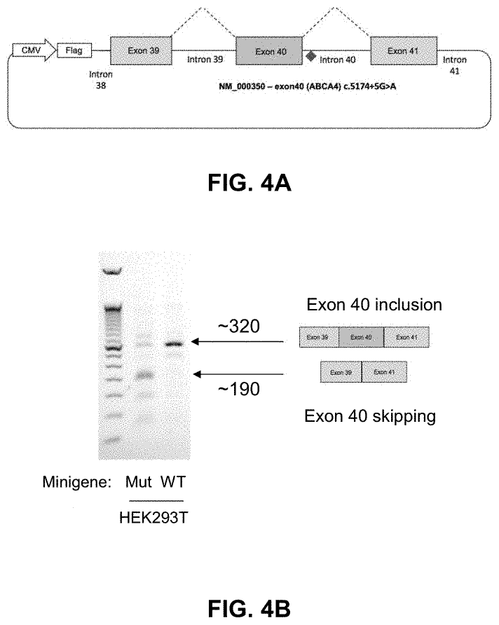

To confirm exon 40 skipping in the chr1: 94476351:C:T [hg19/b37] (c.5714+5G>A) variant, wild type and variant containing minigenes were constructed containing exons 39-41 and the corresponding introns, 38, 39, 40 and 41 (FIG. 4A). Minigenes were then transfected into HEK293T cells to examine the effect of the c.5714+5G>A variant on splicing. As seen in FIG. 4B, wildtype minigenes showed only exon 40 inclusion, represented by the upper band. c.5714+5G>A mutants, however, showed mostly exon 40 exclusion, represented by the lower band, and some exon 40 inclusion indicating the chr1:94476351:C:T [hg19/b37] mutation induces exon 40 skipping.

To examine the ability of antisense oligonucleotides to promote exon 40 inclusion in the c.5714+5G>A variant the minigenes above were co-transfected with antisense oligonucleotides having sequences set forth in SEQ ID NOs: 386-449 (see Table 7). Antisense oligonucleotides were tiled along exon 40 and the surrounding introns. Antisense oligonucleotides were cotransfected with the mutant minigene containing the c.5714+5G>A variant in HEK293T cells. RT-PCR was conducted to analyze the effect on the splicing of the minigene. Samples were measured by capillary electrophoresis. These results were quantified and are set forth in Table 7. Observing Table 7 it is clear that targeting the intronic regions surrounding exon 7 or exon 7 induces exon 7 inclusion in c.5714+5G>A variant minigenes (high percent spliced in/correctly (PSI) and change in PSI as compared to mutant PSI (dPSI)). These observations suggest antisense oligonucleotides targeting these regions or “hotspots” (positions 115149-115205, 115357-115378 and 115384-115450 in SEQ ID NO: 1; chr1: 94476501-94476557, 94476328-94476349 and chr1: 94476256-94476322), e.g., those complementary to a nucleobase sequence in SEQ ID NOs: 390-394 for hotspot 1 and SEQ ID NOs: 438-449 for hotspot 2, may be particularly useful in the treatment of retinal disease associated with exon 40 skipping (e.g., retinitis pigmentosa, cone-rod dystrophy, or Stargardt disease caused by the c.5714+5G>A mutation).

TABLE 7

| | | | | | | Start | Stop | | |
|---|---|---|---|---|---|---|---|---|---|
| | | | | | | on | on | | |
| SEQ | | | | | | SEQ | SEQ | | |
| ID | DG | | | Start Chr1 | End Chr1 | ID | ID | | |
| NO: | ID | PSI | Sequence | [hg19/b37] | [hg19/b37] | NO: 1 | NO: 1 | length | dPSI |
| 386 | 4245 | 0.16351047 | ACCAGG | 94476566 | 94476584 | 115122 | 115140 | 19 | 0.00013772 |
| | | | CCTTAT | | | | | | |
| | | | GTGGGA | | | | | | |
| | | | A | | | | | | |
| 387 | 4255 | 0.15063859 | ACTAGA | 94476561 | 94476580 | 115126 | 115145 | 20 | −0.0127342 |
| | | | CCAGGC | | | | | | |
| | | | CTTATGT | | | | | | |
| | | | G | | | | | | |
| 388 | 4209 | 0.14451858 | CCCACT | 94476558 | 94476574 | 115132 | 115148 | 17 | −0.0188542 |
| | | | AGACCA | | | | | | |
| | | | GGCCT | | | | | | |
| 389 | 4267 | 0.11805199 | GCCACA | 94476544 | 94476564 | 115142 | 115162 | 21 | −0.0453208 |
| | | | GCACAG | | | | | | |
| | | | GGCCCA | | | | | | |
| | | | CTA | | | | | | |
| 390 | 4268 | 0.17173042 | AGACCT | 94476537 | 94476557 | 115149 | 115169 | 21 | 0.00835767 |
| | | | GGCCAC | | | | | | |
| | | | AGCACA | | | | | | |
| | | | GGG | | | | | | |
| 391 | 4246 | 0.16997976 | GCTCAC | 94476526 | 94476544 | 115162 | 115180 | 19 | 0.00660701 |
| | | | CCCACA | | | | | | |
| | | | GACCTG | | | | | | |
| | | | G | | | | | | |
| 392 | 4269 | 0.21080501 | GCCGCC | 94476517 | 94476537 | 115169 | 115189 | 21 | 0.04743226 |
| | | | CCAGCT | | | | | | |
| | | | CACCCC | | | | | | |
| | | | ACA | | | | | | |
| 393 | 4270 | 0.4678908 | CCACTT | 94476509 | 94476529 | 115177 | 115197 | 21 | 0.30451805 |
| | | | CAGCCG | | | | | | |
| | | | CCCCAG | | | | | | |
| | | | CTC | | | | | | |
| 394 | 4271 | 0.18572087 | AATTGA | 94476501 | 94476521 | 115185 | 115205 | 21 | 0.02234812 |
| | | | GTCCAC | | | | | | |
| | | | TTCAGC | | | | | | |
| | | | CGC | | | | | | |
| 395 | 4227 | 0.07507214 | AACAGG | 94476495 | 94476512 | 115194 | 115211 | 18 | −0.0883006 |
| | | | AATTGA | | | | | | |
| | | | GTCCAC | | | | | | |
| 396 | 4247 | 0.04474524 | CATCAA | 94476491 | 94476509 | 115197 | 115215 | 19 | −0.1186275 |
| | | | CAGGAA | | | | | | |
| | | | TTGAGT | | | | | | |
| | | | C | | | | | | |
| 397 | 4210 | 0.03055414 | GGCATC | 94476489 | 94476505 | 115201 | 115217 | 17 | −0.1328186 |
| | | | AACAGG | | | | | | |
| | | | AATTG | | | | | | |
| 398 | 4228 | 0.11932321 | CTGGGC | 94476486 | 94476503 | 115203 | 115220 | 18 | −0.0440495 |
| | | | ATCAAC | | | | | | |
| | | | AGGAAT | | | | | | |
| 399 | 4248 | 0.14264007 | CTCACC | 94476481 | 94476499 | 115207 | 115225 | 19 | −0.0207327 |
| | | | TGGGCA | | | | | | |
| | | | TCAACA | | | | | | |
| | | | G | | | | | | |
| 400 | 4211 | 0.06308102 | CTCCTC | 94476478 | 94476494 | 115212 | 115228 | 17 | −0.1002917 |
| | | | ACCTGG | | | | | | |
| | | | GCATC | | | | | | |
| 401 | 4212 | 0.08435033 | GTGCTC | 94476475 | 94476491 | 115215 | 115231 | 17 | −0.0790224 |
| | | | CTCACC | | | | | | |
| | | | TGGGC | | | | | | |
| 402 | 4256 | 0.08861896 | GCAGAG | 94476470 | 94476489 | 115217 | 115236 | 20 | −0.0747538 |
| | | | TGCTCCT | | | | | | |
| | | | CACCTG | | | | | | |
| | | | G | | | | | | |
| 403 | 4249 | 0.06416822 | GGATTT | 94476464 | 94476482 | 115224 | 115242 | 19 | −0.0992045 |
| | | | GCAGAG | | | | | | |
| | | | TGCTCCT | | | | | | |
| 404 | 4257 | 0.06926567 | CCAGTG | 94476454 | 94476473 | 115233 | 115252 | 20 | −0.0941071 |
| | | | GAACGG | | | | | | |
| | | | ATTTGC | | | | | | |
| | | | AG | | | | | | |
| 405 | 4213 | 0.01971552 | GTCCCA | 94476451 | 94476467 | 115239 | 115255 | 17 | −0.1436572 |
| | | | GTGGAA | | | | | | |
| | | | CGGAT | | | | | | |
| 406 | 4214 | 0.0438786 | AGGTCC | 94476449 | 94476465 | 115241 | 115257 | 17 | −0.1194942 |
| | | | CAGTGG | | | | | | |
| | | | AACGG | | | | | | |
| 407 | 4229 | 0.02575892 | ATCAGG | 94476446 | 94476463 | 115243 | 115260 | 18 | −0.1376138 |
| | | | TCCCAG | | | | | | |
| | | | TGGAAC | | | | | | |
| 408 | 4230 | 0.13447573 | TCCCAA | 94476441 | 94476458 | 115248 | 115265 | 18 | −0.028897 |
| | | | TCAGGT | | | | | | |
| | | | CCCAGT | | | | | | |
| 409 | 4231 | 0.05533741 | TCTTCCC | 94476438 | 94476455 | 115251 | 115268 | 18 | −0.1080353 |
| | | | AATCAG | | | | | | |
| | | | GTCCC | | | | | | |
| 410 | 4272 | 0.01480694 | ACAGGT | 94476432 | 94476452 | 115254 | 115274 | 21 | −0.1485658 |
| | | | TCTTCCC | | | | | | |
| | | | AATCAG | | | | | | |
| | | | GT | | | | | | |
| 411 | 4258 | 0.07816009 | GGCAAA | 94476427 | 94476446 | 115260 | 115279 | 20 | −0.0852127 |
| | | | CAGGTT | | | | | | |
| | | | CTTCCC | | | | | | |
| | | | AA | | | | | | |
| 412 | 4232 | 0.14657467 | CATGGC | 94476424 | 94476441 | 115265 | 115282 | 18 | −0.0167981 |
| | | | AAACAG | | | | | | |
| | | | GTTCTT | | | | | | |
| 413 | 4215 | 0.04375712 | ACCATG | 94476422 | 94476438 | 115268 | 115284 | 17 | −0.1196156 |
| | | | GCAAAC | | | | | | |
| | | | AGGTT | | | | | | |
| 414 | 4216 | 0.02380441 | CCACCA | 94476420 | 94476436 | 115270 | 115286 | 17 | −0.1395683 |
| | | | TGGCAA | | | | | | |
| | | | ACAGG | | | | | | |
| 415 | 4233 | 0.03588861 | CCACCA | 94476417 | 94476434 | 115272 | 115289 | 18 | −0.1274841 |
| | | | CCATGG | | | | | | |
| | | | CAAACA | | | | | | |
| 416 | 4217 | 0.08374322 | CCCTTCC | 94476412 | 94476428 | 115278 | 115294 | 17 | −0.0796295 |
| | | | ACCACC | | | | | | |
| | | | ATGG | | | | | | |
| 417 | 4234 | 0.10068959 | CACCCC | 94476409 | 94476426 | 115280 | 115297 | 18 | −0.0626832 |
| | | | TTCCAC | | | | | | |
| | | | CACCAT | | | | | | |
| 418 | 4235 | 0.0860025 | AGTACA | 94476402 | 94476419 | 115287 | 115304 | 18 | −0.0773703 |
| | | | CCACCC | | | | | | |
| | | | CTTCCA | | | | | | |
| 419 | 4259 | 0.03802999 | GAGGAA | 94476397 | 94476416 | 115290 | 115309 | 20 | −0.1253428 |
| | | | GTACAC | | | | | | |
| | | | CACCCC | | | | | | |
| | | | TT | | | | | | |
| 420 | 4250 | 0.11174784 | GGTCAG | 94476391 | 94476409 | 115297 | 115315 | 19 | −0.0516249 |
| | | | GAGGAA | | | | | | |
| | | | GTACAC | | | | | | |
| | | | C | | | | | | |
| 421 | 4218 | 0.10316017 | CAGGGT | 94476388 | 94476404 | 115302 | 115318 | 17 | −0.0602126 |
| | | | CAGGAG | | | | | | |
| | | | GAAGT | | | | | | |
| 422 | 4219 | 0.21595241 | CCAGCA | 94476384 | 94476400 | 115306 | 115322 | 17 | 0.05257966 |
| | | | GGGTCA | | | | | | |
| | | | GGAGG | | | | | | |
| 423 | 4236 | 0.18745955 | CTGGAC | 94476379 | 94476396 | 115310 | 115327 | 18 | 0.0240868 |
| | | | CAGCAG | | | | | | |
| | | | GGTCAG | | | | | | |
| 424 | 4260 | 0 | GTGGCG | 94476373 | 94476392 | 115314 | 115333 | 20 | −0.1633728 |
| | | | CTGGAC | | | | | | |
| | | | CAGCAG | | | | | | |
| | | | GG | | | | | | |
| 425 | 4237 | 0.09913076 | GAAGAA | 94476367 | 94476384 | 115322 | 115339 | 18 | −0.064242 |
| | | | GTGGCG | | | | | | |
| | | | CTGGAC | | | | | | |
| 426 | 4238 | 0.0837757 | GAGGAA | 94476364 | 94476381 | 115325 | 115342 | 18 | −0.0795971 |
| | | | GAAGTG | | | | | | |
| | | | GCGCTG | | | | | | |
| 427 | 4261 | 0.09184707 | ATTGGG | 94476357 | 94476376 | 115330 | 115349 | 20 | −0.0715257 |
| | | | AGAGGA | | | | | | |
| | | | AGAAGT | | | | | | |
| | | | GG | | | | | | |
| 428 | 4220 | 0.13158774 | CCATTG | 94476355 | 94476371 | 115335 | 115351 | 17 | 31 0.031785 |
| | | | GGAGAG | | | | | | |
| | | | GAAGA | | | | | | |
| 429 | 4239 | 0.08859458 | GTACCA | 94476352 | 94476369 | 115337 | 115354 | 18 | −0.0747782 |
| | | | TTGGGA | | | | | | |
| | | | GAGGAA | | | | | | |
| 430 | 4273 | 0.07765108 | CATGGA | 94476345 | 94476365 | 115341 | 115361 | 21 | −0.0857217 |
| | | | TGTACC | | | | | | |
| | | | ATTGGG | | | | | | |
| | | | AGA | | | | | | |
| 431 | 4251 | 0.04522755 | GTGTGG | 94476339 | 94476357 | 115349 | 115367 | 19 | −0.1181452 |
| | | | CATGGA | | | | | | |
| | | | TGTACC | | | | | | |
| | | | A | | | | | | |
| 432 | 4221 | 0.12038155 | AGGGTG | 94476336 | 94476352 | 115354 | 115370 | 17 | −0.0429912 |
| | | | TGGCAT | | | | | | |
| | | | GGATG | | | | | | |
| 433 | 4252 | 0.18419996 | GGCCCA | 94476331 | 94476349 | 115357 | 115375 | 19 | 0.02082721 |
| | | | GGGTGT | | | | | | |
| | | | GGCATG | | | | | | |
| | | | G | | | | | | |
| 434 | 4240 | 0.29185317 | ACTGGC | 94476328 | 94476345 | 115361 | 115378 | 18 | 0.12848042 |
| | | | CCAGGG | | | | | | |
| | | | TGTGGC | | | | | | |
| 435 | 4262 | 0.09500995 | TGAGCT | 94476318 | 94476337 | 115369 | 115388 | 20 | −0.0683628 |
| | | | GCCCAC | | | | | | |
| | | | TGGCCC | | | | | | |
| | | | AG | | | | | | |
| 436 | 4263 | 0.11642409 | TGCCCT | 94476313 | 94476332 | 115374 | 115393 | 20 | −0.0469487 |
| | | | GAGCTG | | | | | | |
| | | | CCCACT | | | | | | |
| | | | GG | | | | | | |
| 437 | 4264 | 0.06303642 | CTGGAT | 94476308 | 94476327 | 115379 | 115398 | 20 | −0.1003363 |
| | | | GCCCTG | | | | | | |
| | | | AGCTGC | | | | | | |
| | | | CC | | | | | | |
| 438 | 4222 | 0.28020735 | TTCTGG | 94476306 | 94476322 | 115384 | 115400 | 17 | 0.11683459 |
| | | | ATGCCC | | | | | | |
| | | | TGAGC | | | | | | |
| 439 | 4241 | 0.19171274 | GTCCAG | 94476300 | 94476317 | 115389 | 115406 | 18 | 0.02833999 |
| | | | TTCTGG | | | | | | |
| | | | ATGCCC | | | | | | |
| 440 | 4223 | 0.28203905 | TAAGGT | 94476296 | 94476312 | 115394 | 115410 | 17 | 0.1186663 |
| | | | CCAGTT | | | | | | |
| | | | CTGGA | | | | | | |
| 441 | 4242 | 0.18281706 | GTATAA | 94476293 | 94476310 | 115396 | 115413 | 18 | 0.01944431 |
| | | | GGTCCA | | | | | | |
| | | | GTTCTG | | | | | | |
| 442 | 4253 | 0.22976438 | GTGGGT | 94476289 | 94476307 | 115399 | 115417 | 19 | 0.06639163 |
| | | | ATAAGG | | | | | | |
| | | | TCCAGT | | | | | | |
| | | | T | | | | | | |
| 443 | 4243 | 0.24376363 | GAAATG | 94476278 | 94476295 | 115411 | 115428 | 18 | 0.08039088 |
| | | | ACCATG | | | | | | |
| | | | TGGGTA | | | | | | |
| 444 | 4274 | 0.11565453 | TGAGGA | 94476270 | 94476290 | 115416 | 115436 | 21 | −0.0477182 |
| | | | AAGAAA | | | | | | |
| | | | TGACCA | | | | | | |
| | | | TGT | | | | | | |
| 445 | 4254 | 0.18343226 | GCTCCT | 94476265 | 94476283 | 115423 | 115441 | 19 | 0.02005951 |
| | | | GAGGAA | | | | | | |
| | | | AGAAAT | | | | | | |
| | | | G | | | | | | |
| 446 | 4224 | 0.25878428 | GGGCTC | 94476263 | 94476279 | 115427 | 115443 | 17 | 0.09541153 |
| | | | CTGAGG | | | | | | |
| | | | AAAGA | | | | | | |
| 447 | 4244 | 0.19718093 | GTGGGG | 94476260 | 94476277 | 115429 | 115446 | 18 | 0.03380818 |
| | | | CTCCTG | | | | | | |
| | | | AGGAAA | | | | | | |
| 448 | 4225 | 0.22573324 | GAGTGG | 94476258 | 94476274 | 115432 | 115448 | 17 | 0.06236049 |
| | | | GGCTCC | | | | | | |
| | | | TGAGG | | | | | | |
| 449 | 4226 | 0.17536592 | TGGAGT | 94476256 | 94476272 | 115434 | 115450 | 17 | 0.01199317 |
| | | | GGGGCT | | | | | | |
| | | | CCTGA | | | | | | |

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

1.-101. (canceled)

102. An antisense oligonucleotide comprising a nucleobase sequence at least 70% complementary to an ABCA4 pre-mRNA target sequence in a 5′-flanking intron, a 3′-flanking intron, or a combination of an exon and the 5′-flanking intron or the 3′-flanking intron.

103. The antisense oligonucleotide of claim 1, wherein binding of the antisense oligonucleotide to the ABCA4 pre-mRNA target sequence reduces binding of a splicing factor to an intronic splicing silencer in the 5′-flanking intron or the 3′-flanking intron or a splicing enhancer.

104. The antisense oligonucleotide of claim 102, wherein the nucleobase sequence is complementary to a sequence within the 5′-flanking intron of the ABCA4 pre-mRNA.

105. The antisense oligonucleotide of claim 102, wherein the ABCA4 pre-mRNA target sequence is located within the 3′-flanking intron of the ABCA4 pre-mRNA.

106. The antisense oligonucleotide of claim 102, wherein the ABCA4 pre-mRNA target sequence is in a 5′-flanking intron adjacent to exon 6, a 3′-flanking intron adjacent to exon 6, or a combination of the exon 6 and the 5′-flanking intron adjacent to exon 6 or the 3′-flanking intron adjacent to exon 6.

107. The antisense oligonucleotide of claim 102, wherein the ABCA4 pre-mRNA target sequence comprises at least one nucleotide located among positions 27362-27419 in SEQ ID NO: 1.

108. The antisense oligonucleotide of claim 102, wherein the nucleobase sequence has at least 70% sequence identity to any one of SEQ ID NOs: 60-198 and 207.

109. The antisense oligonucleotide of claim 102, wherein the ABCA4 pre-mRNA target sequence is in a 5′-flanking intron adjacent to exon 33, a 3′-flanking intron adjacent to exon 33, or a combination of the exon 33 and the 5′-flanking intron adjacent to exon 33 or the 3′-flanking intron adjacent to exon 33.

110. The antisense oligonucleotide of claim 102, wherein the ABCA4 pre-mRNA target sequence is in a 5′-flanking intron adjacent to exon 40, a 3′-flanking intron adjacent to exon 40, or a combination of the exon 40 and the 5′-flanking intron adjacent to exon 40 or the 3′-flanking intron adjacent to exon 40.

111. The antisense oligonucleotide of claim 102, wherein the sequence identity is at least 90%.

112. The antisense oligonucleotide of claim 102, wherein the antisense oligonucleotide comprises at least one modified nucleobase.

113. The antisense oligonucleotide of claim 102, wherein the antisense oligonucleotide comprises at least one modified internucleoside linkage.

114. The antisense oligonucleotide of claim 102, wherein the antisense oligonucleotide comprises at least one modified sugar nucleoside.

115. The antisense oligonucleotide of claim 114, wherein the at least one modified sugar nucleoside comprises a 2′-modified sugar nucleoside.

116. The antisense oligonucleotide of claim 102, wherein the antisense oligonucleotide is a morpholino oligomer.

117. The antisense oligonucleotide of claim 102, further comprising a targeting moiety.

118. The antisense oligonucleotide of claim 102, wherein the antisense oligonucleotide comprises at least 12 nucleosides and has a total of 50 nucleosides or fewer.

119. A method of increasing the level of exon-containing ABCA4 mRNA molecules in a cell expressing an aberrant ABCA4 gene, the method comprising contacting the cell with the antisense oligonucleotide of claim 1.

120. A method of decreasing the level of intron-containing ABCA4 mRNA molecules in a cell expressing an aberrant ABCA4 gene, the method comprising contacting the cell with the antisense oligonucleotide of claim 1.

121. A method of treating retinitis pigmentosa, cone-rod dystrophy, or Stargardt disease in a subject having an aberrant ABCA4 gene, the method comprising administering a therapeutically effective amount of the antisense oligonucleotide of claim 1 to the subject.
